(19) United States
(12) Patent Application Publication
(10) Pub. No.: US 2004/0053317 A1
(43) Pub. Date: Mar. 18, 2004

(54) Gene segregation and biological sample classification methods

(75) Inventor: Guennadi V. Glinskii, La Jolla, CA (US)

Correspondence Address: FENWICK & WEST LLP, SILICON VALLEY CENTER, 801 CALIFORNIA STREET, MOUNTAIN VIEW, CA, 94041, US

(73) Assignee: Sidney Kimmel Cancer Center, San Diego, CA

(21) Appl. No.: 10/660,434

(22) Filed: Sep. 10, 2003

Related U.S. Application Data
(60) Provisional application No. 60/410,018, filed on Sep. 10, 2002.
(60) Provisional application No. 60/411,155, filed on Sep. 16, 2002.
(60) Provisional application No. 60/429,168, filed on Nov. 25, 2002.
(60) Provisional application No. 60/444,348, filed on Jan. 31, 2003.
(60) Provisional application No. 60/460,826, filed on Apr. 3, 2003.

(51) Int. Cl. C12Q 1/68

(52) U.S. Cl. 435/6

(57) ABSTRACT

General methods of biological sample classification based on gene expression analysis are described. The methods segregate individual samples into distinct classes using quantitative measurements of expression values for selected sets of genes in individual samples compared to a reference standard. Samples displaying positive and negative correlations of the gene expression values with the reference standard samples exhibit distinct behaviors and pathohistological features. Also disclosed are methods for identifying sets of genes whose expression patterns are correlated with a phenotype. Such sets are useful for characterizing cellular differentiation pathways and states and for identifying potential drug discovery targets.

### CROSS-REFERENCE TO RELATED APPLICATIONS

This application claims the benefit of U.S. Provisional Application 60/410,018 filed Sep. 10, 2002, U.S. Provisional Application 60/411,155 filed Sep. 16, 2002, U.S. Provisional Application 60/429,168 filed Nov. 25, 2002, U.S. Provisional Application 60/444,348 filed Jan. 31, 2003, and U.S. Provisional Application 60/460,826 filed Apr. 3, 2003, each of which is incorporated by reference in its entirety.

### STATEMENT REGARDING FEDERALLY SPONSORED RESEARCH

This invention was made using federal funds awarded by the National Institutes of Health, National Cancer Institute under contract number 1RO1CA89827-01. The government has certain rights to this invention.

### FIELD OF THE INVENTION

The present invention relates to methods for gene segregation to identify clusters of genes associated with biological sample phenotypes and for classifying biological samples on the basis of gene expression patterns derived from those samples.

### BACKGROUND OF THE INVENTION

For many years established human cancer cell lines have been used as models to study human cancers because, to a large degree, they faithfully recapitulate many biological features of human tumors. Established human cancer cell lines maintained in vitro are not expected to fully recapitulate the gene expression patterns of human clinical cancers. This essentially precludes their use as model systems for global gene expression analysis of human tumors. It is likely that the longer that cancer cell lines are maintained in vitro, the more they degrade as models for transcription changes in human clinical cancers.

Recent experiments using established human prostate and breast cancer cell line models indicate that this degradation may be at least partly reversed by using established cancer cell lines to generate experimental tumors in mice and to develop xenograft-derived cell lines from these experimental tumors (Glinsky, G. V., Glinskii, A. B., McClelland, M., Krones-Herzig, A., Mercola, D., Welsh, J. 2002. Microarray gene expression analysis of tumor progression in the nude mouse model of human prostate cancer. In Proceedings of the 93^{rd} Annual Meeting of the American Association for Cancer Research, April 6-10, San Francisco, Calif., 43: 462 (Abstract#4480), incorporated herein by reference). Furthermore, the study of differential gene expression observed using cell lines maintained in vitro and in cell line-induced experimental tumors in mice avoids many of the problems associated with cellular heterogeneity and experimental manipulation of clinical samples. It appears that the in vitro and in vivo human prostate cancer progression models partially recapitulate gene expression behavior of clinical prostate tumor samples, at least with respect to the consensus differentially regulated gene class that has been recently defined for multiple xenograft-derived human prostate cancer cell lines (Glinsky, G. V., Glinskii, A. B., McClelland, M., Krones-Herzig, A., Mercola, D., Welsh, J. 2002. Microarray gene expression analysis of tumor progression in the nude mouse model of human prostate cancer. In Proceedings of the 93^{rd} Annual Meeting of the American Association for Cancer Research, April 6-10, San Francisco, Calif., 43: 462 (Abstract#4480), incorporated herein by reference).

While several useful methods of classification of human and other tumors are known, these methods tend to be a highly subjective in nature and at best semi-quantitative. Recent advances in global gene expression analysis of human tumors using cDNA or oligonucleotide microarray technologies set the stage for the development of improved quantitative methods for human tumor classification (see, e.g., Magee, J. A., Araki, T., Patil, S., Ehrig, T., True, L., Humphrey, P. A., Catalona, W. J., Watson, M. A., Milbrandt, J. Expression profiling reveals hepsin overexpression in prostate cancer. Cancer Res., 61: 5692-5696, 2001; Dhanasekaran, S. M., Barrette, T. R., Ghosh, D., Shah, R., Varambally, S., Kurachi, K., Pienta, K. J., Rubin, M. A., Chinnalyan, A. M. Delineation of prognostic biomarkers in prostate cancer. Nature, 412:822-826, 2001; Welsh, J. B., Sapinoso, L. M., Su, A. I., Kern, S. G., Wang-Rodriguez, J., Moskaluk, C. A., Frierson, H. F., Jr., Hampton, G. M. Analysis of gene expression identifies candidate markers and pharmacological targets in prostate cancer. Cancer Res., 61: 5974-5978, 2001; Luo, J., Duggan, D. J., Chen, Y., Sauvageot, J., Ewing, C. M., Bittner, M. L., Trent, J. M., Isaacs, W. B. Human prostate cancer and benign prostatic hyperplasia: molecular dissection by gene expression profiling. Cancer Res., 61: 4683-4688, 2001; Stamey, T A, Warrington, J A, Caldwell, M C, Chen, Z, Fan, Z, Mahadevappa, M, McNeal, J E, Nolley, R, Zhang, Z. Molecular genetic profiling of Gleason grade 4/5 prostate cancers compared to benign prostatic hyperplasia. J. Urol., 166: 2171-2177, 2001; Luo, J., Dunn, T, Ewing, C, Sauvageot, J., Chen, Y, Trent, J, Isaacs, W. Gene expression signature of benign prostatic hyperplasia revealed by cDNA microarray analysis. Prostate, 51: 189-200, 2002; Singh, D., Febbo, P. G., Ross, K., Jackson, D. G., Manola, C. L., Tamayo, P., Renshaw, A. A., D'Amico, A. V., Richie, J. P., Lander, E. S., Loda, M., Kantoff, P. W., Golub, T. R., Sellers, W. R. Gene expression correlates of clinical prostate cancer behavior. Cancer Cell, 1: 203-209, 2002; Rhodes, D. R., Barrette, T. R., Rubin, M. A., Ghosh, D., Chinnaiyan, A. M. Meta-analysis of microarrays: interstudy validation of gene expression profiles reveals pathways dysregulation in prostate cancer. Cancer Res., 62: 4427-4433, 2002; Pollack, J. R., Perou, C. M., Alizadeh, A. A., Eisen, M. B., Pergamenschikov, A., Williams, C. F., Jeffrey, S. S., Botstein, D., Brown, P. O. Genome-wide analysis of DNA-copy number changes using cDNA microarrays. Nature Genetics. 1999. 23: 41-46; Forozan, F., Mahlamaki, E. H., Monni, O., Chen, Y., Veldman, R., Jiang, Y., Gooden, G. C., Ethier, S. P., Kallioniemi, A., Kallioniemi, O-P. Comparative genomic hybridization analysis of 38 breast cancer cell lines: a basis for interpreting complementary DNA microarray data. Cancer Res. 2000. 60: 4519-4525; Perou C M, Jeffrey S S; van de Rijn M, et al. Distinctive gene expression patterns in human mammary epithelial cells and breast cancers. Proc Natl Acad Sci USA. 1999. 96:9212-9217; Perou C M, Sorlie T, Eisen M B, et al. Molecular portrait of human breast tumors. Nature. 2000. 406:747-752; Clark, E A, Golub T R, Lander E S, Hynes R O. Genomic analysis of metastasis reveals an essential role for RhoC. Nature 2000. 406:532-535; Welsh, J. B., Zarrinkar, P. P., Sapinoso, L. M., Kern, S. G., Behling, C. A., Monk, B. J., Lockhart, D. J., Burger, R. A., Hampton, G. M. Analysis of gene expression profiles in normal and neoplastic ovarian tissue samples identifies candidate molecular markers of epithelial ovarian cancer. Proc Natl Acad Sci USA. 2001. 98:1176-1181, incorporated herein by reference). However, direct attempts to identify genes differentially regulated in tumors that are useful for tumor classification, clinical management and prognosis have produced limited success, in part, because of intrinsic cellular heterogeneity and variability in cellular composition of clinical samples, the statistically underdetermined nature of the problem in which the number of variables (e.g., expression data points) exceeds the number of observations (i.e., independent samples from which the data are gathered), and the absence of a uniform, readily accessible and reproducible reference standard against which differential expression can be evaluated.

In the context of clinical tumor samples, an acceptable reference standard against which differential gene expression can be evaluated should meet the following requirements:

Individual clinical tumors should display different degrees of resemblance between their gene expression patterns as compared to the gene expression pattern exhibited by the reference standard samples;

The degree of resemblance between the gene expression patterns in individual clinical samples and that of the reference standard samples should be susceptible to quantitative measurement; and

Quantitative measurements of the degree of resemblance between clinical samples and the reference standard samples should correlate with biological, clinical, and pathohistological features of individual human tumors enabling their use as a basis for classification of clinical tumor samples.

In a more general sense, gene expression drives the acquisition of cellular phenotypes during differentiation of precursor or stem cells. Identification of genes that are differentially expressed between precursor cells and differentiated cells, or between different types of differentiated cells is an important step for understanding the molecular processes underlying differentiation. The ability to control differentiation of precursor or stem cells so as to direct the cells down a desired differentiation pathway is an important goal, as it represents a tissue engineering solution to the problem of alleviating the shortage of tissue and organs useful for grafting and transplantation. Furthermore, normal and transformed cell-type specific markers, useful for, e.g., molecular-recognition-based targeting of therapeutics such as e.g., rituximab and other recognition based therapeutics, can be identified from sets of genes concordantly regulated in particular normal and transformed cell types.

Attempts to identify directly genes that are differentially regulated in various cell lines suffer from some of the same difficulties referenced above for tumor samples. One of the most common problems for the array-based study is that they usually generate vast data sets. For example, gene expression analysis of a single tumor cell line and a single normal epithelial counterpart typically identifies many thousands of transcripts as differentially expressed at a statistically significant level. Up to 40-50% of the surveyed genes will be identified as differentially expressed when one compares gene expression profiles of normal epithelial and stromal cells. Obviously, any meaningful design of follow-up clinical and/or experimental validation experiments would require an application of further data reduction steps. Our work makes contribution to the solution of this problem by providing a convenient and simple data reduction technique. Two possible approaches seem to be appropriate: one can narrow a set of candidate genes identified in cell lines to those that maintain similar transcript abundance (or other type of gene expression) behavior in a relevant set of clinical tumor samples and design a hypothesis-driven study aimed at identifying potential biologically important genes and/or pathways using cell lines as a model system. Alternatively, one can identify or design cell lines that recapitulate gene expression behavior identified in clinical samples and again use the model system for the assessment of the biological relevance of the gene expression changes. During the last two years or so a third approach is rapidly emerging. It is based on simultaneous analysis of gene expression and DNA copy number changes with an aim to identify the genes that acquired mRNA abundance changes due to the amplification or deletion of the corresponding genes. The cancer cell lines are certainly attractive model systems to undertake such validation study. Suitable reference standards also are needed against which gene expression patterns can be evaluated in normal (i.e., not tumor) cells and/or tissues. Here again, acceptable reference standards would be expected to have the following properties:

Different types of normal cells and/or tissues should display different degrees of resemblance between their gene expression patterns as compared to the gene expression pattern exhibited by the reference standard samples;

The degree of resemblance between the gene expression patterns in individual normal cells and that of the reference standard samples should be susceptible to quantitative measurement; and

Quantitative measurements of the degree of resemblance between normal cells and the reference standard samples should correlate with biological features of different normal cell types so as to provide a basis for the classification of differentiation state and cell type.

There thus exist in the art a need for improved methods of biological sample classification, for improved methods of identifying genes that are differentially expressed or regulated in biological samples such as tumors and normal cells, for reference standards that can be used in accordance with these methods, and for identified sets of coordinately regulated genes, the expression patterns of which can be used for classifying samples and for developing cell- or tissue-specific markers. The present invention addresses these and other shortcomings of the art.

### BRIEF SUMMARY OF THE INVENTION

Broadly, it is an object of the invention to provide improved quantitative methods for classifying tumor and normal samples.

It is a further object of the invention to provide useful reference standards for classifying tumors and normal samples.

It is a still further object of the invention to provide methods for classifying tumor and normal samples on the basis of gene expression data.

Thus, in one aspect, the invention provides a method for classifying a sample in which a first reference set of expressed genes is identified, the first reference set consisting of genes that are differentially expressed between a first set of tumor cell lines and a set of control cell lines, a second reference set of expressed genes is identified, the second reference set consisting of genes that are differentially expressed between a first set of samples and a second set of samples, wherein the first and second samples differ with respect to a sample classification, a concordance set of expressed genes is identified, the concordance set consisting of genes that are common to the first and second reference sets and wherein, preferably, the direction of the differential expression is the same in the first and second reference sets, identifying a minimum segregation set of expressed genes within the concordance set, the minimum segregation set consisting of a subset of expressed genes within the concordance set selected so that a first correlation coefficient between an average fold-change or difference of the gene expression data from the lines and an average fold-change or difference of the gene expression data from the samples exceeds a pre-determined value, calculating for the expressed genes within the minimum segregation set a second correlation coefficient between the average fold-change or difference of the gene expression data from the cell lines and a fold-change or difference of the gene expression data from an unclassified sample, and classifying the unclassified sample as belonging to the first set of samples or the second set of samples according to the sign of the second correlation coefficient.

In a preferred embodiment, the first set of samples and the second set of samples comprise tumor cells and/or tissues containing tumor cells, that differ with respect to a tumor classification such as, e.g., benign versus malignant growth, local and/or systemic recurrence, invasiveness, metastatic propensity, metastatic tumors versus localized primary tumors, degree of dedifferentiation (poor, moderate, or well differentiated tumors), tumor grade, Gleason score, survival prognosis, disease free survival, lymph node status, patient age, hormone receptor status, PSA level, and histologic type.

In another embodiment, reference sets are obtained without the use of cell lines, but instead rely solely on the use of clinical samples. In this embodiment, a first reference set is obtained by looking at differential expression among two or more sets of clinical samples, preferably using average expression values, wherein the two or more sets differ with respect to a known phenotype. A concordance set is then obtained by determining concordance between the differentially expressed genes established using the two or more clinical sample groups and one or more individual samples within the group that demonstrate the best fit (highest correlation coefficient) between the individual sample(s) and the average group measurements.

In other preferred embodiments, the gene expression data is selected from the group consisting of mRNA quantification data, cDNA quantification data, cRNA quantification data, and protein quantification data.

In another aspect, the invention provides for a method for identifying a set of genes in which a first reference set of expressed genes is identified, the first reference set consisting of genes that are differentially expressed between a first set of tumor cell lines and a set of control cell lines, a second reference set of expressed genes is identified, the second reference set consisting of genes that are differentially expressed between a first set of samples and a second set of samples, wherein the first and second samples differ with respect to a sample classification, a concordance set of expressed genes is identified, the concordance set consisting of genes that are common to the first and second reference sets and wherein, preferably, the direction of the differential expression is the same in the first and second reference sets, and identifying a minimum segregation set of expressed genes within the concordance set, the minimum segregation set consisting of a subset of expressed genes within the concordance set selected so that a first correlation coefficient between an average fold-change or difference of the gene expression data from the lines and an average fold-change or difference of the gene expression data from the samples exceeds a pre-determined value.

In another embodiment, the minimum segregation set is determined without use of cell line data. This embodiment is preferred when no appropriate cell lines are available. In this embodiment, two or more groups of clinical samples, differing with respect to a known phenotype are used to generate a first reference set. Preferably, this is accomplished by determining average fold expression changes (optionally log transformed), and identifying a set of differentially expressed genes that are consistently (i.e., up- or down-regulated) in one group as compared to another group. The second reference set is obtained by determining for individual sample(s) within a group, fold-expression changes for genes within the first reference set, and finding those genes concordantly over- or under-expressed, in the individual sample(s) cf. the first reference set, and identifying those individual samples for which the individual gene expression values are most highly correlated with the expression of the genes in the first reference set. This essentially consists of calculating phenotype association indices for the individual gene expression measurements within the sample, and selecting as the second reference those genes identified as being concordantly expressed in the most highly correlated individual sample(s).

In yet another preferred embodiment, the invention provides minimum segregation sets of expressed genes. Such sets have utility as tools for, e.g., sample classification or prognostication, and as sources of cell- or tissue-specific markers. The markers can be used as, e.g., targets for delivery of cell- or tissue-specific reagents or drugs, or to monitor drug effects on a molecular scale.

In yet another preferred embodiment, the invention provides a kit comprising a set of reagents useful for determining the expression of a subset of genes identified using the methods of the invention, along with instructions for their use. The reagents can be affixed to a solid support and used in a hybridization reaction, or alternatively can be primers for use in nucleic acid amplification reactions.

Additional advantages and aspects of the present invention are now described with reference to the detailed description and drawings, below.

### BRIEF DESCRIPTION OF THE DRAWINGS

FIG. 1 is a scatter plot showing correlation of the expression profiles in 5 xenograft-derived human prostate carcinoma cell lines and 8 recurrent versus 13 non-recurrent human prostate tumors for 19 genes of the concordance set.

FIG. 2 is a scatter plot showing correlation of the expression profiles in 5 xenograft-derived human prostate carcinoma cell lines and 8 recurrent versus 13 non-recurrent human prostate tumors for 9 genes of the PC3/LNCap recurrence minimum segregation set (recurrence cluster).

FIG. 3 is a graph showing phenotype association indices for 9 genes of the recurrence cluster in individual human prostate tumors exhibiting recurrent (samples 1-8) or non-recurrent (samples 12-24) clinical behavior.

FIG. 4 is a graph showing phenotype association indices for 54 genes of the prostate cancer/normal tissue discrimination minimum segregation set (i.e., cluster).in 24 individual prostate tumors (samples 1-25 [one tumor sample run in duplicate]), 2 normal prostate stroma (NPS) samples (samples 28 and 29), and 9 adjacent normal tissue samples (samples 32-40).

FIG. 5 is a scatter plot showing correlation of the expression profiles in 5 xenograft-derived human prostate carcinoma cell lines and 24 prostate cancer tissue samples versus 9 adjacent normal prostate samples for 54 genes of the concordance set.

FIG. 6 is a graph showing phenotype association indices for 10 genes of the prostate cancer/normal tissue minimum segregation set (i.e. cluster) in 24 prostate tumors (samples 1-25 [one tumor sample run in duplicate]), and 9 adjacent normal tissue samples (samples 29-37).

FIG. 7 is a graph showing phenotype association indices for 5 genes of the prostate cancer/normal tissue minimum segregation set (i.e., cluster) in 24 prostate tumors (samples 1-25 [one tumor sample run in duplicate]), and 9 adjacent normal tissue samples (samples 29-37).

FIG. 8 is a graph showing phenotype association indices for 10 genes of the prostate cancer/normal tissue minimum segregation set (i.e., cluster) in 47 prostate tumors (samples 1-47), and 47 adjacent normal tissue samples (samples 51-97).

FIG. 9 is a graph showing phenotype association indices for 5 genes of the prostate cancer/normal tissue minimum segregation set (i.e., cluster) in 47 prostate tumors (samples 1-47), and 47 adjacent normal tissue samples (samples 51-97).

FIG. 10 is a scatter plot showing correlation of the expression profiles in 5 xenograft-derived human prostate carcinoma cell lines and 14 invasive versus 38 non-invasive human prostate cancer tissue samples for 104 genes of the concordance set.

FIG. 11 is a scatter plot showing correlation of the expression profiles in 5 xenograft-derived human prostate carcinoma cell lines and 14 invasive versus 38 non-invasive human prostate cancer tissue samples for 20 genes of the invasion minimum segregation set 1 (i.e., invasion cluster 1).

FIG. 12 is a graph showing phenotype association indices for 20 genes of invasion cluster 1 in 14 invasive (samples 1-14) and 38 non-invasive (samples 20-57) human prostate tumor samples.

FIG. 13 is a scatter plot showing correlation of the expression profiles in 5 xenograft-derived human prostate carcinoma cell lines and 12 invasive versus 17 non-invasive (surgical margins 1+) human prostate cancer tissue samples for 12 genes of the invasion minimum segregation set 2 (i.e., invasion cluster 2).

FIG. 14 is a graph showing phenotype association indices for 12 genes of invasion cluster 2 in 12 invasive (samples 1-12) and 17 non-invasive (samples 17-33) human prostate tumor samples.

FIG. 15 is a scatter plot showing correlation of the expression profiles in 5 xenograft-derived human prostate carcinoma cell lines and 11 invasive versus 7 non-invasive (invasion clusters 1&2+) human prostate cancer tissue samples for 10 genes of the invasion minimum segregation class 3 (i.e., invasion cluster 3).

FIG. 16 is a graph showing phenotype association indices for 10 genes of invasion cluster 3 in 11 invasive (samples 1-11) and 7 non-invasive (samples 16-22) human prostate tumor samples.

FIG. 17 is a scatter plot showing correlation of the expression profiles in 5 xenograft-derived human prostate carcinoma cell lines and 3 invasive versus 21 non-invasive human prostate cancer tissue samples for 13 genes of the invasion minimum segregation class 4 (i.e., invasion cluster 4).

FIG. 18 is a graph showing phenotype association indices for 13 genes of invasion cluster 4 in 3 invasive (samples 1-3) and 21 non-invasive (samples 8-28) human prostate tumor samples.

FIG. 19 is a scatter plot showing correlation of the expression profiles in 5 xenograft-derived human prostate carcinoma cell lines and 6 high Gleason grade versus 46 low Gleason grade human prostate cancer tissue samples for 58 genes of the concordance set.

FIG. 20 is a scatter plot showing correlation of the expression profiles in 5 xenograft-derived human prostate carcinoma cell lines and 6 high Gleason grade versus 46 low Gleason grade human prostate cancer tissue samples for 17 genes of the high grade minimum segregation set 1 (high grade cluster 1).

FIG. 21 is a scatter plot showing correlation of the expression profiles in 5 xenograft-derived human prostate carcinoma cell lines and 6 high Gleason grade versus 20 low Gleason grade human prostate cancer tissue samples for 12 genes of the high grade minimum segregation set 2 (high grade cluster 2).

FIG. 22 is a scatter plot showing correlation of the expression profiles in 5 xenograft-derived human prostate carcinoma cell lines and 6 high Gleason grade versus 16 low Gleason grade human prostate cancer tissue samples for 7 genes of the high grade minimum segregation set 3 (high grade cluster 3).

FIG. 23 is a scatter plot showing correlation of the expression profiles in 5 xenograft-derived human prostate carcinoma cell lines and 6 high Gleason grade versus 46 low Gleason grade human prostate cancer tissue samples for 38 genes of the ALT high grade minimum segregation set (ALT high grade cluster).

FIG. 24 is a scatter plot showing correlation of the expression profiles in 5 xenograft-derived human prostate carcinoma cell lines and 6 high Gleason grade versus 17 low Gleason grade human prostate cancer tissue samples for 5 genes of the high grade minimum segregation set 4 (high grade cluster 4).

FIG. 25 is a scatter plot showing correlation of the expression profiles in 5 xenograft-derived human prostate carcinoma cell lines and 6 high Gleason grade versus 17 low Gleason grade human prostate cancer tissue samples for 4 genes of the high grade minimum segregation set 5 (high grade cluster 5).

FIG. 26 is a scatter plot showing correlation of the expression profiles in 5 xenograft-derived human prostate carcinoma cell lines and 6 high Gleason grade versus 17 low Gleason grade human prostate cancer tissue samples for 7 genes of the high grade minimum segregation set 6 (high grade cluster 6).

FIG. 27 is a scatter plot showing correlation of the expression profiles in 5 xenograft-derived human prostate carcinoma cell lines and 6 high Gleason grade versus 17 low Gleason grade human prostate cancer tissue samples for 13 genes of the high grade minimum segregation set 7 (high grade cluster 7).

FIG. 28 is a graph showing phenotype association indices for 54 genes of the BPH minimum segregation class (i.e. cluster) in 8 patients with benign prostatic hypertrophy (BPH) (samples 1-8) and 9 patients with prostate cancer (samples 13-21).

FIG. 29 is a graph showing phenotype association indices for 14 genes of the BPH minimum segregation class (i.e. cluster) MAGEA1 in 8 patients with benign prostatic hypertrophy (BPH) (samples 1-8) and 9 patients with prostate cancer (samples 12-20).

FIG. 30 is a graph showing phenotype association indices for 17 genes of the metastasis minimum segregation class 1 (i.e. metastasis cluster 1) in 5 patients with benign prostatic hypertrophy (BPH) (samples 7-11), 3 adjacent normal prostate (ANP) samples (samples 1-3), 1 patient with prostatitis (sample 5), 10 patients with localized prostate cancer (samples 13-22), and 7 patients with metastatic prostate cancer (MPC) (samples 24-30).

FIG. 31 is a graph showing phenotype association indices for 19 genes of the metastasis minimum segregation class 2 (i.e. metastasis cluster 2) in 5 patients with benign prostatic hypertrophy (BPH) (samples 7-11), 3 adjacent normal prostate (ANP) samples (samples 1-3), 1 patient with prostatitis (sample 5), 10 patients with localized prostate cancer (samples 13-22), and 7 patients with metastatic prostate cancer (MPC) (samples 24-30).

FIG. 32 is a graph showing phenotype association indices for 17 genes of the metastasis minimum segregation class 1 (i.e. metastasis cluster 1) in 14 patients with benign prostatic hypertrophy (BPH) (samples 1-14), 4 adjacent normal prostate (ANP) samples (samples 17-20), 1 patient with prostatitis (sample 23), 10 patients with localized prostate cancer (LPC) (samples 26-39), and 20 patients with metastatic prostate cancer (MPC) (samples 42-61).

FIG. 33 is a graph showing phenotype association indices for 19 genes of the metastasis minimum segregation class 2 (i.e. metastasis cluster 2) in 14 patients with benign prostatic hypertrophy (BPH) (samples 1-14), 4 adjacent normal prostate (ANP) samples (samples 17-20), 1 patient with prostatitis (sample 23), 14 patients with localized prostate cancer (LPC) (samples 26-39), and 20 patients with metastatic prostate cancer (MPC) (samples 42-61).

FIG. 34 is a graph showing phenotype association indices for 6 genes of the Q-PCR-based poor prognosis predictor minimum segregation set (i.e. cluster) in 34 patients with breast cancer who developed distant metastases within 5 years of diagnosis (samples 1-34) and in 44 patients who continued to be disease-free for at least five years (samples 37-80).

FIG. 35 is a graph showing phenotype association indices for 14 genes of the Q-PCR-based good prognosis predictor minimum segregation set (i.e. cluster) in 34 patients with breast cancer who developed distant metastases within 5 years of diagnosis (samples 1-34) and in 44 patients who continued to be disease-free for at least five years (samples 37-80).

FIG. 36 is a graph showing phenotype association indices for 13 genes of the Q-PCR-based good prognosis predictor minimum segregation set (i.e. cluster) in 34 patients with breast cancer who developed distant metastases within 5 years of diagnosis (samples 1-34) and in 44 patients who continued to be disease-free for at least five years (samples 37-80).

FIG. 37 is a graph showing phenotype association indices for 13 genes of the Q-PCR-based good prognosis predictor minimum segregation set (i.e. cluster) in 11 patients with breast cancer who developed distant metastases within 5 years of diagnosis (samples 1-11) and in 8 patients who continued to be disease-free for at least five years (samples 14-21).

FIG. 38 is a graph showing phenotype association indices for 11 genes of the ovarian cancer poor prognosis predictor minimum segregation set (i.e. cluster) in 3 poorly differentiated tumors (samples 1-3) and in 11 tumors of well and moderate differentiation (samples 6-16).

FIG. 39 is a graph showing phenotype association indices for 10 genes of the ovarian cancer good prognosis predictor minimum segregation set (i.e. cluster) in 3 poorly differentiated tumors (samples 1-3) and in 11 tumors of well and moderate differentiation (samples 6-16).

FIG. 40 is a scatter plot showing correlation of the expression profiles in non small cell lung carcinoma (“NSCLC”) cell lines and normal bronchial epithelial cells versus 139 human adenocarcinoma tissue samples versus 17 normal human lung samples for 13 genes of the human lung adenocarcinoma minimum segregation set 1 (lung adenocarcinoma cluster 1).

FIG. 41 is a scatter plot showing correlation of the expression profiles in non small cell lung carcinoma (“NSCLC”) cell lines and normal bronchial epithelial cells and 139 human adenocarcinoma tissue samples versus 17 normal human lung samples for 26 genes of the human lung adenocarcinoma minimum segregation set 2 (lung adenocarcinoma cluster 2).

FIG. 42 is a graph showing phenotype association indices for 13 genes of the lung adenocarcinoma minimum segregation set 1 (lung adenocarcinoma cluster 1) in 17 normal lung specimens (samples 1-17) and 139 patients with lung adenocarcinoma (samples 20-158).

FIG. 43 is a graph showing phenotype association indices for 26 genes of the lung adenocarcinoma minimum segregation set 2 (lung adenocarcinoma cluster 2) in 17 normal lung specimens (samples 1-17) and 139 patients with lung adenocarcinoma (samples 20-158).

FIG. 44 is a scatter plot showing correlation of the expression profiles in non small cell lung carcinoma (“NSCLC”) cell lines and normal bronchial epithelial cells and 34 human NSCLC patients with poor prognosis tissue samples versus 16 human NSCLC patients with good prognosis tissue samples for 38 genes of the lung adenocarcinoma poor prognosis minimum segregation set 1 (poor prognosis cluster 1).

FIG. 45 is a graph showing phenotype association indices for 38 genes of the lung adenocarcinoma poor prognosis minimum segregation set 1 (poor prognosis cluster 1) in 34 human NSCLC patients with poor prognosis (samples 1-34) 16 human NSCLC patients with good prognosis (samples 37-52).

FIG. 46. Xenografts of human prostate cancer derived from the PC-3M-LN4 highly metastatic cell variant and growing in a metastasis promoting orthotopic setting exhibit pro-invasive and pro-angiogenic gene expression profiles. Expression profiling of the 12,625 transcripts in the orthotopic (“OR”) and subcutaneous (“s.c.” or “SC”) xenografts derived from the cell variants of the PC-3 lineage was carried out. (A1-A4) Expression pattern of the matrix metalloproteinases (MMPs). (B1-B4) Expression pattern of the components of plasminogen/plasminogen activator system. (C1-C4) Pro-angiogenic switch in PC-3M-LN4 orthotopic xenografts: increased levels of expression of interleukin 8, angiopoietin-2, and osteopontin and decreased level of expression of a protease and angiogenesis inhibitor maspin. (D1-D4) Cadherin switch in PC-3M-LN4 orthotopic xenografts: increased level of expression of non-epithelial cadherins (OB-cadherin-2 and VE-cadherin) and decreased level of expression of epithelial E-cadherin.

FIG. 47. Correlation of gene expression profiles 8-gene prostate cancer recurrence signature cluster (A) in highly metastatic orthotopic xenografts and the recurrent versus non-recurrent prostate tumors or 5-gene prostate cancer invasion signature in invasive versus non-invasive human prostate tumors (B).

FIG. 48. Correlation of expression profiles in orthotopic xenografts and clinical samples for 131-gene prostate cancer metastasis signature cluster (A), 37-gene prostate cancer metastasis signature (B), 12-gene prostate cancer metastasis signature (C), 9-gene prostate cancer metastasis signature (D).

FIG. 49. Gene expression patterns of selected gene clusters in highly metastatic orthotopic xenografts are discriminators of the metastatic and primary human prostate carcinomas. The classification accuracy of the clinical samples is shown for clusters of 131 genes (A), 37 genes (B), 9 genes (C), and a family of 6 metastasis segregation clusters (D).

FIG. 50 Gene expression patterns of the selected gene clusters in highly metastatic orthotopic xenografts are discriminators of invasive (FIG. 50A) and recurrent (FIG. 50B) phenotypes of human prostate tumors. FIG. 50A, phenotype association indices for 5 gene prostate cancer invasion predictor. Bars 1-8 tumors with positive surgical margins and prostate capsule penetration (“PSM & PCP”); bars 11-16 tumors with positive surgical margins (“PSM”); bars 19-30 tumors with prostate capsule penetration (“PCP”); bars 33-58 non-invasive tumors. FIG. 50B, phenotype association indices for 8 gene prostate cancer recurrence predictor. Bars 1-8 recurrent tumors; bars 11-23 non-recurrent tumors.

FIG. 51. Gene expression profiles of selected gene clusters in highly metastatic PC3MLN4 orthotopic xenografts are concordant with the expression patterns of these genes in the recurrent (A), invasive (B), and metastatic (C) human prostate tumors. For each figure, bars show average fold change in gene expression compared to respective control for individual genes within clusters.

FIG. 52. Gene expression profiles of the 25-gene recurrence predictor signature in highly metastatic PC3MLN4 orthotopic xenografts are concordant with the expression patterns of these genes in the recurrent human prostate tumors. FIG. 52A—correlation of expression profiles in orthotopic xenografts and clinical samples for 25-gene prostate cancer recurrence predictor cluster. FIG. 52B—Change in expression for each transcript are plotted as Log10Fold Change Average expression level in PC-3MLN4OR versus Average expression level in PC-3MLN4SC and Log10 Fold Change Average expression level in recurrent prostate tumors versus Average expression level in non-recurrent prostate tumors.

FIG. 53 is a bar graph illustrating phenotypic association indices for transcripts of the 25 genes prostate cancer recurrence predictor cluster in 8 recurrent and 13 non-recurrent human prostate tumors.

FIG. 54 is a bar graph illustrating expression profile of the 12 gene recurrence predictor signature in PC-3MLN4 orthotopic xenografts and recurrent human prostate tumors.

FIG. 55 is a scatter plot illustrating correlation of the expression profiles of the 12 genes recurrence predictor cluster in PC-3MLN4 orthotopic xenografts and recurrent human prostate tumors.

FIG. 56 is a bar graph illustrating phenotypic association indices for transcripts of the 12 genes prostate cancer recurrence predictor cluster in 8 recurrent and 13 non-recurrent human prostate tumors.

FIG. 57. Phenotype association indices (PAIs) defined by the expression profile of the prostate cancer recurrence predictor signature 1 for 21 prostate carcinoma samples comprising a signature discovery (training) data set.

FIG. 58. Kaplan-Meier analysis of the probability that patients would remain disease-free among 21 prostate cancer patients comprising a signature discovery group according to whether they had a good-prognosis or poor-prognosis signatures defined by the recurrence predictor signature 1 (FIG. 58A), recurrence predictor signature 2 (FIG. 58B), recurrence predictor signature 3 (FIG. 58C), and the recurrence predictor algorithm that takes into account calls from all three signatures (FIG. 58D).

FIG. 59. Kaplan-Meier analysis of the probability that patients would remain disease-free among 79 prostate cancer patients comprising a signature validation group for all patients (FIG. 59A), patients with high (FIG. 59B) or low (FIG. 59C) preoperative PSA level in blood according to whether they had a good-prognosis or poor-prognosis signatures defined by the recurrence predictor algorithm or whether they had high or low preoperative PSA level in the blood (FIG. 59D).

FIG. 60. Kaplan-Meier analysis of the probability that patients would remain disease-free among prostate cancer patients with Gleason sum 6 & 7 tumors (FIG. 60A) and patients with Gleason sum 8 & 9 tumors (FIG. 60B) according to whether they had a good-prognosis or poor-prognosis signatures defined by the recurrence predictor algorithm or whether they had Gleason sum 8 & 9 or Gleason sum 6 & 7 prostate tumors (FIG. 60C).

FIG. 61. Kaplan-Meier analysis of the probability that patients would remain disease-free among 79 prostate cancer patients comprising a signature validation group for all patients (FIG. 61A), patients with poor prognosis (FIG. 61B) or good prognosis (FIG. 60C) defined by the Kattan nomogram according to whether they had a good-prognosis or poor-prognosis signatures defined by the recurrence predictor algorithm (FIGS. 61B and 61C) or whether they had poor or good prognosis defined by the Kattan nomogram (FIG. 61A).

FIG. 62. Kaplan-Meier analysis of the probability that patients would remain disease-free among prostate cancer patients with stage 1C tumors (FIG. 62A) and patients with stage 2A tumors (FIG. 62B) according to whether they had a good-prognosis or poor-prognosis signatures defined by the recurrence predictor algorithm.

FIG. 63. Kaplan Meier survival curves. FIG. 63A Survival of 151 breast cancer patients with lymph node negative disease (stratified by 14 gene signature). FIG. 63B Survival of 109 breast cancer patients with estrogen receptor positive tumors and lymph node negative disease (stratified by 14 gene signature); FIG. 63C Survival of 42 breast cancer patients with estrogen receptor negative tumors and lymph node negative disease (stratified by 4 and/or 3 gene signatures).

FIG. 64. Kaplan Meier survival curves. FIG. 64A Survival of breast cancer patients with estrogen receptor positive and estrogen receptor negative tumors; FIG. 64B Survival or 69 breast cancer patients with estrogen receptor negative tumors (stratified by 5 and/or three gene signatures).

FIG. 65. Metastasis-free survival of 78 breast cancer patients. FIG. 65A survival stratified by 4 gene signature; FIG. 65B survival stratified by 6 gene signature; FIG. 65C, survival stratified by 13 gene signature; FIG. 65D survival stratified by 14 gene signature.

FIG. 66. Survival of breast cancer patients classified into subgroups using gene signatures. FIG. 66A Survival of 144 breast cancer patients with lymph node positive disease stratified according to 14 gene survival predictor cluster; FIG. 66B Survival of 117 breast cancer patients with estrogen receptor positive tumors and lymph node positive disease stratified according to 14 gene survival predictor cluster; FIG. 66C Survival of 27 breast cancer patients with estrogen receptor negative tumors and lymph node positive disease stratified according to 4 and 3 gene signatures.

FIG. 67. Survival of estrogen receptor positive breast cancer patients. FIG. 67A stratified according to positive and negative 14 gene signature; FIG. 67B stratified according to relative values of 14 gene signature.

FIG. 68. Survival of breast cancer patients. FIG. 68A Survival of 295 breast cancer patients with positive and negative 14 gene signature (0.00 cut off); FIG. 68B Survival of 295 breast cancer patients with positive and negative 14 gene signature (−0.55 cut off); FIG. 68C Survival of breast cancer patients with positive and negative 14-gene signature; FIG. 68D Survival of breast cancer patients with positive and negative 14 gene signature; FIG. 68E Survival of breast cancer patients classified based on relative values of the 14 gene signature.

### DETAILED DESCRIPTION OF THE PREFERRED EMBODIMENTS

Definitions

All terms, unless specifically defined below, are intended to have their ordinary meanings as understood by those of skill in the art. Claimed masses and volumes are intended to encompass variations in the sated quantities compatible with the practice of the invention. Such variations are contemplated to be within, e.g. about +10-20 percent of the stated quantities. In case of conflict between the specific definitions contained in this section and the ordinary meanings as understood by those of skill in the art, the definitions supplied below are to control.

“Identifying a set of expressed genes” refers to any method now known or later developed to assess gene expression, including but not limited to measurements relating to the biological processes of nucleic acid amplification, transcription, RNA splicing, and translation. Thus, direct and indirect measures of gene copy number (e.g., as by fluorescence in situ hybridization or other type of quantitative hybridization measurement, or by quantitative PCR), transcript concentration (e.g., as by Northern blotting, expression array measurements or quantitative RT-PCR), and protein concentration (e.g., by quantitative 2-D gel electrophoresis, mass spectrometry, Western blotting, ELISA, or other method for determining protein concentration) are intended to be encompassed within the scope of the definition.

“Differentially expressed” refers to the existence of a difference in the expression level of a gene as compared between two sample classes. Differences in the expression levels of “differentially expressed” genes preferably are statistically significant.

“Tumor” is to be construed broadly to refer to any and all types of solid and diffuse malignant neoplasias including but not limited to sarcomas, carcinomas, leukaemias, lymphomas, etc., and includes by way of example, but not limitation, tumors found within prostate, breast, colon, lung, and ovarian tissues.

A “tumor cell line” refers to a transformed cell line derived from a tumor sample. Usually, a “tumor cell line” is capable of generating a tumor upon explant into an appropriate host. A “tumor cell line” line usually retains, in vitro, properties in common with the tumor from which it is derived, including, e.g., loss of differentiation, loss of contact inhibition, and will undergo essentially unlimited cell divisions in vitro.

A “control cell line” refers to a non-transformed, usually primary culture of a normally differentiated cell type. In the practice of the invention, it is preferable to use a “control cell line” and a “tumor cell line” that are related with respect to the tissue of origin, to improve the likelihood that observed gene expression differences are related to gene expression changes underlying the transformation from control cell to tumor.

An “unclassified sample” refers to a sample for which classification is obtained by applying the methods of the present invention. An “unclassified sample” may be one that has been classified previously using the methods of the present invention, or through the use of other molecular biological or pathohistological analyses. Alternatively, an “unclassified sample” may be one on which no classification has been carried out prior to the use of the sample for classification by the methods of the present invention.

“According to the sign of” a correlation coefficient refers to a determination based on the sign, i.e., positive or negative, of the referenced correlation coefficient. For example, a sample may be classified as belonging to a first set of samples if the sign of the correlation coefficient is positive, or as belonging to a second set of samples if the correlation coefficient is negative.

“Orthotopic” refers to the placement of cells in an organ or tissue of origin, and is intended to encompass placement within the same species or in a different species from which the cells are originally derived.

“Ectopic” refers to the placement of cells in an organ or tissue other than the organ or tissue of origin, and is intended to encompass placement within the same species or in a different species from which the cells are originally derived.

Introduction

Completion of the draft sequence of the human genome offers an unprecedented opportunity to study the genetic basis of human cancer progression. During malignant progression, genomic instability leads to continuously emerging phenotypic diversity, clonal evolution, and clonal selection resulting in the remarkable cellular heterogeneity of tumors. The phenotypic diversity of cancer cells is associated with significant mutation-driven changes in gene expression, although not all mutations and differences in gene expression are crucial or even relevant to the malignant phenotype. It therefore is important to identify expression changes that are highly relevant and characteristic of malignant phenotypes and progression pathways, more than one of which may exist (Hanahan, D., Weinberg, R. A. The hallmarks of cancer. Cell. 2000. 100: 57-70, incorporated herein by reference.). The methods of the present invention address this goal by providing analytical techniques to identify those expression changes highly correlated with and indeed predictive of certain clinically relevant features of malignant phenotypes and progression pathways.

In a broad and general sense, as applied to the analysis of tumor samples, the methods of the invention use gene expression data from a set of tumor cell lines and compare those data with gene expression data from a set of control cell lines to identify those genes that are differentially expressed in the tumor cell lines as compared to the control cell lines. In preferred embodiments, each of these sets includes more than a single member, although it is contemplated to be within the scope of the present invention to practice embodiments in which either or both of the set of tumor cell lines and the set of control cell lines includes only one member. The identified genes are referred to as a first reference set of expressed genes. Preferably, the control cell line and the tumor cell lines are related insofar as the control cell lines represent physiologically normal cells from the tissue or organ from which the tumor represented by the tumor cell lines arose. For example, if the tumor cell lines are derived from a prostate tumor, the control cell lines preferably are primary cultures of normal prostate epithelial cells. In the preferred embodiments, more than one tumor cell line and more than one control cell line is used to generate the reference set so as to reduce the number of genes in the first reference set by eliminating those genes that are not consistently differentially expressed between the tumor and control cell lines.

In other embodiments, the method may be practiced using only one tumor cell line and one control cell line, and identifying the set of genes differentially expressed between the tumor cell line and the control cell line. However, by carrying out a series of comparison between multiple control cell lines and multiple tumor cell lines the first reference set is more likely to contain only those genes that are consistently differentially expressed between the normal and tumor classes of cell lines (i.e., a gene is included within the first reference set if its expression level is always higher in each of the tumor cell lines examined as compared to each of the control cell lines examined, or if its expression level is always lower in each of the tumor cell lines examined as compared to each of the control cell lines examined).

In yet another embodiment, exemplified below as Example 6, the methods of the invention may be practiced without the use of cell lines, using instead data derived only from clinical samples. In a similar manner, the methods of the invention may be practiced using only data derived from cell lines.

For example, consider an embodiment in which the first reference set is derived using data obtained from three separate control cell lines and six separate tumor cell lines. For each gene considered for inclusion within the first reference set, pairwise comparisons are carried out for each of the 3×6 or 18 pairwise combinations between control cell lines and tumor cell lines. A candidate gene will be included in the first reference set if each of the 18 pairwise comparisons reveals the gene to be consistently differentially expressed (i.e., gene expression always is higher in the control cell line or always higher in the tumor cell line for each of the 18 pairwise comparisons). As one of ordinary skill readily will appreciate, it may sometimes be necessary to scale the datasets prior to carrying out the pairwise comparisons. Such scaling may be routinely implemented in the analysis software provided by commercial suppliers of expression arrays or array readers (such as, e.g., Affymetrix, Santa Clara, Calif.). For a general discussion of data scaling for and differential gene expression analysis, see, e.g., Affymetrix Microarray Suite 4.0 User Guide, Affymetrix, Santa Clara, Calif., incorporated herein by reference.

The first reference set therefore is a set of genes that have met a screening criterion requiring that the genes be differentially expressed between tumor and control cell lines. This criterion reflects the hypothesis that differences in the tumor and control cell phenotypes are driven, at least in part, by differences in gene expression patterns in the tumor and control cells. In the practice of the invention, generating a first reference set typically results in an order of magnitude or greater reduction in the number of genes that remain under consideration for inclusion in a cluster or for use in the sample classification methods.

Because the tumor and control cell lines have at some point been cultured in vitro, their gene expression patterns likely will not exactly correspond with the expression patterns of their counterparts grown in vivo. Consequently, the methods of the invention use additional steps to establish a second reference set of expressed genes that are differentially expressed in cells of biological samples that differ with respect to a classification. The classification may be an outcome predictor or cellular phenotype or any type of classification that may be used for classifying biological samples. The classification may be binary (i.e., for two mutually exclusive classes such as, e.g., invasive/non-invasive, metastatic/non-metastatic, etc.), or may be continuously or discretely variable (i.e., a classification that can assume more than two values such as, e.g., Gleason scores, survival odds, etc.) The only requirement is that the classified trait must be something that can be observed and characterized by the assignment of a variable or other type of identifier so that samples belonging to the same class may be grouped together during the analysis.

The second reference set of expressed genes may be obtained following essentially the same techniques described above for the first reference set, except sets of samples obtained from in vivo sources are used instead of sets of cell lines. In embodiments of the invention directed toward tumor analysis, classification or prognostication, the sample sets preferably consist of tumor samples obtained from patients that are analyzed without any intervening tissue culturing steps so that the gene expression patterns reflect as closely as possible the pattern within cells growing in their undisturbed, in vivo environment. Here again, the goal is to obtain a reference set that includes genes differentially expressed between samples belonging to different classifications. As is the case with the first reference set, it is preferable to include several independent samples within a classified set and to carry out a plurality of pairwise comparisons to identify differentially expressed genes for inclusion into the second reference set.

For example, assume the classification of interest is invasiveness (e.g., turning on whether tumor-free surgical margins are observed). It is preferable to use as the sample sets a number of invasive samples and a number of non-invasive samples. The number of pairwise comparisons that can be carried out is of course equal to the product of the numbers of independent samples in each category. Ideally, each of these pairwise comparisons is carried out and the same consistently differentially expressed criterion described above is used to select genes for inclusion into the second reference set.

It is contemplated, that in certain instances, especially, e.g., when the variance within a sample set is low, it will not be necessary to carry out all pairwise comparisons to select genes for inclusion into the first or second reference set. In practice, one of ordinary skill can readily determine whether it is advantageous to carry out all pairwise comparisons, or fewer than all pairwise comparisons by examining the convergence behavior of the reference sets as additional comparisons are carried out. If the sets apparently converge prior to completion of all possible pairwise comparisons, then the added benefit of exhaustive comparison may be small and so can be avoided.

Similar principles drive the selection of the numbers of cell lines and cell samples used to derive the first and second reference sets as apply to the study of other cell and molecular biological phenomena. One of ordinary skill readily will appreciate that the accuracy of the reference sets can increase as more cell lines and samples are used so that statistical noise is minimized. It currently is contemplated that preferred numbers of different cell lines and samples per set used for calculating reference sets be in the range of 2 to 50 per set, or in the range of 2 to 25, or in the range of 2 to 10, or in the range of 3 to 5 per set. While not preferred, it also is contemplated to be within the scope of the present invention to use sets consisting of a single type of cell in one or more of the four sets of input cells used to calculate the first and second reference sets (i.e., tumor cell lines, control cell lines, first sample, and second sample). Direct statistical analysis using T-test and/or Mann-Whitney test for identification of genes differentially expressed in sets of biological samples that differ with respect to a classification is also applicable to the methods of the present invention. The average expression values for genes across the first and second sets of biological samples that differ with respect to a classification are used for calculation of fold expression changes (see below).

After the first and second reference sets of differentially expressed genes are identified, a concordance set of expressed genes is identified. The concordance set is obtained by comparing the first and second reference sets. Two criteria preferably are used to identify genes for inclusion into the concordance set: 1) the candidate gene is present in first and second reference sets; 2) the direction of the candidate gene's differential is the same in the first and second reference sets. Again, as one of ordinary skill readily will recognize, there is a certain degree of arbitrariness to the sign of the differential, as it is determined by, e.g., the direction of the comparison between samples [sample 1/sample 2, cf. sample 2/sample 1, or alternatively, sample 1-sample 2, cf. sample 2-sample 1]. In any event, the arbitrariness does not affect the results because the direction of the comparison is the same across the entire set of expressed genes. The first criterion is, in general, required for inclusion of a gene within the concordance set, while the second criterion is preferred, but optional. In practical terms, identification of a single reference set of differentially expressed genes could serve as a starting point for identification of a concordant set of transcripts. For example, one can identify a reference set of differentially regulated genes in a panel of biological samples subject to a classification and proceed directly to identification of a concordant set of differentially regulated genes in cell lines.

Once the concordance set has been established, information about the rank order of expression differences is used to establish another subset of genes. This subset is referred to as the minimum segregation set. The minimum segregation set may conveniently be selected by generating a scatter plot from which may be determined correlations between the −fold expression change or difference in the cell lines and the samples. In preferred embodiments, the −fold expression change is used, and is calculated by obtaining for gene x the ratio of the average expression value obtained across all tumor cell lines and across all control cell lines, and across the first and in the second sample sets, i.e.,

−fold change=<expression>₁/<expression>₂

where <expression>₁ is the average expression for gene x across all observations in set 1, and likewise, <expression>₂ is the average expression for gene x across all observations in set 2. Explicitly,

$$⟨ expression ⟩ = \frac{1}{N} {\sum }_{n = 1}^{N} {E}_{n} ,$$

where N equals the number of observations of expression value E for gene x in the set. In the case of the cell line data, set 1 preferably correspond to the tumor cell line set, and set 2 preferably corresponds to the control cell line set. Similarly, for the sample data, set 1 preferably corresponds to the first set of samples and set 2 preferably corresponds to the second set of samples.

In another preferred embodiment, differences in expression values are used and are calculated as:

difference=<expression>₁−<expression>₂,

where <expression>₁ and <expression>₂ have the same meanings as in the −fold change expression.

In other embodiments, preferred if the number of observations of gene x expression in each set is small, (i.e., on the order of one or two), a modified average fold change across all observations, <expression>ₘ, can be used in lieu of <expression>₁/<expression>₂ to improve the performance of the method. The modified average fold change <expression>ₘ explicitly is defined as:

<expression>ₘ=<expression>₁/<expression₁+expression₂>

which is equal to:

$${⟨ expression ⟩}_{m} = \frac{\frac{1}{N} {\sum }_{n = 1}^{N} {E}_{n}}{\frac{1}{N + M} {\sum }_{n = 1}^{N + M} {E}_{n}} ,$$

where there are N observations of expression value E for gene x from set 1 and M observations of expression value E for gene x from set 2. Improvement in the method performance can be determined using samples of known classification, and assessing the overall accuracy of the method in classifying known samples using <expression>ₘ in lieu of <expression>₁/<expression>₂.

Consider the following observations of expression values E for gene x in which N=M=5:

Expression Values, E, for gene x

| Set 1 Data | Set 2 Data |
|---|---|
| 5 | 1 |
| 4 | 2 |
| 8 | 1 |
| 7 | 4 |
| 3 | 2 |
| sum = 27 | sum = 10 |
| <expression>₁ = 27/5 = 5.4 | <expression>₂ = 10/5 = 2 |

<expression>₁/<expression>₂ = 5.4/2 = 2.7
<expression>ₘ = <expression>₁/
<expression₁ + expression₂> = 5.4/3.7 = 1.5

A scatter plot can be generated for genes within the concordance set in which each gene is assigned a point in the scatter plot. The (x,y) location of that point will be, or will be proportional to, the −fold expression change or difference in the cell line data (e.g., x) and the −fold expression change or difference in the sample data (e.g., y). Of course, the selection of the data assigned to be plotted on the abscissa and that to be plotted on the ordinate is arbitrary, so that one could have the x value correspond to the sample data and the y value correspond to the cell line data. In preferred embodiments, the −fold expression change or difference data is logarithmically transformed prior to plotting said data on the scatter plot.

The scatter plot potentially will be populated by data points that fall within any of the four quadrants of a graph in which the axes intersect at (0,0). Define quadrant I as negative x, positive y, quadrant II as positive x, positive y, quadrant m as positive x, negative y, and quadrant IV as negative x, negative y. The minimum segregation class is selected so as to include genes that fall within quadrants II and IV, and preferably to include only those genes within quadrants II and IV whose −fold expression changes or differences are highly positively correlated between the cell line and sample data. Alternatively, the minimum segregation class may be selected so as to include genes that fall within quadrants I and III, and preferably to include only those genes within quadrants I and III whose −fold expression changes or differences are highly negatively correlated between the cell line and sample data.

The scatter plots described above provide a convenient graphical representation of the data used in the clustering and classification methods of the present invention, although it is not necessary to generate such plots in the practice of the invention. Correlation coefficients can be generated for arrays of data without first plotting the data as described above. The expression data can be sorted by the values of the fold expression changes or differences and subsets of highly correlated data can be selected visually or with the aid of, e.g., regression analysis. Correlation coefficients may then be calculated on the subset of data.

Genes whose expression changes are highly correlated (positively or negatively) between the cell line and sample data may be identified by calculating a correlation coefficient for one or more subsets of genes that fall within quadrants II and IV (or alternatively for those that fall within quadrants I and III) of a scatter plot, and selecting as the minimum segregation set, those genes for which the correlation coefficient exceeds a predetermined value. Any one of a number of commonly used correlation coefficients may be used, including correlation coefficients generated for linear and non-linear regression lines through the data. Representative correlation coefficients include the correlation coefficient, ρ_{x,y,} that ranges between −1 and+1, such as is generated by Microsoft Excel's CORREL function, the Pearson product moment correlation coefficient, r, that also ranges between−1 and+1, that that reflects the extent of a linear relationship between two data sets, such as is generated by Microsoft Excel's PEARSON function, or the square of the Pearson product moment correlation coefficient, r², through data points in known y's and known x's, such as is generated by Microsoft Excel's RSQ function. The r² value can be interpreted as the proportion of the variance in y attributable to the variance in x.

In a preferred embodiment, the −fold expression change or difference data are logarithmically transformed (e.g., log₁₀ transformed), and the minimum segregation set is selected so that the correlation coefficient, ρ_{x,y}, is greater than or equal to 0.8, or is greater than or equal to 0.9, or is greater than or equal to 0.95, or is greater than or equal to 0.995. One of ordinary skill can readily work out equivalent values for other types of transformations (e.g. natural log transformations) and other types of correlation coefficients either mathematically, or empirically using samples of known classification.

The method can be terminated at the step of selecting the minimum segregation set. This set will consist of a collection or cluster of genes that is coordinately regulated during processes that result in phenotypic changes between the types of samples that comprise the sample sets.

The method may be continued, as described immediately below, to classify a sample as belonging to the first sample set or to the second sample set. The classification method uses a minimum segregation set of expressed genes to calculate a second correlation coefficient referred to as a “phenotype association index.” The method contemplates several different embodiments for calculating the second correlation coefficient. In a preferred embodiment, the second correlation coefficient is calculated by determining for an individual sample for which classification is sought, the −fold expression change for each gene x within the minimum segregation set. Preferably, the −fold expression change is determined with respect to the average value of expression for gene x across all samples used to identify the minimum segregation set. In the table above, assume set 1 data correspond to a first set of samples and that set 2 data correspond to a second set of samples. The average expression value for gene x across these samples is equal to 3.7. In this preferred embodiment, the −fold expression change is determined by computing the ratio of the expression value for gene x in the individual sample to the 3.7 average value across all the samples used to identify the minimum segregation set. For example, if the observed gene x expression value in the sample is 7, then the −fold expression change calculated according to this embodiment is 7/3.7=1.9. If the data were logarithmically transformed prior to identifying the minimum segregation set, then the same logarithmic transformation is carried out on the individual sample data prior to calculating the correlation coefficient.

In this preferred embodiment the classification is made according to the sign of this second correlation coefficient (phenotype association index). Given the setup outlined above, using −fold expression changes <expression>₁/<expression₁+expression₂> for the sample sets to calculate the minimum segregation set, a positive correlation coefficient obtained for the classified sample indicates that the sample is a member of sample set 1, while a negative correlation coefficient indicates the sample belongs to sample set 2.

In a refinement of this preferred embodiment, the magnitude of the correlation coefficient can be used as a threshold for classification. The larger the magnitude of the correlation coefficient, the greater the confidence that the classification is accurate. As one of ordinary skill readily will appreciate, the appropriate threshold can be determined through the use of test data that seek to classify samples of known classification using the methods of the present invention. The threshold is adjusted so that a desired level of accuracy (e.g., greater than about 70% or greater than about 80%, or greater than about 90% or greater than about 95% or greater than about 99% accuracy is obtained). This accuracy refers to the likelihood that an assigned classification is correct. Of course, the tradeoff for the higher confidence is an increase in the fraction of samples that are unable to be classified according to the method. That is, the increase in confidence comes at the cost of a loss in sensitivity.

In another preferred embodiment, multiple minimum segregation sets can be identified and used to increase the sensitivity of the method. Here again, test data from samples of known classification are used to identify the minimum segregation sets and classify the individual samples. In a preferred embodiment, successive minimum segregation classes are identified using expression data from true positive and false positive samples. The expression data from these samples is again broken down into two sample sets, with the true positives assigned to, e.g., sample set 1, and the false positives assigned to sample set 2. The re-apportioned expression data are used to identify another concordance set and another minimum segregation set. This additional minimum segregation set is used to re-score the samples with particular attention paid to the ability of the set to properly classify the false positives.

Several such iterations can be done, and criteria developed to improve the accuracy of the method by evaluating the behavior of known samples against a number of minimum segregation sets. Such analysis can be used to show, e.g., that true positives score with the correct phenotype association index in, e.g., 3 of 3 minimum segregation sets.

As one of ordinary skill will recognize, a similar approach can be used with false negatives, wherein the true negatives and the false negatives are used in an iterative embodiment of the invention, with the false negatives re-assigned to sample set 1 and the true negatives assigned to sample set 2. Blended methods also may be used in which, e.g., the true positives and false negatives are assigned to sample set 1 and the true negatives and false positives assigned to sample set 2, or any other logical combination that uses mis-classified samples to iteratively obtain minimum segregation sets that are used either alone or in conjunction with other sets to improve the accuracy of the classification methods of the present invention.

While the clustering and classification methods have been described primarily with reference to tumor samples, they are readily applicable to any biological analysis for which appropriate cell lines and samples can be obtained. These include by way of example, but not limitation, omnipotent stem cells, pluripotent precursor cells, various terminally differentiated cells, etc. The clustering methods applied to cell differentiation analyses will identify gene clusters that are coordinately regulated in differentiation programs. These genes are useful not only from a basic research point of view (e.g., to identify novel transcription factors or response elements), but also to identify gene products specifically expressed in one but not another cell type. Such gene products are useful for, e.g., targeting of therapeutic molecules using reagents that have affinity for the specifically expressed gene products.

Application of the methods of the present invention to the study and classification of cancers represents an important advance made possible in large part by the ready availability of gene expression data. Recent gene expression analysis data revealed that direct comparison of expression profiles for individual tumors to identify the transcriptome of human cancer progression is extremely challenging. Continuous phenotypic changes in cancer cells during tumor progression, individual phenotypic variations, intrinsic cellular heterogeneity, and variability in cellular composition of the primary and metastatic tumors render extremely problematic the selection of the gene expression changes relevant to tumor progression and metastasis. Furthermore, the use of human tumors and metastatic material, itself, limits the direct manipulation of variables that might otherwise reveal regulatory defects that are not apparent in the ground state expression patterns of in vivo tumors.

A complementary experimental approach to the extensive clinical sampling was developed employing gene expression analysis of selected cancer cell lines representing divergent clinically relevant variants of cancer progression (Table 1). These cell lines were surveyed under various in vitro and in vivo conditions that model microenvironments favorable to the malignant phenotype, including differential serum withdrawal responsiveness in vitro and induction of experimental tumors in nude mice, ultimately to identify expression changes characteristic of human cancer progression. These cell lines provide a representative group of tumor cell lines that can be used in the practice of the methods of the invention (although other transformed cell lines, such as are readily available from depositories such as ATCC or commercial suppliers also can be used). The methods of the invention also may be practiced using, e.g., one or more of the 38 human breast cancer cell lines described in Forozan, F., Mahlamaki, E. H., Monni, O., Chen, Y., Veldman, R., Jiang, Y., Gooden, G. C., Ethier, S. P., Kallioniemi, A., Kallioniemi, O-P. Comparative genomic hybridization analysis of 38 breast cancer cell lines: a basis for interpreting complementary DNA microarray data. Cancer Res. 2000. 60: 4519-4525, incorporated herein by reference. The methods of the invention also may be practiced using one or more of the 60 human cancer cell lines representing multiple forms of human cancer and utilized in the National Cancer Institute's screen for anti-cancer drug was described in Ross, T D, Scherf, U, Eisen, M B, Perou, C M, Rees, C, Spellman, P, Iyer, V, Jeffrey, S S, Van de Rijn, M, Waltham, M, Pergamenschikov, A, Lee, J C F, Lashkari, D, Shalon, D, Myers, T G, Weinstein, J N, Botstein, D, Brown, P O. Systematic variation in gene expression patterns in human cancer cell lines. Nature Genetics, 24: 227-235, 2000, incorporated herein by reference. Classification of the human cancer cell lines based on the observed gene expression profiles revealed a correspondence to the tissue of origins of the corresponding tumors from which the cell lines were derived (Ross, D T, et al, 2000).

Each cell line and experimental condition provided a criterion that a gene met in order to be retained in the next step of analysis. Thus, the cancer cell lines represented in Table 1 are especially useful for the practice of the clustering and classification methods of the invention. Each step in the gene selection process (i.e., identification of a first and a second reference set, identification of a concordance set and finally, identification of a minimum segregation set) can be thought of as a cut-off criterion that allows genes to pass to the next stage in the analysis. The identified set of candidate genes that satisfies these criteria comprises genes, the differential expression of which is associated with certain features of the malignant phenotype and that is relatively insensitive to significant alterations in cell type and environmental context. Consequently, these genes represent reliable starting points for identifying genes that are commonly altered in human cancer and represent a consensus transcriptome of cancer progression. Other cell line combinations suitable for practicing the methods of the present invention are set forth in Tables 2- 4. Table 2 lists representative cell line combinations for normal cells and certain cancers (e.g.., breast, prostate, lung). These combinations are especially useful for identifying genetic markers that serve as diagnostics for a malignant phenotype. Such markers, in addition to providing diagnostic information, can also provide drug discovery targets. Table 2 also lists representative cell line combinations for precursor and differentiated cells, useful for identifying differentiation markers. Such markers can be used to screen for agents that activate differentiation programs to further basic research, as well as tissue engineering work. Table 3 lists additional tumor cell/ control cell line combinations useful for practicing the methods of the invention to identify markers of malignant phenotype for diagnostic as well as drug discovery purposes. Table 4 provides additional primary tumor/ metastatic tumor cell line combinations useful for practicing the methods of the invention to identify markers of metastatic potential for diagnostic, prognostic and therapeutic applications.

TABLE 1
Model Human Cancer Cell Systems
Exhibiting Graded Metastatic Potential

| | | METASTATIC | |
|---|---|---|---|
| CELLS | DEFINITION | POTENTIAL | REMARKS |
| Breast | A panel of human | Metastatic potential | This series of cells |
| Cancer | breast carcinoma cell | varies from 0 (MDA- | exhibits differential |
| (metastatic | lines of graded | MB-361) to 10-90% | metastatic potential in |
| potential) | metastatic potential. | (MDA-MB-435 and | nude mice, differential |
| MDAMB- | High met variant | variants) incidence of | homotypic aggregation |
| 361 (0) | (lung2), low met | lung metastasis in | and clonogenic growth |
| MDAMB- | revertant (Br), and | nude mice following | properties, differential |
| 468 (5%) | blood-survival variant | orthotopic | sensitivity toward |
| MDAMB- | (B13) were derived | implantation. | apoptosis, in vivo and |
| 231 (30%) | from parental MB-435 | | vitro sensitivity to |
| MDA-MB- | cells. | | glycoamines, galectin- |
| 435 (60%) | | | dependent adhesion. |
| MB- | | | |
| 435lung2 | | | |
| (90%) | | | |
| MB-435Br | | | |
| (10%) | | | |
| MB- | | | |
| 435B13 (?) | | | |
| PC3 | Parental, 1 in vivo | Poorly metastatic | High metastatic |
| System | passage | Small prostate tumors | potential is associated |
| (Prostate-1) | 4 in vivo serial | Metastatic | with high resistance |
| PC-3M | passages in prostate | Highly metastatic | toward apoptosis. |
| PC-3M- | 4 in vivo serial | | Glycoamine-sensitive |
| Pro4 | passage; LN4 > Pro4 | | cell lines. From liver |
| PC-3M- | | | met. of splenic PC3 |
| LN4 | | | implant. |
| | | | Exhibit rapid large |
| | | | prostate tumor growth. |
| | | | Exhibit small prostate |
| | | | tumors, large LN |
| | | | metastatic tumors. |
| LNCap | parental | Poorly metastatic | Only androgen-sensitive |
| System | 5 in vivo serial | Highly metastatic | system. This panel |
| (Prostate-2) | passages in prostate | | exhibits differential |
| LNCaP | 3 in vivo serial | | metastatic potential, |
| LNCaP- | passages; LN3 > Pro5 | | differential sensitivity |
| Pro5 | | | toward apoptosis, and in |
| LNCaP- | | | vitro glycoamine |
| LN3 | | | sensitivity. LN3 exhibit |
| | | | decreased androgen |
| | | | dependency, increased |
| | | | PSA level, high |
| | | | frequency and load of |
| | | | regional LN metastasis. |
| BPE | SV40 large T antigen | Approximately 11% | Cell line system suitable |
| System | immortalized benign | tumorigenicity with 6 | for determination of the |
| (Prostate-3) | prostate epithelial | mo. latency. | gene expression changes |
| P69 | cells (BPE). | Lung and diaphragm | associated with |
| 2182 | 3 serial passages in | metastases. | alterations within major |
| M12 | vivo as xenograft | | tumor suppressor |
| | | | pathways. |
| Colon | Colon carcinoma cell | Differential capability | High metastatic |
| cancer | lines selected from a | to generate liver | potential within this cell |
| KM12-C | single parental cell | metastasis following | line system is associated |
| KM12-SP | line for differential | intrasplenic | with increased |
| KM12-SM | metastatic potential | implantation in nude | expression of a sialyl |
| KM12-L4 | through in vivo | mice. | Lewis family of |
| | passages in nude mice. | | glycoantigens and |
| | | | higher selectin-mediated |
| | | | adhesion. |

References: Pettaway, C. et al. Clin. Cancer Res., 2: 1627, 1996; Bae, V. et al. Int. J. Cancer, 58:721, 1994; Plymate, et al. J. Clin. Endocrinol., Met. 81: 3709, 1996; Morikawa et al. Cancer Res., 48: 1943, 1988; Morikawa et al. Cancer Res., 48: 6863, 1988; Schackert et al. Am. J. Pathol., 136: 95, 1990; Zhang et al. Cancer Res., 51: 2029, 1991; Zhang et al. Invasion Metastasis, 11: 204, 1991; Price et al. Cancer Res., 50: 717, 1990; Mukhopadhyay et al. Clin Exp Met., 17: 325, 1999; Glinsky et al. Clin. Exper. Metastasis, 14: 253, 1996; Glinsky et al. Cancer Res., 56: 5319, 1996; Glinsky et al. Cancer Lett., 115: 185, 1997; McConkey et al. Cancer Res., 56: 5594, 1996; Glinsky et al. Transf Med Rev 14: 326, 2000 (incorporated herein by reference).

TABLE 2
Representative Cell Line Combinations

| | | |
|---|---|---|
| Tumor Cell Line | Control Cell Line | Reference/comments |

Breast Cancer

| | | |
|---|---|---|
| See Table 1 | Clonetics ™ human mammary | ATCC collection, |
| | epithelial cells | incorporated herein by |
| | (Cat. # CC2551 from | reference; Cambrex, Inc. |
| | Cambrex, Inc., East | 2002 Biotech Catalog, |
| | Rutherford, NJ) | incorporated herein by |
| | | reference |

Prostate Cancer

| | | |
|---|---|---|
| Tumor Cell Line | Control Cell Line | Reference/comments |
| See Table 1 | Clonetics ™ prostate | ATCC collection, |
| | epithelial cells (Cat. # | incorporated herein by |
| | CC2555 from Cambrex, | reference; Cambrex, Inc. |
| | Inc., East Rutherford, NJ) | 2002 Biotech Catalog, |
| | | incorporated herein by |
| | | reference |

Lung Cancer

| | | |
|---|---|---|
| Tumor Cell Line | Control Cell Line | Reference/comments |
| See Table 3 | ATCC# CCL-256.1; NCI- | ATCC collection, |
| | BL2126; peripheral blood; | incorporated herein by |
| | Clonetics ™ bronchial | reference; |
| | epithelial cells (Cat. # | Cambrex, Inc. 2002 |
| | CC2540 from Cambrex, | Biotech Catalog, |
| | Inc., East Rutherford, NJ); | incorporated herein by |
| | Clonetics ™ small airway | reference |
| | epithelial cells (Cat. # | |
| | CC2547 from Cambrex, | |
| | Inc., East Rutherford, NJ); | |
| | See Table 3 | |

Other types of cancers

| | | |
|---|---|---|
| Tumor Cell Line | Control Cell Line | Reference/comments |
| See Table 3 | See Table 3 | |

Differentiation Pathway

| | | |
|---|---|---|
| Precursor/Stem Cell Line | Differentiated Cell Line | Reference/comments |
| CD133+ cells Cat. # 2M- | mononuclear cells Cat | ATCC collection, |
| 102A - bone marrow | #2M-125C; CD4+ T-cells | incorporated herein by |
| derived; Cat # 2G102 - G- | Cat. # 1C-200; human | reference; Cambrex, Inc. |
| CSF derived; Cat. # 2L- | astrocytes Cat. # CC2565; | 2002 Biotech Catalog, |
| 102A - fetal liver derived; | human hepatocytes Cat. # | incorporated herein by |
| CD36+ erythroid | CC2591; NHEM neonatal | reference |
| progenitors Cat # 2C-250; | melanocytes Cat. # | |
| cord blood CD19+ B cells | CC2513; SkMC - Skeletal | |
| Cat # 1C-300; dendritic | Muscle Cells Cat. # | |
| cell precursors Cat # 2P- | CC2561 (all from | |
| 105; NHNP neural | Cambrex, Inc., East | |
| progenitor cells Cat. # | Rutherford, NJ) | |
| CC2599; hMSC - | | |
| mesenchymal stem cells, | | |
| human bone marrow Cat. # | | |
| PT-2501 (all from | | |
| Cambrex, Inc., East | | |
| Rutherford, NJ) | | |

TABLE 3
Representative Tumor/Control Cell Line
Combinations Available from American Type Culture Collection (ATCC)

| Tumor Cell Line | | | | Control Cell Line | | |
|---|---|---|---|---|---|---|
| ATCC | | Cancer | Tissue | ATCC | | Tissue |
| No. | Name | Type | Source | No. | Name | Source |
| CCL-256 | NCI-H2126 | carcinoma; non- | lung | CCL-256.1 | NCI-BL2126 | peripheral |
| | | small cell lung | | | | blood |
| | | cancer | | | | |
| CRL-5868 | NCI-H1395 | adenocarcinoma | lung | CRL-5957 | NCI-BL1395 | peripheral |
| | | | | | | blood |
| CRL-5882 | NCI-H1648 | adenocarcinoma | lung | CRL-5954 | NCI-BL1648 | peripheral |
| | | | | | | blood |
| CRL-5911 | NCI-H2009 | adenocarcinoma | lung | CRL-5961 | NCI-BL2009 | peripheral |
| | | | | | | blood |
| CRL-5985 | NCI-H2122 | adenocarcinoma | pleural | CRL-5967 | NCI-BL2122 | peripheral |
| | | | effusion | | | blood |
| CRL-5922 | NCI-H2087 | adenocarcinoma | lymph node | CRL-5965 | NCI-BL2087 | peripheral |
| | | | (metastasis) | | | blood |
| CRL-5886 | NCI-H1672 | carcinoma; | lung | CRL-5959 | NCI-BL1672 | peripheral |
| | | classic small | | | | blood |
| | | cell lung cancer | | | | |
| CRL-5929 | NCI-H2171 | carcinoma; | lung | CRL-5969 | NCI-BL2171 | peripheral |
| | | small cell lung | | | | blood |
| | | cancer | | | | |
| CRL-5931 | NCI-H2195 | carcinoma; | lung | CRL-5956 | NCI-BL2195 | peripheral |
| | | small cell lung | | | | blood |
| | | cancer | | | | |
| CRL-5858 | NCI-H1184 | carcinoma; | lymph node | CRL-5949 | NCI-BL1184 | peripheral |
| | | small cell lung | (metastasis) | | | blood |
| | | cancer | | | | |
| HTB-172 | NCI-H209 | carcinoma; | bone | CRL-5948 | NCI-BL209 | peripheral |
| | | small cell lung | marrow | | | blood |
| | | cancer | (metastasis) | | | |
| CRL-5983 | NCI-H2107 | carcinoma; | bone | CRL-5966 | NCI-BL2107 | peripheral |
| | | small cell lung | marrow | | | blood |
| | | cancer | (metastasis) | | | |
| HTB-120 | NCI-H128 | carcinoma; | pleural | CRL-5947 | NCI-BL128 | peripheral |
| | | small cell lung | effusion | | | blood |
| | | cancer | | | | |
| CRL-5915 | NCI-H2052 | mesothelioma | pleural | CRL-5963 | NCI-BL2052 | peripheral |
| | | | effusion | | | blood |
| CRL-5893 | NCI-H1770 | neuroendocrine | lymph node | CRL-5960 | NCI-BL1770 | peripheral |
| | | carcinoma | (metastasis) | | | blood |
| HTB-126 | Hs 578T | ductal | mammary | HTB-125 | Hs 578Bst | mammary |
| | | carcinoma | gland; | | | gland; |
| | | | breast | | | breast |
| CRL-2320 | HCC1008 | ductal | mammary | CRL-2319 | HCC1007 BL | peripheral |
| | | carcinoma | gland; | | | blood |
| | | | breast | | | |
| CRL-2338 | HCC1954 | ductal | mammary | CRL-2339 | HCC1954 BL | peripheral |
| | | carcinoma | gland; | | | blood |
| | | | breast | | | |
| CRL-2314 | HCC38 | primary ductal | mammary | CRL-2346 | HCC38 BL | peripheral |
| | | carcinoma | gland; | | | blood |
| | | | breast | | | |
| CRL-2321 | HCC1143 | primary ductal | mammary | CRL-2362 | HCC1143 BL | peripheral |
| | | carcinoma | gland; | | | blood |
| | | | breast | | | |
| CRL-2322 | HCC1187 | primary ductal | mammary | CRL-2323 | HCC1187 BL | peripheral |
| | | carcinoma | gland; | | | blood |
| | | | breast | | | |
| CRL-2324 | HCC1395 | primary ductal | mammary | CRL-2325 | HCC1395 BL | peripheral |
| | | carcinoma | gland; | | | blood |
| | | | breast | | | |
| CRL-2331 | HCC1599 | primary ductal | mammary | CRL-2332 | HCC1599 BL | peripheral |
| | | carcinoma | gland; | | | blood |
| | | | breast | | | |
| CRL-2336 | HCC1937 | primary ductal | mammary | CRL-2337 | HCC1937 BL | peripheral |
| | | carcinoma | gland; | | | blood |
| | | | breast | | | |
| CRL-2340 | HCC2157 | primary ductal | mammary | CRL-2341 | HCC2157 BL | peripheral |
| | | carcinoma | gland; | | | blood |
| | | | breast | | | |
| CRL-2343 | HCC2218 | primary ductal | mammary | CRL-2363 | HCC2218 BL | peripheral |
| | | carcinoma | gland; | | | blood |
| | | | breast | | | |
| CRL-7345 | Hs 574.T | ductal | mammary | CRL-7346 | Hs 574.Sk | skin |
| | | carcinoma | gland; | | | |
| | | | breast | | | |
| CRL-7482 | Hs 742.T | scirrhous | mammary | CRL-7481 | Hs 742.Sk | skin |
| | | adenocarcinoma | gland; | | | |
| | | | breast | | | |
| CRL-7365 | Hs 605.T | carcinoma | mammary | CRL-7364 | Hs 605.Sk | skin |
| | | | gland; | | | |
| | | | breast | | | |
| CRL-7368 | Hs 606 | carcinoma | mammary | CRL-7367 | Hs 606.Sk | skin |
| | | | gland; | | | |
| | | | breast | | | |
| CRL-1974 | COLO 829 | malignant | skin | CRL-1980 | COLO 829BL | peripheral |
| | | melanoma | | | | blood |
| CRL-7762 | TE 354.T | basal cell | skin | CRL-7761 | TE 353.Sk | skin |
| | | carcinoma | | | | |
| CRL-7677 | Hs 925.T | pagetoid | skin | CRL-7676 | Hs 925.Sk | skin |
| | | sarcoma | | | | |
| CRL-7672 | Hs 919.T | benign osteoid | bone | CRL-7671 | Hs 919.Sk | skin |
| | | osteoma | | | | |
| CRL-7554 | Hs 821.T | giant cell | bone | CRL-7553 | Hs 821.Sk | skin |
| | | sarcoma | | | | |
| CRL-7552 | Hs 820.T | heterophilic | bone | CRL-7551 | Hs 820.Sk | skin |
| | | osteofication | | | | |
| CRL-7444 | Hs 704.T | osteosarcoma | bone | CRL-7443 | Hs 704.Sk | skin |
| CRL-7448 | Hs 707(A).T | osteosarcoma | bone | CRL-7449 | Hs 707(B).Ep | skin |
| CRL-7471 | Hs 735.T | osteosarcoma | bone | CRL-7865 | Hs 735.Sk | skin |
| CRL-7595 | Hs 860.T | osteosarcoma | bone | CRL-7519 | Hs 791.Sk | skin |
| CRL-7622 | Hs 888.T | osteosarcoma | bone | CCL-211 | Hs888Lu | lung |
| CRL-7626 | Hs 889.T | osteosarcoma | bone | CRL-7625 | Hs 889.Sk | skin |
| CRL-7628 | Hs 890.T | osteosarcoma | bone | CRL-7627 | Hs 890.Sk | skin |
| CRL-7453 | Hs 709.T | periostitis; | bone | CRL-7452 | Hs 709.Sk | skin |
| | | granuloma | | | | |
| CRL-7886 | Hs 789.T | transitional cell | ureter | CRL-7518 | Hs 789.Sk | skin |
| | | carcinoma | | | | |
| CRL-7547 | Hs 814.T | giant cell | vertebral | CRL-7546 | Hs 814.Sk | skin |
| | | sarcoma | column | | | |

TABLE 4
Representative Primary Tumor/Metastatic Tumor Cell Line
Combinations Available from American Type Culture Collection (ATCC)

| Primary Cell Line | | | | Metastatic Cell Line | | |
|---|---|---|---|---|---|---|
| ATCC | | | | ATCC | | |
| No. | Name | Disease | Tissue | No. | Name | Tissue |
| CCL-228 | SW480 | colorectal | colon | CCL-227 | SW620 | lymph |
| | | adenocarcinoma | | | | node |
| CRL-1864 | RF-1 | gastric | stomach | CRL-1863 | RF-48 | ascites |
| | | adenocarcinoma | | | | |
| CRL-1675 | WM-115 | melanoma | skin | CRL-1676 | WM-266-4 | n/a |
| CRL-7425 | Hs | melanoma | skin | CRL-7426 | Hs 688(B).T | lymph |
| | 688(A).T | | | | | node |

Application of the methods of the invention to the study of particular cancers is described generally below, and is followed by specific working examples demonstrating aspects of the invention.

Prostate Cancer

As many as 50% of men, aged 70 years and over have microscopic foci of prostate cancer without clinical evidence of disease (Trump, D. L., Robertson, C. N., Holland, J. F., Frei, E., Bast, R. C., Kufe, D. W., Morton, D. L., and Weishselbaum, R. R. Neoplasms of the prostate. In: D. L. Trump, C. N. Robertson, J. F. Holland, E. Frei, R. C. Bast, D. W. Kufe, D. L. Morton, and R. R. Weishselbaum (eds.), Cancer Med, Vol. 3, pp. 1562-86. Philadelphia: Lea & Febiger, 1993.). Although some prostate cancers remain indolent and confined to the gland, other prostate cancers behave more aggressively and metastasize if not adequately treated. Prostate cancer is the second most lethal neoplasia in males after lung cancer. Because of widespread screening programs utilizing serum PSA values, many more cases of early stage disease are being diagnosed. In 1988 approximately 50% of patients were diagnosed with early stage disease (stage I and II). Today, about 75% of patients have early stage disease that is potentially curable.

Unfortunately, the only potentially curative therapy for prostate cancer consists of radical prostatectomy or other local therapies such as external irradiation, implanted irradiation seeds, or cryotherapy. The use of prostatectomy has increased in step with the amount of diagnosed early stage prostate cancer. SEER data indicates an increase in prostatectomies from 17.4 per 100,000 in 1988 to 54.6 per 100,000 in 1992. Insufficient treatment leads to local disease extension and metastasis. Current methods, such as Gleason scores are not perfectly reliably correlated with whether a tumor is aggressive or indolent. Thus, developing a treatment strategy appropriate for any individual is difficult. The recognition of those genetic changes that portend metastatic prostate cancer would, therefore, be a breakthrough. The methods of the present invention readily identify such genetic changes.

Breast Cancer

Breast cancer is the most common cancer among women in North America and Western Europe and is the second leading cause of female cancer death in the United States. In the United States, age-adjusted breast cancer incidence rates have considerably increased during last century. Approximately 40% of patients diagnosed with breast cancer have disease that has regional or distant metastases and, at present, there is no efficient curative therapy for breast cancer patients with advanced metastatic disease. Thus, developing a treatment strategy appropriate for any individual with early stage disease is difficult and insufficient treatment leads to local disease extension and metastasis. Therefore, there is an urgent clinical need for novel diagnostic methods that would allow early identification of those breast cancer patients who are likely to develop metastatic disease and would require the most aggressive and advanced forms of therapy for increased chance of survival. The identification of those genetic changes that distinguish aggressive metastatic disease and predict metastatic behavior would, therefore, be a breakthrough. The methods of the present invention provide information that allows prognostication of aggressive metastatic disease.

Recent gene expression analysis of human tumor samples employing cDNA microarray technology underscores the difficulties in identification of the cellular origin of differentially expressed transcripts in clinical samples due to the remarkable cellular heterogeneity and variability in cellular compositions of human tumors (Golub, T. R., Slonim, D. K., Tamayo, P., Huard, C., Gaasenbeek, M., Mesirov, J. P., Coller, H., Loh, M. L., Downing, J. R., Caliguri, M. A., Bloomfield, C. D., Lander, E. S. 1999. Molecular classification of cancer: class discovery and class prediction by gene expression monitoring. Science, 286: 531-537; Perou C M, Jeffrey S S, van de Rijn M, et al. Distinctive gene expression patterns in human mammary epithelial cells and breast cancers. Proc Natl Acad Sci USA. 1999. 96:9212-9217; Perou C M, Sorlie T, Eisen M B, et al. Molecular portrait of human breast tumors. Nature. 2000. 406:747-752, incorporated herein by reference). However, a cDNA microarray analysis of gene expression in melanoma cell lines of distinct metastatic potential, was successfully employed for identification of RhoC as an essential gene for the acquisition of metastatic phenotype by melanoma cells (Clark, E A, Golub T R, Lander E S, Hynes R O. Genomic analysis of metastasis reveals an essential role for RhoC. Nature 2000. 406:532-535, incorporated herein by reference). Established human cancer cell lines were utilized for parallel comparisons of the alterations in DNA copy number and gene expression associated with human breast cancer (Pollack, J. R., Perou, C. M., Alizadeh, A. A., Eisen, M. B., Pergamenschikov, A., Williams, C. F., Jeffrey, S. S., Botstein, D., Brown, P. O. Genome-wide analysis of DNA-copy number changes using cDNA microarrays. Nature Genetics. 1999. 23: 41-46; Forozan, F., Mahlamaki, E. H., Monni, O., Chen, Y., Veldman, R., Jiang, Y., Gooden, G. C., Ethier, S. P., Kallioniemi, A., Kallioniemi, O-P. Comparative genomic hybridization analysis of 38 breast cancer cell lines: a basis for interpreting complementary DNA microarray data. Cancer Res. 2000. 60: 4519-4525, incorporated herein by reference). Thus, model systems are a reasonable source of gene candidates to be studied in the much more heterogeneous environment of real human tumors.

Analysis of gene expression in normal and neoplastic ovarian human tissues using methods of the present invention revealed that high malignant potential ovarian cancers exhibited gene expression profile somewhat similar to the ovarian cancer cell lines (Welsh, J. B., Zarrinkar, P. P., Sapinoso, L. M., Kern, S. G., Behling, C. A., Monk, B. J., Lockhart, D. J., Burger, R. A., Hampton, G. M. Analysis of gene expression profiles in normal and neoplastic ovarian tissue samples identifies candidate molecular markers of epithelial ovarian cancer. Proc Natl Acad Sci USA. 2001. 98:1176-1181, incorporated herein by reference), further validating the complementary gene expression analysis approach utilizing selected established cancer cell lines and clinical samples.

Metastasis

Cancer cells have exceedingly low survival rates in the circulation (reviewed in [Glinsky, G. V. 1993. Cell adhesion and metastasis: is the site specificity of cancer metastasis determined by leukocyte-endothelial cell recognition and adhesion? Crit. Rev. Onc./Hemat., 14: 229-278, incorporated herein by reference). Even if the bloodstream contains many cancer cells, there may be no clinical or pathohistological evidence of metastatic dissemination into the target organs (Williams, W. R. The theory of Metastasis. In The Natural History of Cancer. 1908; 442-448; Goldmann, E. 1907. The growth of malignant disease in man and the lower animals, with special reference to the vascular system. Proc. R. Soc. Med., 1: 1-13; Schmidt, M. B. In Die Verbreitungswege der Karzinome und die bezienhung generalisiertes sarkome su den leukamischen neubildungen. Fischer, Jena, 1903, incorporated herein by reference). The levels of metastatic efficiency at the intramicrovascular (postintravasation) phase of metastatic dissemination were shown to be only 0.2% and 0.003% in high and low metastatic variants of B16 melanoma cells, respectively, injected at a concentration of 105 cells into the tail veins of laboratory mice (Weiss, L. 1990. Metastatic inefficiency. Adv. Cancer Res., 54: 159-211; Weiss, L., Mayhew, E., Glaves-Rapp, D., Holmes, J. C. 1982. Metastatic inefficiency in mice bearing B16 melanomas. Br. J. Cancer, 45: 44-53, incorporated herein by reference). The fate of cancer cells in the circulation is a rapid phase of intramicrovascular cancer cell death, which is completed in <5 minutes and accounts for 85% of arrested cancer cells. This is followed by a slow phase of cell death, which accounts for the vast majority of the remainder (Weiss, L. 1988. Biomechanical destruction of cancer cells in the hart: a rate regulator of hematogenous metastasis. Invas. Metastasis, 8: 228-237; Weiss, L., Orr, F. W., Honn, K. V. 1988. Interactions of cancer cells with the microvasculature during metastasis. FSEB J., 2: 12-21; Weiss, L., Harlos, J. P., Elkin, G. 1989. Mechanism of mechanical trauma to Ehrlich ascites tumor cells in vitro and its relationship to rapid intravascular death during metastasis. Int. J. Cancer, 44: 143-148, incorporated herein by reference).

For example, the number of tumor cells in the lungs declined very rapidly after intravenous injection i.e., 90-99% had disappeared after 24 hours (Hewitt, H. B., Blake, A. 1975. Quantitative studies of translymphonodal passage of tumor cells naturally disseminating from a nonimmunogenic murine squamous carcinoma. Br. J. Cancer, 31: 25-35; Fidler, I. J. 1970. Metastasis: quantitative analysis of distribution and fate of tumor emboli labeled with 125I-5 iodo-2′-deoxyuridine. J. Natl. Cancer Inst., 45: 773-782; Proctor, J. W. 1976. Rat sarcoma model supports both soil seed and mechanical theories of metastatic spread. Br. J. Cancer, 34: 651-654; Proctor, J. W., Auclair, B. G., Rudenstam, C. M. 1976. The distribution and fate of blood-born 125IudR-labeled tumor cells in immune syngeneic rats. Int. J. Cancer, 18: 255-262; Weston, B. J., Carter, R. L., Eastry, G. C., Connell, D. I., Davies, A. J. C. 1974. The growth and metastasis of an allografted lymphoma in normal, deprived and reconstituted mice. Int. J. Cancer, 14: 176-185; Kodama, M., Kodama, T. 1975. Enhancing effect of hydrocortisone on hematogenous metastasis of Ehrlich ascites tumor in mice. Cancer Res., 35: 1015-1021, incorporated herein by reference) and after 3 days generally less than 1% remained (Fidler, I. J. 1970. Metastasis: quantitative analysis of distribution and fate of tumor emboli labeled with 125I-5 iodo-2′-deoxyuridine. J. Natl. Cancer Inst., 45: 773-782; Weston, B. J., Carter, R. L., Eastry, G. C., Connell, D. I., Davies, A. J. C. 1974. The growth and metastasis of an allografted lymphoma in normal, deprived and reconstituted mice. Int. J. Cancer, 14: 176-185; Kodama, M., Kodama, T. 1975. Enhancing effect of hydrocortisone on hematogenous metastasis of Ehrlich ascites tumor in mice. Cancer Res., 35: 1015-1021, incorporated herein by reference). This decline is due to a rapid degeneration of cancer cells (Fidler, I. J. 1970. Metastasis: quantitative analysis of distribution and fate of tumor emboli labeled with 125I-5 iodo-2′-deoxyuridine. J. Natl. Cancer Inst., 45: 773-782; Roos, E., Dingemans, K. P. 1979. Mechanisms of metastasis. Biochim. Biophys. Acta, 560: 135-166, incorporated herein by reference). Therefore, the individual ‘average’ cancer cell survives only a short time in the circulation. The successful metastatic cancer cells are able to find a largely unknown survival and escape route. Patients at high risk for metastatic disease could be better managed if gene expression patterns correlated with a clinical metastatic phenotype are identified. The methods of the present invention identify such gene expression patterns. Patients' tumor samples can be tested to see whether the gene expression pattern is associated with an increased risk of metastasis, and if so, the patients can be treated with more aggressive therapies to lower the risk of metastasis. As explained in greater detail below, the present invention provides for methods that allow identification of such gene expression patterns, and sample classification based on those patterns.

Models of Human Cancer Metastasis of Graded Metastatic Potential

We have acquired several well-established and characterized model human cancer cell systems of graded metastatic potential (Table 1). The collection of these human cancer cell line panels provides different backgrounds upon which increased metastatic potential is superimposed. We have studied these cell line systems extensively for many years both in vitro and in vivo (Glinsky, G. V. 1998. Failure of Apoptosis and Cancer Metastasis. Berlin/Heidelberg: Springer-Verlag, pp. 178 et seq.; Glinsky, G. V., Mossine, V. V., Price, J. E., Bielenberg, D., Glinsky, V. V., Ananthaswamy, H. N., Feather, M. S. 1996. Inhibition of colony formation in agarose of metastatic human breast carcinoma and melanoma cells by synthetic glycoamines. Clin. Exp. Metastasis, 14: 253-267; Glinsky, G. V., Price, J. E., Glinsky, V. V., Mossine, V. V., Kiriakova, G., Metcalf, J. B. 1996. Inhibition of human breast cancer metastasis in nude mice by synthetic glycoamines. Cancer Res., 56: 5319-5324; Glinsky, G. V., Glinsky, V. V. 1996. Apoptosis and metastasis: a superior resistance of metastatic cancer cells to the programmed cell death. Cancer Lett., 101: 43-51; Glinsky, G. V., Glinsky, V. V., Ivanova, A. B., Hueser, C. N. 1997. Apoptosis and metastasis: increased apoptosis resistance of metastatic cancer cells is associated with the profound deficiency of apoptosis execution mechanisms. Cancer Lett., 115: 185-193, incorporated herein by reference) and, therefore, have considerable experience in the maintenance of cell lines preserving graded metastatic potentials. These models provide an excellent opportunity to test whether concordant changes in gene expression underlie the metastasis process and to test the efficacy of drugs designed to block one or more crucial targets.

Four important features of the selected models have been documented (Glinsky, G. V. 1997. Apoptosis in metastatic cancer cells. Crit. Rev. Onc/Hemat., 25:175-186; Glinsky, G. V. 1998. Anti-adhesion cancer therapy. Cancer and Metastasis Reviews, 17: 171-185.Glinsky, G. V. 1998. Failure of Apoptosis and Cancer Metastasis. Berlin/Heidelberg: Springer-Verlag, pp 178 et seq.; Glinsky, G. V., Mossine, V. V., Price, J. E., Bielenberg, D., Glinsky, V. V., Ananthaswamy, H. N., Feather, M. S. 1996. Inhibition of colony formation in agarose of metastatic human breast carcinoma and melanoma cells by synthetic glycoamines. Clin. Exp. Metastasis, 14: 253-267; Glinsky, G. V., Price, J. E., Glinsky, V. V., Mossine, V. V., Kiriakova, G., Metcalf, J. B. 1996. Inhibition of human breast cancer metastasis in nude mice by synthetic glycoamines. Cancer Res., 56: 5319-5324; Glinsky, G. V., Glinsky, V. V. 1996. Apoptosis and metastasis: a superior resistance of metastatic cancer cells to the programmed cell death. Cancer Lett., 101: 43-51; Glinsky, G. V., Glinsky, V. V., Ivanova, A. B., Hueser, C. N. 1997. Apoptosis and metastasis: increased apoptosis resistance of metastatic cancer cells is associated with the profound deficiency of apoptosis execution mechanisms. Cancer Lett., 115: 185-193, incorporated herein by reference): a) highly metastatic cell variants possess an increased survival ability, high clonogenic growth potential, and enhanced resistance to apoptosis compared to parental or poorly metastatic counterparts; b) treatment of highly metastatic cell variants with certain synthetic glycoamine analogues caused inhibition of clonogenic growth and survival and reversal of apoptosis resistance in vitro, as well as significant reduction of metastatic potential in vivo; c) these cell lines maintain their distinct in vivo metastatic potentials during in vitro passage for at least several months, indicating that metastatic ability is preserved in vitro; d) differential transcription profiles of four metastasis-associated genes between high and low metastatic cell variants was shown to be similar in vitro and in vivo (Greene, G. F., Kitadai, Y., Pettaway, C. A., von Eschenbach, A. C., Bucana, C. D., Fidler, I. J. 1997. Correlation of metastasis-related gene expression with metastatic potential in human prostate carcinoma cells implanted in nude mice using an in situ messenger RNA hybridization technique. American J. Pathology, 150: 1571-1582, incorporated herein by reference) indicating the potential relevance of in vitro gene expression patterns to the metastatic phenotype. Thus, in accordance with the methods of the present invention, these cellular systems can be used to identify relevant gene expression patterns associated with phenotypes of interest (such as, e.g., metastasis, invasiveness, etc.) by comparing patterns of differential gene expression in one or more independently selected cell line variants with those in different types of clinical human cancer samples.

Orthotopic Model of Human Cancer Metastasis in Nude Mice

When human tumor cells are injected into ectopic sites in nude mice most do not metastasize (Fidler, I. J. The nude mouse model for studies of human cancer metastasis. In: V. Schirrmacher and R. Schwartz-Abliez (eds.). pp. 11-17. Berlin: Springer-Verlag, 1989; Fidler, I. J. Critical factors in the biology of human cancer metastasis. 1990. Cancer Res., 50, 6130-6138, incorporated herein by reference). The normal host tissue environment influences metastatic ability of cancer cells in such a way that many human and animal tumors transplanted into nude mice metastasize only if placed in the orthotopic organ (Fidler, I. J. The nude mouse model for studies of human cancer metastasis. In: V. Schirrmacher and R. Schwartz-Abliez (eds.). pp. 11-17. Berlin: Springer-Verlag, 1989; Fidler, I. J. Critical factors in the biology of human cancer metastasis. 1990. Cancer Res., 50, 6130-6138; Fidler, I. J., Naito, S., Pathak, S. 1990. Orthotopic implantation is essential for the selection, growth and metastasis of human renal cell cancer in nude mice. Cancer Metastasis Rev., 9, 149-165; Giavazzi, R., Campbell, D. E., Jessup, J. M., Cleary, K., and Fidler, I. J. 1986. Metastatic behavior of tumor cells isolated from primary and metastatic human colorectal carcinomas implanted into different sites in nude mice. Cancer Res., 46: 1928-1948; Naito, S., von Eschenbach, A. C., Giavazzi, R., and Fidler, I. J. 1986. Growth and metastasis of tumor cells isolated from a renal cell carcinoma implanted into different organs of nude mice. Cancer Res., 46: 4109-4115; McLemore, T. L., et al. 1987. Novel intrapulmonary model for orthotopic propagation of human lung cancer in athymic nude mice. Cancer Res., 47: 5132-5140, incorporated herein by reference). These observations pointed out the unique opportunity to study gene expression changes associated with aggressive metastatic phenotype. A comparison of gene expression patterns using the same high metastatic variant implanted at orthotopic (metastasis promoting model) and ectopic (metastasis suppressing model) sites should provide unique information regarding differential gene expression profiles associated with metastatic behavior in vivo.

Several orthotopic models of human cancer metastasis have been developed (Fu, X., Herrera, H., and Hoffman, R. M. 1992. Orthotopic growth and metastasis of human prostate carcinoma in nude mice after transplantation of histologically intact tissue. Int. J. Cancer, 52: 987-990; Stephenson, R. A., Dinney, C. P. N., Gohji, K., Ordonez, N. G., Killion, J. J., and Fidler, I. J. 1992. Metastatic model for human prostate cancer using orthotopic implantation in nude mice. J. Natl. Cancer Inst., 84: 951-957; Pettaway, C. A., Stephenson, R. A., and Fidler, I. J. 1993. Development of orthotopic models of metastatic human prostate cancer. Cancer Bull. (Houst.), 45: 424-429; An, Z., Wang, X., Geller, J., Moossa, A. R., and Hoffman, R. M. 1998. Surgical orthotopic implantation allows high lung and lymph node metastasis expression of human prostate carcinoma cell line PC-3 in nude mice. The Prostate, 34: 169-174; Wang, X., An, Z., Geller, J., and Hoffman, R. M. 1999. High-malignancy orthotopic mouse model of human prostate cancer LNCaP. The Prostate, 39: 182-186; Yang, M., Jiang, P., Sun, F.-X., Hasegawa, S., Baranov, E., Chishima, T., Shimada, H., Moosa, A. R., and Hofman, R. M. 1999. A fluorescent orthotopic bone metastasis model of human prostate cancer. Cancer Res., 59: 781-786, incorporated herein by reference). The orthotopic model of human cancer metastasis in nude mice was used for in vivo selection of highly and poorly metastatic cell variants, employing either established panels of human cancer cell lines or cell variants derived from the same parental cell lines (Giavazzi, R., Campbell, D. E., Jessup, J. M., Cleary, K., and Fidler, I. J. 1986. Metastatic behavior of tumor cells isolated from primary and metastatic human colorectal carcinomas implanted into different sites in nude mice. Cancer Res., 46: 1928-1948; Morikawa, K., Walker, S. M., Jessup, J. M., Cleary, K., and Fidler, I. J. 1988. In vivo selection of highly metastatic cells from surgical specimens of different primary human colon carcinoma implanted in nude mice. Cancer Res., 48: 1943-1948; Dinney, C. P. N. et al. 1995. Isolation and characterization of metastatic variants from human transitional cell carcinoma passaged by orthotopic implantation in athymic nude mice. J. Urol., 154: 1532-1538, incorporated herein by reference).

This approach was successfully applied to develop a human breast cancer model of graded metastatic potential (see Glinsky, G. V., Mossine, V. V., Price, J. E., Bielenberg, D., Glinsky, V. V., Ananthaswamy, H. N., Feather, M. S. 1996. Inhibition of colony formation in agarose of metastatic human breast carcinoma and melanoma cells by synthetic glycoamines. Clin. Exp. Metastasis, 14: 253-267; Glinsky, G. V., Price, J. E., Glinsky, V. V., Mossine, V. V., Kiriakova, G., Metcalf, J. B. 1996. Inhibition of human breast cancer metastasis in nude mice by synthetic glycoamines. Cancer Res., 56: 5319-5324, incorporated herein by reference) as well as three independent panels of human prostate cancer cell lines with distinct metastatic potential (Pettaway, C. A., Pathak, S., Greene, G., Ramirez, E., Wilson, M. R., Killion, J. J., and Fidler, I. J. 1996. Selection of highly metastatic variants of different human prostatic carcinomas using orthotopic implantation in nude mice. Clinical Cancer Res., 2: 1627-1636; Bae, V. L., Jackson-Cook, C. K., Brothman, A. R., Maygarden, S. J., and Ware, J. Tumorugenicity of SV40 T antigen immortalized human prostate epithelial cells: association with decreased epidermal growth factor receptor (EGFR) expression. Int. J. Cancer 1994;58:721-29; Plymate, et al., The effect of the IGF system in human prostate epithelial cells of immortalization and transformation by SV-40 T antigen. J. Clin. Endocrinol. Met. 1996:81;3709-16; Jackson-Cook, C., Bae, V., Edelman W., Brothman, A., and Ware, J. Cytogenetic characterization of the human prostate cancer cell line P69SV40T and its novel tumorigenic sublines M2182 and M15. Cancer Genet. & Cytogenet 1996;87:14-23; Bae, V. L., Jackson-Cook, C. K., Maygarden, S. J., Plymate, S. R., Chen, J., and Ware, J. L. Metastatic subline of an SV40 large T antigen immortalized human prostate epithelial cell line. Prostate 1998;34:275-82, incorporated herein by reference). Recent experimental evidence indicates that enhancement of metastatic capability of human cancer cells transplanted orthotopically is associated with differential expression of several metastasis-associated genes that have been implicated earlier in certain key features of the metastatic phenotype (Greene, G. F., Kitadai, Y., Pettaway, C. A., von Eschenbach, A. C., Bucana, C. D., Fidler, I. J. 1997. Correlation of metastasis-related gene expression with metastatic potential in human prostate carcinoma cells implanted in nude mice using an in situ messenger RNA hybridization technique. American J. Pathology, 150: 1571-1582, incorporated herein by reference). These data support the rationale for the methods of the present invention to identify gene expression profiles associated with the phenotypes of clinical tumor samples based on a combination of in vitro gene expression analysis in one or more cell lines having a phenotype of interest (e.g., metastatic potential, invasiveness, etc.) and gene expression analysis of clinical samples.

A similar rationale supports the use of the methods of the present invention to identify gene expression patterns correlated with specific differentiation pathways associated with defined cell types (e.g., liver, skin, bone, muscle, blood, etc.), although in this instance, the preferred relevant comparisons are the gene expression profiles of one or more stem cell lines with that of the terminally differentiated cell type. (See, e.g., Table 2, supra.) In a related method of the present invention, expression analysis may be carried out on one or more different cell types using sets of genes (i.e., gene clusters) previously identified in, e.g., a biological sample analysis experiment such as the described tumor classification methods, to identify concordantly regulated genes that can be used as tissue-specific markers, or to screen for agents that may affect cellular differentiation or other aspects of cellular phenotype. Phenotype association indices can be calculated for normally differentiated tissue samples by calculating a correlation coefficient for a particular normally differentiated tissue sample against, e.g., −fold expression changes or expression differences for a minimum segregation set identified in a cancer analysis, as described above. The −fold expression changes or expression differences for the normally differentiated tissue sample can be calculated with reference to average values of gene x expression across a collection of different normal tissue samples. Expression data derived from the large collections of normal human and mouse tissue samples are available as supplemental data reported by Su, A. I. et al. Large-scale analysis of the human and mouse transcriptomes. PNAS 99: 4465-4470, 2002, incorporated herein by reference, and are available from the publicly accessible website http://expression.gnf.org, incorporated herein by reference.

Three possible outcomes are observed. In the first, no correlation is observed between the minimum segregation set and the normal tissue sample expression data implying that the regulatory pathway represented by the transcript abundance rank order within the minimum segregation set is not active. In the second, a positive correlation is seen between the −fold expression changes or differences in the minimum segregation set and the normal tissue sample implying that the regulatory pathway represented by the transcript abundance rank order within the minimum segregation set is active. In this outcome, the minimum segregation set represents a cluster of genes involved in a differentiation program and/or regulatory pathway that operates in the normal tissue sample and in the tumor cell lines. In the third outcome, a negative correlation is seen between the −fold expression changes or differences in the minimum segregation set and the normal tissue sample implying that the alternative regulatory pathway to one represented by the transcript abundance rank order within the minimum segregation set is active. In this outcome, the minimum segregation set represents a cluster of genes co-regulated in a differentiation program and/or regulatory pathway that operates in the normal tissue samples but that has failed in the tumor cell lines. Because the expression rank order of the genes within the minimum segregation class was derived from a comparison of the fold expression changes in tumor cell lines versus normal epithelial cells of the organ of cancer origin, this scenario may serve as an indicator of an active tumor suppression pathway.

Gene Expression Profiles of Human Normal Prostate Epithelial Cells and Prostate Cancer Cell Lines in Culture

To identify genes expression of which is consistently altered in human prostate cancer cell lines, we searched for genes whose differential expression is retained as cells diverge through mutation, genomic instability, and possibly epigenetic mechanisms during repeated cycles of in vivo prostate cancer growth and progression in nude mice. To model this behavior, cell lines established from LNCap- and PC3-derived human prostate carcinoma xenografts were studied. Parental LNCap and PC3 cell lines represent divergent clinically relevant prostate cancer progression variants. LNCap is a relatively less aggressive, androgen-dependent cell line with wild-type p53, and PC3 is an aggressive, p53 mutated (21), and androgen independent cell line. The five cell lines, LNCapLN3, LNCapPro5, PC3M, PC3MLN4, PC3MPro4 (Pettaway, C. A., Pathak, S., Greene, G., Ramirez, E., Wilson, M. R., Killion, J. J. and Fidler, I. J. Selection of highly metastatic variants of different human prostatic carcinomas using orthotopic implantation in nude mice. Clin Cancer Res. 1996;2:1627-36, incorporated herein by reference) represent lineages that have been derived from xenografts passaged repeatedly in the mouse to model prostate cancer growth and metastatic progression (see Table 1 and accompanying legend). The number of successive in vivo progression and in vitro expansion cycles varied from 1 to 5 in different lineages (Table 1).

The model design was based on the following considerations. Genes regulated similarly in five lineages would be expected to biased towards those genes that are relatively insensitive to the individual genetic differences in the cell's in vitro regulatory program. Furthermore, genes that are sensitive to environmental perturbations may be a source of changes that are stress-induced or are handling artifacts. This consideration also is relevant for changes associated with surgically-derived samples isolated from patients. We chose the early response to serum starvation (two hours) as a convenient method to identify and remove genes that are sensitive to environmental perturbations. Following these criteria, we identified 214 transcripts that are differentially expressed in the same direction in all five prostate cancer cell lines, relative to normal prostate epithelium (NPE), regardless of the presence or absence of serum (vs. 292 observed using data from high serum alone). 43 of these genes were consistently up-regulated and 171 were consistently down-regulated at least two-fold in all five cancer cell lines relative to NPE.

Of the 78 genes excluded by this experimental condition, only the Id3 protein and two alternatively spliced transcripts from the Id1 gene showed a common differential response to serum withdrawal within all five PC3- and LNCap-derived cell lines. Id1 and Id3 gene products are dominant negative regulators of the HLH transcription factors (Lyden, D., Young, A. Z., Zagzag, D., Yan, W., Gerald, W., O'Reilly, R., Bader, B. L., Hynes, R. O., Zhuang, Y., Manova, K., Benezra, R. Id1 and Id3 are required for neurogenesis, angiogenesis and vascularization of tumor xenografts. Nature 1999;401:670-77, incorporated herein by reference). The remaining 75 genes were differentially regulated with respect to serum withdrawal in ways that depended on the cell type. This is consistent with the view that the serum withdrawal criterion removes genes that are sensitive to both external environmental variables and internal cell line-specific context.

Gene Expression Profiles of PC3-Derived Orthotopic Tumors

To test whether the altered gene expression pattern of 214 genes identified in vitro is maintained in vivo, the common set of differentially expressed genes identified in the five cell lines relative to NPE were compared with genes that were differentially expressed in orthotopic tumors induced in nude mice using donor tumors for the PC3 lineage.

We identified a concordant gene expression profile for two tumors each independently derived from the three cell lines PC3 parental, PC3M, and PC3MLN4. 79% (170 of 214 genes) of the transcripts differentially expressed in five prostate cancer cell lines in vitro were also differentially regulated in the same direction in vivo in all six orthotopic tumors. This gene set is exhaustively authenticated in thirty separate comparisons, which should, theoretically, put their regulation in these systems beyond doubt. Nevertheless, a sample of twelve up- and two down-regulated genes was tested using Q-PCR on an ABI7900 using the vendor's recommended protocols available at http://www.appliedbiosystems.com/support/tutorials/ (incorporated herein by reference). This PCR experiment used a further new batch of RNA from normal human prostate epithelial cell line and PC3M cells and human transcript-specific pairs of PCR primers. For several genes two separate sets of primers were designed and tested. Regulation was confirmed in the correct direction for these 14 genes, although the arrays tended to underestimate the magnitude of the change.

Therefore, the differential expression pattern of many of the prostate cancer-associated transcripts of PC3/LNCap consensus class identified in vitro using cell line concordance and media shift refractivity is retained in vivo in orthotopic human prostate tumors in mice. In the context of present invention, these data suggest that human prostate carcinoma xenografts may serve as a useful source of samples for identification of the reference standard data sets.

In Vivo Versus In Vitro Selection of Human Prostate Cancer-Associated Genes

To determine whether the consensus set of 214 differentially expressed genes identified here is retained in the parental cell lines, the PC3 and LNCaP cell lines that have not been serially passaged through mice were examined by microarray analysis, both in high and low serum. When concordance analysis was performed comparing the consensus list of 214 genes and genes that were differentially regulated relative to NPE in parental PC3 and LNCap cell lines, the majority of the down-regulated transcripts (133 genes; 78%) were similarly down-regulated in all 7 cell lines. However, only a small fraction (10 genes; 23%) of up-regulated transcripts was similarly differentially regulated in both parental cell lines. Thus, when compared with the five tumor-derived cell lines, PC3 and LNCAP parental cell lines have substantially smaller similarity with respect to the up-regulated transcripts, indicating that the transcripts with increased mRNA abundance levels in a set of 214 genes do not reflect in vitro selection. The significant degree of conservation of the consensus set of 214 genes in both xenograft-derived and plastic-maintained series of cancer cell lines supports the notion that plastic maintained cancer cell lines may serve as a useful source of samples for identification of the reference standard data sets.

Comparison with Clinical Human Prostate Tumors

While the genes described here are of undoubted interest as their expression is consistently altered in the multiple mouse model systems of human prostate cancer, it is not possible to say, as yet, whether they are of relevance to human disease. However, the expression levels of the genes in our stable set were analyzed published data from a group of clinical samples (Welsh, J. B., Sapinoso, L. M., Su, A. I., Kern, S. G., Wang-Rodriguez, J., Moskaluk, C. A., Frierson, H. F., Jr., Hampton, G. M. Analysis of gene expression identifies candidate markers and pharmacological targets in prostate cancer. Cancer Res., 61: 5974-5978, 2001, (supplemental data obtained from http://www.gnf.org/cancer/prostate), incorporated herein by reference).

These data must be treated with caution because the human clinical samples are highly heterogeneous, consisting of different amounts of cells of epithelial, stromal, and other origins. Nevertheless, of the genes that could be cross-referenced, 31 out of 41 up-regulated genes (76%) were more highly expressed in the majority of 24 human tumors than in a normal epithelial cell line. 32 of these genes were more highly expressed in the majority of tumors than the average expression found in nine adjacent normal prostate tissue samples. Similarly, 141 of 166 down-regulated genes (88%) were down regulated in tumors relative to normal epithelial cells, and 122 were down-regulated in tumors relative to adjacent normal prostate tissue. The similarity in the altered regulation of many of these genes in clinical tumors is an indication that these genes are relevant to the human disease.

Materials and Methods

Cell Culture.

Cell lines used in this study are described in Table 1. The PC3- and LNCap-derived cell lines were developed by consecutive serial orthotopic implantation, either from metastases to the lymph node (for the LN series), or reimplanted from the prostate (Pro series). This procedure generated cell variants with differing tumorigenicity, frequency and latency of regional lymph node metastasis (Pettaway, C. A., Pathak, S., Greene, G., Ramirez, E., Wilson, M. R., Killion, J. J. and Fidler, I. J. Selection of highly metastatic variants of different human prostatic carcinomas using orthotopic implantation in nude mice. Clin Cancer Res. 1996;2:1627-36, incorporated herein by reference). The LNCaP and PC-3 panels of human prostate carcinoma cell lines of graded metastatic potential were provided by Dr. C. Pettaway (M. D. Anderson Cancer Center, Houston, Tex.) and described earlier (Pettaway, C. A., Pathak, S., Greene, G., Ramirez, E., Wilson, M. R., Killion, J. J. and Fidler, I. J. Selection of highly metastatic variants of different human prostatic carcinomas using orthotopic implantation in nude mice. Clin Cancer Res. 1996;2: 1627-36, incorporated herein by reference). A third progression model is represented by the P69 cell line, an SV40 large T-antigen-immortalized prostate epithelial line, and M12, a metastatic derivative of P69 (Bae, V. L., Jackson-Cook, C. K., Brothman, A. R., Maygarden, S. J., and Ware, J. Tumorugenicity of SV40 T antigen immortalized human prostate epithelial cells: association with decreased epidermal growth factor receptor (EGFR) expression. Int. J. Cancer 1994;58:721-29; Jackson-Cook, C., Bae, V., Edelman W., Brothman, A., and Ware, J. Cytogenetic characterization of the human prostate cancer cell line P69SV40T and its novel tumorigenic sublines M2182 and M15. Cancer Genet. & Cytogenet 1996;87:14-23; Bae, V. L., Jackson-Cook, C. K., Maygarden, S. J., Plymate, S. R., Chen, J., and Ware, J. L. Metastatic subline of an SV40 large T antigen immortalized human prostate epithelial cell line. Prostate 1998;34:275-82, incorporated herein by reference). The P69 cell line and M12 cell line were obtained from Dr. S. Plymate and Dr. J. Ware. Two primary human prostate epithelial and one primary human prostate stromal cell line were obtained from Clonetics/BioWhittaker (San Diego, Calif.) and grown in complete prostate epithelial and stromal growth medium provided by the supplier. Except where noted, other cell lines were grown in RPMI1640 supplemented with 10% fetal bovine serum and gentamycin (Gibco BRL) to 70-80% confluence and subjected to serum starvation as described (14-16), or maintained in fresh complete media, supplemented with 10% FBS.

RNA Extraction

For gene expression analysis, cells were harvested in lysis buffer 2 hrs after the last media change at 70-80% confluence and total RNA or mRNA was extracted using the RNeasy (Qiagen, Chatsworth, Calif.) or FastTract kits (Invitrogen, Carlsbad, Calif.). Cell lines were not split more than 5 times, except where noted.

Orthotopic Xenografts

Orthotopic xenografts of human prostate PC3 cells and sublines (Table 1) were developed by surgical orthotopic implantation as previously described (An, Z., Wang, X., Geller, J., Moossa, A. R., Hoffman, R. M. Surgical orthotopic implantation allows high lung and lymph node metastatic expression of human prostate carcinoma cell line PC-3 in nude mice. Prostate 1998;34:169-74, incorporated herein by reference). Briefly, 2×10⁶ cultured PC3 cells, PC3M cells, or PC3M sublines were injected subcutaneously into male athymic mice, and allowed to develop into firm palpable and visible tumors over the course of 2-4 weeks. Intact tissue was harvested from a single subcutaneous tumor and surgically implanted in the ventral lateral lobes of the prostate gland in a series of six athymic mice per cell line subtype. The mice were examined periodically for suprapubic masses, which appeared for all subline cell types, in the order PC3MLN4>PC3M>>PC3. Tumor-bearing mice were sacrificed by CO₂ inhalation over dry ice and necropsy was carried out in a 2-4° C. cold room. Typically, bilaterally symmetric prostate gland tumors in the shape of greatly distended prostate glands were apparent. Prostate tumor tissue was excised and snap frozen in liquid nitrogen. The elapsed time from sacrifice to snap freezing was <20 min. A systematic gross and microscopic post mortem examination was carried out.

Tissue Processing for mRNA Isolation

Fresh frozen orthotopic tumor was examined by use of hematoxylin and eosin stained frozen sections. Orthotopic tumors of all sublines exhibited similar morphology consisting of sheets of monotonous closely packed tumor cells with little evidence of differentiation interrupted by only occasional zones of largely stromal components, vascular lakes, or lymphocytic infiltrates. Fragments of tumor judged free of these non-epithelial clusters were used for mRNA preparation. Frozen tissue (1-3mm×1-3mm) was submerged in liquid nitrogen in a ceramic mortar and ground to powder. The frozen tissue powder was dissolved and immediately processed for mRNA isolation using a Fast Tract kit for mRNA extraction (Invitrogen, Carlsbad, Calif., see above) according to the manufacturers instructions.

Affymetrix Arrays

The protocol for mRNA quality control and gene expression analysis was that recommended by the array manufacturer, Affymetrix, Inc. (Santa Clara, Calif. http://www.affymetrix.com). In brief, approximately one microgram of mRNA was reverse transcribed with an oligo(dT) primer that has a T7 RNA polymerase promoter at the 5′ end. Second strand synthesis was followed by cRNA production incorporating a biotinylated base. Hybridization to Affymetrix Hu6800 arrays representing 7,129 transcripts or Affymetrix U95Av2 array representing 12,626 transcripts overnight for 16 h was followed by washing and labeling using a fluorescently labeled antibody. The arrays were read and data processed using Affymetrix equipment and software (Lockhart, D. J., Dong, H., Byrne, M. C., Follettie, M. T., Gallo, M. V., Chee, M. S., Mittmann, M., Wang, C., Kobayashi, M., Horton, H. and Brown, E. L. Expression monitoring by hybridization to high-density oligonucleotide arrays [see comments]. Nat. Biotechnol. 1996;14:1675-80, incorporated herein by reference). Detailed protocols for data analysis and documentation of the sensitivity, reproducibility and other aspects of the quantitative microarray analysis using Affymetrix technology have been reported (Lockhart, D. J., Dong, H., Byrne, M. C., Follettie, M. T., Gallo, M. V., Chee, M. S., Mittmann, M., Wang, C., Kobayashi, M., Horton, H. and Brown, E. L. Expression monitoring by hybridization to high-density oligonucleotide arrays [see comments]. Nat. Biotechnol. 1996;14:1675-80, incorporated herein by reference).

To determine the quantitative difference in the mRNA abundance levels between two samples, in each individual sample for each gene the average expression differences were calculated from intensity measurements of perfect match (PM) probes minus corresponding control probes representing a single nucleotide mismatch (MM) oligonucleotides for each gene-specific set of 20 PM/MM pairs of oligonucleotides, after discarding the maximum, the minimum, and any outliers beyond 3 standard deviations (SD) from the average. The averages of pairwise comparisons for each individual gene were made between the samples, and the corresponding expression difference calls (see below) were made with Affymetrix software. Microsoft Access was used for other aspects of data management and storage. For each gene, a matrix-based decision concerning the difference in the mRNA abundance level between two samples was made by the software and reported as a “Difference call” (No change (NC), Increase (I), Decrease (D), Marginal increase (MI), and Marginal decrease (MD)) and the corresponding fold change ratio was calculated. 40-50% of the surveyed genes were called present by the Affymetrix software in these experiments. The concordance analysis of differential gene expression across the data set was performed using Microsoft Access and Affymetrix MicroDB software. For experiments involving study of prostate cancer, three of the normal prostate epithelial (NPE) microarrays are used as controls, and referred to as the NPE expression profile. Thus, when a gene is required to show a 2-fold or greater change relative to NPE, this must occur in all three microarrays, for either positive or negative changes. These stringent criteria exclude genes for which one of the three microarrays is in error. The strategy in this study is based on the idea that expression differences will not be called by chance in the same direction in multiple arrays (see below for statistical justification). Each gene in the final list of the 214 differentially expressed genes was required to be called exclusively as either concordantly up- or down-regulated in 30 separate comparisons (5 prostate cancer cell lines×2 experimental serum conditions×3 NPE controls) or 15 separate comparisons (5 prostate cancer cell lines×1 experimental serum condition×3 NPE controls).

Statistical Analysis and Quality Performance Criteria

We used a stringent analytical approach to test the hypothesis that there are common genes with altered mRNA abundance levels which appear to be significantly associated with the studied phenotypes. The Affymetrix MicroDB and Affymetrix DMT software was used to identify in any given comparison of two chips only genes that are determined to be expressed at statistically significantly different (p<0.05) levels. These transcripts are called as differentially expressed. To be included in our final differentially regulated gene class the given transcript was required to be determined as differentially regulated in the same direction (up or down) at the statistically significant levels (p<0.05) e.g., in 30 independent comparisons (5 experimental cell lines×2 experimental conditions×3 control cell lines). To be recognized as differentially regulated in the orthotopic tumors any given gene of the PC3/LNCap consensus class was required to be determined differentially regulated in the same direction at the statistically significant level (p<0.05) in 18 additional independent comparisons (6 orthotopic tumors×3 controls). Despite that identified set of 214 genes is differentially expressed in described experimental systems with the extremely high level of confidence, we carried out Q-PCR confirmation analysis for a sub-set of identified genes and confirmed their differential expression in all instances using an additional independent normal human prostate epithelial cell line as a control.

Quality performance criteria adopted for the Affymetrix GeneChip system and applied in this study. 40-50% of the surveyed genes were called present by the Affymetrix software in these experiments. This is at the high end of the required standard adopted in many peer-reviewed publications using the same experimental system. Transcripts that are called present by the Affymetrix software in any given experiment were determined to have the signal intensities higher in the perfect match probe sets compared to single-nucleotide mismatch probe sets and background at the statistically significant level. This analysis was performed for each individual transcript using unique set of 20 perfect matches versus 20 single nucleotide mismatch probes. In our final list of 214 genes all transcripts were called present in at least one experimental setting. The inclusion error associated with two mRNA samples from identical cell lines was 2.7% for a difference called by the Affymetrix software. Thus, two independently obtained mRNA from the same cell lines will have 2.7% false positives. When a third independently derived epithelial cell line was included, only 4 genes (0.06%) out of 7,129 were called differentially expressed. The expression profiles of the normal prostate epithelial cell lines used in our experiments were determined to be indistinguishable. Therefore, controls are not likely source of errors in gene expression analysis performed in this study. This is particularly important, since the strategy adopted in this study is based on the idea that expression differences will not be called statistically significant by chance in the same direction in multiple arrays and during multiple independent comparisons of different phenotypes and variable experimental conditions. To impose additional stringent restrictions on possibility of a gene to be detected as concordantly differentially regulated by chance, we apply the use of multiple experimental models and vastly variable experimental settings such as in vitro and in vivo growth and varying growth conditions. Similar strategy for identification of consistent gene expression changes based on a concordant behavior of the differentially regulated genes using Affymetrix GeneChip system and software was applied and validated in several peer-reviewed published papers (see for example, Lee C. K., Klopp, R. G., Weindruch, R., Prolla, T. A. Gene expression profile of aging and its retardation by caloric restriction. Science 1999; 285: 1390-1393; Ishida, S., Huang, E., Zuzan, H., Spang, R., Leone, G., West, M., Nevins, J. R. Role for E2F in control of both DNA replication and mitotic function as revealed from DNA microarray analysis. Mol Cell Biol 2001; 21: 4684-4699, incorporated herein by reference). We applied more stringent criteria in our study requiring a concordance in at least 30 of 30 experiments compared to 6 of 6 comparisons in (Lee C. K., Klopp, R. G., Weindruch, R., Prolla, T. A. Gene expression profile of aging and its retardation by caloric restriction. Science 1999; 285: 1390-1393, incorporated herein by reference); and 4 of 6 comparisons in (Ishida, S., Huang, E., Zuzan, H., Spang, R., Leone, G., West, M., Nevins, J. R. Role for E2F in control of both DNA replication and mitotic function as revealed from DNA microarray analysis. Mol Cell Biol 2001; 21:4684-4699, incorporated herein by reference). Ishida, et al. (Ishida, S., Huang, E., Zuzan, H., Spang, R., Leone, G., West, M., Nevins, J. R. Role for E2F in control of both DNA replication and mitotic function as revealed from DNA microarray analysis. Mol Cell Biol 2001; 21: 4684-4699, incorporated herein by reference) provided a formal statistical justification that four or more concordant calls out of six comparisons cannot be explained by chance, with the probability in the range of 1 in 10^{4.}

Q-PCR confirmation analysis of the differentially regulated genes To confirm differential regulation of the transcripts comprising a PC3/LNCap-consensus class using an independent method a sample of 14 genes (12 up-regulated and 2 down-regulated) was tested using Q-PCR on an ABI7900 according to the vendor's recommended protocols (available at http://www.appliedbiosystems.com/support/tutorials/). This PCR experiment used a further new batch of RNA from a third normal human prostate epithelial cell line and human transcript-specific pairs of PCR primers.

### EXAMPLE 1

### Classification of Human Prostate Tumors

A. General

A first reference set for human prostate tumors was obtained by obtaining gene expression data from five prostate cancer cell lines (cell lines used were LNCapLN3; LNCapPro5; PC3M; PC3MLN4; PC3Mpro4; see Table 1) and two different normal human prostate epithelial cell lines were obtained from Clonetics/BioWhittaker (San Diego, Calif.) and grown in complete prostate epithelial growth medium provided by the supplier. An original and a replicate data set was obtained for the first normal cell line, and the second cell line represented an independent data set from an independent epithelial cell line. Each of the tumor cell lines was derived from aggressively metastatic human prostate tumors. Consequently, we expected that these tumor cell lines should have an “invasive” phenotype because had they not been “invasive,” they would not have penetrated the prostate capsule, a step pre-requisite to metastasis.

The expression data were obtained using an Affymetrix Human Genome-U95Av2 (“HG-U95Av2”) expression array chip (Affymetrix, Santa Clara, Calif.). The HG-U95Av2 Array represents approximately 10,000 full-length genes. Data were obtained from the HG-U95Av2 according to the manufacturer's suggested protocols, as outlined in the Materials & Methods Section above

The original data set thus comprised a total of eight separate sets of gene expression data, five from the set of tumor cell lines and three from the set of epithelial cell lines. Fifteen separate pairwise comparisons were carried out to identify a first reference set of genes that were differentially expressed in the tumor cell lines and the epithelial cell lines. Differential expression was determined using Affymetrix's Microarray Suite software (versions 4.0 and 5.0). To be included in the first reference set, a candidate gene needed to meet two criteria: 1) the candidate gene was shown to be differentially expressed in each of the 15 pairwise comparisons; and 2) the direction of the differential (i.e. greater expression in the tumor cell lines cf. the epithelial cell lines or vice-versa) was consistent in each of the 15 pairwise comparisons. The first reference set comprised of 629 genes.

B. Recurrence Predictor Cluster and Sample Classification

The methods of the invention were used to identify gene clusters associated with increased likelihood of tumor recurrence. A second reference set was obtained using expression data obtained from clinical human prostate tumor samples. These data were the supplemental data reported in Singh, D., Febbo, P. G., et al., “Gene Expression Correlates of Clinical Prostate Cancer Behavior,” Cancer Cell Mar. 2002 1:203-209, incorporated herein by reference. The clinical human prostate tumor samples were divided into two groups, recurrent and non-recurrent, as reported in Singh, et al. (2002). Data from twenty-one patients were evaluable with respect to recurrence following surgery. Recurrence was defined as two successive PSA values >0.2ng/ml. Of the twenty-one patients, eight had recurrences, and thirteen patients remained relapse-free for at least four years.

Affymetrix MicroDB (version 3.0) and Affymetrix Data Mining Tools (DMT) (version 3.0) data analysis software were used to identify genes that were differentially regulated in recurrence group compared to relapse-free group of patients at the statistically significant level (p<0.05; Student T-test). Candidate genes were included in the second reference set if they were identified by the DMT software as having p values of 0.05 or less both for up-regulated and down-regulated genes. 316 genes were identified as being members of the second reference set.

A concordance set of genes was identified from the first and second reference sets. Genes were included in the concordance set if they met the following criteria: 1) the gene was identified as a member of both the first and the second reference sets; and 2) the direction of the differential was consistent in the first and the second reference sets (i.e., the gene transcript was more abundant in the tumor cell lines cf. the control cell lines and more abundant in the recurrent cf. the non-recurrent samples, or the gene transcript was less abundant in the tumor cell lines cf. the control cell lines and less abundant in the recurrent cf. the non-recurrent samples). The first criterion provides a way of minimizing the number of genes for which the pairwise comparisons are carried out for the sample data. Only those genes that are members of the first reference set need to be compared for generating the second reference set because the first criterion requires that the candidate gene be a member of both the first and second reference sets. The concordance set comprises of 19 genes.

The minimum segregation set was obtained as follows. For each gene in the concordance set, the −fold expression changes (as determined by the ratio of the relative transcript abundance levels) was determined. This was done for the cell line data by computing for each gene in the concordance set the ratio of the average expression in the tumor cell lines to the average expression in the control cell lines, and similarly the ratio of the average expression in the samples obtained from patients who relapsed (recurrent population) from those who did not relapse (non-recurrent population). Using the notation described above, this corresponds to calculating <expression>₁/<expression>₂ for the cell line and clinical samples data. For the cell line data, <expression>₁ corresponds to the average expression value for gene x over all tumor cell lines and <expression>₂ corresponds to the average expression value for gene x over all control cell lines. For the clinical sample data, <expression>₁ corresponds to the average expression value for gene x over all samples from patients who relapsed and <expression>₂ corresponds to the average expression value for gene x over all samples from patients who did not relapse.

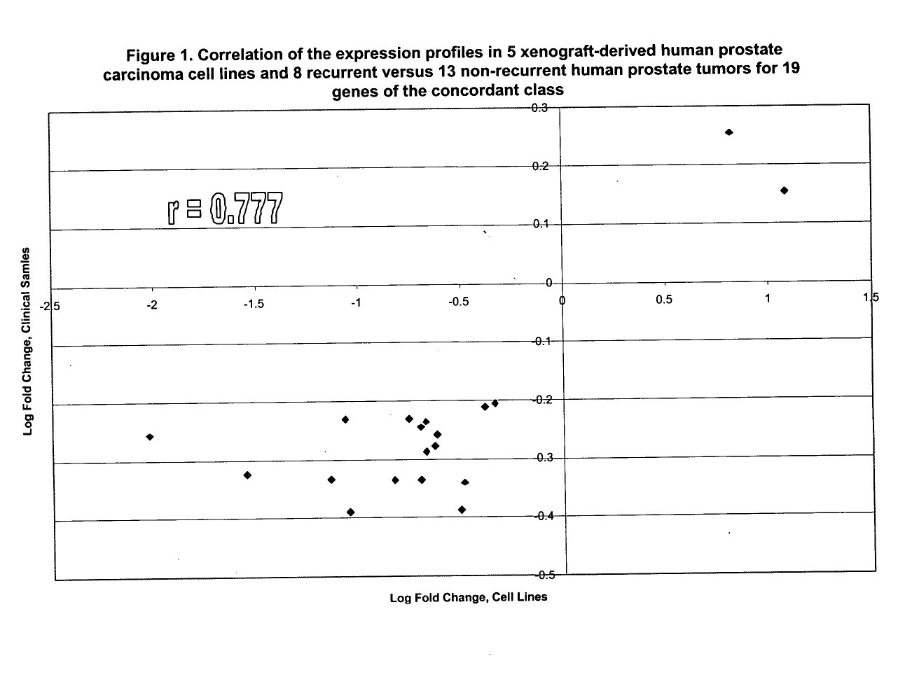

The −fold expression change data were log₁₀ transformed and the transformed data were entered as two arrays in a Microsoft Excel spreadsheet. The Excel CORREL function was used to generate a correlation coefficient that characterizes the degree to which the concordance set −fold expression changes were correlated between the cell line and clinical sample data. Typically, we observe correlation coefficients at this stage of the analysis in the range of about 0.7 to about 0.9. A scatter plot showing the relationship between the log-transformed −fold expression changes in the cell line and clinical sample data is shown in FIG. 1. In the scatter plot, each point represents an individual gene belonging to the concordance set. The correlation coefficient for this concordance set was 0.777.

A minimum segregation set was selected from the concordance set. This set was chosen by looking at the scatter plot (FIG. 1) and manually selecting sub-sets of genes within the concordance set whose representative points fell closest to an imaginary regression line drawn through the data. Of course, this procedure can be automated. A second correlation coefficient was calculated using the Microsoft Excel CORREL function for several sub-sets of genes within the concordance set to arrive at a highly-correlated sub-set. These genes are members of the minimum segregation set, and represent genes whose −fold expression changes are most highly correlated between the cell line and clinical sample data. Typically, we identified minimum segregation sets that comprised on the order of from about 3 to about 20 genes and that produced correlation coefficients on the order of ≧0.98.

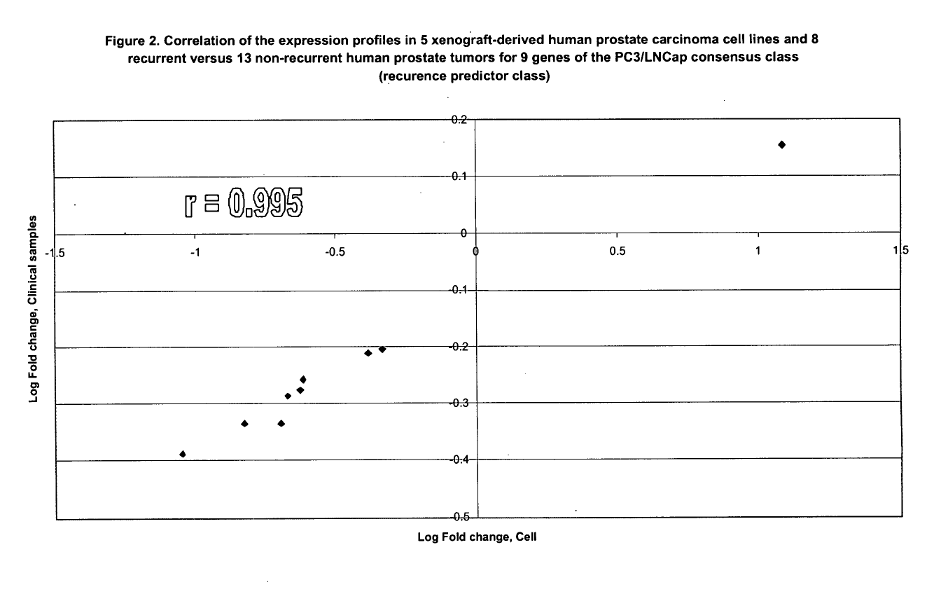

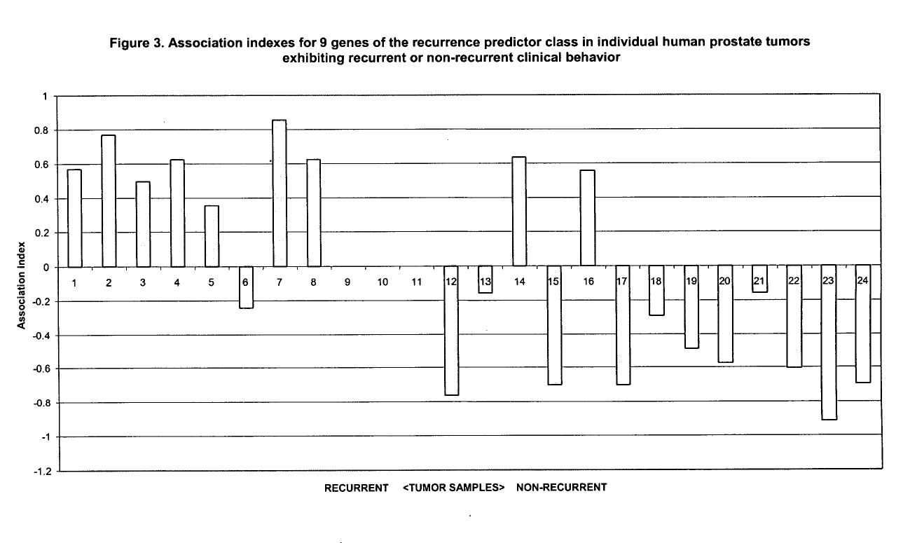

Using this method, a total of nine genes was selected for the recurrence predictor minimum segregation set. This recurrence predictor minimum segregation set had a correlation coefficient of 0.995 for the cell line and sample −fold expression change differences. See FIG. 2. Members of this recurrence predictor minimum segregation set are shown in Table 5.

TABLE 5
Prostate Tumor Recurrence
Predictor Minimum Segregation Set.

| | LocusLink | |
|---|---|---|
| Affymetrix Probe Set ID | Identifier¹ | Description² |
| 41435_at | 8541 | PPFIA3: protein tyrosine |
| | | phosphatase, receptor type, |
| | | f polypeptide (PTPRF), |
| | | interacting protein (liprin), |
| | | alpha 3 |
| 33228_g_at | 3588 | IL10RB: interleukin 10 |
| | | receptor, beta |
| 40522_at | 2752 | GLUL: glutamate- |
| | | ammonia ligase (glutamine |
| | | synthase) |
| 37026_at | 1316 | COPEB: core promoter |
| | | element binding protein |
| 33436_at | 6662 | SOX9: SRY (sex |
| | | determining region Y)-box |
| | | 9 (campomelic dysplasia, |
| | | autosomal sex-reversal) |
| 39631_at | 2013 | EMP2: epithelial |
| | | membrane protein 2 |
| 1915_s_at | 2353 | FOS: v-fos FBJ murine |
| | | osteosarcoma viral |
| | | oncogene homolog |
| 37286_at | 3726 | JUNB: jun B proto- |
| | | oncogene |
| 40448_at | 7538 | ZFP36: zinc finger protein |
| | | 36, C3H type, homolog |
| | | (mouse) |
| ¹LocusLink provides a single query interface to curated sequence and descriptive information about genetic loci. It presents information on official nomenclature, aliases, sequence accessions, phenotypes, EC numbers, MIM numbers, UniGene clusters, homology, map locations, and related web sites. It may be accessed through the National Center for Biotechnology Information (NCBI) website at http://www.ncbi.nlm.nih.gov/LocusLink/. | | |
| ²The first entry in each cell of this column corresponds to the HUGO Gene Nomenclature Committee (“HGNC”) Approved Symbol for the gene corresponding to the Affymetrix Probe Set and LocusLink Identifiers within the same row. Information for the subject gene, associated cDNA, mRNA, and protein | | |
| #sequences may be obtained using the LocusLink identifier or the HGNC Approved Symbol by querying the search page at http://www.ncbi.nlm.nih.gov/LocusLink. Note, the footnotes associated with Table 5 apply to every table in this specification that follows the same or similar format as Table 3 (i.e., column 1 | | |
| #contains information on the Affymetrix Probe Set ID, column 2 contains the LocusLink Identifier, and column 3 contains the gene description. | | |

The recurrence predictor minimum segregation set was used to calculate a phenotype association indices for each of the twenty-one tumors removed from the patients described in Singh, et al. (2002) that were evaluated for recurrence. The phenotype association index was obtained by calculating for each individual tumor sample, the −fold expression change for each of the nine genes in the recurrence predictor minimum segregation set. The −fold expression change was calculated as:

expression/<expression₁+expression₂>

where “expression” is the observed expression level for gene x for the individual tumor, and “<expression₁+expression₂>” is the average gene expression level for gene x across the set of 21 tumors used to generate the recurrence predictor minimum segregation set. The −fold expression changes for these nine genes were log₁₀ transformed, the transformed data entered as an array in a Microsoft Excel spreadsheet, and the Excel CORREL function was used to generate a correlation coefficient between the individual tumor data array and the corresponding log₁₀ transformed data for the average −fold expression changes in the cell lines for the same nine genes (i.e., log₁₀(<expression>₁/<expression>₂). This second correlation coefficient is the phenotype association index. The phenotype association index has the surprising and unexpected property of allowing the samples to be classified according to the sign of the index. FIG. 3 shows the phenotype association index for each of the twenty-one tumors classified using the recurrence predictor minimum segregation class described above. 7 out of 8 tumors associated with recurrences had positive association indices, while 11 out of 13 tumors associated with no recurrence had negative association indices. Thus, the method correctly classified 18/21 or 86% of the tumors.

B-1. Prostate Cancer Predictor Clusters and Sample Classification

The methods of the invention were used to identify gene clusters associated with the presence of prostate carcinoma cells in a tissue sample compared to the adjacent normal tissue samples that were determined to be cancer cell free. The first reference data set was derived as described above in A. A second reference set was obtained using expression data obtained from clinical human prostate tumor samples. These data were two independent sets of the supplemental data reported in Welsh, J. B., et al., “Analysis of gene expression identifies candidate markers and pharmacological targets in prostate cancer,” Cancer Research, 2001, 61: 5974-5978; and Singh, D., Febbo, P. G., et al., “Gene Expression Correlates of Clinical Prostate Cancer Behavior,” Cancer Cell March 2002 1:203-209, incorporated herein by reference. The clinical human prostate tumor samples were divided into two groups, cancer samples and adjacent normal tissue samples, as reported in Welsh, et al. (2001). Data from twenty-five cancer samples (analysis of one tumor samples was carried out in duplicate) and nine adjacent normal tissue samples were used to identify the concordance gene set with high correlation coefficient and significant sample segregation power thus comprising genes with the properties of the minimum segregation class.

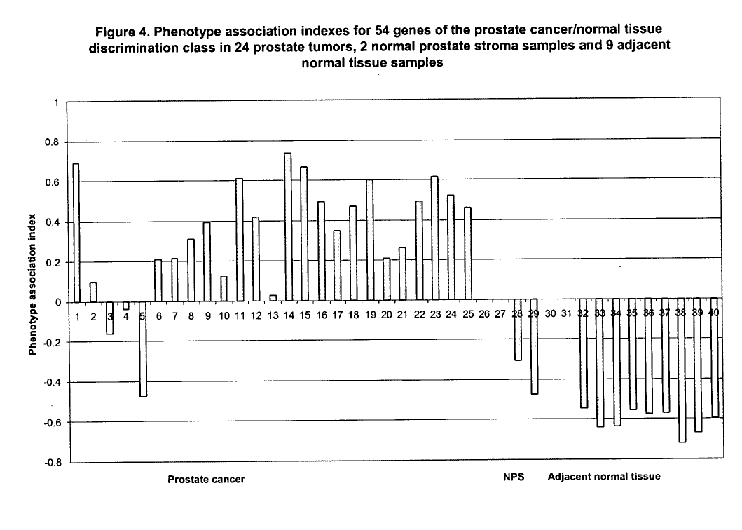

Genes were included in the concordance set if the direction of the differential was consistent in the first reference set and in the clinical samples (i.e., the gene transcript was more abundant in the tumor cell lines cf. the control cell lines and more abundant in the cancer samples cf. the adjacent normal tissue (ANT) samples, or the gene transcript was less abundant in the tumor cell lines cf. the control cell lines and less abundant in the cancer samples cf. the ANT samples. The concordance set comprising 54 genes was identified with correlation coefficient 0.823. Members of this concordance set are shown in Table 6. When applied to individual clinical samples, this gene set yielded sample segregation power of 91%. 30 of 33 clinical samples were classified correctly; 9 of 9 ANT samples displayed negative phenotype association indices while 21 of 24 cancer samples had positive phenotype association indices (FIG. 4).

TABLE 6
54 genes of the prostate cancer/normal tissue concordant set.

| | Affymetrix | | | |
|---|---|---|---|---|
| Affymetrix Probe | Probe Set ID | UniGene | LocusLink | |
| Set ID (HuFL6800) | (U95Av2) | Identifier | Identifier | Description |
| U03735_f_at | 34575_f_at | Hs. 36978 | MAGEA3 | MAGE-3 antigen |
| | | | | (MAGE-3) gene |
| L77701_at | 40427_at | Hs. 16297 | COX17 | COX17 mRNA |
| X70940_s_at | 35175_f_at | Hs. 2642 | EEF1A2 | mRNA for elongation |
| | | | | factor 1 alpha-2 |
| U33053_at | 175_s_at | Hs. 2499 | PRKCL1 | lipid-activated protein |
| | | | | kinase PRK1 mRNA |
| L18920_f_at | 34575_f_at | Hs. 36980 | MAGEA2 | MAGE-2 gene exons 1-4 |
| M77140_at | 35879_at | Hs. 1907 | GAL | pro-galanin mRNA |
| X92896_at | 40891_f_at | Hs. 18212 | DXS9879E | mRNA for ITBA2 |
| | | | | protein |
| L18877_f_at | 34575_f_at | Hs. 169246 | MAGEA12 | MAGE-12 protein gene |
| M77481_mal_f_at | 36302_f_at | Hs. 72879 | MAGEA12 | antigen (MAGE-1) |
| | | | | gene |
| U77413_at | 38614_s_at | Hs. 100293 | OGT | O-linked GlcNAc |
| | | | | transferase mRNA |
| U73514_at | 40778_at | Hs. 171280 | HADH2 | short-chain alcohol |
| | | | | dehydrogenase |
| | | | | (XH98G2) mRNA |
| U39840_at | 37141_at | Hs. 299867 | HNF3A | hepatocyte nuclear |
| | | | | factor-3 alpha (HNF-3 |
| | | | | alpha) mRNA |
| L41559_at | 34352_at | Hs. 3192 | PCBD | pterin-4a- |
| | | | | carbinolamine |
| | | | | dehydratase (PCBD) |
| | | | | mRNA |
| U90907_at | 37961_at | Hs. 88051 | PIK3R3 | clone 23907 mRNA |
| | | | | sequence |
| D00860_at | 36489_at | Hs. 56 | PRPS1 | mRNA for |
| | | | | phosphoribosyl |
| | | | | pyrophosphate |
| | | | | synthetase (EC 2.7.6.1) |
| | | | | subunit I |
| U81599_at | 40327_at | Hs. 66731 | HOXB13 | homeodomain protein |
| | | | | HOXB13 mRNA |
| M80254_at | 40840_at | Hs. 173125 | PPIF | cyclophilin isoform |
| | | | | (hCyP3) mRNA |
| HG1612-HT1612_at | 36174_at | Hs. 75061 | MACMARCKS | Macmarcks |
| D85131_s_at | 1764_s_at | Hs. 7647 | MAZ | mRNA for Myc- |
| | | | | associated zinc-finger |
| | | | | protein ofislet |
| U79274_at | 31838_at | Hs. 150555 | HSU79274 | clone 23733 mRNA |
| Z22548_at | 39729_at | Hs. 146354 | PRDX2 | thiol-specific |
| | | | | antioxidant protein |
| | | | | mRNA |
| HG4312- | 36188_at | Hs. 75113 | GTF3A | Transcription Factor |
| HT4582_s_at | | | | IIIa |
| J04444_at | 1160_at | Hs. 289271 | CYC1 | cytochrome c-1 gene |
| X79865_at | 39812_at | Hs. 109059 | MRPL12 | Mrp17 mRNA |
| U37022_rnal_at | 1942_s_at | Hs. 95577 | CDK4 | cyclin-dependent |
| | | | | kinase 4 (CDK4) gene |
| U07424_at | 34291_at | Hs. 23111 | FARSL | putative tRNA |
| | | | | synthetase-like protein |
| | | | | mRNA |
| U79287_at | 40955_at | Hs. 19555 | PTOV1 | clone 23867 mRNA |
| | | | | sequence |
| M34338_s_at | 241_g_at | Hs. 76244 | SRM | spermidine synthase |
| | | | | mRNA |
| L37936_at | 39659_at | Hs. 340959 | TSFM | nuclear-encoded |
| | | | | mitochondrial |
| | | | | elongation factor Ts |
| | | | | (EF-Ts) mRNA |
| X07979_at | 32808_at | Hs. 287797 | ITGB1 | mRNA for fibronectin |
| | | | | receptor beta subunit |
| X54232_at | 33929_at | Hs. 2699 | GPC1 | mRNA for heparan |
| | | | | sulfate proteaglycan |
| | | | | (glypican) |
| M55210_at | 232_at | Hs. 214982 | LAMC1 | laminin B2 chain |
| | | | | (LAMB2) gene |
| S74017_at | 853_at | Hs. 155396 | NFE2L2 | Nrf2 = NF-E2-like basic |
| | | | | leucine zipper |
| | | | | transcriptional activator |
| | | | | [human |
| U90913_at | 39416_at | Hs. 12956 | TIP-1 | clone 23665 mRNA |
| | | | | sequence |
| X52425_at | 404_at | Hs. 75545 | IL4R | IL-4-R mRNA for the |
| | | | | interleukin 4 receptor |
| U90878_at | 36937_s_at | Hs. 75807 | PDLIM1 | LIM domain protein |
| | | | | CLP-36 mRNA |
| X86163_at | 39310_at | Hs. 250882 | BDKRB2 | mRNA for B2- |
| | | | | bradykinin receptor |
| U73377_at | 38118_at | Hs. 81972 | SHC1 | p66shc (SHC) mRNA |
| Z29083_at | 368_at | Hs. 82128 | TPBG | 5T4 gene for 5T4 |
| | | | | Oncofetal antigen |
| M31013_at | 39738_at | Hs. 146550 | MYH9 | nonmuscle myosin |
| | | | | heavy chain (NMHC) |
| | | | | mRNA |
| M77349_at | 1385_at | Hs. 118787 | TGFBI | transforming growth |
| | | | | factor-beta induced |
| | | | | gene product (BIGH3) |
| | | | | mRNA |
| U04636_rnal_at | 1069_at | Hs. 196384 | PTGS2 | cyclooxygenase-2 |
| | | | | (hCox-2) gene |
| X15414_at | 36589_at | Hs. 75313 | AKR1B1 | mRNA for aldose |
| | | | | reductase (EC 1.1.1.2) |
| M65292_s_at | 32249_at | Hs. 278568 | HFL1 | factor H homologue |
| | | | | mRNA |
| X07438_s_at | 38634_at | Hs. 101850 | RBP1 | DNA for cellular |
| | | | | retinol binding protein |
| | | | | (CRBP) exons 3 and 4/ |
| | | | | gb = X07438/ |
| | | | | ntype = DNA/ |
| | | | | annot = exon |
| X79882_at | 38064_at | Hs. 80680 | MVP | lrp mRNA |
| M11433_at | 38634_at | Hs. 101850 | RBP1 | cellular retinol-binding |
| | | | | protein mRNA |
| U60060_at | 37743_at | Hs. 79226 | FEZ1 | FEZ1 mRNA |
| X04412_at | 32612_at | Hs. 290070 | GSN | mRNA for plasma |
| | | | | gelsolin |
| X93510_at | 32610_at | Hs. 79691 | RIL | mRNA for 37 kDa LIM |
| | | | | domain protein |
| M12125_at | 32313_at | Hs. 300772 | TPM2 | fibroblast muscle-type |
| | | | | tropomyosin mRNA |
| L13210_at | 37754_at | Hs. 79339 | LGALS3BP | Mac-2 binding protein |
| | | | | mRNA |
| M21186_at | 35807_at | Hs. 68877 | CYBA | neutrophil cytochrome |
| | | | | b light chain p22 |
| | | | | phagocyte b- |
| | | | | cytochrome mRNA |
| L13720_at | 1598_g_at | Hs. 78501 | GAS6 | growth-arrest-specific |
| | | | | protein (gas) mRNA |

The minimum segregation set was obtained as follows. For each gene in the concordance set, the −fold expression changes (as determined by the ratio of the relative transcript abundance levels) was determined. This was done for the cell line data by computing for each gene in the concordance set the ratio of the average expression in the tumor cell lines to the average expression in the control cell lines, and similarly the ratio of the average expression values in the samples obtained from cancer samples (malignant population) from those from ANT samples (non-malignant population). Using the notation described above, this corresponds to calculating <expression>₁/<expression>₂ for the cell line and clinical samples data. For the cell line data, <expression>₁ corresponds to the average expression value for gene x over all tumor cell lines and <expression>₂ corresponds to the average expression value for gene x over all control cell lines. For the clinical sample data, <expression>₁ corresponds to the average expression value for gene x over all cancer samples and <expression>₂ corresponds to the average expression value for gene x over all ANT samples.

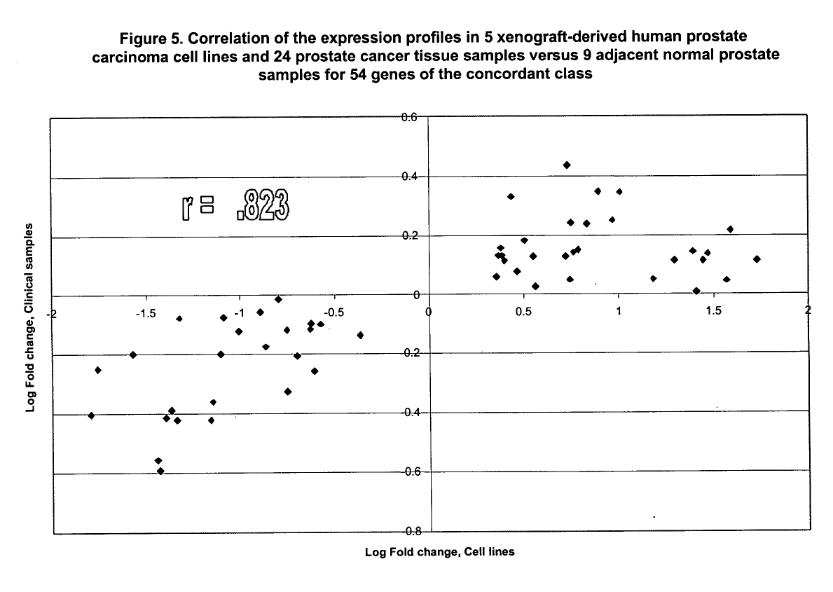

The −fold expression change data were log₁₀ transformed and the transformed data were entered as two arrays in a Microsoft Excel spreadsheet. The Excel CORREL function was used to generate a correlation coefficient that characterizes the degree to which the concordance set −fold expression changes were correlated between the cell line and clinical sample data. Typically, we observe correlation coefficients at this stage of the analysis in the range of about 0.7 to about 0.9. A scatter plot showing the relationship between the log-transformed −fold expression changes in the cell line and clinical samples data for the 54 genes of a concordance set is shown in FIG. 5. In the scatter plot, each point represents an individual gene belonging to the concordance set. The correlation coefficient for this concordance set was 0.823.

A minimum segregation set was selected from the concordance set. This set was chosen by looking at the scatter plot (FIG. 5) and manually selecting sub-sets of genes within the concordance set whose representative points fell closest to an imaginary regression line drawn through the data. Of course, this procedure can be automated. A second correlation coefficient was calculated using the Microsoft Excel CORREL function for several sub-sets of genes within the concordance set to arrive at a highly-correlated sub-set. These genes are members of the minimum segregation cluster, and represent genes whose −fold expression changes are most highly correlated between the cell line and clinical sample data. Typically, we identified minimum segregation clusters that comprised on the order of from about 3 to about 20 genes and that produced correlation coefficients on the order of ≧0.98.

Using this method, a total of ten genes were selected for the prostate cancer/normal tissue predictor minimum segregation set 1 (i.e. cluster 1) and a total of five genes was selected for the prostate cancer/normal tissue minimum segregation set 2 (i.e., cluster 2). These prostate cancer predictor minimum segregation clusters had a correlation coefficient of 0.995 (cluster 1) and 0.997 (cluster 2) for the cell line and sample −fold expression change differences. Members of these two prostate cancer minimum segregation clusters are shown in Table 7.

TABLE 7
The genes comprising prostate cancer minimum segregation set 1 (cluster
1) (ten genes) and minimum segregation set 2 (cluster 2) (five genes).

| | | | |
|---|---|---|---|
| Affymetrix | | | |
| Probe Set | Affymetrix | | |
| ID | Probe Set ID | | Short |
| (U95Av2) | (HuFL6800) | Description | Description |

10 genes (r = 0.99)

| | | | |
|---|---|---|---|
| 1160_at | J04444_at | J04444/FEATURE = cds/ | cytochrome c-1 |
| | | DEFINITION = HUMCYC1A Human | |
| | | cytochrome c-1 gene, complete cds. | |
| 38614_s_at | U77413_at | Cluster Incl. U77413: Human O-linked | O-linked GlcNAc |
| | | GlcNAc transferase mRNA, complete | transferase |
| | | cds/cds = (265,3027)/gb = U77413/ | |
| | | gi = 2266993/ug = Hs. 100293/len = 3084 | |
| 37141_at | U39840_at | Cluster Incl. U39840: Human hepatocyte | hepatocyte |
| | | nuclear factor-3 alpha (HNF-3 alpha) | nuclear factor-3 |
| | | mRNA, complete cds/cds = (87,1508)/ | alpha (HNF-3 |
| | | gb = U39840/gi = 1066121/ | alpha) |
| | | ug = Hs. 105440/len = 2872 | |
| 34352_at | L41559_at | Cluster Incl. AA631698: np79a08.s1 | dimerization |
| | | Homo sapiens cDNA/clone = IMAGE- | cofactor of |
| | | 1132502/gb = AA631698/gi = 2554309/ | hepatocyte |
| | | ug = Hs. 3192/len = 640 | nuclear factor 1 |
| | | | alpha (TCF1) |
| 40327_at | U81599_at | Cluster Incl. U57052: Human Hoxb-13 | homeodomain |
| | | mRNA, complete cds/cds = (54,908)/ | protein HOXB13 |
| | | gb = U57052/gi = 1519039/ | |
| | | ug = Hs. 66731/len = 1026 | |
| 39729_at | Z22548_at | Cluster Incl. L19185: Human natural | peroxiredoxin 2 |
| | | killer cell enhancing factor (NKEFB) | |
| | | mRNA, complete cds/cds = (124,720)/ | |
| | | gb = L19185/gi = 440307/ug = Hs. 146354/ | |
| | | len = 980 | |
| 34291_at | U07424_at | Cluster Incl. U07424: Human putative | phenylalanine- |
| | | tRNA synthetase-like protein mRNA, | tRNA synthetase- |
| | | complete cds/cds = (12,1538)/ | like |
| | | gb = U07424/gi = 2098578/ | |
| | | ug = Hs. 23111/len = 1807 | |
| 36937_s_at | U90878_at | Cluster Incl. U90878: Homo sapiens | carboxy terminal |
| | | carboxyl terminal LIM domain protein | LIM domain |
| | | (CLIM1) mRNA, complete cds/ | protein 1 |
| | | cds = (142,1131)/gb = U90878/ | |
| | | gi = 2957144/ug = Hs. 75807/len = 1480 | |
| 38634_at | X07438_s_at | Cluster Incl. M11433: Human cellular | cellular retinol |
| | | retinol-binding protein mRNA, | binding protein |
| | | complete cds/cds = (125,532)/ | (CRBP) |
| | | gb = M11433/gi = 190947/ | |
| | | ug = Hs. 101850/len = 716 | |
| 32313_at | M12125_at | Cluster Incl. M12125: Human fibroblast | tropomyosin 2 |
| | | muscle-type tropomyosin mRNA, | (beta) |
| | | complete cds/cds = (118,972)/ | |
| | | gb = M12125/gi = 339951/ | |
| | | ug = Hs. 180266/len = 1044 | |

5 genes (r = 0.998)

| | | | |
|---|---|---|---|
| 36174_at | HG1612- | Cluster Incl. X70326: H. sapiens | Macmarcks |
| | HT1612_at | MacMarcks mRNA/cds = (13,600)/ | |
| | | gb = X70326/gi = 38434/ug = Hs. 75061/ | |
| | | len = 1334 | |
| 39812_at | X79865_at | Cluster Incl. X79865: H. sapiens Mrp17 | ribosomal protein, |
| | | mRNA/cds = (137,733)/gb = X79865/ | mitochondrial, |
| | | gi = 1313961/ug = Hs. 109059/len = 1008 | L12 |
| 39310_at | X86163_at | Cluster Incl. X86163: H. sapiens mRNA | bradykinin |
| | | for B2-bradykinin receptor, 3/ | receptor B2 |
| | | cds = (0,41)/gb = X86163/gi = 1220163/ | |
| | | ug = Hs. 239809/len = 2582 | |
| 38634_at | M11433_at | Cluster Incl. M11433: Human cellular | retinol-binding |
| | | retinol-binding protein mRNA, | protein 1, cellular |
| | | complete cds/cds = (125,532)/ | |
| | | gb = M11433/gi = 190947/ | |
| | | ug = Hs. 101850/len = 716 | |
| 37743_at | U60060_at | Cluster Incl. U60060: Human FEZ1 | fasciculation and |
| | | mRNA, complete cds/cds = (99,1277)/ | elongation protein |
| | | gb = U60060/gi = 1927201/ | zeta 1 (zygin I) |
| | | ug = Hs. 79226/len = 1619 | |

The prostate cancer/normal tissue minimum segregation clusters were used to calculate phenotype association indices for each of the thirty-three samples from the patients described in Welsh, et al. (2001). The phenotype association index was obtained by calculating for each individual clinical sample, the −fold expression change for each of the ten and five genes in the prostate cancer predictor minimum segregation set 1 and 2. The −fold expression change was calculated as:

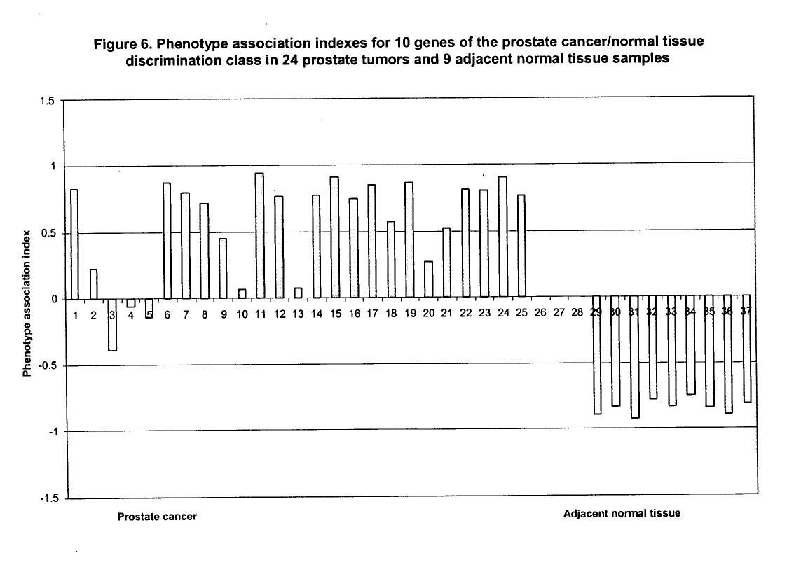

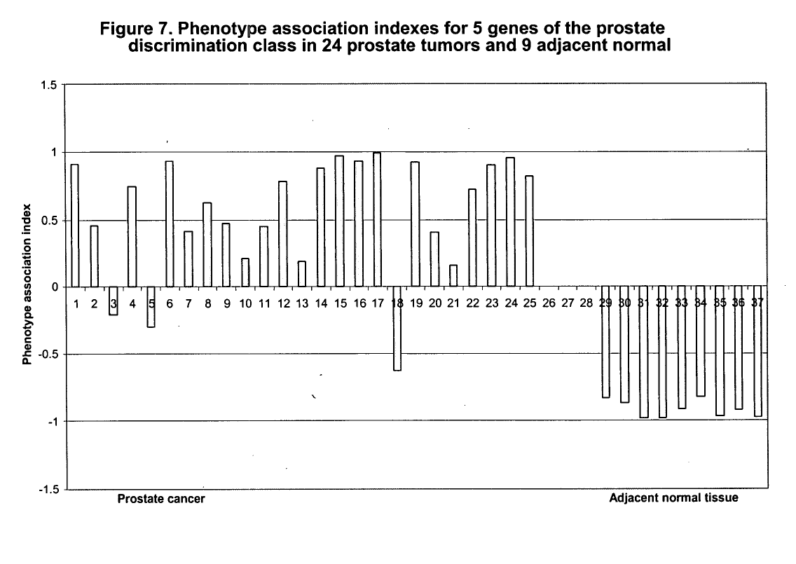

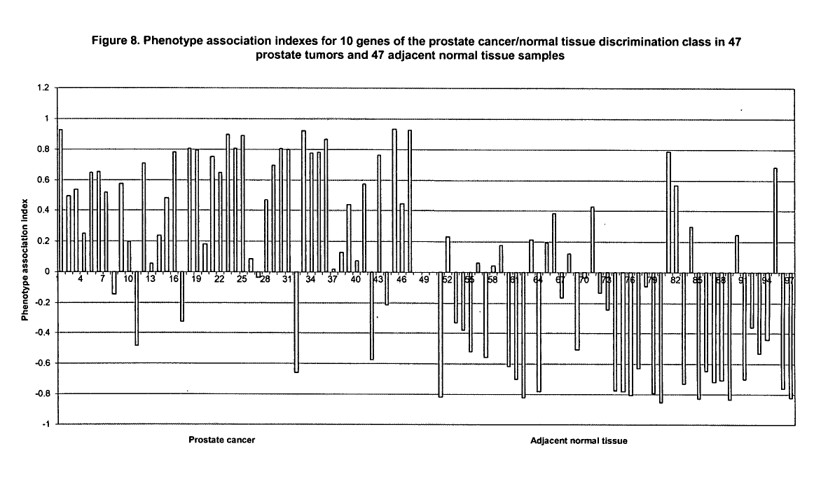

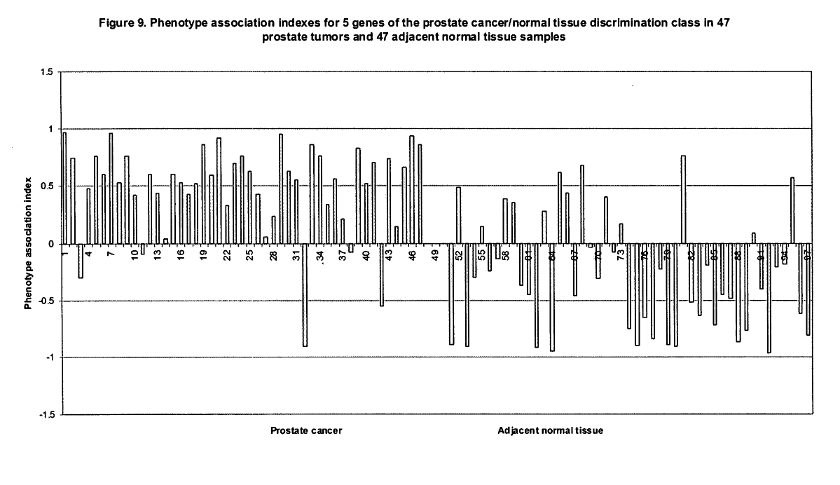

expression/<expression₁+expression₂>

where “expression” is the observed expression level for gene x for the individual tumor, and “<expression₁+expression₂>” is the average gene expression level for gene x across the set of 33 samples used to generate the prostate cancer predictor minimum segregation sets. The −fold expression changes for these ten and five genes were log₁₀ transformed, the transformed data entered as an array in a Microsoft Excel spreadsheet, and the Excel CORREL function was used to generate a correlation coefficient between the individual tumor data array and the corresponding log₁₀ transformed data for the average −fold expression changes in the cell lines for the same ten and five genes (i.e., log₁₀(<expression>₁/<expression>₂). This second correlation coefficient is the phenotype association index. The phenotype association indices had the surprising and unexpected property of allowing the samples to be classified according to the sign of the index. FIG. 6 and FIG. 7 show the phenotype association index for each of the thirty-three samples classified using the prostate cancer/normal tissue minimum segregation sets described above. In both instances, using either cluster 1 (ten genes) or cluster 2 (five genes), 9 out of 9 ANT samples had negative association indices, while 21 out of 24 cancer samples had positive association indices. Thus, the method correctly classified 30/33 or 91% of the samples.

To test the performance of prostate cancer/normal tissue minimum segregation sets or clusters on independent data sets, we applied the method to classify 94 ANT and cancer samples described in Singh, D., Febbo, P. G., et al., “Gene Expression Correlates of Clinical Prostate Cancer Behavior,” Cancer Cell March 2002 1:203-209, incorporated herein by reference. This set of samples comprises of 47 cancer samples and 47 adjacent normal tissue samples obtained in each instances from the same patients. The phenotype association index was obtained by calculating for each individual clinical sample, the −fold expression change for each of the ten and five genes in the prostate cancer predictor minimum segregation set 1 and 2. The −fold expression change was calculated as:

expression/<expression₁+expressions₂>

where “expression” is the observed expression level for gene x for the individual tumor, and “<expression₁+expression₂>” is the average gene expression level for gene x across the set of 94 samples. The −fold expression changes for these ten and five genes were log₁₀ transformed, the transformed data entered as an array in a Microsoft Excel spreadsheet, and the Excel CORREL function was used to generate a correlation coefficient between the individual tumor data array and the corresponding log₁₀ transformed data for the average −fold expression changes in the cell lines for the same ten and five genes (i.e., log₁₀(<expression>₁/<expression>2).

FIG. 8 and FIG. 9 show the phenotype association index for each of the ninety-four samples classified using the prostate cancer predictor minimum segregation clusters described above. Using cluster 1 (ten genes), 34 of 47 ANT samples had negative association indices, while 40 of 47 cancer samples had positive association indices. Thus, the method correctly classified 74/94 or 79% of the samples in independent data set. Using cluster 2 (five genes), 34 of 47 ANT samples had negative association indices, while 42 of 47 cancer samples had positive association indices. Thus, the method correctly classified 76/94 or 81% of the samples in an independent data set.

C. Invasion Clusters and Sample Classification

The methods of the invention were used along with the data reported by Singh, et al. (2002) to identify gene clusters associated with an invasive phenotype. Invasive phenotype was assessed by determining the presence or absence of positive surgical margins. The same first reference set described above in part A was used to generate the concordance and minimum segregation sets for invasiveness. The second reference set was obtained following the procedures described above in part B, using the supplemental data reported in Singh, et al. (2002) for fourteen invasive and 38 non-invasive human prostate tumors. Thus, the second reference set was obtained by using the Affymetrix MicroDB (version 3.0) and Affymetrix Data Mining Tools (DMT) (version 3.0) data analysis software to identify genes that were differentially regulated in invasion group compared to non-invasive group of patients at the statistically significant level (p<0.05; Student T-test). Candidate genes were included in the second reference set if they were identified by the DMT software as having p values of 0.05 or less both for up-regulated and down-regulated genes. 3869 genes were identified as being members of the second reference set.

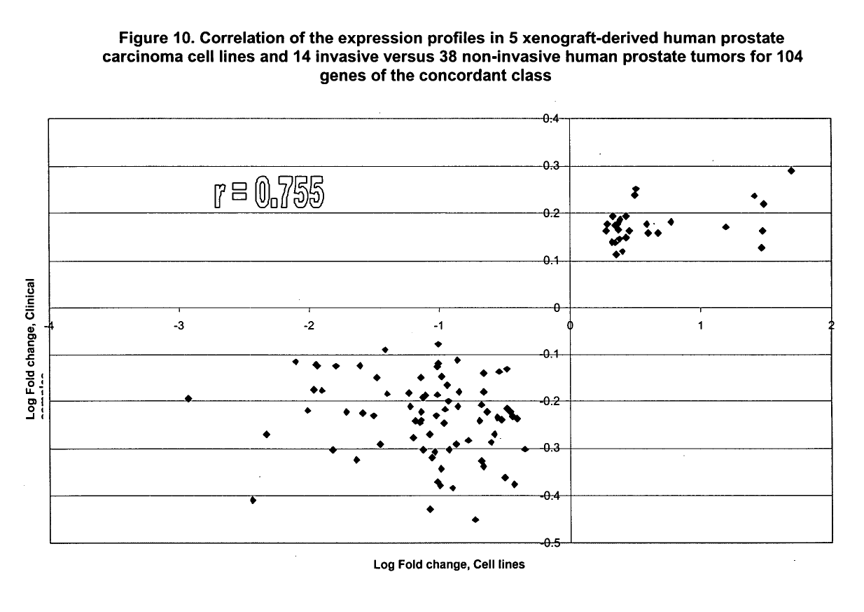

The concordance set was obtained by selecting only those genes having a consistent direction of the differential in both the first and the second reference sets (i.e., greater gene expression in the tumor lines cf the control lines and greater gene expression in the invasive tumor samples cf. the non-invasive tumor samples or vice-versa). The concordance set comprised 104 genes with an overall correlation coefficient of 0.755 (FIG. 10).

A minimum segregation set was selected following the procedures described above in section B. A scatter plot was generated of the log₁₀ transformed average −fold expression change in the cell line and average −fold expression change in the sample data. For the clinical sample data, <expression>₁ corresponds to the average expression value for gene x over all samples from patients who had invasive tumors and <expression>₂ corresponds to the average expression value for gene x over all samples from patients who had non-invasive tumors. The overall correlation coefficient for the invasiveness concordance set was 0.755 . . . . The invasiveness concordance set is shown in FIG. 10.

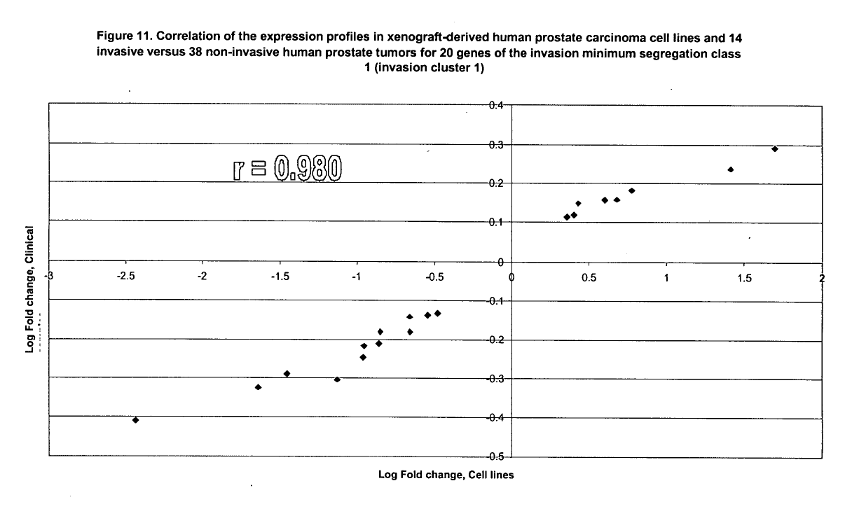

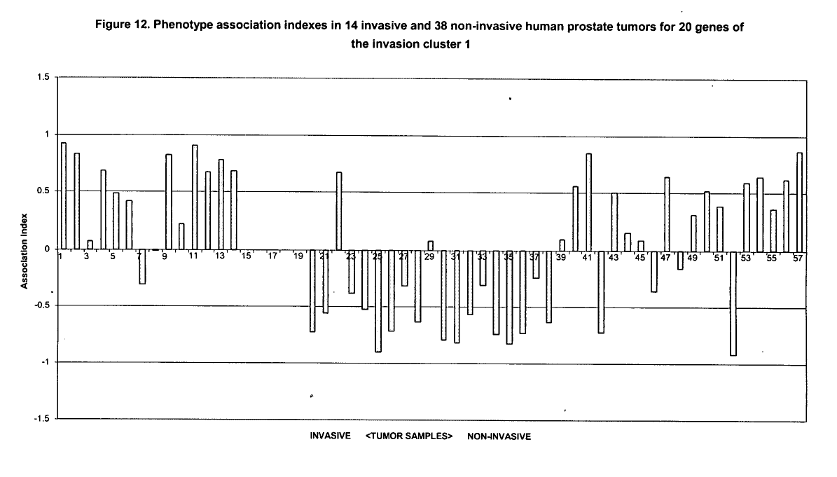

A minimum segregation set was identified by selecting a subset of the highly correlated genes from the invasiveness concordance set. This minimum segregation set (invasion minimum segregation set 1 or invasion cluster 1) included 20 genes listed below in Table 8. The overall correlation coefficient between the cell lines and clinical samples for invasion cluster 1 was 0.980. FIG. 11 shows the scatter plot for invasion cluster 1.

TABLE 8
Prostate Cancer Invasion Minimum Segregation Set 1.

| Affymetrix | | |
|---|---|---|
| Probe Set ID | LocusLink | |
| (U95Av2) | Identifier | Description |
| 33904_at | 1365 | CLDN3: claudin 3 |
| 1842_at | 2521 | FUS: fusion, derived from |
| | | t(12; 16) malignant |
| | | liposarcoma |
| 37741_at | 5831 | PYCR1: pyrroline-5- |
| | | carboxylate reductase 1 |
| 36174_at | 65108 | MACMARCKS: |
| | | macrophage myristoylated |
| | | alanine-rich C kinase |
| | | substrate |
| 1287_at | 142 | ADPRT: ADP- |
| | | ribosyltransferase (NAD+; |
| | | poly (ADP-ribose) |
| | | polymerase) |
| 39729_at | 7001 | PRDX2: peroxiredoxin 2 |
| 39020_at | 10572 | SIVA: CD27-binding |
| | | (Siva) protein |
| 40074_at | 10797 | MTHFD2: methylene |
| | | tetrahydrofolate dehydrogenase |
| | | (NAD+ dependent), |
| | | methenyltetrahydrofolate |
| | | cyclohydrolase |
| 502_s_at | 2709 | GJB5: gap junction |
| | | protein, beta 5 (connexin |
| | | 31.1) |
| 41817_g_at | 355 | TNFRSF6: tumor necrosis |
| | | factor receptor |
| | | superfamily, member 6 |
| 40847_at | 3675 | ITGA3: integrin, alpha 3 |
| | | (antigen CD49C, alpha 3 |
| | | subunit of VLA-3 |
| | | receptor) |
| 41641_at | 578 | BAK1: BCL2- |
| | | antagonist/killer 1 |
| 40031_at | 8626 | TP63: tumor protein p63 |
| 38608_at | 5099 | PCDH7: BH- |
| | | protocadherin(brain-heart) |
| 38288_at | N/A [Genbank Accession | KRT6E: keratin 6E |
| | No. L42611] | |
| 34853_at | 2263 | FGFR2: fibroblast growth |
| | | factor receptor 2 (bacteria- |
| | | expressed kinase, |
| | | keratinocyte growth factor |
| | | receptor, craniofacial |
| | | dysostosis 1, Crouzon |
| | | syndrome, Pfeiffer |
| | | syndrome, Jackson-Weiss |
| | | syndrome) |
| 209_at | 2263 | FGFR2 fibroblast growth |
| | | factor receptor 2 (bacteria- |
| | | expressed kinase, |
| | | keratinocyte growth factor |
| | | receptor, craniofacial |
| | | dysostosis 1, Crouzon |
| | | syndrome, Pfeiffer |
| | | syndrome, Jackson-Weiss |
| | | syndrome) 10q26 |
| 32719_at | 27350 | APOBEC3C: |
| | | apolipoprotein B mRNA |
| | | editing enzyme, catalytic |
| | | polypeptide-like 3C |
| 1898_at | 3084 | NRG1: neuregulin 1 |
| 115_at | 2263 | FGFR2: fibroblast growth |
| | | factor receptor 2 (bacteria- |
| | | expressed kinase, |
| | | keratinocyte growth factor |
| | | receptor, craniofacial |
| | | dysostosis 1, Crouzon |
| | | syndrome, Pfeiffer |
| | | syndrome, Jackson-Weiss |
| | | syndrome) |

Note that three entries in the table correspond to the same genes, i.e., 34853_at, 209_at, and 115_at. They most likely represent the splice variants of the same gene (Hs.31989). According to Affymetrix annotation, the 34853_at is an alternative splice 3 variant of the FGFR2.

Individual phenotype association indices were calculated for each of the 14 invasive and each of the 38 non-invasive human prostate tumors according to the methods described in section B, above, using data for the 20 genes that make up invasion cluster 1. The phenotype association index for each tumor sample was calculated using the average −fold expression change data for the tumor cell line data and the individual −fold expression change data for the tumor sample. The data were log₁₀ transformed and a correlation coefficient (phenotype association index) was calculated. The results are shown in FIG. 12. Application of the classification method using invasion cluster 1 resulted in 12/14 invasive tumors having positively signed association indices, and so were correctly classified, while 21/38 of the non-invasive tumors had negative association indices and so were correctly classified. Thus, invasion cluster 1 accurately classified 33/52=63% of the tumors in this sample set.

The greatest percentage of misclassifications obtained using invasion cluster 1 involved false positives, i.e., 17/38=44% of the non-invasive tumors were mis-classified as having an expression profile associated with the invasive phenotype. To improve the overall accuracy of the method, the sample set was re-structured so as to include data only from the twelve invasive tumors correctly classified using invasion cluster 1, and from the seventeen tumors mis-classified as false positives. (The false positives were considered to be non-invasive tumors (as, in fact they were) in carrying out the method steps to generate the second reference set, the concordance set, and the minimum segregation set.) Using this set of twenty-nine samples, another second reference set was generated by using the Affymetrix MicroDB (version 3.0) and Affymetrix Data Mining Tools (DMT) (version 3.0) data analysis software to identify genes that were differentially regulated in invasion group compared to non-invasive group of patients at the statistically significant level (p<0.05; Student T-test). Candidate genes were included in the second reference set if they were identified by the DMT software as having p values of 0.05 or less both for up-regulated and down-regulated genes. 458 genes were identified as being members of the second reference set.

Once the second reference set was generated, it was used to generate a concordance set by applying the criterion that the direction of the differential was consistent in the cell line and the clinical sample data. That is, the concordance set included only those genes present in the first and second reference sets whose expression was always greater in the tumor cell line cf. the control cell line and always greater in the invasive tumor sample cf. the non-invasive tumor sample, or vice-versa. The concordance set comprised 23 genes (r=0.809).

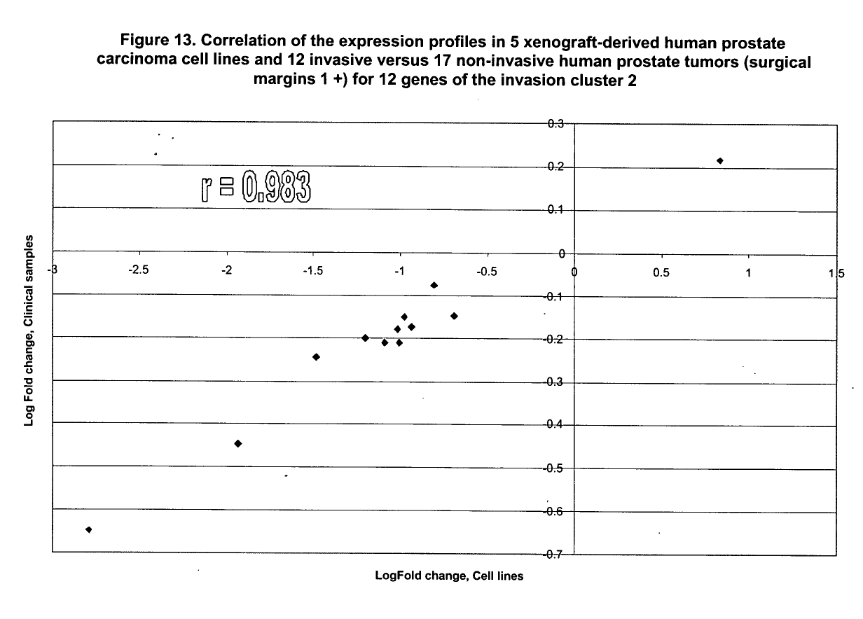

Once the concordance set was obtained using the data from the 29-member set of clinical samples, average expression values for genes within the concordance set were generated for the tumor cell lines, the control cell lines, the invasive tumors, and the non-invasive tumors. Average −fold expression changes were obtained, log₁₀ transformed, and used to generate scatter plots and first correlation coefficients, as described above. A second minimum segregation set (invasion cluster 2) was identified by selecting a subset of genes from the concordance set whose −fold expression changes were highly correlated in the cell line and clinical samples. Invasion cluster 2 included 12 genes, and had an overall correlation coefficient of 0.983. See FIG. 13. The genes that were selected as invasion cluster 2 (invasion minimum segregation set 2) are listed in Table 9.

TABLE 9
Prostate Cancer Invasion Minimum Segregation Set 2.
12 genes (r = 0.983)

| Affymetrix Probe | |
|---|---|
| Set ID (U95Av2) | Description |
| 1018_at | U81787/FEATURE = /DEFINITION = HSU81787 Human Wnt10B |
| | mRNA, complete cds |
| 38336_at | Cluster Incl. AB023230: Homo sapiens mRNA for KIAA1013 |
| | protein, partial cds/cds = (0,3188)/gb = AB023230/gi = 4589675/ |
| | ug = Hs. 96427/len = 4783 |
| 41619_at | Cluster Incl. AL022398: dJ434O14.4 (Interferon Regulatory Factor 6)/ |
| | cds = (68,1471)/gb = AL022398/gi = 3355547/ug = Hs. 11801/ |
| | len = 4077 |
| 33369_at | Cluster Incl. AI535653: P9-C4.T3.P9.D4 Homo sapiens cDNA, 3 end/ |
| | clone_end = 3/gb = AI535653/gi = 4449788/ug = Hs. 223018/len = 590 |
| 37978_at | Cluster Incl. D78177: Homo sapiens mRNA for quinolinate |
| | phosphoribosyl transferase, complete cds/cds = (0,893)/gb = D78177/ |
| | gi = 1060906/ug = Hs. 8935/len = 894 |
| 377_g_at | AB000220/FEATURE = /DEFINITION = AB000220 Homo sapiens |
| | mRNA for semaphorin E, complete cds |
| 39411_at | Cluster Incl. AL080156: Homo sapiens mRNA; cDNA |
| | DKFZp434J214 (from clone DKFZp434J214)/cds = (0,1081)/ |
| | gb = AL080156/gi = 5262614/ug = Hs. 12813/len = 2697 |
| 38772_at | Cluster Incl. Y11307: H. sapiens CYR61 mRNA/cds = (223,1368)/ |
| | gb = Y11307/gi = 2791897/ug = Hs. 8867/len = 2052 |
| 39248_at | Cluster Incl. N74607: za55a01.s1 Homo sapiens cDNA, 3 end/ |
| | clone = IMAGE-296424/clone_end = 3/gb = N74607/gi = 1231892/ |
| | ug = Hs. 234642/len = 487 |
| 41193_at | Cluster Incl. AB013382: Homo sapiens mRNA for DUSP6, complete |
| | cds/cds = (351,1496)/gb = AB013382/gi = 3869139/ug = Hs. 180383/ |
| | len = 2390 |
| 672_at | J03764/FEATURE = cds/DEFINITION = HUMPAIA Human, |
| | plasminogen activator inhibitor-1 gene, exons 2 to 9 |
| 39052_at | Cluster Incl. J00124: Homo sapiens 50 kDa type I epidermal keratin |
| | gene, complete cds/cds = (61,1479)/gb = J00124/gi = 186704/ |
| | ug = Hs. 117729/len = 1634 |

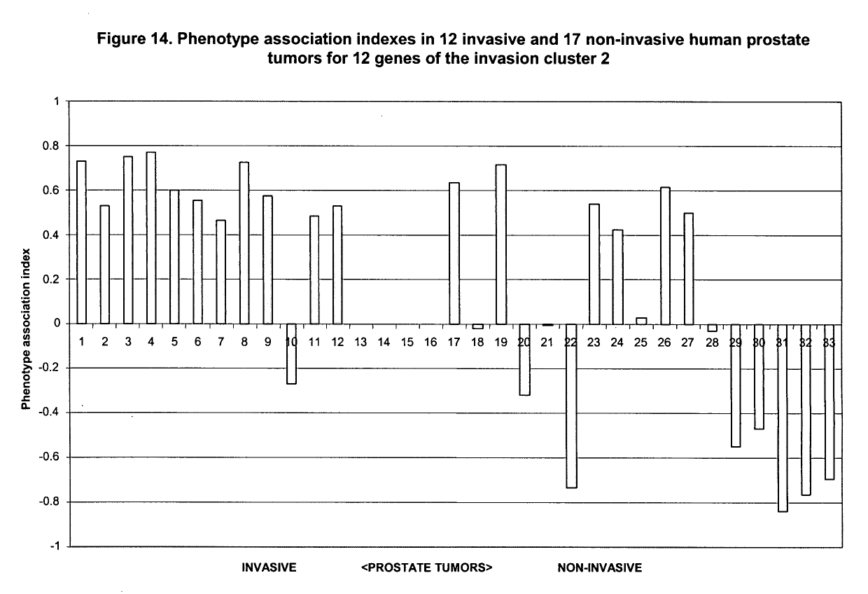

Individual phenotype association indices were calculated for each of the 12 invasive and each of the 17 non-invasive human prostate tumors used to generate invasion cluster 2 according to the methods described in section B, above, using data for the 12 genes that make up invasion cluster 2. The phenotype association index for each tumor sample was calculated using the average −fold expression change data for the tumor cell line data and the individual −fold expression change data for the tumor sample. The data were log₁₀ transformed and a correlation coefficient (phenotype association index) was calculated. The results are shown in FIG. 14. Application of the classification method using invasion cluster 2 resulted in 11/12 invasive tumors having positively signed association indices, and so were correctly classified, while 10/17 of the non-invasive tumors had negative association indices and so were correctly classified. There thus were 7 false positives identified using invasion cluster 2. Overall, invasion cluster 2 accurately classified 21/29=72% of the tumors in this sample set.

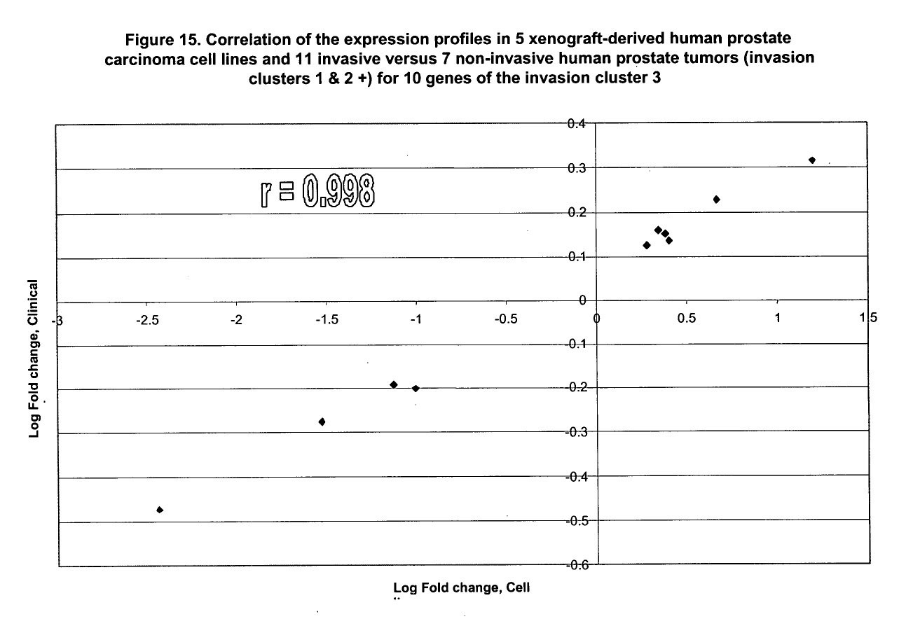

The method was iterated using the 11 properly classified invasive tumors and the 7 non-invasive tumors mis-classified as false positives using invasion cluster 2. Using the expression data from these 18 tumors (11 invasive and 7 non-invasive) and following the identical procedures as outlined above, a new second reference set of 449 genes, concordance set of 16 genes (r =0.908), and minimum segregation set (minimum segregation set 3 or invasion cluster 3) were generated. Invasion cluster 3 includes the 10 genes listed in Table 10, and had an overall correlation coefficient of 0.998, as shown in FIG. 15.

TABLE 10
Prostate Cancer Invasion Minimum Segregation Set 3.
10 genes (r = 0.998)

| Affymetrix Probe | |
|---|---|
| Set ID (U95Av2) | Description |
| 35704_at | Cluster Incl. X92814: H. sapiens mRNA for rat HREV107-like protein/ |
| | cds = (407,895)/gb = X92814/gi = 1054751/ug = Hs. 37189/len = 1070 |
| 41850_s_at | Cluster Incl. U63825: Human hepatitis delta antigen interacting |
| | protein A (dipA) mRNA, complete cds/cds = (28,636)/gb = U63825/ |
| | gi = 1488313/ug = Hs. 66713/len = 879 |
| 39072_at | Cluster Incl. L07648: Human MXI1 mRNA, complete cds/ |
| | cds = (208,894)/gb = L07648/gi = 506626/ug = Hs. 118630/len = 2400 |
| 38771_at | Cluster Incl. D50405: Human mRNA for RPD3 protein, complete cds/ |
| | cds = (63,1511)/gb = D50405/gi = 1665722/ug = Hs. 88556/len = 2091 |
| 34987_s_at | Cluster Incl. X79536: H. sapiens mRNA for hnRNPcore protein A1/ |
| | cds = (26,988)/gb = X79536/gi = 496897/ug = Hs. 151604/len = 1198 |
| 37040_at | Cluster Incl. D42041: Human mRNA for KIAA0088 gene, partial cds/ |
| | cds = (0,2832)/gb = D42041/gi = 577294/ug = Hs. 76847/len = 3820 |
| 851_s_at | S62539/FEATURE=/DEFINITION = S62539 insulin receptor |
| | substrate-1 [human, skeletal muscle, mRNA, 5828 nt] |
| 209_at | M94167/FEATURE=/DEFINITION = HUMHERGC Human |
| | heregulin-beta2 gene, complete cds |
| 936_s_at | Protein Phosphatase Inhibitor Homolog |
| 115_at | X14787/FEATURE = cds/DEFINITION = HSTS Human mRNA for |
| | thrombospondin |

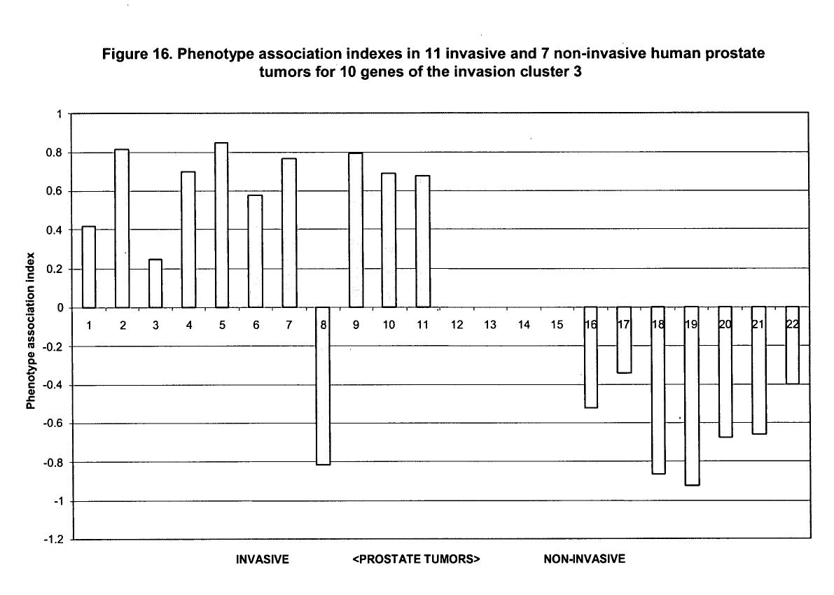

As was done with the previous invasion clusters, individual phenotype association indices were calculated for each of the 11 invasive and each of the 7 non-invasive human prostate tumors used to generate invasion cluster 3 according to the methods described in section B, above, using data for the 10 genes that make up invasion cluster 3. The results are shown in FIG. 16. Application of the classification method using invasion cluster 3 resulted in 10/11 invasive tumors having positively signed association indices, and so were correctly classified, while 7/7 of the non-invasive tumors had negative association indices and so were correctly classified. There thus were 0 false positives identified using invasion cluster 3. Overall, invasion cluster 3 accurately classified 17/18=94% of the tumors in this sample set.

Of the fourteen invasive tumors comprising the original data set, 10/14=71% scored positive phenotype association indices in all three invasion clusters, 3/14=21% scored positive phenotype association indices in two of the three invasion clusters, and 1/14=7% scored a positive phenotype association index in only a single of the three invasion clusters. These data are summarized in Table 11.

TABLE 11
Classification of Invasive Prostate
Tumors using Invasion Clusters 1-3.

| | Invasion | Invasion | Invasion | No. of Correct |
|---|---|---|---|---|
| Tumor | Cluster 1 | Cluster 2 | Cluster 3 | Classifications |
| T33 | 0 | 1 | 0 | 1 |
| T46 | 0 | 1 | 1 | 2 |
| T54 | 1 | 1 | 0 | 2 |
| T58 | 1 | 0 | 1 | 2 |
| T01 | 1 | 1 | 1 | 3 |
| T10 | 1 | 1 | 1 | 3 |
| T24 | 1 | 1 | 1 | 3 |
| T29 | 1 | 1 | 1 | 3 |
| T30 | 1 | 1 | 1 | 3 |
| T32 | 1 | 1 | 1 | 3 |
| T47 | 1 | 1 | 1 | 3 |
| T57 | 1 | 1 | 1 | 3 |
| T59 | 1 | 1 | 1 | 3 |
| T62 | 1 | 1 | 1 | 3 |
| No. Genes in | 20 | 12 | 10 | |
| Cluster | | | | |
| Correlation | 0.98 | 0.983 | 0.998 | |
| Coefficient of | | | | |
| Cluster | | | | |

Note:
1 = Positive phenotype association index;
0 = negative phenotype association index.

A similar analysis can be carried out for the 38 non-invasive tumors that comprised the original sample set. Of these thirty eight non-invasive tumors, 17/38=45% scored a positive phenotype association index in one of the three invasion clusters (one non-invasive tumor (T5) scored negatively in all three invasion clusters and included in this group), and 21/38=55% scored a positive phenotype association index in two of the three invasion clusters. These data are summarized in Table 12.

TABLE 12
Classification of Non-Invasive Prostate
Tumors using Invasion Clusters 1-3.

| | Invasion | Invasion | Invasion | No. of Correct |
|---|---|---|---|---|
| Tumor | Cluster 1 | Cluster 2 | Cluster 3 | Classifications |
| T5 | 0 | 0 | 0 | 3 |
| T3 | 0 | 1 | 0 | 2 |
| T6 | 0 | 1 | 0 | 2 |
| T11 | 0 | 1 | 0 | 2 |
| T15 | 0 | 1 | 0 | 2 |
| T17 | 0 | 1 | 0 | 2 |
| T18 | 0 | 1 | 0 | 2 |
| T19 | 0 | 1 | 0 | 2 |
| T20 | 0 | 1 | 0 | 2 |
| T21 | 0 | 1 | 0 | 2 |
| T22 | 0 | 1 | 0 | 2 |
| T23 | 0 | 1 | 0 | 2 |
| T26 | 0 | 1 | 0 | 2 |
| T34 | 0 | 1 | 0 | 2 |
| T41 | 0 | 1 | 0 | 2 |
| T49 | 0 | 1 | 0 | 2 |
| T55 | 1 | 0 | 0 | 2 |
| T2 | 0 | 1 | 1 | 1 |
| T4 | 1 | 1 | 0 | 1 |
| T13 | 0 | 1 | 1 | 1 |
| T14 | 0 | 1 | 1 | 1 |
| T16 | 1 | 0 | 1 | 1 |
| T25 | 0 | 1 | 1 | 1 |
| T27 | 1 | 1 | 0 | 1 |
| T28 | 1 | 0 | 1 | 1 |
| T31 | 1 | 0 | 1 | 1 |
| T36 | 1 | 0 | 1 | 1 |
| T37 | 1 | 1 | 0 | 1 |
| T38 | 1 | 1 | 0 | 1 |
| T39 | 0 | 1 | 1 | 1 |
| T40 | 1 | 1 | 0 | 1 |
| T42 | 1 | 1 | 0 | 1 |
| T43 | 1 | 1 | 0 | 1 |
| T45 | 1 | 0 | 1 | 1 |
| T50 | 1 | 0 | 1 | 1 |
| T53 | 1 | 0 | 1 | 1 |
| T56 | 1 | 0 | 1 | 1 |
| T60 | 1 | 0 | 1 | 1 |
| No. Genes in | 20 | 12 | 10 | |
| Cluster | | | | |
| Correlation | 0.98 | 0.983 | 0.998 | |
| Coefficient of | | | | |
| Cluster | | | | |

Note:
1 = Positive phenotype association index;
0 = negative phenotype association index.

Three of the invasive tumors scored positively in two of the three invasion clusters, and twenty-one of the non-invasive tumors also scored positively in two of the three invasion clusters. We iterated the method, as described above, using this group of three invasive and twenty-one non-invasive tumors to generate another second reference set, concordance set and minimum segregation set (minimum segregation set 4 or invasion cluster 4). The purpose of this experiment was to determine how well invasion cluster 4 could differentiate this set of three invasive and twenty-one non-invasive prostate tumors.

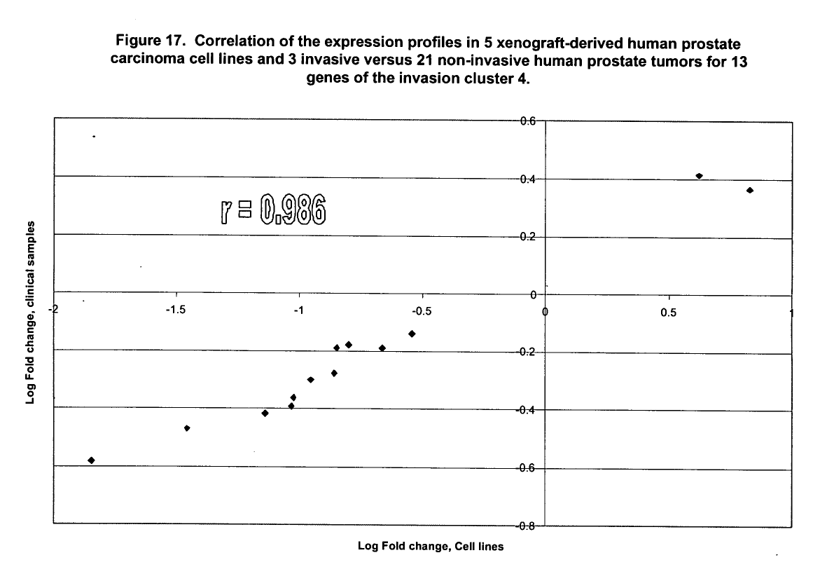

Invasion cluster 4 includes the 13 genes listed in Table 13, and had an overall correlation coefficient of 0.986, as shown in FIG. 17.

TABLE 13
Prostate Cancer Invasion Minimum Segregation Set 4.
13 genes (r = 0.986)

| Affymetrix | |
|---|---|
| Probe Set ID | |
| (U95Av2) | Description |
| 1375_s_at | M32304/FEATURE = /DEFINITION = HUMMET Human |
| | metalloproteinase inhibitor mRNA, complete cds |
| 41393_at | Cluster Incl. AF003540: Homo sapiens Krueppel family zinc finger |
| | protein (znfp 104) mRNA, complete cds/cds = (45,1934)/ |
| | gb = AF003540/gi = 2384652/ug = Hs. 104382/len = 2394 |
| 870_f_at | M93311/FEATURE = cds/DEFINITION = HUMMETIII Human |
| | metallothionein-III gene, complete cds |
| 39594_f_at | J04152/FEATURE = mRNA/DEFINITION = HUMGA733A Human |
| | gastrointestinal tumor-associated antigen GA733-1 protein gene, |
| | complete cds, clone 05516 |
| 609_f_at | S62539/FEATURE = /DEFINITION = S62539 insulin receptor |
| | substrate-1 [human, skeletal muscle, mRNA, 5828 nt] |
| 40031_at | L33930/FEATURE = /DEFINITION = HUMCD24B Homo sapiens |
| | CD24 signal transducer mRNA, complete cds and 3 region |
| 38608_at | Cluster Incl. M10943: Human metallothionein-If gene (hMT-If)/ |
| | cds = (0,185)/gb = M10943/gi = 187540/ug = Hs. 203936/len = 186 |
| 38288_at | AB000220/FEATURE = /DEFINITION = AB000220 Homo sapiens mRNA |
| | for semaphorin E, complete cds |
| 36883_at | Cluster Incl. L41827: Homo sapiens sensory and motor neuron derived |
| | factor (SMDF) mRNA, complete cds/cds = (500,1390)/gb = L41827/ |
| | gi = 862422/ug = Hs. 172816/len = 1860 |
| 36130_f_at | Cluster Incl. M74542: Human aldehyde dehydrogenase type III |
| | (ALDHIII) mRNA, complete cds/cds = (42,1403)/gb = M74542/ |
| | gi = 178401/ug = Hs. 575/len = 1636 |
| 35577_at | Cluster Incl. AF027866: Homo sapiens megsin mRNA, complete cds/ |
| | cds = (364,1506)/gb = AF027866/gi = 3769372/ug = Hs. 138202/ |
| | len = 2249 |
| 32719_at | L20852/FEATURE = /DEFINITION = HUMGLVR2X Human |
| | leukemia virus receptor 2 (GLVR2) mRNA, complete cds |
| 291_s_at | Cluster Incl. U40038: Human GTP-binding protein alpha q subunit |
| | (GNAQ) mRNA, complete cds/cds = (42,1121)/gb = U40038/ |
| | gi = 1181670/ug = Hs. 180950/len = 1450 |

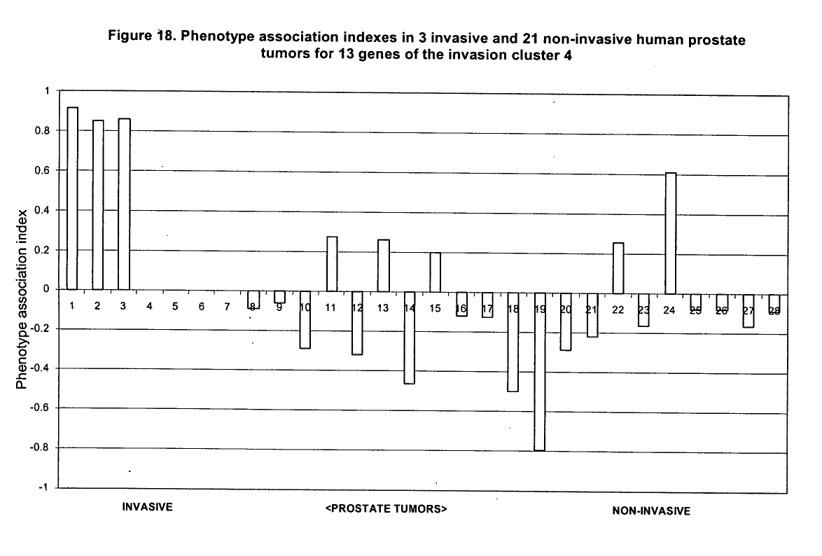

As shown in FIG. 18, when phenotype association indices were calculated for this set of samples applying genes of the invasion cluster 4, 3/3 invasive and 16/21 non-invasive tumors were correctly classified. Overall, 19 of 24 (79%) samples in this data set were correctly classified. As one skilled in art may determine from the FIG. 18, adjustment of the discrimination threshold (requiring, e.g., a positive association index of at least about 0.4) would yield a more accurate classification close to 100% accuracy.

D. Gleason Score Clusters and Sample Classifications

The methods of the invention were used along with the data reported by Singh, et al. (2002) to identify gene clusters capable of distinguishing tumor samples having a Gleason score of 6 or 7 (low grade tumors) from those having a Gleason score of 8 or 9 (high grade tumors). The same first reference set described above in part A was used to generate concordance and minimum segregation sets for Gleason score stratification. The second reference set was obtained following the procedures described above in part B, using the supplemental data reported in Singh, et al. (2002) for 46 low grade tumors and six high-grade tumors. Thus, the second reference set was generated by using the Affymetrix MicroDB (version 3.0) and Affymetrix Data Mining Tools (DMT) (version 3.0) data analysis software to identify genes that were differentially regulated in high grade group compared to low grade group of patients at the statistically significant level (p<0.05; Student T-test). Candidate genes were included in the second reference set if they were identified by the DMT software as having p values of 0.05 or less both for up-regulated and down-regulated genes. 2144 genes were identified as being members of the second reference set.

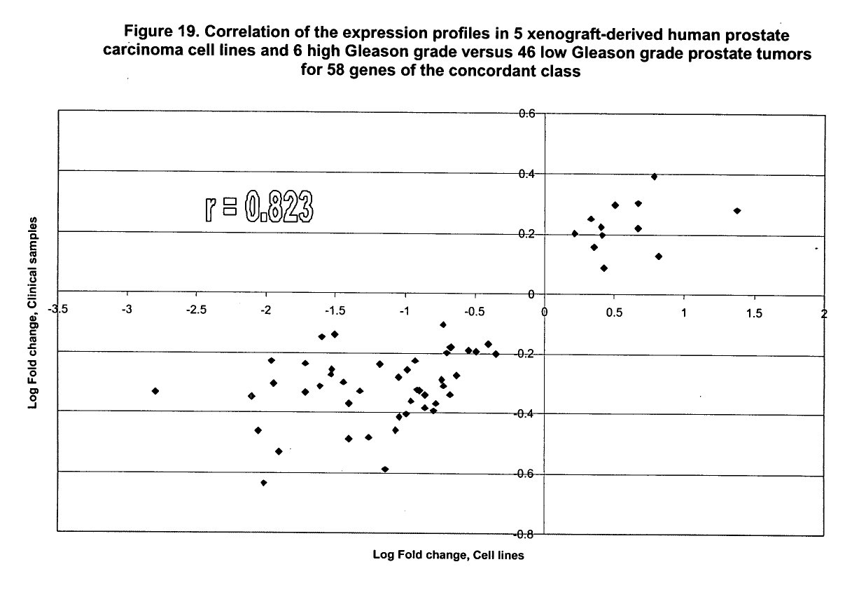

The concordance set was obtained by selecting only those genes having a consistent direction of the differential in both the first and the second reference sets (i.e., greater gene expression in the tumor lines cf the control lines and greater gene expression in the high grade cf. the low-grade tumor samples or vice-versa). The concordance set comprised 58 genes with an overall correlation coefficient equal to 0.823 (see FIG. 19).

A minimum segregation set was selected following the procedures described above in section B. A scatter plot was generated of the log₁₀ transformed average −fold expression change in the cell line and average −fold expression change in the sample data. For the clinical sample data, <expression>₁ corresponds to the average expression value for gene x over all samples from patients who had tumors with Gleason scores of 8 or 9 (high grade) and <expression>₂ corresponds to the average expression value for gene x over all samples from patients who had tumors with Gleason scores of 6 or 7 (low grade). The overall correlation coefficient for the high grade concordance set was 0.823. The high grade concordance set is shown in FIG. 19.

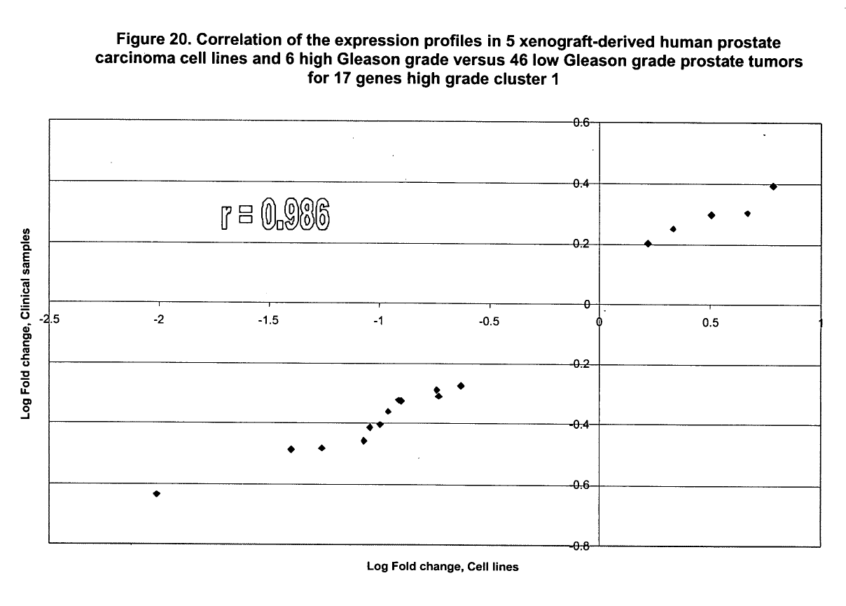

A minimum segregation set was identified by selecting a subset of the highly correlated genes from the high grade concordance set. This minimum segregation set (Gleason Score 8/9 minimum segregation set 1 or high grade cluster 1) included 17 genes listed below in Table 14. The overall correlation coefficient between the cell lines and clinical samples for high grade cluster 1 was 0.986. FIG. 20 shows the scatter plot for high grade cluster 1.

TABLE 14
Prostate Cancer Gleason Score 8/9 Minimum Segregation Set 1.
17 genes (r = 0.986)

| Affymetrix Probe | |
|---|---|
| Set ID (U95Av2) | Description |
| 34801_at | Cluster Incl. AB014610: Homo sapiens mRNA for KIAA0710 |
| | protein, complete cds/cds = (203,3550)/gb = AB014610/gi = 3327233/ |
| | ug = Hs. 4198/len = 4607 |
| 35627_at | Cluster Incl. U40571: Human alpha1-syntrophin (SNT A1) mRNA, |
| | complete cds/cds = (37,1554)/gb = U40571/gi = 1145727/ |
| | ug = Hs. 31121/len = 2110 |
| 33132_at | Cluster Incl. U37012: Human cleavage and polyadenylation |
| | specificity factor mRNA, complete cds/cds = (51,4379)/gb = U37012/ |
| | gi = 1045573/ug = Hs. 83727/len = 4463 |
| 39812_at | Cluster Incl. X79865: H. sapiens Mrp17 mRNA/cds = (137,733)/ |
| | gb = X79865/gi = 1313961/ug = Hs. 109059/len = 1008 |
| 34366_g_at | Cluster Incl. AF042386: Homo sapiens cyclophilin-33B (CYP-33) |
| | mRNA, complete cds/cds = (60,950)/gb = AF042386/gi = 2828150/ |
| | ug = Hs. 33251/len = 1099 |
| 33436_at | Cluster Incl. Z46629: Homo sapiens SOX9 mRNA/cds = (359,1888)/ |
| | gb = Z46629/gi = 758102/ug = Hs. 2316/len = 3923 |
| 1143_s_at | Fibroblast Growth Factor Receptor K-Sam, Alt. Splice 3, K-Sam III |
| 39407_at | Cluster Incl. M22488: Human bone morphogenetic protein 1 (BMP-1) |
| | mRNA/cds = (29,2221)/gb = M22488/gi = 179499/ug = Hs. 1274/ |
| | len = 2487 |
| 1343_s_at | S66896/FEATURE = /DEFINITION = S66896 squamous cell |
| | carcinoma antigen = serine protease inhibitor [human, mRNA, 1711 |
| | nt] |
| 2073_s_at | L34058/FEATURE = /DEFINITION = HUMCA13A Homo sapiens |
| | cadherin-13 mRNA, complete cds |
| 33272_at | Cluster Incl. AA829286: of08a01.s1 Homo sapiens cDNA, 3 end/ |
| | clone = IMAGE-1420488/clone_end = 3/gb = AA829286/gi = 2902385/ |
| | ug = Hs. 181062/len = 559 |
| 1440_s_at | X83490/FEATURE = exon/DEFINITION = HSFAS34 H. sapiens |
| | mRNA for Fas/Apo-1 (clone pCRTM11-Fasdelta(3,4)) |
| 32382_at | Cluster Incl. AB015234: Homo sapiens mRNA for uroplakin 1b, |
| | complete cds/cds = (0,782)/gb = AB015234/gi = 3721857/ |
| | ug = Hs. 198650/len = 783 |
| 988_at | X16354/FEATURE = /DEFINITION = HSTM1CEA Human mRNA |
| | for transmembrane carcinoembryonic antigen BGPa (formerly TM1- |
| | CEA) |
| 779_at | D21337/FEATURE = /DEFINITION = HUMCO Human mRNA for |
| | collagen |
| 39721_at | Cluster Incl. U09303: Human T cell leukemia LERK-2 (EPLG2) |
| | mRNA, complete cds/cds = (701,1741)/gb = U09303/gi = 1783360/ |
| | ug = Hs. 144700/len = 2895 |
| 37989_at | Cluster Incl. J03802: Human renal carcinoma parathgrad hormone- |
| | like peptide mRNA, complete cds/cds = (303,830)/gb = J03802/ |
| | gi = 190717/ug = Hs. 89626/len = 1595 |

Individual phenotype association indices were calculated for each of the six high grade and each of the 46 low grade human prostate tumors used to generate high grade cluster 1 according to the methods described in section B, above, using data for the 17 genes that make up high grade cluster 1 (data not shown). Application of the classification method using high grade cluster 1 resulted in 6/6 high grade tumors having positively signed association indices, and so were correctly classified, while 26/46 of the low grade tumors had negative association indices and so were correctly classified. There thus were 20 false positives (i.e., low grade tumors improperly classified as high grade tumors) identified using high grade cluster 1. Overall, high grade cluster 1 accurately classified 32/52=62% of the tumors in this sample set.

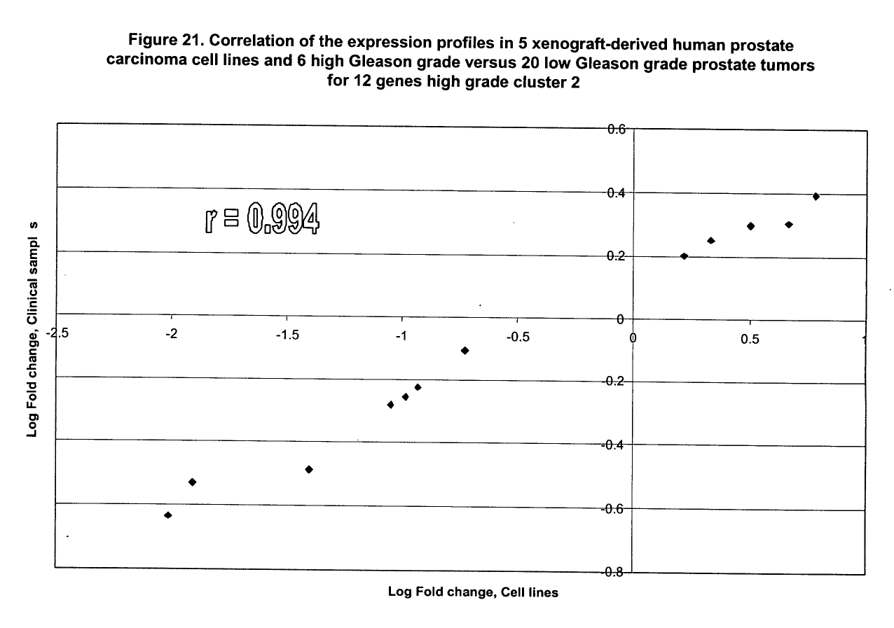

To improve the accuracy of the method, we selected from the concordance set of 58 genes additional minimum segregation sets and tested their ability to classify tumor samples. A second minimum segregation set was identified by selecting a smaller subset of the highly correlated genes from the high grade minimum segregation cluster 1. This minimum segregation set (Gleason Score 8/9 minimum segregation set 2 or high grade cluster 2) included 12 genes listed below in Table 15. The overall correlation coefficient between the cell lines and clinical samples for high grade cluster 2 was 0.994. FIG. 21 shows the scatter plot for high grade cluster 2.

TABLE 15
Prostate Cancer Gleason Score 8/9 Minimum Segregation Set 2.
12 genes (r = 0.994)

| Affymetrix Probe | |
|---|---|
| Set ID (U95Av2) | Description |
| 34801_at | Cluster Incl. AB014610: Homo sapiens mRNA for KIAA0710 |
| | protein, complete cds/cds = (203,3550)/gb = AB014610/gi = 3327233/ |
| | ug = Hs. 4198/len = 4607 |
| 35627_at | Cluster Incl. U40571: Human alpha1-syntrophin (SNT A1) mRNA, |
| | complete cds/cds = (37,1554)/gb = U40571/gi = 1145727/ |
| | ug = Hs. 31121/len = 2110 |
| 33132_at | Cluster Incl. U37012: Human cleavage and polyadenylation |
| | specificity factor mRNA, complete cds/cds = (51,4379)/gb = U37012/ |
| | gi = 1045573/ug = Hs. 83727/len = 4463 |
| 39812_at | Cluster Incl. X79865: H. sapiens Mrp17 mRNA/cds = (137,733)/ |
| | gb = X79865/gi = 1313961/ug = Hs. 109059/len = 1008 |
| 34366_g_at | Cluster Incl. AF042386: Homo sapiens cyclophilin-33B (CYP-33) |
| | mRNA, complete cds/cds = (60,950)/gb = AF042386/gi = 2828150/ |
| | ug = Hs. 33251/len = 1099 |
| 40712_at | Cluster Incl. D26579: Homo sapiens mRNA for transmembrane |
| | protein, complete cds/cds = (9,2483)/gb = D26579/gi = 1864004/ |
| | ug = Hs. 86947/len = 3236 |
| 38903_at | Cluster Incl. AF099731: Homo sapiens connexin 31.1 (GJB5) gene, |
| | complete cds/cds = (27,848)/gb = AF099731/gi = 4009521/ |
| | ug = Hs. 198249/len = 1370 |
| 1687_s_at | X84213/FEATURE = cds/DEFINITION = HSCEBP1 H. sapiens BAK |
| | mRNA for BCl-2 homologue |
| 40448_at | Cluster Incl. M92843: H. sapiens zinc finger transcriptional regulator |
| | mRNA, complete cds/cds = (59,1039)/gb = M92843/gi = 183442/ |
| | ug = Hs. 1665/len = 1746 |
| 39721_at | Cluster Incl. U09303: Human T cell leukemia LERK-2 (EPLG2) |
| | mRNA, complete cds/cds = (701,1741)/gb = U09303/gi = 1783360/ |
| | ug = Hs. 144700/len = 2895 |
| 36543_at | Cluster Incl. J02931: Human placental tissue factor (two forms) |
| | mRNA, complete cds/cds = (111,998)/gb = J02931/gi = 339501/ |
| | ug = Hs. 62192/len = 2141 |
| 37989_at | Cluster Incl. J03802: Human renal carcinoma parathgrad hormone- |
| | like peptide mRNA, complete cds/cds = (303,830)/gb = J03802/ |
| | gi = 190717/ug = Hs. 89626/len = 1595 |

Individual phenotype association indices were calculated for each of the six high grade and each of the 46 low grade human prostate tumors according to the methods described in section B, above, using data for the 12 genes that make up high grade cluster 2 (data not shown). Application of the classification method using high grade cluster 2 resulted in 6/6 high grade tumors having positively signed association indices, and so were correctly classified, while 30/46 of the low grade tumors had negative association indices and so were correctly classified. There thus were 16 false positives (i.e., low grade tumors improperly classified as high grade tumors) identified using high grade cluster 2. Overall, high grade cluster 2 accurately classified 36/52=69% of the tumors in this sample set.

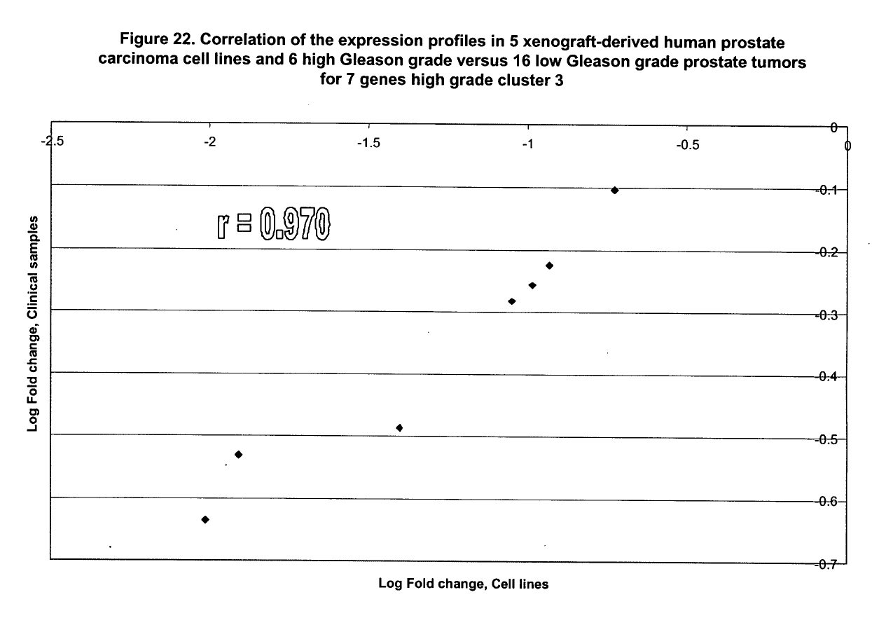

A third minimum segregation set was identified by selecting a smaller subset of the highly correlated genes from the high grade minimum segregation cluster 2. This minimum segregation set (Gleason Score 8/9 minimum segregation set 3 or high grade cluster 3) included the 7 genes listed below in Table 16. The overall correlation coefficient between the cell lines and clinical samples for high grade cluster 3 was 0.970 (FIG. 22).

TABLE 16
Prostate Cancer Gleason Score 8/9 Minimum Segregation Set 3.
7 genes (r = 0.97)

| Affymetrix Probe | |
|---|---|
| Set ID (U95Av2) | Description |
| 40712_at | Cluster Incl. D26579: Homo sapiens mRNA for transmembrane |
| | protein, complete cds/cds = (9,2483)/gb = D26579/gi = 1864004/ |
| | ug = Hs. 86947/len = 3236 |
| 38903_at | Cluster Incl. AF099731: Homo sapiens connexin 31.1 (GJB5) gene, |
| | complete cds/cds = (27,848)/gb = AF099731/gi = 4009521/ |
| | ug = Hs. 198249/len = 1370 |
| 1687_s_at | X84213/FEATURE = cds/DEFINITION = HSCEBP1 H. sapiens BAK |
| | mRNA for BCl-2 homologue |
| 40448_at | Cluster Incl. M92843: H. sapiens zinc finger transcriptional regulator |
| | mRNA, complete cds/cds = (59,1039)/gb = M92843/gi = 183442/ |
| | ug = Hs. 1665/len = 1746 |
| 39721_at | Cluster Incl. U09303: Human T cell leukemia LERK-2 (EPLG2) |
| | mRNA, complete cds/cds = (701,1741)/gb = U09303/gi = 1783360/ |
| | ug = Hs. 144700/len = 2895 |
| 36543_at | Cluster Incl. J02931: Human placental tissue factor (two forms) |
| | mRNA, complete cds/cds = (111,998)/gb = J02931/gi = 339501/ |
| | ug = Hs. 62192/len = 2141 |
| 37989_at | Cluster Incl. J03802: Human renal carcinoma parathgrad hormone- |
| | like peptide mRNA, complete cds/cds = (303,830)/gb = J03802/ |
| | gi = 190717/ug = Hs. 89626/len = 1595 |

Individual phenotype association indices were calculated for each of the six high grade and each of the 46 low grade human prostate tumors according to the methods described in section B, above, using data for the 7 genes that make up high grade cluster 3 (data not shown). Application of the classification method using high grade cluster 3 again resulted in 6/6 high grade tumors having positively signed association indices, and so were correctly classified, while 17/46 of the low grade tumors had negative association indices and so were correctly classified. There thus were 29 false positives (i.e., low grade tumors improperly classified as high grade tumors) identified using high grade cluster 3. Overall, high grade cluster 3 accurately classified 23/52=44% of the tumors in this sample set.

A summary of the accuracy with which the first three high grade clusters distinguished high grade (Gleason score 8 or 9) from low grade (Gleason score 6 or 7) tumors is provided in Table 17.

TABLE 17
Classification of High Grade & Low Grade Prostate Tumors using
High Grade Clusters 1-3.

| | | | | |
|---|---|---|---|---|
| | High Grade | High Grade | High Grade | No. of Correct |
| Tumor | Cluster 1 | Cluster 2 | Cluster 3 | Classifications |

Gleason Score 8 or 9 (high grade) Tumors

| | | | | |
|---|---|---|---|---|
| T26 | 1 | 1 | 1 | 3 |
| T31 | 1 | 1 | 1 | 3 |
| T45 | 1 | 1 | 1 | 3 |
| T57 | 1 | 1 | 1 | 3 |
| T58 | 1 | 1 | 1 | 3 |
| T59 | 1 | 1 | 1 | 3 |

Gleason Score 6 or 7 (low grade) Tumors

| | | | | |
|---|---|---|---|---|
| T01 | 1 | 1 | 1 | 0 |
| T02 | 0 | 0 | 1 | 2 |
| T03 | 0 | 0 | 0 | 3 |
| T04 | 0 | 0 | 0 | 3 |
| T05 | 0 | 0 | 1 | 2 |
| T06 | 0 | 0 | 0 | 3 |
| T10 | 1 | 1 | 0 | 1 |
| T11 | 0 | 0 | 1 | 2 |
| T13 | 0 | 0 | 1 | 2 |
| T14 | 0 | 0 | 1 | 2 |
| T15 | 0 | 0 | 0 | 3 |
| T16 | 1 | 0 | 0 | 2 |
| T17 | 0 | 0 | 1 | 2 |
| T18 | 0 | 0 | 1 | 2 |
| T19 | 0 | 0 | 0 | 3 |
| T20 | 0 | 0 | 0 | 3 |
| T21 | 0 | 0 | 1 | 2 |
| T22 | 0 | 0 | 0 | 3 |
| T23 | 0 | 0 | 1 | 2 |
| T24 | 0 | 0 | 1 | 2 |
| T25 | 1 | 0 | 1 | 1 |
| T27 | 0 | 0 | 0 | 3 |
| T28 | 1 | 1 | 1 | 0 |
| T29 | 1 | 1 | 1 | 0 |
| T30 | 1 | 0 | 0 | 2 |
| T32 | 0 | 0 | 0 | 3 |
| T33 | 0 | 0 | 1 | 2 |
| T34 | 0 | 0 | 1 | 2 |
| T36 | 1 | 1 | 1 | 0 |
| T37 | 0 | 0 | 1 | 2 |
| T38 | 0 | 0 | 1 | 2 |
| T39 | 0 | 0 | 1 | 2 |
| T40 | 1 | 1 | 1 | 0 |
| T41 | 1 | 0 | 0 | 2 |
| T42 | 1 | 1 | 1 | 0 |
| T43 | 1 | 1 | 1 | 0 |
| T46 | 1 | 1 | 0 | 1 |
| T47 | 1 | 1 | 1 | 0 |
| T49 | 0 | 0 | 1 | 2 |
| T50 | 0 | 0 | 0 | 3 |
| T53 | 1 | 1 | 1 | 0 |
| T54 | 1 | 1 | 1 | 0 |
| T55 | 1 | 1 | 1 | 0 |
| T56 | 1 | 1 | 0 | 1 |
| T60 | 1 | 1 | 1 | 0 |
| T62 | 1 | 1 | 0 | 1 |
| No. Genes in | 17 | 12 | 7 | |
| Cluster | | | | |
| Correlation | 0.986 | 0.994 | 0.97 | |
| Coefficient | | | | |
| of Cluster | | | | |

Note:
1 = Positive phenotype association index;
0 = negative phenotype association index.

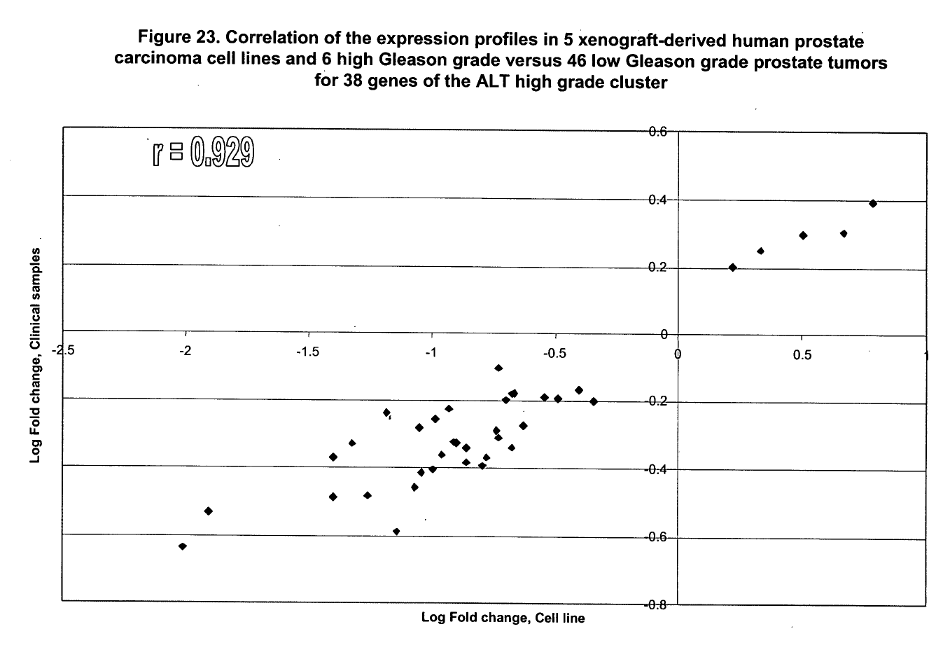

Since the overall classification accuracy of high grade cluster 3 was lower than that of high grade cluster 1 and 2, additional high grade clusters were generated from a high grade concordance set of 58 genes. The resulting alternative minimum segregation set (ALT high grade cluster) included a total of 38 genes listed below in Table 18. The overall correlation coefficient between the cell line and clinical samples for this high grade cluster (Gleason Score 8/9 ALT high grade cluster) was 0.929 (FIG. 23). Phenotype association indices were calculated for each of the 6 high grade and each of the 46 low grade tumors to determine how well this high grade cluster would classify the samples. All six of the high grade tumors were correctly classified, while 26/46 of the low grade tumors were correctly classified. Thus overall, this minimum segregation set correctly classified 32/52=62% of the samples.

TABLE 18
Prostate Cancer Gleason Score 8/9
ALT High Grade Minimum Segregation Set (38 genes).
38 genes (r = 0.929)

| Affymetrix | |
|---|---|
| Probe Set ID | |
| (U95Av2) | Description |
| 34801_at | Cluster Incl. AB014610: Homo sapiens mRNA for KIAA0710 protein, |
| | complete cds/cds = (203,3550)/gb = AB014610/gi = 3327233/ |
| | ug = Hs. 4198/len = 4607 |
| 35627_at | Cluster Incl. U40571: Human alpha1-syntrophin (SNT A1) mRNA, |
| | complete cds/cds = (37,1554)/gb = U40571/gi = 1145727/ug = Hs. 31121/ |
| | len = 2110 |
| 33132_at | Cluster Incl. U37012: Human cleavage and polyadenylation specificity |
| | factor mRNA, complete cds/cds = (51,4379)/gb = U37012/gi = 1045573/ |
| | ug = Hs. 83727/len = 4463 |
| 39812_at | Cluster Incl. X79865: H. sapiens Mrp17 mRNA/cds = (137,733)/ |
| | gb = X79865/gi = 1313961/ug = Hs. 109059/len = 1008 |
| 34366_g_at | Cluster Incl. AF042386: Homo sapiens cyclophilin-33B (CYP-33) |
| | mRNA, complete cds/cds = (60,950)/gb = AF042386/gi = 2828150/ |
| | ug = Hs. 33251/len = 1099 |
| 32545_r_at | Cluster Incl. L12535: Human RSU-1/RSP-1 mRNA, complete cds/ |
| | cds = (827,1660)/gb = L12535/gi = 434050/ug = Hs. 75551/len = 2194 |
| 35899_at | Cluster Incl. AF109401: Homo sapiens neurotrophic factor artemin |
| | precursor (ARTN) mRNA, complete cds/cds = (298,960)/ |
| | gb = AF109401/gi = 4071352/ug = Hs. 194689/len = 1003 |
| 32855_at | Cluster Incl. L00352: Human low density lipoprotein receptor gene/ |
| | cds = (93,2675)/gb = L00352/gi = 460289/ug = Hs. 213289/len = 5175 |
| 41817_g_at | Cluster Incl. AL049851: Human DNA sequence from clone 889J22B |
| | on chromosome 22q13.1/cds = (0,1000)/gb = AL049851/gi = 4826526/ |
| | ug = Hs. 57973/len = 1798 |
| 33436_at | Cluster Incl. Z46629: Homo sapiens SOX9 mRNA/cds = (359,1888)/ |
| | gb = Z46629/gi = 758102/ug = Hs. 2316/len = 3923 |
| 41663_at | Cluster Incl. AF038202: Homo sapiens clone 23570 mRNA sequence/ |
| | cds = UNKNOWN/gb = AF038202/gi = 2795923/ug = Hs. 12311/ |
| | len = 1742 |
| 188_at | U09303/FEATURE = /DEFINITION = HSU09303 Human T cell |
| | leukemia LERK-2 (EPLG2) mRNA, complete cds |
| 38822_at | Cluster Incl. AB011420: Homo sapiens mRNA for DRAK1, complete |
| | cds/cds = (117,1361)/gb = AB011420/gi = 3834353/ug = Hs. 9075/ |
| | len = 2641 |
| 38913_at | Cluster Incl. U60319: Homo sapiens haemochromatosis protein (HLA- |
| | H) mRNA, complete cds/cds = (221,1267)/gb = U60319/gi = 1469789/ |
| | ug = Hs. 20019/len = 2716 |
| 1143_s_at | Fibroblast Growth Factor Receptor K-Sam, Alt. Splice 3, K-Sam III |
| 40712_at | Cluster Incl. D26579: Homo sapiens mRNA for transmembrane |
| | protein, complete cds/cds = (9,2483)/gb = D26579/gi = 1864004/ |
| | ug = Hs. 86947/len = 3236 |
| 39407_at | Cluster Incl. M22488: Human bone morphogenetic protein 1 (BMP-1) |
| | mRNA/cds = (29,2221)/gb = M22488/gi = 179499/ug = Hs. 1274/ |
| | len = 2487 |
| 34044_at | Cluster Incl. AB007131: Homo sapiens mRNA for HSF2BP, complete |
| | cds/cds = (332,1336)/gb = AB007131/gi = 3345673/ug = Hs. 97624/ |
| | len = 1898 |
| 39320_at | Cluster Incl. U13697: Human interleukin 1-beta converting enzyme |
| | isoform beta (IL1BCE) mRNA, complete cds/cds = (0,1151)/ |
| | gb = U13697/gi = 717039/ug = Hs. 2490/len = 1185 |
| 38608_at | Cluster Incl. AA010777: ze22f06.r1 Homo sapiens cDNA, 5 end/ |
| | clone = IMAGE-359747/clone_end = 5/gb = AA010777/gi = 1471804/ |
| | ug = Hs. 99923/len = 521 |
| 35194_at | Cluster Incl. X53463: Human mRNA for glutathione peroxidase-like |
| | protein/cds = (51,623)/gb = X53463/gi = 31894/ug = Hs. 2704/len = 951 |
| 1343_s_at | S66896/FEATURE = /DEFINITION = S66896 squamous cell |
| | carcinoma antigen = serine protease inhibitor [human, mRNA, 1711 nt] |
| 2073_s_at | L34058/FEATURE = /DEFINITION = HUMCA13A Homo sapiens |
| | cadherin-13 mRNA, complete cds |
| 38903_at | Cluster Incl. AF099731: Homo sapiens connexin 31.1 (GJB5) gene, |
| | complete cds/cds = (27,848)/gb = AF099731/gi = 4009521/ |
| | ug = Hs. 198249/len = 1370 |
| 33272_at | Cluster Incl. AA829286: of08a01.s1 Homo sapiens cDNA, 3 end/ |
| | clone = IMAGE-1420488/clone_end = 3/gb = AA829286/gi = 2902385/ |
| | ug = Hs. 181062/len = 559 |
| 1687_s_at | X84213/FEATURE = cds/DEFINITION = HSCEBP1 H. sapiens BAK |
| | mRNA for BCl-2 homologue |
| 1440_s_at | X83490/FEATURE = exon/DEFINITION = HSFAS34 H. sapiens |
| | mRNA for Fas/Apo-1 (clone pCRTM11-Fasdelta(3,4)) |
| 32382_at | Cluster Incl. AB015234: Homo sapiens mRNA for uroplakin 1b, |
| | complete cds/cds = (0,782)/gb = AB015234/gi = 3721857/ |
| | ug = Hs. 198650/len = 783 |
| 40448_at | Cluster Incl. M92843: H. sapiens zinc finger transcriptional regulator |
| | mRNA, complete cds/cds = (59,1039)/gb = M92843/gi = 183442/ |
| | ug = Hs. 1665/len = 1746 |
| 988_at | X16354/FEATURE = /DEFINITION = HSTM1CEA Human mRNA |
| | for transmembrane carcinoembryonic antigen BGPa (formerly TM1- |
| | CEA) |
| 41481_at | Cluster Incl. X17033: Human mRNA for integrin alpha-2 subunit/ |
| | cds = (48,3593)/gb = X17033/gi = 33906/ug = Hs. 1142/len = 5373 |
| 35444_at | Cluster Incl. AC004030: Homo sapiens DNA from chromosome 19, |
| | cosmid F21856/cds = (0,2039)/gb = AC004030/gi = 2804590/ |
| | ug = Hs. 169508/len = 2040 |
| 779_at | D21337/FEATURE = /DEFINITION = HUMCO Human mRNA for |
| | collagen |
| 38746_at | Cluster Incl. AF011375: Homo sapiens integrin variant beta4E |
| | (ITGB4) mRNA, complete cds/cds = (0,2894)/gb = AF011375/ |
| | gi = 2293520/ug = Hs. 85266/len = 2895 |
| 32821_at | Cluster Incl. AI762213: wi54d04.x1 Homo sapiens cDNA, 3 end/ |
| | clone = IMAGE-2394055/clone_end = 3/gb = AI762213/gi = 5177880/ |
| | ug = Hs. 204238/len = 677 |
| 39721_at | Cluster Incl. U09303: Human T cell leukemia LERK-2 (EPLG2) |
| | mRNA, complete cds/cds = (701,1741)/gb = U09303/gi = 1783360/ |
| | ug = Hs. 144700/len = 2895 |
| 36543_at | Cluster Incl. J02931: Human placental tissue factor (two forms) |
| | mRNA, complete cds/cds = (111,998)/gb = J02931/gi = 339501/ |
| | ug = Hs. 62192/len = 2141 |
| 37989_at | Cluster Incl. J03802: Human renal carcinoma parathgrad hormone-like |
| | peptide mRNA, complete cds/cds = (303,830)/gb = J03802/gi = 190717/ |
| | ug = Hs. 89626/len = 1595 |

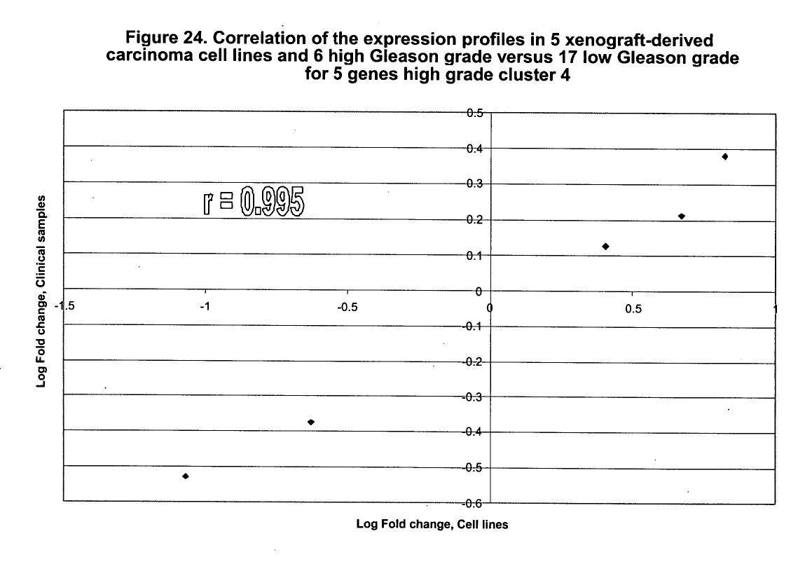

To further improve the overall classification accuracy, additional high grade clusters were generated by culling a subset of sample data made up of all the true positives (i.e., the 6 high grade tumors correctly classified using each of the first three high grade clusters) and the set of 12 low grade tumors that scored as false positives in 3/3 of the first 3 high grade clusters (i.e., all the Gleason score 6 & 7 tumors that had a “0” in the “No. of Correct Classifications” column in Table 15). This subset was used to generate another second reference set, and concordance set using the same procedures outlined above. From this concordance set of 33 genes (r =0.731), a fourth minimum segregation set was identified by selecting a subset of the highly correlated genes from the new high grade concordance set. This minimum segregation set (Gleason Score 8/9 minimum segregation set 4 or high grade cluster 4) included 5 genes listed below in Table 19. The overall correlation coefficient between the cell lines and clinical samples for high grade cluster 4 was 0.995. FIG. 24 shows the scatter plot for high grade cluster 4.

TABLE 19
Prostate Cancer Gleason Score 8/9 Minimum Segregation Set 4.
5 genes (r = 0.995)

| Affymetrix Probe | |
|---|---|
| Set ID (U95Av2) | Description |
| 1733_at | M60315/FEATURE = /DEFINITION = HUMTGFBC |
| | Human transforming growth factor-beta (tgf-beta) |
| | mRNA, complete cds |
| 41850_s_at | Cluster Incl. U63825: Human hepatitis delta |
| | antigen interacting protein A (dipA) mRNA, complete |
| | cds/cds = (28,636)/gb = U63825/ |
| | gi = 1488313/ug = Hs. 66713/len = 879 |
| 39020_at | Cluster Incl. U82938: Human CD27BP (Siva) mRNA, |
| | complete cds/cds = (252,821)/gb = |
| | U82938/gi = 2228596/ug = Hs. |
| | 112058/len = 1034 |
| 33436_at | Cluster Incl. Z46629: Homo sapiens |
| | SOX9 mRNA/cds = (359,1888)/gb = |
| | Z46629/gi = 758102/ug = |
| | Hs. 2316/len = 3923 |
| 988_at | X16354/FEATURE = /DEFINITION = |
| | HSTM1CEA Human mRNA for transmembrane |
| | carcinoembryonic antigen BGPa (formerly TM1- |
| | CEA) |

Phenotype association indices were calculated using the average cell line and individual sample −fold change expression data for the genes in high grade cluster 4. The sample included the 6 high grade tumors and the set of 17 low grade tumors that scored as false positives in 2/3 or 3/3 of the first three high grade clusters (i.e., all the Gleason score 6&7 tumors that had a “0” or “1” in the “No. of Correct Classifications” column in Table 17).

High grade cluster 4 correctly classified 6/6 high grade tumors, and 12/17 low grade tumors. Overall, high grade cluster 4 accurately characterized 18/23=78% of the tumors in this set.

To improve the accuracy of the classification, several additional minimum segregation sets of highly correlated genes were selected. Gleason Score 8/9 minimum segregation set 5, or high grade cluster 5, was used to generate phenotype association indices for the 6 high grade tumors (true positives) and the set of 17 low grade tumors that scored as false positives in 2/3 or 3/3 of the first three high grade clusters (i.e., all the Gleason score 6&7 tumors that had a “0” or “1” in the “No. of Correct Classifications” column in Table 17). High grade cluster 5 correctly classified 6/6 high grade tumors and 9/17 low grade tumors. Overall, high grade cluster 5 correctly classified 15/23=65% of the samples in this set.

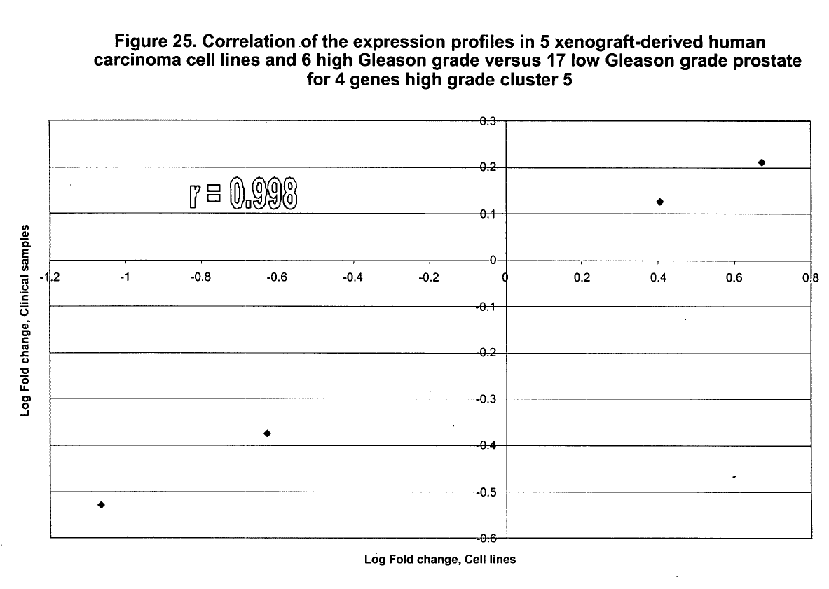

High grade cluster 5 included 4 genes listed below in Table 20. The overall correlation coefficient between the cell lines and clinical samples for high grade cluster 5 was 0.998. FIG. 25 shows the scatter plot for high grade cluster 5.

TABLE 20
Prostate Cancer Gleason Score 8/9 Minimum Segregation Set 5.
4 genes (r = 0.998)

| Affymetrix Probe | |
|---|---|
| Set ID (U95Av2) | Description |
| 41850_s_at | Cluster Incl. U63825: Human hepatitis |
| | delta antigen interacting protein A (dipA) mRNA, |
| | complete cds/cds = (28,636)/gb = U63825/ |
| | gi = 1488313/ug = Hs. 66713/len = 879 |
| 39020_at | Cluster Incl. U82938: Human CD27BP (Siva) mRNA, |
| | complete cds/cds = (252,821)/gb = U82938/gi = |
| | 2228596/ug = Hs. 112058/len = 1034 |
| 33436_at | Cluster Incl. Z46629: Homo sapiens SOX9 |
| | mRNA/cds = (359,1888)/gb = Z46629/gi = |
| | 758102/ug = Hs. 2316/len = 3923 |
| 988_at | X16354/FEATURE = /DEFINITION = |
| | HSTM1CEA Human mRNA for transmembrane |
| | carcinoembryonic antigen BGPa (formerly TM1- |
| | CEA) |

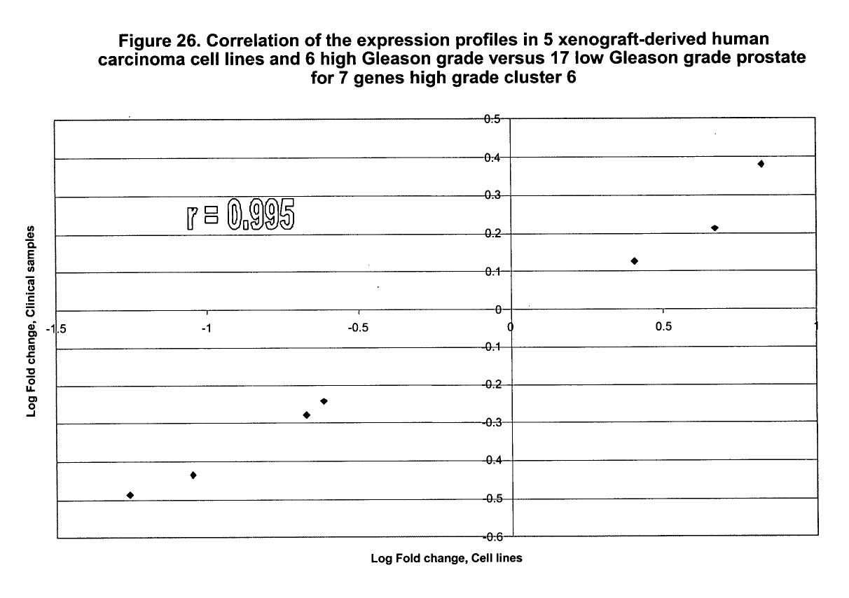

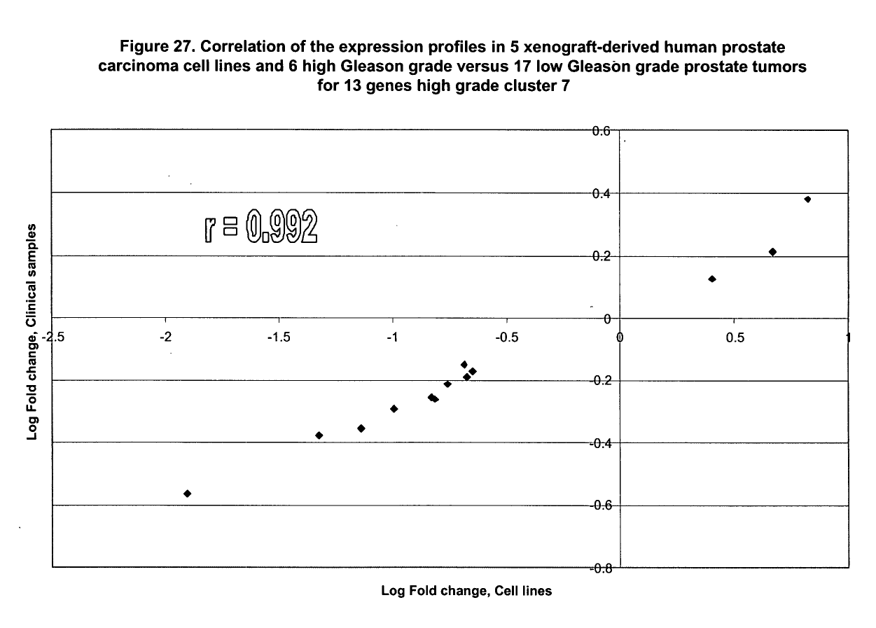

High grade cluster 6 included 7 genes and had an overall correlation coefficient of 0.995 (FIG. 26). High grade cluster 7 included 13 genes and had an overall correlation coefficient of 0.992 (FIG. 27). High grade cluster 6 correctly classified 6/6 of the high grade tumors, and 13/17 of the low grade tumors. Overall, high grade cluster 6 correctly classified 19/23=83% of the samples in this set. High grade cluster 7 correctly classified 6/6 of the high grade tumors and 14/17 of the low grade tumors. Overall, high grade cluster 7 correctly classified 20/23=87% of the samples in this set. Tables 21 and 22 list the genes that make up high grade cluster 6 and high grade cluster 7. A summary of the accuracy with which high grade clusters 4-7 distinguished high grade (Gleason score 8 or 9) from the “false positive” subset of seventeen low grade (Gleason score 6 or 7) tumors is provided in Table 23.

TABLE 21
Prostate Cancer Gleason Score 8/9 Minimum Segregation Set 6.
7 genes (r = 0.995)

| Affymetrix Probe | |
|---|---|
| Set ID (U95Av2) | Description |
| 1733_at | M60315/FEATURE = /DEFINITION = HUMTGFBC Human |
| | transforming growth factor-beta (tgf-beta) mRNA, complete cds |
| 41850_s_at | Cluster Incl. U63825: Human hepatitis delta antigen interacting |
| | protein A (dipA) mRNA, complete cds/cds = (28,636)/gb = U63825/ |
| | gi = 1488313/ug = Hs. 66713/len = 879 |
| 39020_at | Cluster Incl. U82938: Human CD27BP (Siva) mRNA, complete cds/ |
| | cds = (252,821)/gb = U82938/gi = 2228596/ug = Hs. 112058/len = 1034 |
| 37026_at | Cluster Incl. AF001461: Homo sapiens Kruppel-like zinc finger |
| | protein Zf9 mRNA, complete cds/cds = (30,881)/gb = AF001461/ |
| | gi = 3378030/ug = Hs. 76526/len = 1354 |
| 32587_at | Cluster Incl. U07802: Human Tis11d gene, complete cds/ |
| | cds = (291,1739)/gb = U07802/gi = 984508/ug = Hs. 78909/len = 3655 |
| 40448_at | Cluster Incl. M92843: H. sapiens zinc finger transcriptional regulator |
| | mRNA, complete cds/cds = (59,1039)/gb = M92843/gi = 183442/ |
| | ug = Hs. 1665/len = 1746 |
| 779_at | D21337/FEATURE = /DEFINITION = HUMCO Human mRNA for |
| | collagen |

TABLE 22
Prostate Cancer Gleason Score 8/9 Minimum Segregation Set 7.
13 genes (r = 0.992)

| Affymetrix Probe | |
|---|---|
| Set ID (U95Av2) | Description |
| 1733_at | M60315/FEATURE=/DEFINITION = HUMTGFBC Human |
| | transforming growth factor-beta (tgf-beta) mRNA, complete cds |
| 41850_s_at | Cluster Incl. U63825: Human hepatitis delta antigen interacting |
| | protein A (dipA) mRNA, complete cds/cds = (28,636)/gb = U63825/ |
| | gi = 1488313/ug = Hs. 66713/len = 879 |
| 39020_at | Cluster Incl. U82938: Human CD27BP (Siva) mRNA, complete cds/ |
| | cds = (252,821)/gb = U82938/gi = 2228596/ug = Hs. 112058/len = 1034 |
| 33936_at | Cluster Incl. D86181: Homo sapiens DNA for galactocerebrosidase/ |
| | cds = (146,2155)/gb = D86181/gi = 2897770/ug = Hs. 273/len = 3869 |
| 39631_at | Cluster Incl. U52100: Human XMP mRNA, complete cds/ |
| | cds = (63,566)/gb = U52100/gi = 2474095/ug = Hs. 29191/len = 690 |
| 38617_at | Cluster Incl. D45906: Homo sapiens mRNA for LIMK-2, complete |
| | cds/cds = (114,2030)/gb = D45906/gi = 1805593/ug = Hs. 100623/ |
| | len = 3668 |
| 35703_at | Cluster Incl. X06374: Human mRNA for platelet-derived growth |
| | factor PDGF-A/cds = (403,993)/gb = X06374/gi = 35363/ug = Hs. 37040/ |
| | len = 2305 |
| 41257_at | Cluster Incl. D16217: Human mRNA for calpastatin, complete cds/ |
| | cds = (162,2288)/gb = D16217/gi = 303598/ug = Hs. 226067/len = 2493 |
| 32786_at | Cluster Incl. X51345: Human jun-B mRNA for JUN-B protein/ |
| | cds = (253,1296)/gb = X51345/gi = 34014/ug = Hs. 198951/len = 1797 |
| 1052_s_at | M83667/FEATURE = mRNA/DEFINITION = HUMNFIL6BA |
| | Human NF-IL6-beta protein mRNA, complete cds |
| 231_at | M55153/FEATURE=/DEFINITION = HUMTGASE Human |
| | transglutaminase (TGase) mRNA, complete cds |
| 31792_at | Cluster Incl. M20560: Human lipocortin-III mRNA, complete cds/ |
| | cds = (46,1017)/gb = M20560/gi = 186967/ug = Hs. 1378/len = 1339 |
| 36543_at | Cluster Incl. J02931: Human placental tissue factor (two forms) |
| | mRNA, complete cds/cds = (111,998)/gb = J02931/gi = 339501/ |
| | ug = Hs. 62192/len = 2141 |

TABLE 23
Classification of High Grade & “False Positive” Low
Grade Prostate Tumors using High Grade Clusters 4-7.

| | | | | | |
|---|---|---|---|---|---|
| | | | | | No. of |
| | High | High | High | High | Correct |
| | Grade | Grade | Grade | Grade | Clas- |
| Tumor | Cluster 4 | Cluster 5 | Cluster 6 | Cluster 7 | sifications |

Gleason Score 8 or 9 (high grade) Tumors

| | | | | | |
|---|---|---|---|---|---|
| T26 | 1 | 1 | 1 | 1 | 4 |
| T31 | 1 | 1 | 1 | 1 | 4 |
| T45 | 1 | 1 | 1 | 1 | 4 |
| T57 | 1 | 1 | 1 | 1 | 4 |
| T58 | 1 | 1 | 1 | 1 | 4 |
| T59 | 1 | 1 | 1 | 1 | 4 |

Gleason Score 6 or 7 (low grade) Tumors

| | | | | | |
|---|---|---|---|---|---|
| T01 | 1 | 0 | 0 | 0 | 3 |
| T10 | 1 | 1 | 1 | 0 | 1 |
| T25 | 0 | 0 | 0 | 1 | 3 |
| T28 | 0 | 0 | 0 | 0 | 4 |
| T29 | 0 | 0 | 1 | 1 | 2 |
| T36 | 0 | 0 | 0 | 0 | 4 |
| T40 | 0 | 0 | 1 | 0 | 3 |
| T42 | 0 | 1 | 0 | 0 | 3 |
| T43 | 1 | 0 | 0 | 1 | 2 |
| T46 | 1 | 1 | 0 | 0 | 2 |
| T47 | 0 | 1 | 0 | 0 | 3 |
| T53 | 1 | 1 | 0 | 0 | 2 |
| T54 | 0 | 0 | 0 | 0 | 4 |
| T55 | 0 | 0 | 0 | 0 | 4 |
| T56 | 0 | 1 | 0 | 0 | 3 |
| T60 | 0 | 1 | 0 | 0 | 3 |
| T62 | 0 | 1 | 0 | 0 | 3 |
| No. Genes | 5 | 4 | 7 | 13 | |
| in Cluster | | | | | |
| Correlation | 0.995 | 0.998 | 0.995 | 0.992 | |
| Coefficient | | | | | |
| of Cluster | | | | | |

Note:
1 = Positive phenotype association index;
0 = negative phenotype association index.

Application of the methods of present invention to classification of human prostate tumors according to Gleason grade revealed that high grade tumors can be readily distinguished from the majority of low grade prostate cancers based on gene expression analysis of small discrete clusters of genes. However, there is a significant fraction of low grade tumors that closely resemble transcriptional profiles of more advanced and aggressive high grade tumors suggesting that these low grade tumors may represent a precursor of aggressive metastatic disease.

D. Benign Prostatic Hyperplasia (BPH) Sample Classification

Applying method of present invention we identified a BPH vs. prostate cancer discrimination cluster comprising 14 genes listed in Table 22. In this example we utilized human prostate carcinoma cell line gene expression data to develop a first reference set and clinical sample data set presented in Stamey T A, Warrington J A, Caldwell M C, Chen Z, Fan Z, Mahadevappa M, McNeal J E, Nolley R, Zhang Z. Molecular genetic profiling of Gleason grade 4/5 prostate cancers compared to benign prostatic hyperplasia. J Urol 2001 166(6):2171-2177, 2001; incorporate herein by reference. The clinical data set consists of 17 samples obtained from 8 patients with BPH and 9 patients with prostate cancer (Stamey, T. A., et al., 2001).

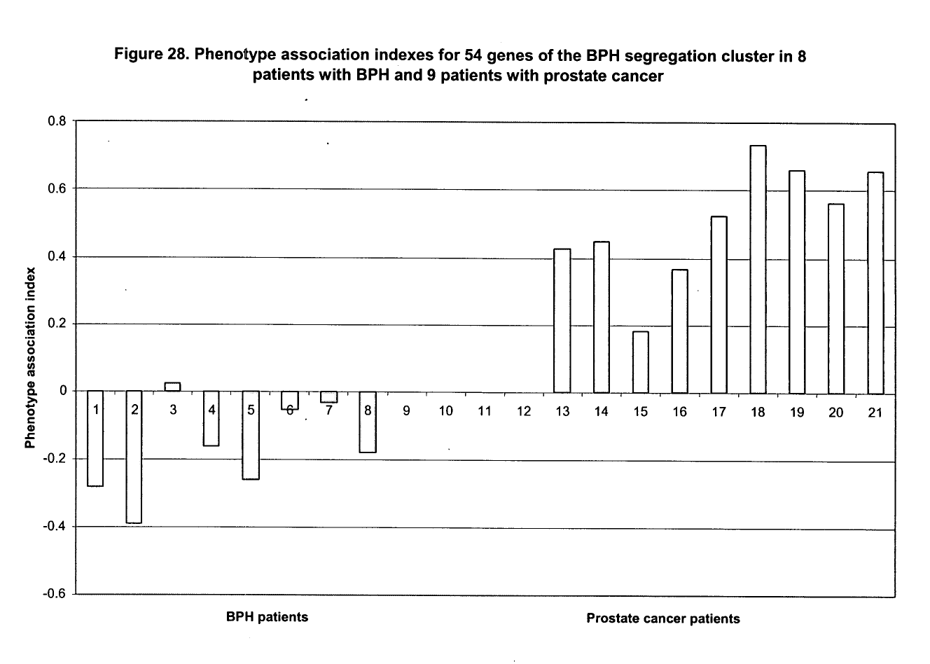

We identified a concordance set of 54 genes (r=0.842) exhibiting concordant gene expression changes between prostate cancer cell lines vs. normal prostate epithelial cells and clinical samples of prostate cancer vs. BPH. As shown in FIG. 28, 7 of 8 samples from the BPH group had negative phenotype association indices, whereas 9 of 9 samples from the prostate cancer group had positive phenotype association indices yielding overall accuracy of 94% in sample classification.

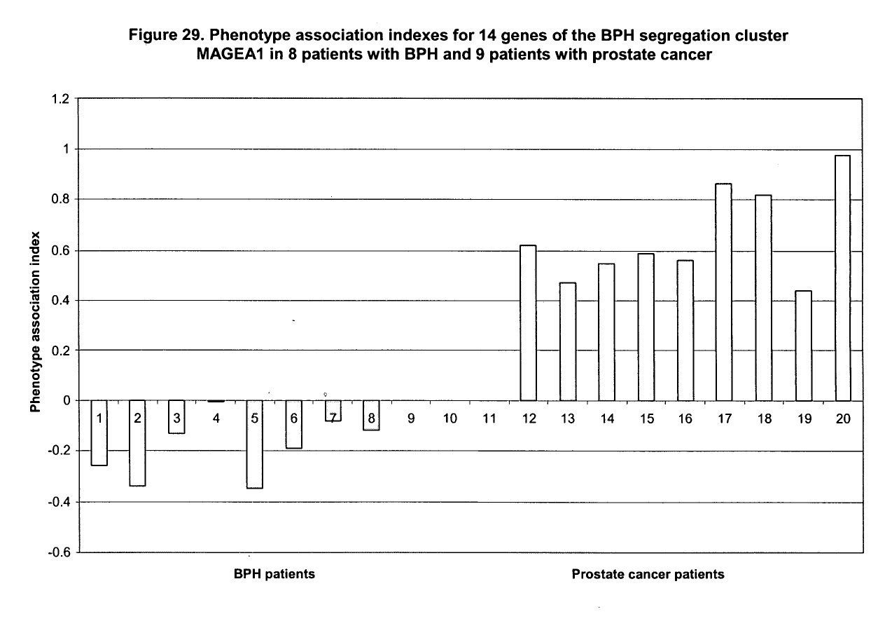

Applying the methods of the present invention, we next identified a minimum segregation set of genes (BPH minimum segregation set 1 or BPH cluster 1 (MAGE- 1 cluster)—Table 24) that is able accurately discriminates between BPH and prostate cancer in clinical tissue samples derived from human prostate. This BPH vs. prostate cancer discrimination cluster comprises 14 genes displaying a high correlation coefficient of −fold expression changes in prostate cancer cell lines vs. normal prostate epithelial cells and clinical samples of prostate cancer vs. BPH (r=0.990) and high accuracy of sample classification. As shown in FIG. 29, of 8 samples from the BPH group had negative phenotype association indices, whereas 9 of 9 samples from the prostate cancer group had positive phenotype association indices yielding overall accuracy of 100% in sample classification.

TABLE 24
BPH Minimum Segregation Set 1.
14 genes (r = 0.990) [BPH segregation cluster (MAGE-1 cluster)]

| Affymetrix Probe Set ID | |
|---|---|
| (U95Av2) | Description |
| M77481_rnal_f_at | MAGE-1 |
| U73514_at | hydroxyacyl-Coenzyme A dehydrogenase, |
| | type II |
| U39840_at | hepatocyte nuclear factor-3 alpha |
| | (HNF-3 alpha) |
| L41559_at | dimerization cofactor of hepatocyte |
| | nuclear factor 1 alpha (TCF1) |
| U90907_at | clone 23907 |
| D00860_at | phosphoribosyl pyrophosphate synthetase |
| | subunit I |
| U81599_at | homeodomain protein HOXB13 |
| X91247_at | thioredoxin reductase 1 |
| U79274_at | clone 23733 |
| J03473_at | poly(ADP-ribose) synthetase |
| HG4312-HT4582_s_at | Transcription Factor IIIa |
| M55593_at | matrix metalloproteinase 2 (gelatinase A, |
| | 72 kD gelatinase, 72 kD type IV collagenase) |
| M11433_at | retinol-binding protein 1, cellular |
| X93510_at | LIM domain protein |

E. Metastatic Prostate Cancer Sample Classification

Applying method of present invention we identified two gene clusters comprising 17 and 19 genes useful for classifying prostate cancer metastases. In this example we utilized human prostate carcinoma cell line gene expression data and clinical sample data set presented in Dhanasekaran, S. M., Barrette, T. R., Ghosh, D., Shah, R., Varambally, S., Kurachi, K., Pienta, K. J., Rubin, M. A., Chinnalyan, A. M. Delineation of prognostic biomarkers in prostate cancer. Nature, 412:822-826, 2001, incorporated herein by reference. As a starting gene set we utilized a set of 242 genes that was identified using a combination of statistical and clustering analyses approach in Dhanasekaran, S. M., et al., 2001 and was found to be useful in classification of various clinical samples using hierarchical clustering algorithm. Our initial analysis applying the methods of the present invention was performed on a small training data set comprising three human prostate cancer cell lines (LNCap; PC3; DU145), three samples of adjacent to cancer normal prostate, one sample of prostatitis, five samples of BPH, ten samples of hormone dependent localized prostate cancer, and seven samples of hormone refractory metastatic prostate cancer.

The original gene expression data were presented as log transformed−fold expression changes of a gene in a sample compared to normal human prostate. For the set of 242 genes we calculated average gene expression values for three prostate cancer cell lines (first reference set) and average expression values for group of metastatic prostate tumors vs. localized prostate tumors (second reference set). The initial set of 242 genes displayed only a weak correlation coefficient of the−fold expression changes in prostate cancer cell lines and clinical samples of metastatic prostate cancer vs. localized prostate cancer (r=0.323).

Applying the methods of the present invention, we identified a concordance set of 72 genes (r=0.866) exhibiting concordant gene expression changes between prostate cancer cell lines and clinical samples of metastatic prostate cancer vs. localized prostate cancer. When we utilized genes of this concordance set to calculate the phenotype association indices in individual clinical samples, 3 of 3 samples from ANP group, 5 of 5 samples from the BPH group, one sample of prostatitis, and five of ten samples of localized prostate cancer had negative phenotype association indices, whereas 7 of 7 samples from the metastatic prostate cancer group had positive phenotype association indices yielding overall accuracy of 84% in sample classification.

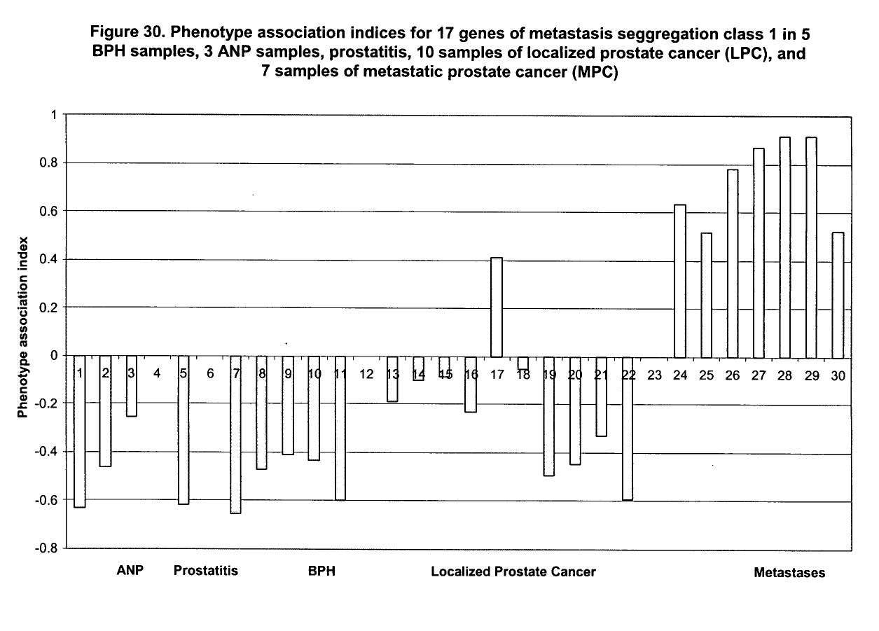

Applying the methods of the present invention, we next identified two minimum segregation sets of genes capable of accurately discriminating between metastatic prostate cancer and localized prostate cancer in clinical tissue samples derived from human prostate. The first metastatic prostate cancer (MPC) vs. localized prostate cancer (LPC) minimum segregation set or cluster (metastasis minimum segregation set 1) comprises 17 genes displaying a high correlation coefficient of fold expression changes in prostate cancer cell lines and clinical samples of metastatic prostate cancer prostate cancer vs. localized prostate cancer (r=0.988) and is highly accurate in discriminating among these different types of samples. As shown in FIG. 30, 3 of 3 samples from ANP group, 5 of 5 samples from the BPH group, one sample of prostatitis, and nine of ten samples of localized prostate cancer had negative phenotype association indices, whereas 7 of 7 samples from the metastatic prostate cancer group had positive phenotype association indices yielding overall accuracy of 96% in sample classification.

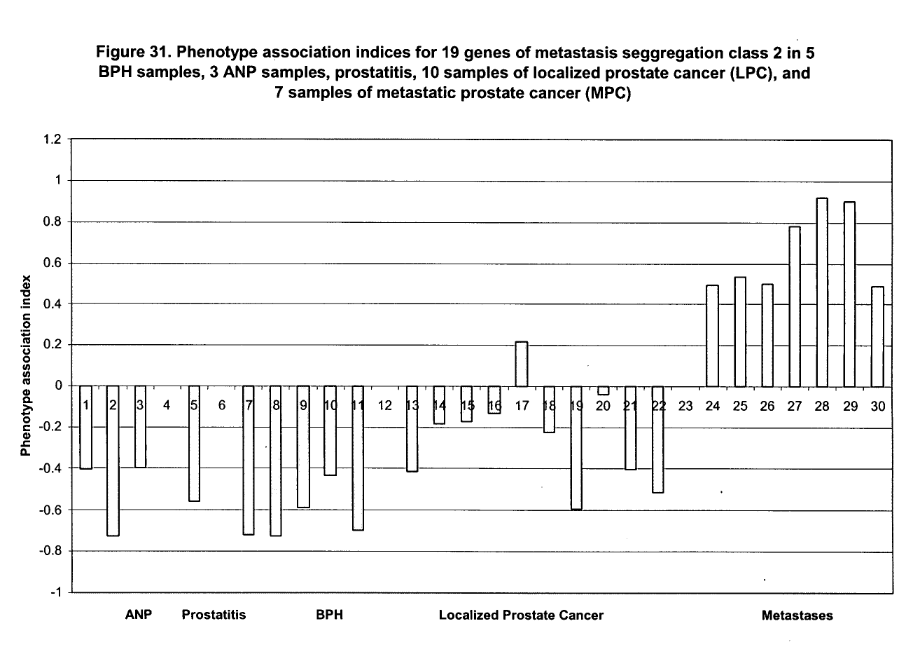

The second metastatic prostate cancer vs. localized prostate cancer discrimination cluster (metastasis minimum segregation set 2) comprises 19 genes displaying a high correlation coefficient of−fold expression changes in prostate cancer cell lines and clinical samples of metastatic prostate cancer prostate cancer vs. localized prostate cancer (r=0.988) and also is highly accurate in discriminating among these different types of samples. As shown in FIG. 31, 3 of 3 samples from ANP group, 5 of 5 samples from the BPH group, one sample of prostatitis, and nine of ten samples of localized prostate cancer had negative phenotype association indices, whereas 7 of 7 samples from the metastatic prostate cancer group had positive phenotype association indices yielding overall accuracy of 96% in sample classification.

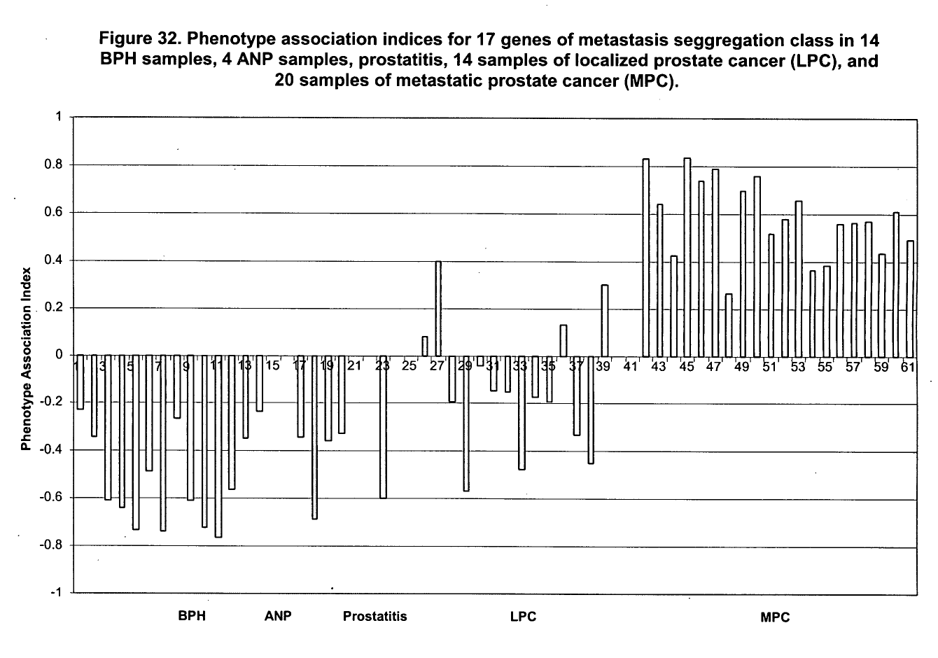

To further validate the sample classification accuracy using an independent data set, we tested the performance of the two metastatic prostate cancer discrimination clusters on a larger set of clinical samples consisting of four samples of adjacent to cancer normal prostate (ANP), one sample of prostatitis, fourteen samples of BPH, fourteen samples of hormone dependent localized prostate cancer (LPC), and twenty samples of hormone refractory metastatic prostate cancer. As shown in FIG. 32, when metastasis minimum segregation set 1 (i.e., the cluster of 17 genes) was utilized, 4 of 4 samples from ANP group, 14 of 14 samples from the BPH group, one sample of prostatitis, and 10 of 14 samples of localized prostate cancer had negative phenotype association indices, whereas 20 of 20 samples from the metastatic prostate cancer group had positive phenotype association indices yielding overall accuracy of 92% in sample classification.

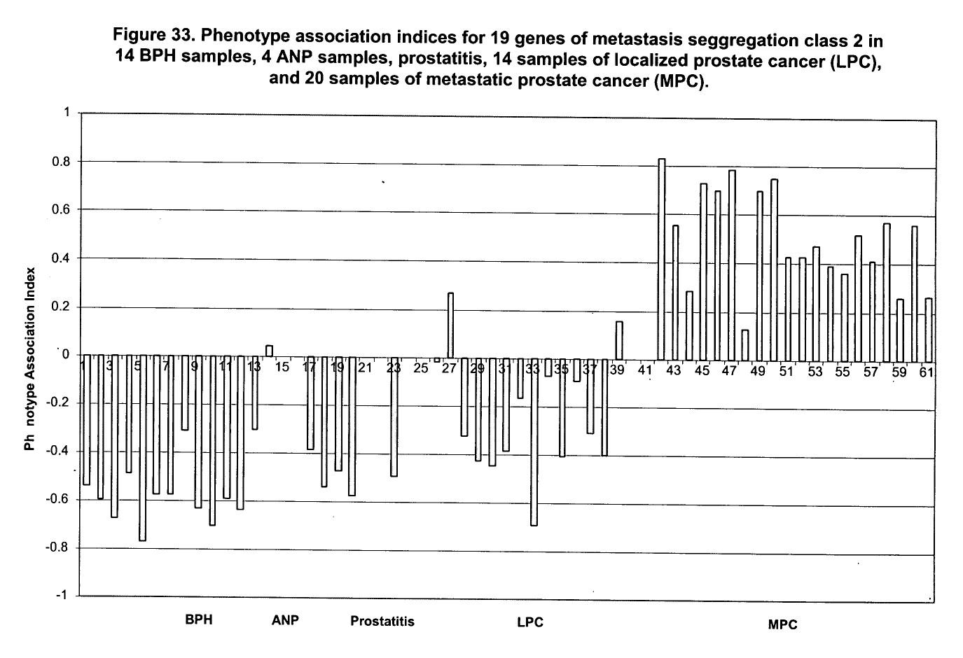

As shown in FIG. 33, when metastasis minimum segregation set 2 (i.e., the cluster of 19 genes) was utilized, 4 of 4 samples from ANP group, 13 of 14 samples from the BPH group, one sample of prostatitis, and 12 of 14 samples of localized prostate cancer had negative phenotype association indices, whereas 20 of 20 samples from the metastatic prostate cancer group had positive phenotype association indices yielding overall accuracy of 94% in sample classification. The genes comprising prostate cancer metastasis minimum segregation sets 1 and 2 are set forth in Tables 25 and 26.

TABLE 25
Prostate Cancer Metastasis Minimum Segregation Set 1.
17 genes (r = 0.988)

| Clone | UniGene | | | Gene | |
|---|---|---|---|---|---|
| ID | Cluster | Accession | NID | Symbol | NAME |
| 469954 | Hs. 169449 | AA030029 | g1496255 | PRKCA | protein kinase C, alpha |
| 308041 | Hs. 3847 | W24429 | g1301379 | PNUTL1 | peanut (Drosophila)-like 1 |
| 83605 | Hs. 50966 | T61078 | g664115 | CPS1 | carbamoyl-phosphate |
| | | | | | synthetase 1, mitochondrial |
| 123755 | Hs. 45514 | R01304 | g751040 | ERG | v-ets avian erythroblastosis |
| | | | | | virus E26 oncogene related |
| 810512 | Hs. 87409 | AA464630 | g2189514 | THBS1 | thrombospondin 1 |
| 811028 | Hs. 9946 | AA485373 | g2214592 | | ESTs |
| 767828 | Hs. 83951 | AA418773 | g2080583 | HPS | Hermansky-Pudlak syndrome |
| 417711 | Hs. 180255 | W88967 | g1404003 | HLA- | major histocompatibility |
| | | | | DRB1 | complex, class II, DR beta 1 |
| 727251 | Hs. 1244 | AA412053 | g2070642 | CD9 | CD9 antigen (p24) |
| 214990 | Hs. 80562 | H72027 | g1043843 | GSN | gelsolin (amyloidosis, Finnish |
| | | | | | type) |
| 788566 | Hs. 80296 | AA452966 | g2166635 | PCP4 | Purkinje cell protein 4 |
| 205049 | Hs. 111676 | H57494 | g1010326 | | ESTs, Weakly similar to heat |
| | | | | | shock protein 27 [H. sapiens] |
| 81289 | Hs. 77443 | T60048 | g661885 | ACTG2 | actin, gamma 2, smooth |
| | | | | | muscle, enteric |
| 77915 | Hs. 76422 | T61323 | g664360 | PLA2G2A | phospholipase A2, group IIA |
| | | | | | (platelets, synovial fluid) |
| 898092 | Hs. 75511 | AA598794 | | CTGF | connective tissue growth factor |
| 343646 | Hs. 2969 | W69471 | | SKI | v-ski avian sarcoma viral |
| | | | | | oncogene homolog |
| 134422 | Hs. 200499 | R31679 | g787522 | | ESTs |

TABLE 26
Prostate Cancer Metastasis Minimum Segregation Set 2.
19 genes (r = 0.988)

| Clone | UniGene | | | Gene | |
|---|---|---|---|---|---|
| ID | Cluster | Accession | NID | Symbol | NAME |
| 469954 | Hs. 169449 | AA030029 | g1496255 | PRKCA | protein kinase C, alpha |
| 308041 | Hs. 3847 | W24429 | g1301379 | PNUTL1 | peanut (Drosophila)-like 1 |
| 83605 | Hs. 50966 | T61078 | g664115 | CPS1 | carbamoyl-phosphate synthetase |
| | | | | | 1, mitochondrial |
| 123755 | Hs. 45514 | R01304 | g751040 | ERG | v-ets avian erythroblastosis virus |
| | | | | | E26 oncogene related |
| 784959 | Hs. 90408 | AA447658 | g2161328 | NEO1 | neogenin (chicken) homolog 1 |
| 130977 | Hs. 23437 | R22926 | g777814 | | Homo sapiens mRNA; cDNA |
| | | | | | DKFZp586G0623 (from clone |
| | | | | | DKFZp586G0623) |
| 80109 | Hs. 198253 | T63324 | g667189 | HLA- | major histocompatibility complex, |
| | | | | DQA1 | class II, DQ alpha 1 |
| 768370 | Hs. 204354 | AA495846 | g2229167 | ARHB | ras homolog gene family, member B |
| 795758 | Hs. 179972 | AA460304 | g2185120 | G1P3 | interferon, alpha-inducible protein |
| | | | | | (clone IFI-6-16) |
| 839736 | Hs. 1940 | AA504943 | g2241103 | CRYAB | crystallin, alpha B |
| 783696 | Hs. 75485 | AA446819 | g2159484 | OAT | omithine aminotransferase (gyrate |
| | | | | | atrophy) |
| 50506 | Hs. 75465 | H17504 | g883744 | MAPK6 | mitogen-activated protein kinase 6 |
| 773771 | Hs. 85050 | AA427940 | g2112058 | PLN | phospholamban |
| 813712 | Hs. 181101 | AA453849 | g2167518 | ATP5F1 | ATP synthase, H+ transporting, |
| | | | | | mitochondrial F0 complex, |
| | | | | | subunit b, isoform 1 |
| 502326 | Hs. 184567 | AA156674 | g1728353 | | ESTs |
| 188036 | Hs. 620 | H44784 | g920836 | BPAG1 | bullous pemphigoid antigen 1 |
| | | | | | (230/240 kD) |
| 840942 | Hs. 814 | AA486627 | g2216791 | HLA- | major histocompatibility complex, |
| | | | | DPB 1 | class II, DP beta 1 |
| 208718 | Hs. 78225 | H63077 | g1017878 | ANXA1 | annexin A1 |
| 753104 | Hs. 240217 | AA478553 | g2207187 | DCT | dopachrome tautomerase |
| | | | | | (dopachrome delta-isomerase, |
| | | | | | tyrosine-related protein 2) |

### EXAMPLE 2

### Classification of Human Breast Cancers

A recent study on gene expression profiling of breast cancer identifies 70 genes whose expression pattern is strongly predictive of a short post-diagnosis and treatment interval to distant metastases (van't Veer, L. J., et al., “Gene expression profiling predicts clinical outcome of breast cancer,” Nature, 415: 530-536, 2002, incorporated herein by reference). The expression pattern of these 70 genes discriminates with 81% (optimized sensitivity threshold) or 83% (optimal accuracy threshold) accuracy the patient's prognosis in the group of 78 young women diagnosed with sporadic lymph-node-negative breast cancer. This group comprises 34 patients who developed distant metastases within 5 years and 44 patients who continued to be disease-free after a period of at least 5 years; they constitute a poor prognosis and good prognosis group, correspondingly.

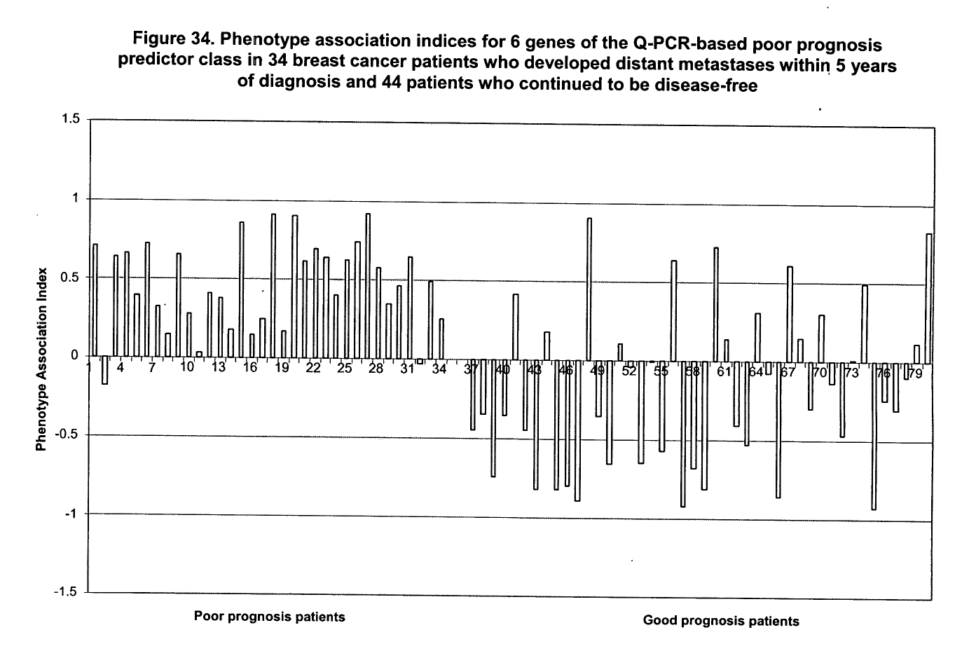

We applied the methods of the present invention to further reduce the number of genes whose expression patterns represent genetic signatures of breast cancers with “poor prognosis” or “good prognosis.” Measurements of mRNA expression levels of 70 genes in established human breast carcinoma cell lines (MCF7; MDA-MB-435; MDA-MB-468; MDA-MB-231; MDA-MB-435Br1; MDA-MB-435BL3) and primary cultures of normal human breast epithelial cells were performed utilizing Q-PCR method, which generally is accepted as the current most reliable method of gene expression analysis and unambiguous confirmation of gene identity. Applying the methods of the present invention, for each breast cancer cell line, concordant sets of genes were identified exhibiting both positive and negative correlation between−fold expression changes in cancer cell lines versus control cell line and the poor prognosis group versus the good prognosis group. Minimum segregation sets were selected from corresponding concordance sets and individual phenotype association indices were calculated. Three top-performing breast cancer metastasis predictor gene clusters are listed in Tables 27-29, and corresponding phenotype association indices are presented in FIGS. 34-36.

A breast cancer poor prognosis predictor cluster comprising 6 genes was identified (r=0.981) using MDA-MB-468 cell line gene expression profile as a reference standard (FIG. 34). 32 of 34 samples from the poor prognosis group had positive phenotype association indices, whereas 29 of 44 samples from the good prognosis group had negative phenotype association indices yielding an overall sample classification accuracy of 78%.

TABLE 27
Breast Cancer Poor Prognosis Minimum Segregation Set 1.
6 genes (MDA-MB-468; Q-PCR) (r = 0.981)

| Systematic | Gene | |
|---|---|---|
| name | name | Sequence description |
| NM_002019 | FLT1 | fms-related tyrosine kinase 1 (vascular |
| | | endothelial growth factor/vascular permeability |
| | | factor receptor) |
| U82987 | BBC3 | Bcl-2 binding component 3 |
| NM_003239 | TGFB3 | transforming growth factor, beta 3 |
| AF201951 | MS4A7 | high affinity immunoglobulin epsilon receptor |
| | | beta subunit |
| NM_000849 | GSTM3 | glutathione S-transferase M3 (brain) |
| NM_003862 | FGF18 | fibroblast growth factor 18 |

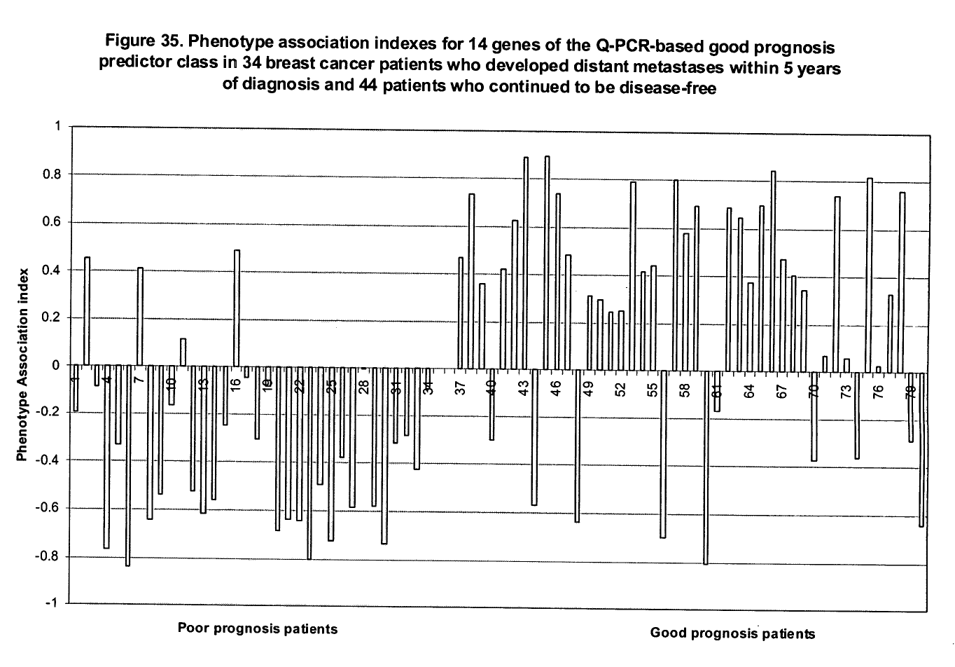

A breast cancer good prognosis predictor cluster comprising 14 genes was identified (r=−0.952) using MDA-MB-435Br1 cell line gene expression profile as a reference standard (FIG. 35). 30 of 34 samples from the poor prognosis group had negative phenotype association indices, whereas 34 of 44 samples from the good prognosis group had positive phenotype association indices yielding an overall sample classification accuracy of 82%.

TABLE 28
Breast Cancer Good Prognosis Minimum Segregation Set 1.
MDA-MB-435Br1 (14 genes; Q-PCR) (r = −0.952)

| Systematic name | Gene name | Sequence description |
|---|---|---|
| AF201951 | MS4A7 | high affinity immunoglobulin |
| | | epsilon receptor beta subunit |
| NM_003239 | TGFB3 | transforming growth factor, beta 3 |
| U82987 | BBC3 | Bcl-2 binding component 3 |
| NM_001282 | AP2B1 | adaptor-related protein complex 2, |
| | | beta 1 subunit |
| NM_003748 | ALDH4A1 | aldehyde dehydrogenase 4 (glutamate |
| | | gamma-semialdehyde dehydrogenase; |
| | | pyrroline-5-carboxylate dehydrogenase) |
| NM_018354 | FLJ11190 | hypothetical protein FLJ11190 |
| NM_020188 | DC13 | DC13 protein |
| NM_003875 | GMPS | guanine monphosphate synthetase |
| Contig57258_RC | AKAP2 | ESTs |
| NM_000788 | DCK | deoxycytidine kinase |
| Contig25991 | ECT2 | epithelial cell transforming sequence |
| | | 2 oncogene |
| Contig38288_RC | | ESTs, Weakly similar to |
| NM_000436 | OXCT | 3-oxoacid CoA transferase |
| NM_000127 | EXT1 | exostoses (multiple) 1 |

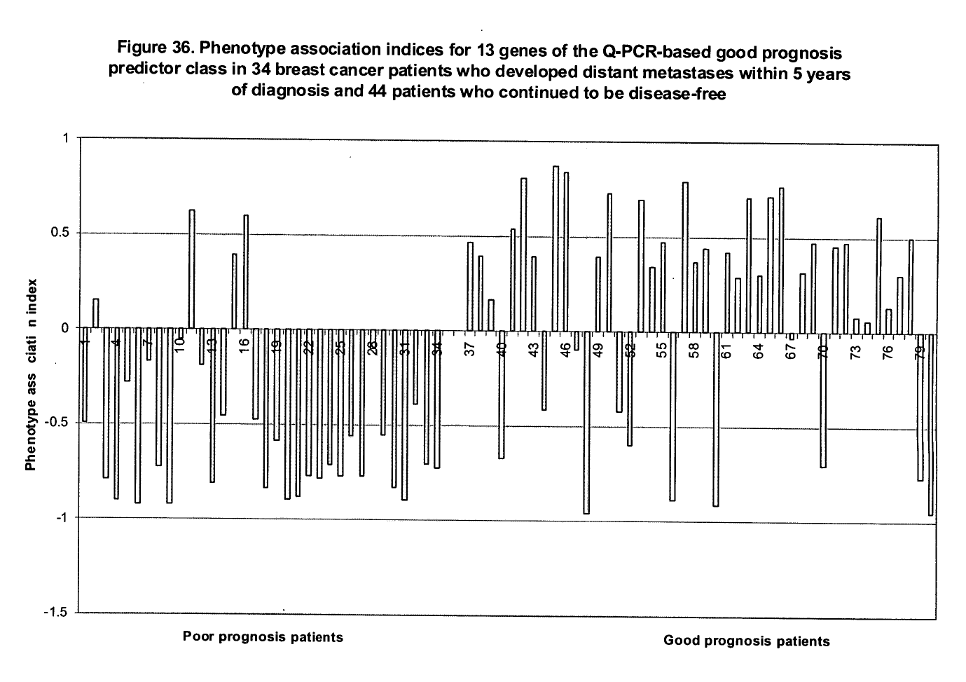

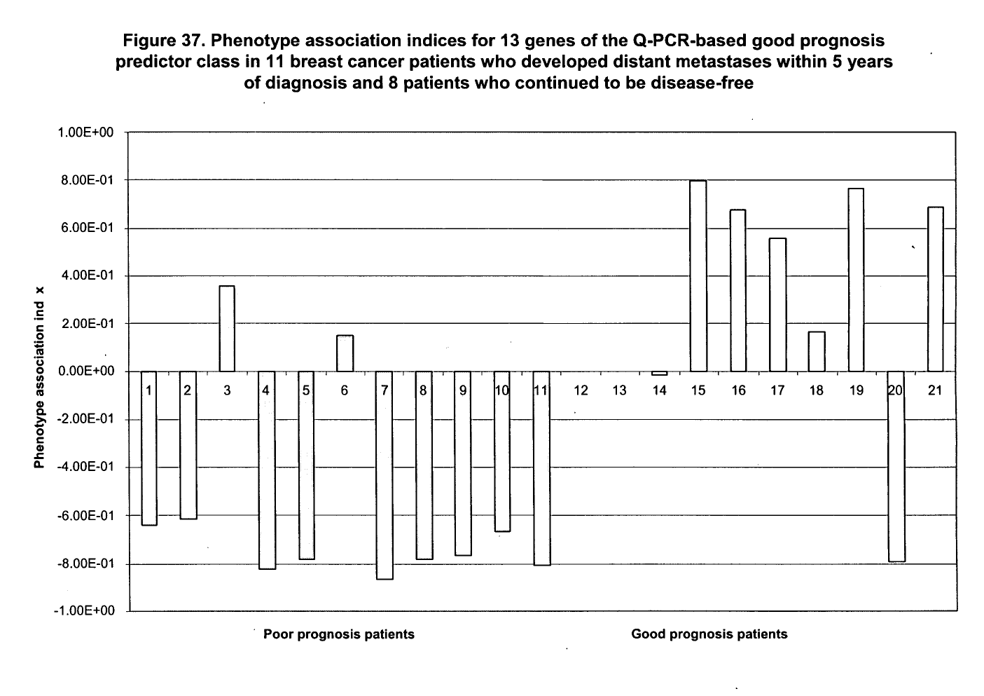

Another breast cancer good prognosis minimum segregation set 2 comprising 13 genes (r=−0.992) was identified using MCF7 cell line gene expression profile as a reference standard (FIG. 36). 30 of 34 samples from the poor prognosis group had negative phenotype association indices, whereas 32 of 44 samples from the good prognosis group had positive phenotype association indices yielding overall sample classification accuracy of 79%.

TABLE 29
Breast Cancer Good Prognosis Minimum Segregation Set 2.

| r = −0.992 | System 1 (MCF7) | | | | |
|---|---|---|---|---|---|
| Locus Link | | | Systematic | Gene | |
| Symbol | GenBank | UniGene | name | name | Gene Description |

| CEGP1 | | Hs. 222399 | NM_020974 | CEGP1 | Homo sapiens CEGP1 |
|---|---|---|---|---|---|
| | | | | | protein (CEGP1), mRNA. |
| FGF18 | | Hs. 49585 | NM_003862 | FGF18 | fibroblast growth factor 18 |
| GSTM3 | | Hs. 2006 | NM_000849 | GSTM3 | glutathione S-transferase |
| | | | | | M3 (brain) |
| TGFB3 | | Hs. 2025 | NM_003239 | TGFB3 | transforming growth |
| | | | | | factor, beta 3 |
| CFFM4 or | | Hs. 11090 | AF201951 | MS4A7 | high affinity |
| MS4A7 | | | | | immunoglobulin epsilon |
| | | | | | receptor beta subunit |
| | AI918032 | Hs. 5521 | Contig55377_RC | | ESTs |
| AP2B1 | | Hs. 74626 | NM_001282 | AP2B1 | adaptor-related protein |
| | | | | | complex 2, beta 1 subunit |
| CCNE2 | | Hs. 30464 | NM_004702 | CCNE2 | cyclin E2 |
| KIAA0175 | | Hs. 184339 | NM_014791 | K1AA0175 | KIAA0175 gene product |
| EXT1 | | Hs. 184161 | NM_000127 | EXT1 | exostoses (multiple) 1 |
| | AI813331 | Hs. 283127 | Contig46218_RC | | ESTs |
| PK428 | | Hs. 44708 | NM_003607 | PK428 | Ser-Thr protein kinase |
| | | | | | related to the myotonic |
| | | | | | dystrophy protein kinase |

| | | | |
|---|---|---|---|
| AI554061 | Hs. 309165 | Contig38288_RC | ESTs, Weakly similar to |
| | | | quiescin [H. sapiens] |

To validate the classification accuracy using an independent data set, we tested performance of the 13 genes good prognosis predictor cluster (good prognosis minimum segregation set 2) on a set of 19 samples obtained from 11 breast cancer patients who developed distant metastases within five years after diagnosis and treatment and 8 patients who remained disease free for at least five years (van't Veer et al., 2002). As shown in FIG. 37, 9 of 11 samples from the poor prognosis group had negative phenotype association indices, whereas 6 of 8 samples from the good prognosis group had positive phenotype association indices yielding overall sample classification accuracy of 79%.

### EXAMPLE 3

### Classification of Human Ovarian Cancer

Lack of effective diagnostic and prognostic markers is generally considered a major problem in the clinical management of ovarian cancer—an epithelial neoplasm that has one of the worst prognoses among epithelial malignancies in women and is the leading cause of death from gynecologic cancer. The clinical utility of the most widely used biomarker of ovarian cancer, CA125, is largely limited to follow-up the response to therapy and progression of the disease and considered to be less efficient in diagnostic and prognostic applications (Meyer, T., Rustin, G. J. Br. J. Cancer, 82: 1535-1538, 2000, incorporated herein by reference).

We applied the methods of the present invention to identify gene expression profiles distinguishing poorly differentiated ovarian epithelial tumors, often exhibiting invasive, highly malignant phenotype, from less aggressive, well and moderately differentiated ovarian epithelial malignancies. Both clinical and cell line data sets utilized in this example were published in Welsh, J. B., et al., “Analysis of gene expression profiles in normal and neoplastic ovarian tissue samples identifies candidate molecular markers of epithelial ovarian cancer,” PNAS, 98: 1176-1181, 2001, incorporated herein by reference. As a starting point for identification of the concordant set of genes for established ovarian cancer cell lines and ovarian tumor tissue samples we utilized a set of the top 501 genes selected by a multidimensional statistical metric that was devised to identify genes with an expression pattern considered ideal for the molecular detection of epithelial ovarian cancer (Welsh et al., 2001). There determined that there was no significant correlation between the−fold changes in the expression levels of these 501 genes in the three cancer cell lines (SKOV8; MDA2774; CAOV3) compared to a control sample (HuOVR) and three poorly differentiated tumors (OVR_{—}11; OVR_{—}12; OVR_{—}27) compared to eleven moderately and well differentiated tumors (OVR_{—}1; _{—}2; _{—}5; _{—}8; _{—}10; _{—}13; _{—}16; _{—}19; _{—}22; _{—}26; _{—}28), (r=0.101).

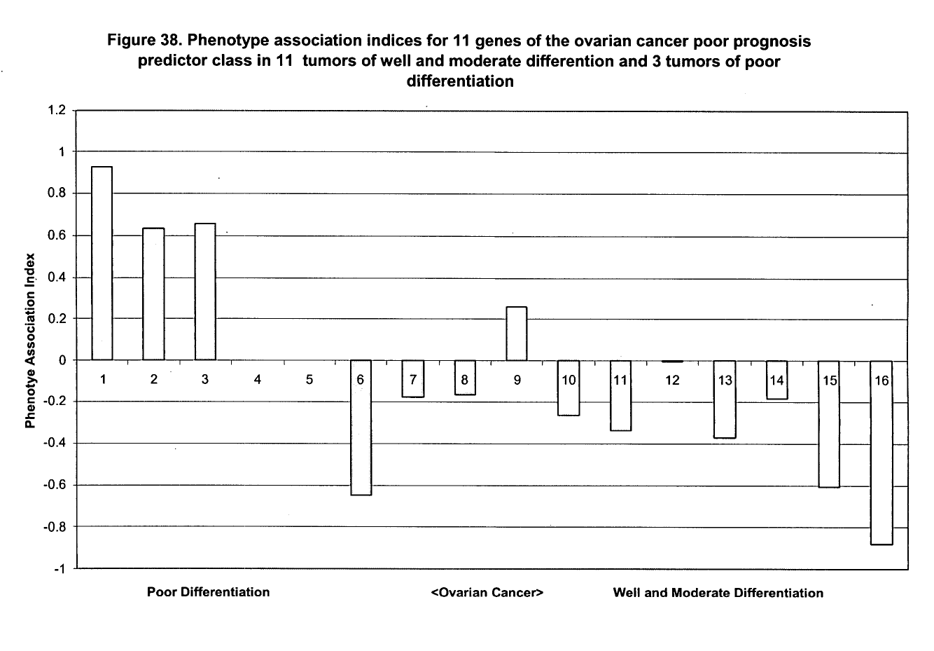

According to the methods of present invention, we selected from the set of 501 genes two concordant sets of genes: concordant set 1 comprising 251 genes and exhibiting positive correlation (r=0.504) between cell lines and tissue samples data sets and concordant set 2, comprising 248 genes and exhibiting negative correlation (r=-0.296) between cell lines and clinical samples. We selected from concordance set 1 a set of 11 genes (ovarian cancer poor prognosis minimum segregation set 1) (ovarian cancer poor prognosis cluster—see Table 30) displaying a high positive correlation (r=0.988) between the cell lines and tissue samples data sets and exhibiting a 93% success rate in clinical sample classification based on individual phenotype association indices. As shown in FIG. 38, all three poorly differentiated tumors had positive phenotype association indices, whereas 10/11 well and moderately differentiated tumors displayed negative phenotype association indices.

TABLE 30
Ovarian Cancer Poor Prognosis Minimum Segregation Set 1.
Poor Prognosis Predictor
Performance: 93% (13/14)
r = 0.988

| Affymetrix Probe Set | |
|---|---|
| ID (HuFL6800) | Description |
| L22524_s_at | L22524, class B, 18 probes, 15 in L22524cds 462-734: 3 in |
| | reverseSequence, 46-197, Human matrilysin gene |
| U47077_at | U47077, class A, 20 probes, 20 in U47077 13025-13463, Human |
| | DNA-dependent protein kinase catalytic subunit (DNA-PKcs) |
| | mRNA, complete cds |
| U46006_s_at | U46006, class A, 20 probes, 20 in U46006 140-620, Human |
| | smooth muscle LIM protein (h-SmLIM) mRNA, complete cds./ |
| | gb = U46006/ntype = RNA |
| L40357_at | L40357, class A, 20 probes, 20 in L40357 mRNA 7-463, Homo |
| | sapiens thyroid receptor interactor (TRIP7) mRNA, 3′ end of cds |
| M64098_at | M64098, class A, 20 probes, 20 in M64098 3873-4305, Human |
| | high density lipoprotein binding protein (HBP) mRNA, complete |
| | cds |
| D79993_at | D79993, class A, 20 probes, 20 in D79993 2741-3167, Human |
| | mRNA for KIAA0171 gene, complete cds |
| U15085_at | U15085, class A, 20 probes, 20 in U15085 821-1289, Human |
| | HLA-DMB mRNA, complete cds |
| U60975_at | U60975, class A, 20 probes, 20 in U60975 6398-6824, Human |
| | hybrid receptor gp250 precursor mRNA, complete cds |
| M79462_at | M79462, class A, 20 probes, 20 in M79462 3853-4333, Human |
| | PML-1 mRNA, complete CDS |
| Z23090_at | Z23090, class A, 20 probes, 17 in Z23090cds 277-589: 3 in |
| | reverseSequence, 1086-1098, H. sapiens mRNA for 28 kDa heat |
| | shock protein |
| X03635_at | X03635, class C, 20 probes, 20 in all_X03635 5885-6402, |
| | Human mRNA for oestrogen receptor |

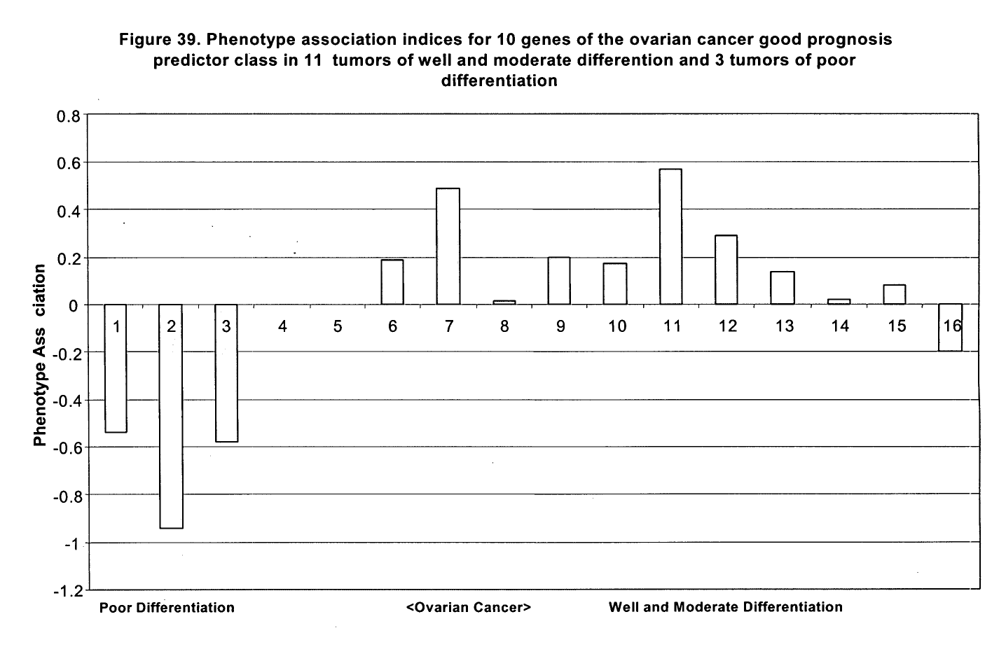

Applying the methods of the present invention, we selected from concordance set 2 a set of 10 genes (ovarian cancer good prognosis minimum segregation set 1) (ovarian cancer good prognosis cluster—see Table 31) displaying a high negative correlation (r=−0.964) between the tumor cell lines and clinical samples data sets and exhibiting a 93% success rate in clinical sample classification based on individual phenotype association indices. As shown in FIG. 39, all three poorly differentiated tumors had negative phenotype association indices, whereas 10/11 well and moderately differentiated tumors displayed positive phenotype association indices.

TABLE 31
Ovarian Cancer Good Prognosis Minimum Segregation Set 1
Good Prognosis Predictor
Performance: 93% (13/14)
r = −0.964

| Affymetrix | |
|---|---|
| Probe | |
| Set ID | |
| (HuFL6800) | Description |
| U90551_at | U90551, class A, 20 probes, 20 in U90551 1071-1623, |
| | Human histone 2A-like protein (H2A/1) |
| | mRNA, complete cds |
| L19779_at | L19779, class A, 20 probes, 20 in L19779 7-496, |
| | Homo sapiens histone H2A.2 mRNA, complete |
| | cds |
| M90657_at | M90657, class A, 20 probes, 20 in M90657 581-1163, |
| | Human tumor antigen (L6) mRNA, complete |
| | cds |
| M13755_at | M13755, class A, 20 probes, 20 in M13755mRNA |
| | 33-591, Human interferon-induced 17-kDa/15-kDa |
| | protein mRNA, complete cds |
| U90915_at | U90915, class A, 20 probes, 20 in U90915 122-674, |
| | Human clone 23600 cytochrome c oxidase |
| | subunit IV mRNA, complete cds |
| Z74792_s_at | Z74792, class A, 20 probes, 20 in Z74792mRNA |
| | 1470-1917, H. sapiens mRNA for CCAAT |
| | transcription binding factor subunit gamma. |
| X99325_at | X99325, class C, 20 probes, 20 in all_X99325 |
| | 1482-1927, H. sapiens mRNA for Ste20-like kinase |
| HG2614- | Collagen, Type Viii, Alpha 1 |
| HT2710_at | J03242, class A, 20 probes, 20 in J03242 1155-1324, |
| J03242_s_at | Human insulin-like growth factor II mRNA, |
| | complete cds |
| D86983_at | D86983, class A, 20 probes, 20 in D86983 5131-5485, |
| | Human mRNA for KIAA0230 gene, partial |
| | cds |

### EXAMPLE 4

### Classification of Human Lung Cancer

Lung cancer accounts for more than 150,000 cancer-related deaths every year in the United States, thus exceeding the combined mortality caused by breast, prostate, and colorectal cancers (Greenlee, R. T., Hill-Harmon, M. B., Murray, T., Thun, M. C A Cancer J. Clin. 51: 15-36, 2001, incorporated herein by reference). Late stage of cancer at diagnosis and lack of efficient diagnostic and prognostic biomarkers are significant factors that adversely affect the clinical management of lung cancer (Mountain, C. F. Revisions in the international system for staging lung cancer. Chest, 111:1710-1717, 1997; Ihde, D. C. Chemotherapy of lung cancer. N. Engl. J. Med., 327:1434-1441, 1992; Sugita, M., Geraci, M., Gao, B., Powell, R. L., Hirsch, F. R., Johnson, G., Lapadat, R., Gabrielson, E., Bremnes, R., Bunn, P. A., Franklin, W. A. Combined use of oligonucleotide and tissue microarrays identifies cancer/testis antigens as biomarkers in lung cancer. Cancer Res., 62:3971-3979, 2002). Non-small-cell lung carcinoma (NSCLC) is a clinically and histopathologically distinct major form of lung cancer and is further classified as adenocarcinoma (most common form of NSCLC), squamous cell carcinoma, and large-cell carcinoma (Travis, W. D., Travis, L. B., Devesa, S. S. Cancer, 75:191-202, 1995).

We applied the methods of the present invention to identify gene expression profiles distinguishing lung adenoracinoma samples from normal lung specimens as well as a highly malignant phenotype of lung adenocarcinoma, associated with short survival after diagnosis and therapy, from less aggressive lung cancers, associated with longer patient survival. Both clinical and cell line data sets utilized in this example were published (Clinical data: Bhattacharjee, A., Richards, W. G., Staunton, J., Li, C., Monti, S., Vasa, P., Ladd, C., Beheshti, J., Bueno, R., Gillette, M., Loda, M., Weber, G., Mark, E. J., Lander, E. S., Wong, W., Johnson, B. E., Golub, T. R., Sugarbaker, D. J., Meyerson, M. Classification of human lung carcinomas by mRNA expression profiling reveals distinct adenocarcinoma subclasses. PNAS, 98: 13790-13795, 2001; incorporated herein by reference; Cell line data: Sugita, M., Geraci, M., Gao, B., Powell, R. L., Hirsch, F. R., Johnson, G., Lapadat, R., Gabrielson, E., Bremnes, R., Bunn, P. A., Franklin, W. A. Combined use of oligonucleotide and tissue microarrays identifies cancer/testis antigens as biomarkers in lung cancer. Cancer Res., 62:3971-3979, 2002; incorporated herein by reference. As a starting point for identification of the concordant set of genes for established lung cancer cell lines and lung cancer tissue samples we utilized a set of the 675 transcripts selected based on a statistical analysis of the quality of the dataset and variability of gene expression across dataset (Bhattacharje et al., 2001). Initial analysis showed that there was no significant correlation between the−fold changes in the expression levels of these 675 genes in the two NSCLC cancer cell lines (H647 and A549 cell lines) compared to a control sample (normal bronchial epithelial cell cultures obtained from a healthy 48-year-old donor) and 139 samples of lung adenoracinomas compared to the 17 normal lung specimens (r=0.163).

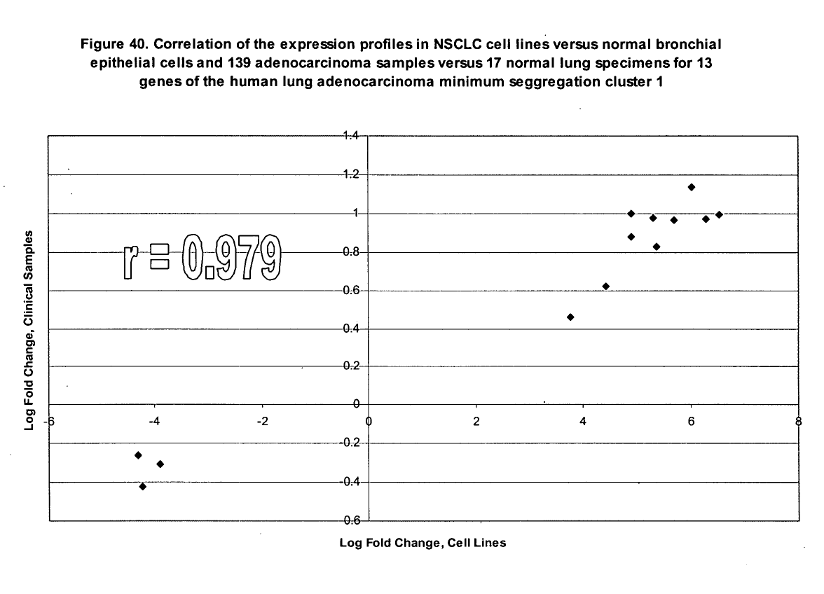

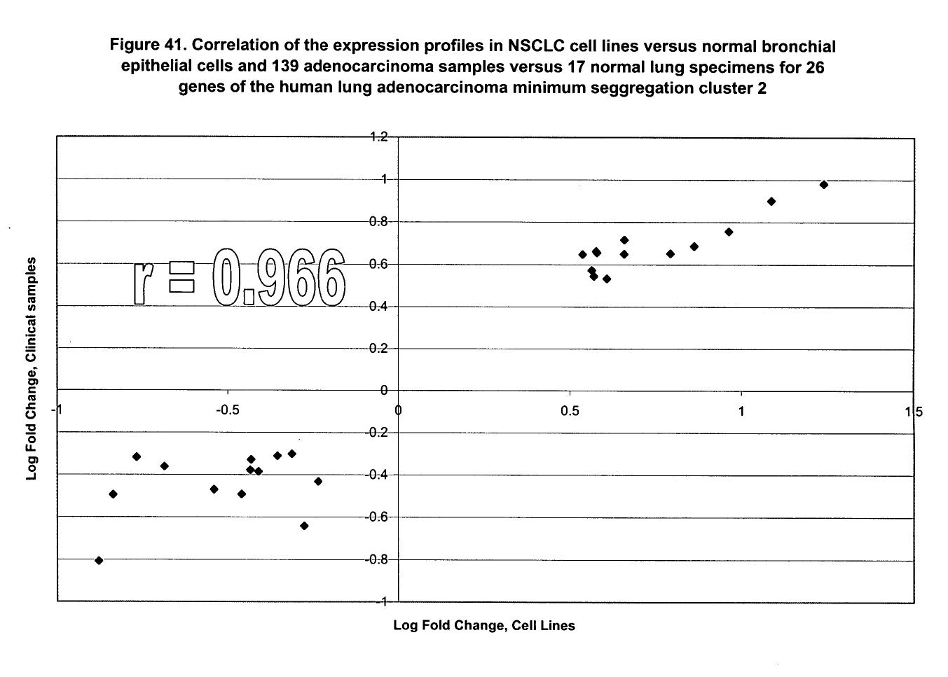

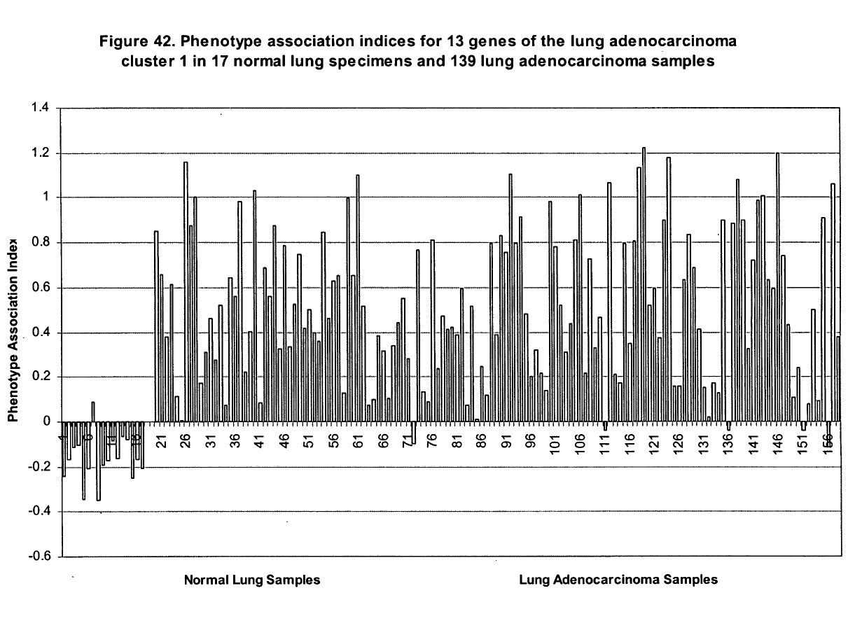

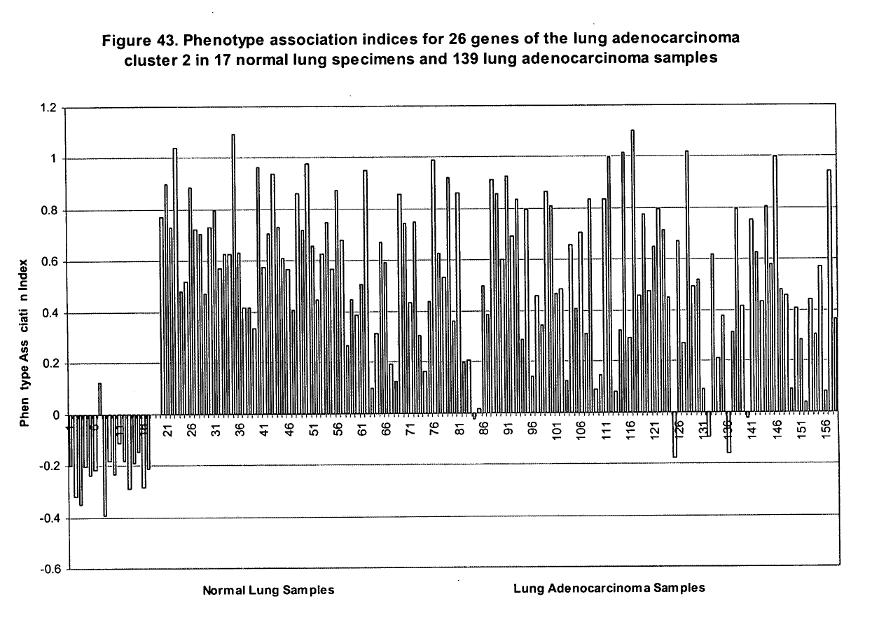

According to the methods of present invention, we selected from the set of 675 genes a concordant set of transcripts comprising 355 genes and exhibiting positive correlation (r=0.523) between cell lines and tissue samples data sets. Next we selected from the concordant set of 355 genes two minimum segregation sets of genes: a set of 13 genes (lung adenoracinoma minimum segregation set 1, also referred to as lung adenocarcinoma cluster 1—see Table 32) and a set of 26 genes (lung adenoracinoma minimum segregation set 2, also referred to as lung adenocarcinoma cluster 2—see Table 33) both displaying high positive correlation (r=0.979 and r=0.966, respectively) between the cell lines and tissue samples data sets (FIGS. 40 and 41). For each minimum segregation set we calculated the individual phenotype association indices for 17 normal lung samples and 139 lung adenocarcinoma samples. After adjustment of the dataset by subtracting 0.52 from all the phenotype association indices, both gene clusters exhibited a 96% success rate in clinical sample classification based on individual phenotype association indices (FIGS. 42 and 43). The adjustment was made following visual inspection of the raw data indicating that 0.52 was a useful threshold for discriminating normal lung samples from lung adenocarcinoma samples, and had the added benefit of allowing classification to be carried out according to the sign of the phenotype association index. Without wishing to be bound by theory, it appears likely that the adjustment was necessary because the published datasets used for constructing this example were derived from different groups using non-identical data reduction methods. As shown in FIGS. 42 and 43, 16/17 normal lung samples had negative phenotype association indices, whereas 134/139 of lung adenocarcinoma specimens displayed positive phenotype association indices. When scores from the two clusters were considered and a criterion of at least one positive phenotype association index was adopted for assigning a lung adenocarcinoma classification, the classification success rate was 99%. 16/17 (94%) normal lung samples had two negative phenotype association indices, whereas 131/139 of lung adenocarcinoma specimens displayed two positive phenotype association indices, seven of 139 had at least one positive phenotype association index, and only a single lung adenocarcinoma specimen had two negative phenotype association indices. Thus, 154/156 (99%) of clinical lung adenocarcinima samples were correctly classified using this strategy.

TABLE 32
Lung adenocarcinoma minimum segregation set 1.
13 genes (r = 0.979)

| Affymetrix Probe | |
|---|---|
| Set ID (U95Av2) | Description |
| 34342_s_at | secreted phosphoprotein 1 (osteopontin, bone sialoprotein I, early T- |
| | lymphocyte activation 1) |
| 2092_s_at | secreted phosphoprotein 1 (osteopontin, bone sialoprotein I, early T- |
| | lymphocyte activation 1) |
| 31798_at | Cluster Incl AA314825: EST186646 Homo sapiens cDNA, 5 end/ |
| | clone = ATCC-111986/clone_end = 5″/gb = AA314825/gi = 1967154/ |
| | ug = Hs. 1406/len = 574″ |
| 668_s_at | matrix metalloproteinase 7 (matrilysin, uterine) |
| 31599_f_at | melanoma antigen, family A, 6 |
| 39008_at | ceruloplasmin (ferroxidase) |
| 31844_at | homogentisate 1,2-dioxygenase (homogentisate oxidase) |
| 31477_at | trefoil factor 3 (intestinal) |
| 38825_at | fibrinogen, A alpha polypeptide |
| 32306_g _at | collagen, type I, alpha 2 |
| 32773_at | Cluster Incl AA868382: ak41e04.s1 Homo sapiens cDNA, 3 end/ |
| | clone = IMAGE-1408542/clone_end = 3″/gb = AA868382/ |
| | gi = 2963827/ug = Hs. 198253/len = 936″ |
| 36623_at | Cluster Incl AB011406: Homo sapiens mRNA for alkalin |
| | phosphatase, complete cds/cds = (176,1750)/gb = AB011406/ |
| | gi = 3401944/ug = Hs. 75431/len = 2510 |
| 31870_at | CD37 antigen |

TABLE 33
Lung adenocarcinoma minimum segregation set 2.
26 genes (r = 0.966)

| Affymetrix Probe | |
|---|---|
| Set ID (U95Av2) | Description |
| 33904_at | claudin 3 |
| 1481_at | matrix metalloproteinase 12 (macrophage elastase) |
| 38261_at | ATP-binding cassette, sub-family C (CFTR/MRP), member 3 |
| 1586_at | insulin-like growth factor binding protein 3 |
| 38066_at | diaphorase (NADH/NADPH) (cytochrome b-5 reductase) |
| 34575_f_at | melanoma antigen, family A, 5 |
| 41583_at | flap structure-specific endonuclease 1 |
| 32787_at | v-erb-b2 avian erythroblastic leukemia viral oncogene homolog 3 |
| 1788_s_at | dual specificity phosphatase 4 |
| 32805_at | aldo-keto reductase family 1, member C1 (dihydrodiol |
| | dehydrogenase 1; 20-alpha (3-alpha)-hydroxysteroid dehydrogenase) |
| 39260_at | solute carrier family 16 (monocarboxylic acid transporters), member 4 |
| 41748_at | Cluster Incl AA196476: zp99g10.r1 Homo sapiens cDNA, 5 end/ |
| | clone = IMAGE-628386/clone_end = 5″/gb = AA196476/gi = 1792058/ |
| | ug = Hs. 182421/len = 697″ |
| 38656_s_at | Cluster Incl W27939: 39g3 Homo sapiens cDNA/gb = W27939/ |
| | gi = 1307887/ug = Hs. 103834/len = 862 |
| 823_at | small inducible cytokine subfamily D (Cys-X3-Cys), member 1 |
| | (fractalkine, neurotactin) |
| 32052_at | hemoglobin, beta |
| 36979_at | solute carrier family 2 (facilitated glucose transporter), member 3 |
| 40367_at | bone morphogenetic protein 2 |
| 36937_s_at | PDZ and LIM domain 1 (elfin) |
| 40567_at | Tubulin, alpha, brain-specific |
| 33900_at | follistatin-like 3 (secreted glycoprotein) |
| 34320_at | Cluster Incl AL050224: Homo sapiens mRNA; cDNA |
| | DKFZp586L2123 (from clone DKFZp586L2123)/cds = UNKNOWN/ |
| | gb = AL050224/gi = 4884466/ug = Hs. 29759/len = 1250 |
| 37027_at | AHNAK nucleoprotein (desmoyokin) |
| 31622_f_at | metallothionein 1F (functional) |
| 609_f_at | metallothionein 1B (functional) |
| 37951_at | deleted in liver cancer 1 |
| 31687_f_at | hemoglobin, beta |

Next we applied the methods of the present invention to identify gene expression profiles distinguishing highly malignant phenotype of lung adenocarcinoma, associated with short patient survival after diagnosis and therapy, from less aggressive lung cancers, associated with longer patient survival. Using the clinical data set and associated clinical history published in Bhattacharje et al., 2001, we selected two groups of adenocarcinoma patients having markedly distinct survival after diagnosis and therapy: poor prognosis group 1 comprising 34 patients with median survival of 8.5 months (range 0.1-17.3 months) and good prognosis group 2 comprising 16 patients with median survival of 84 months (range 75.4-106.1 months).

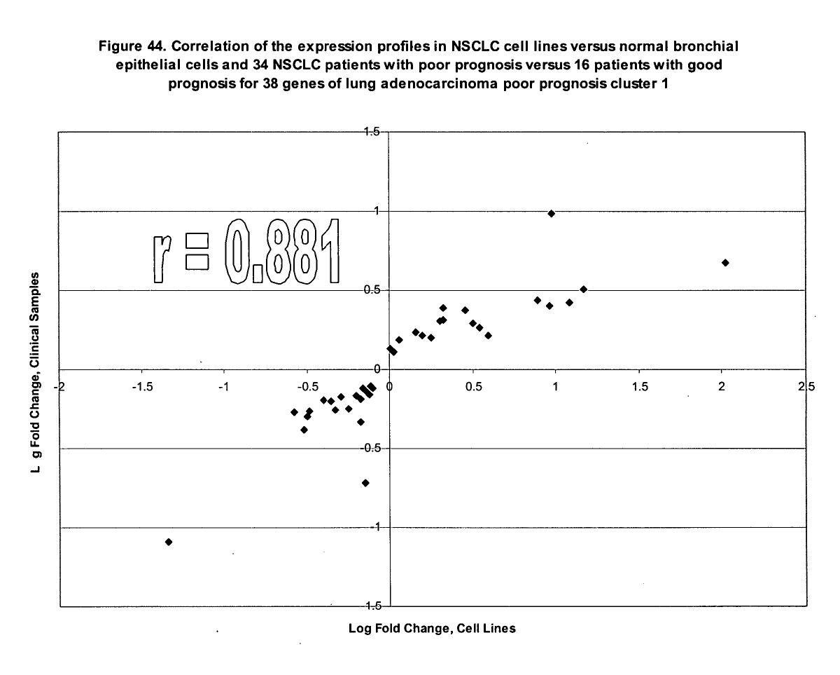

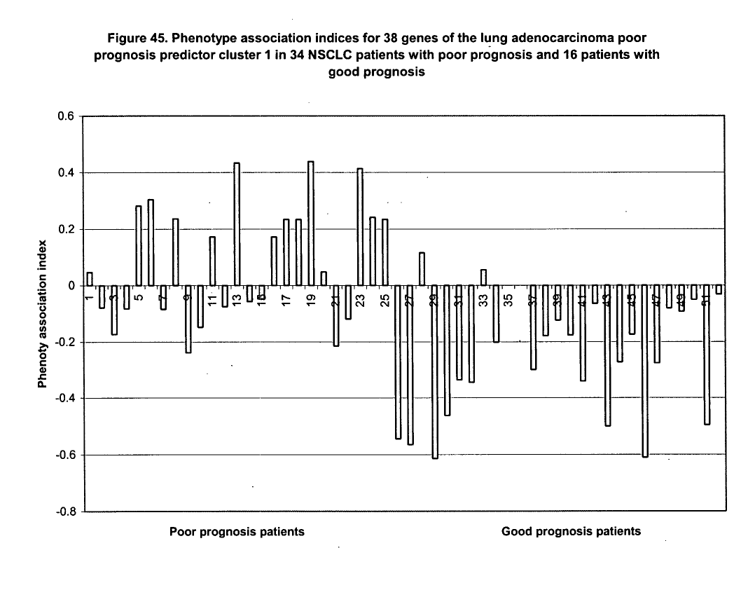

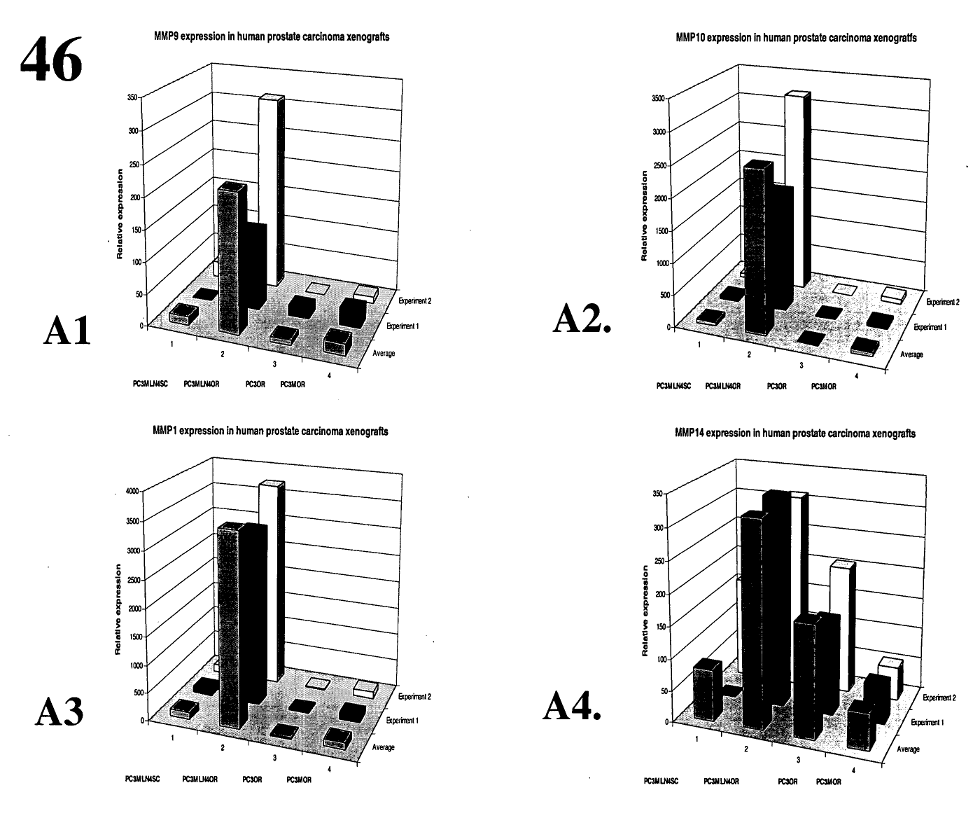

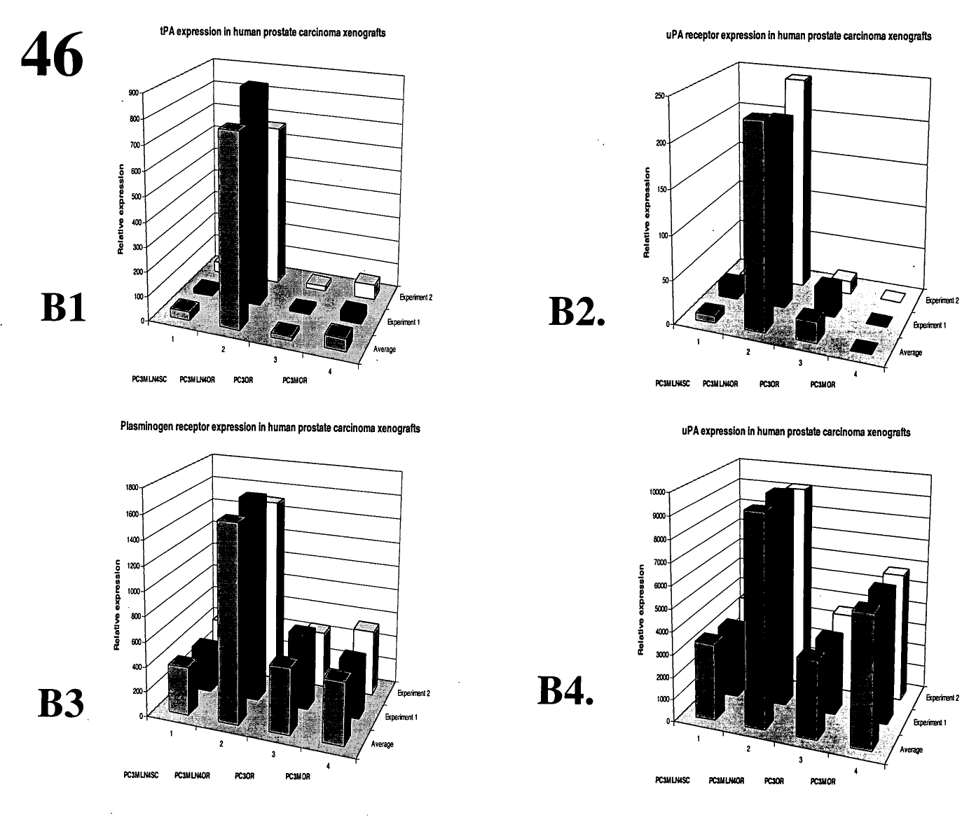

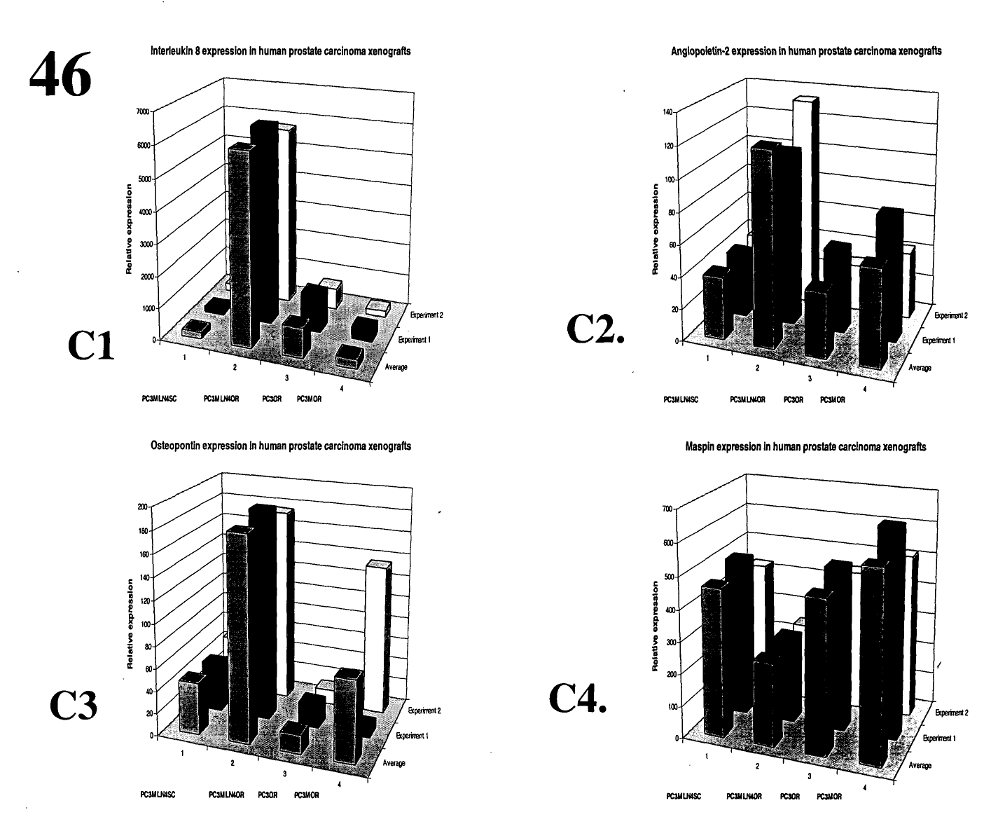

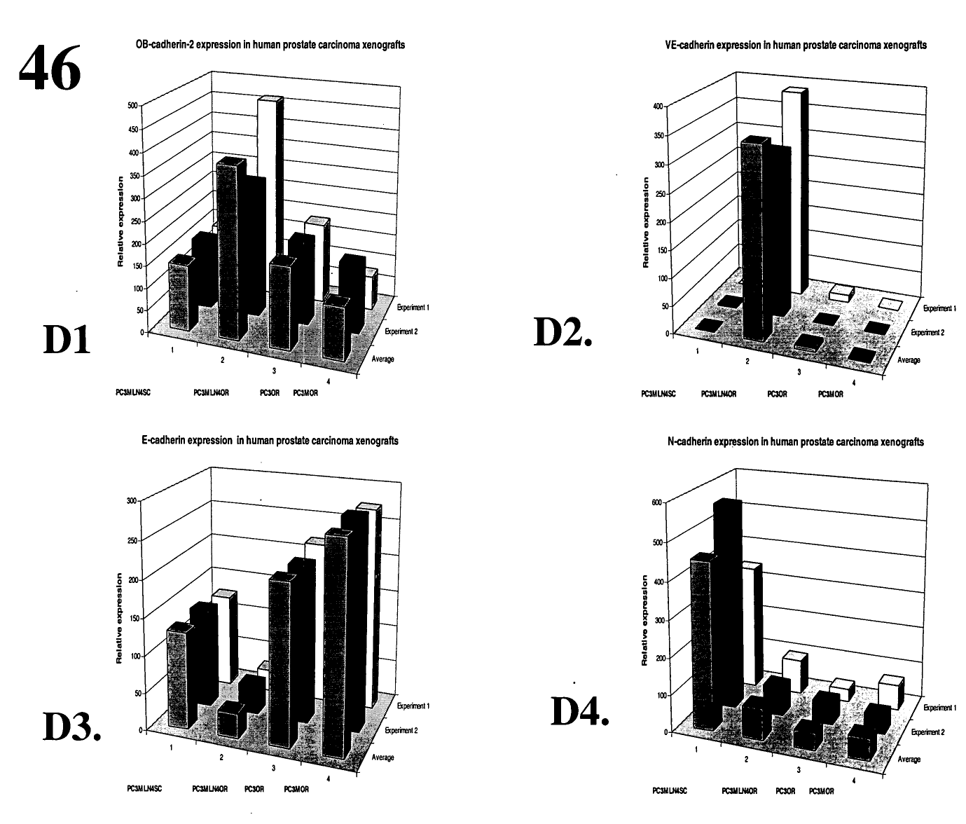

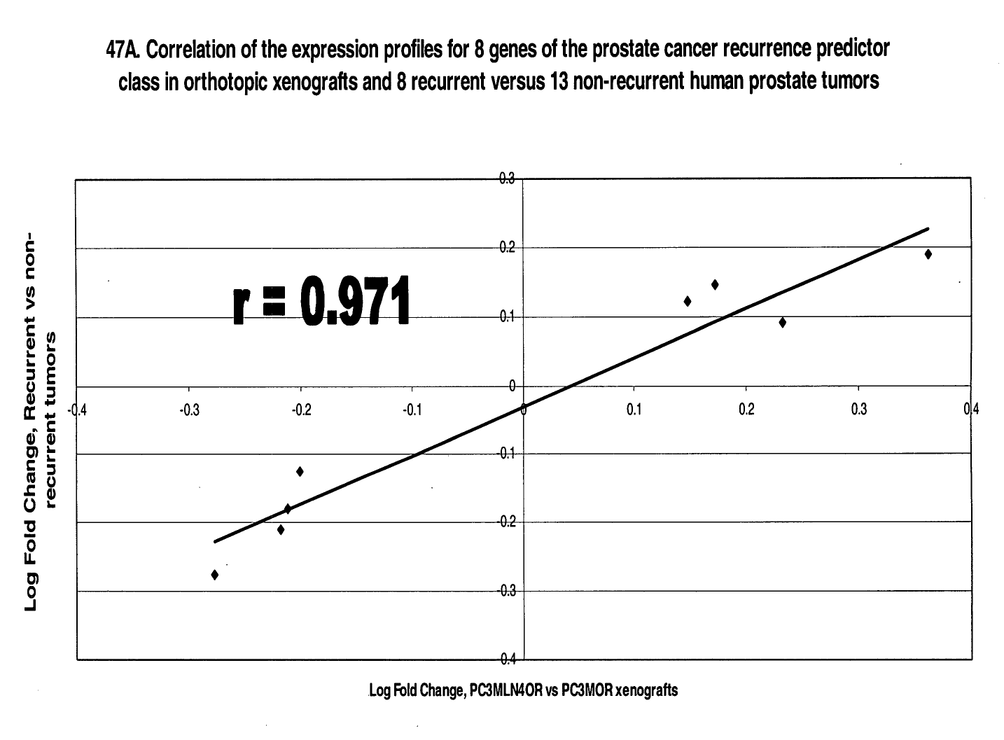

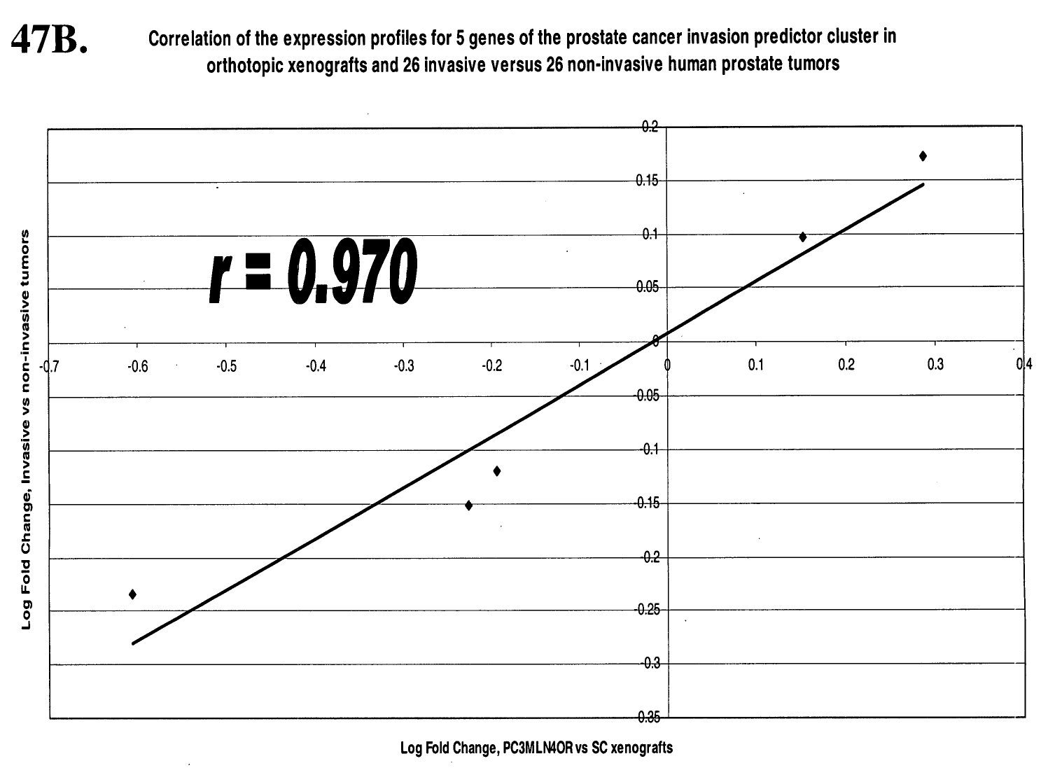

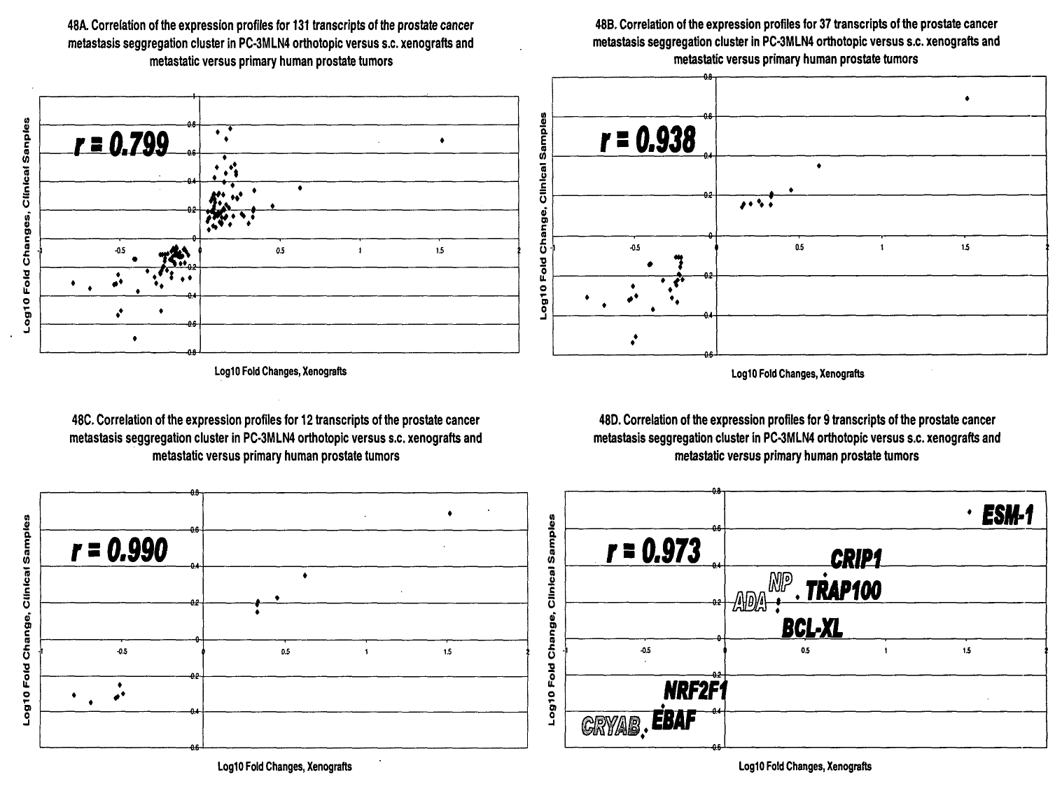

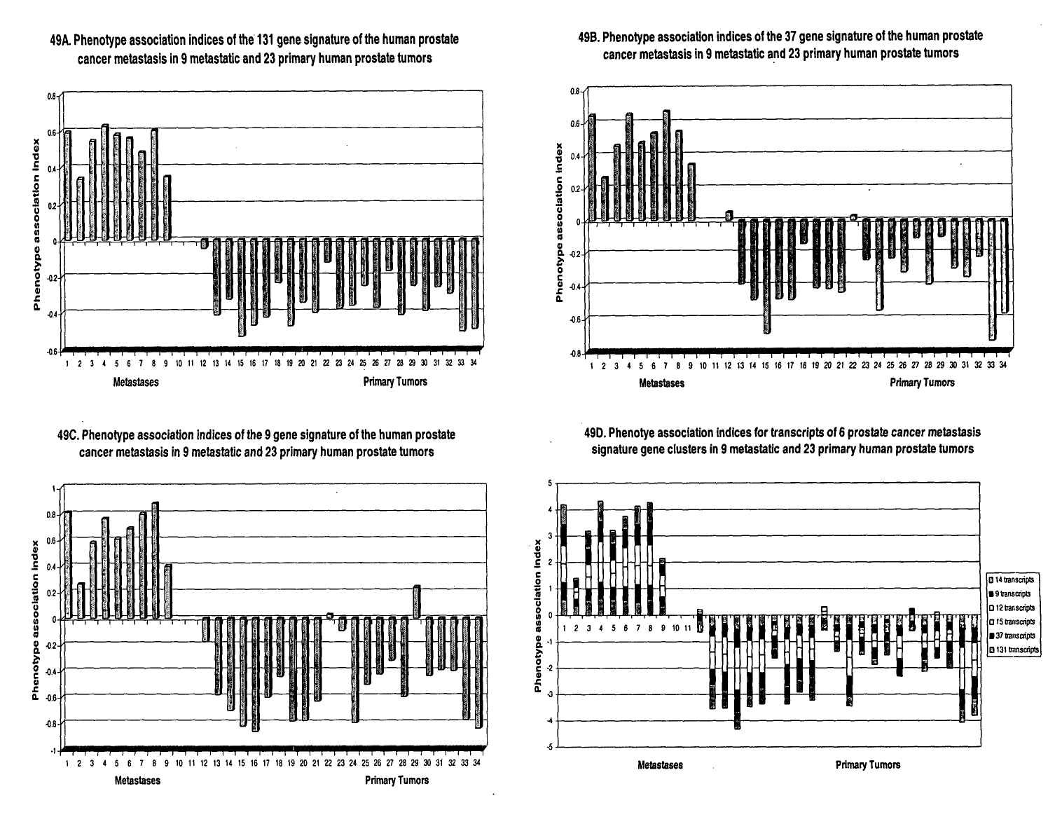

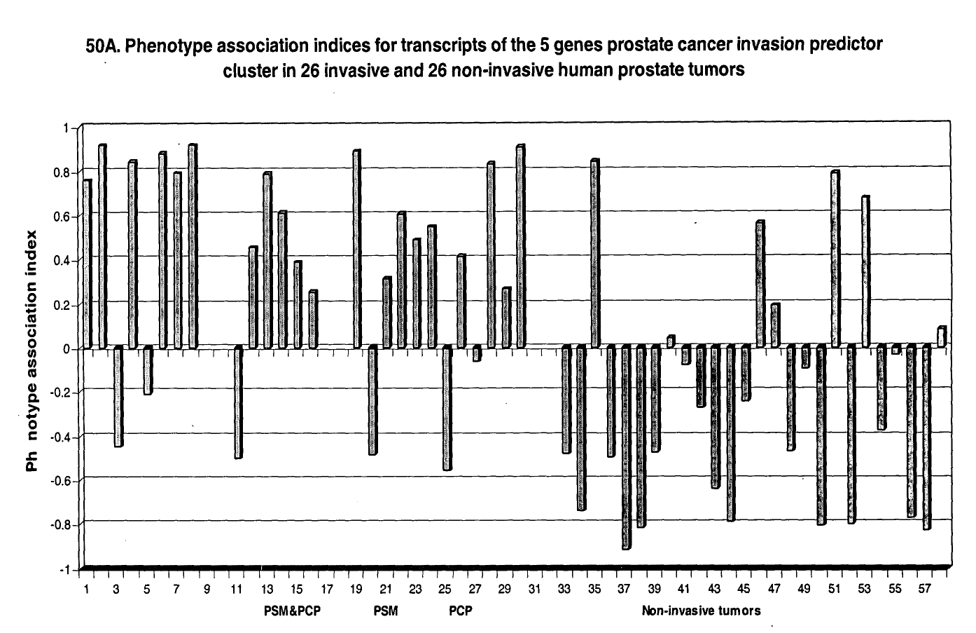

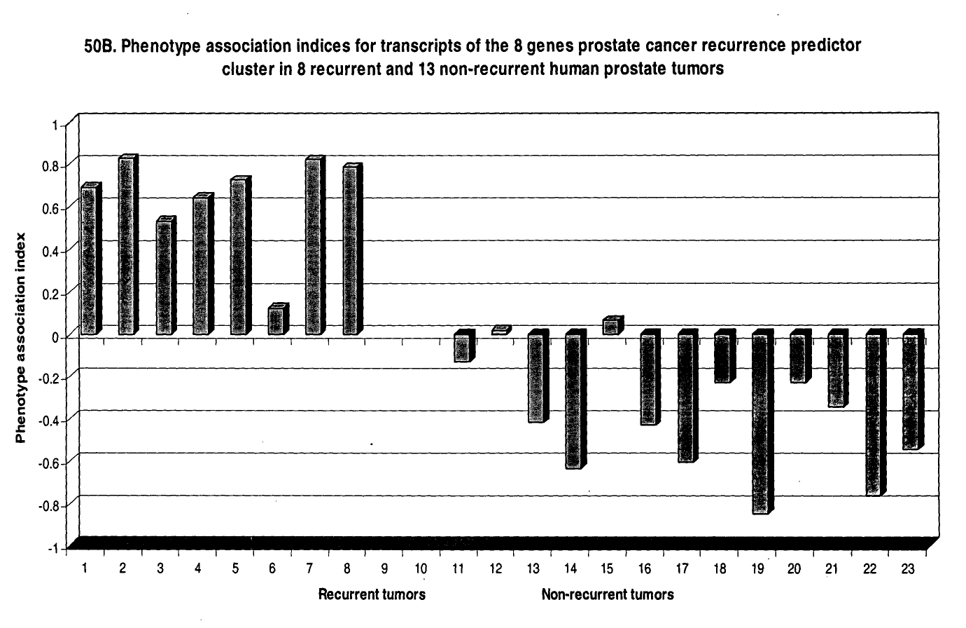

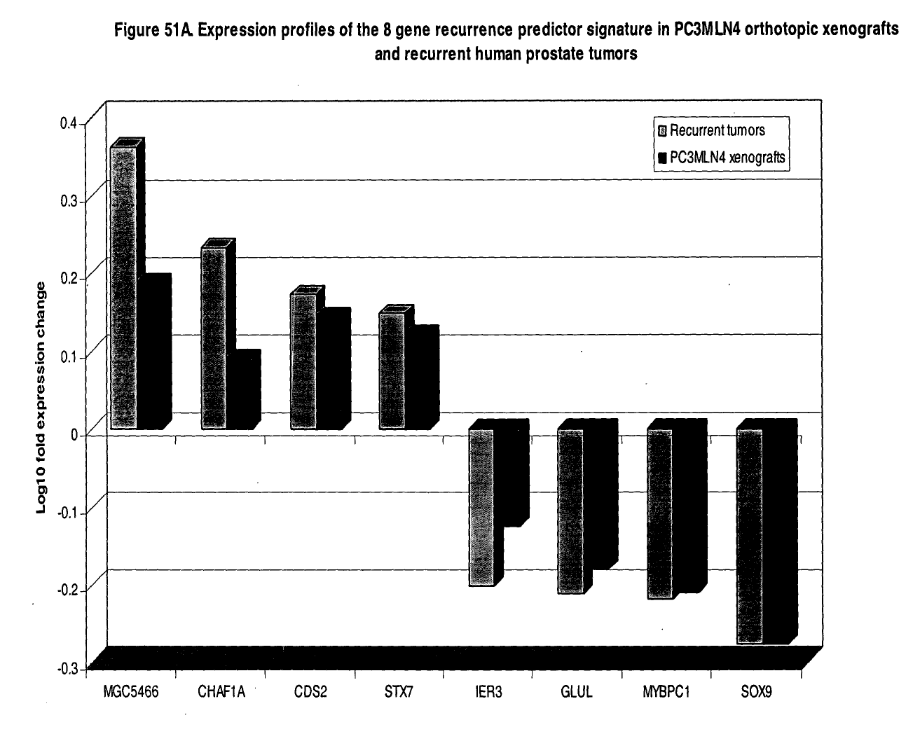

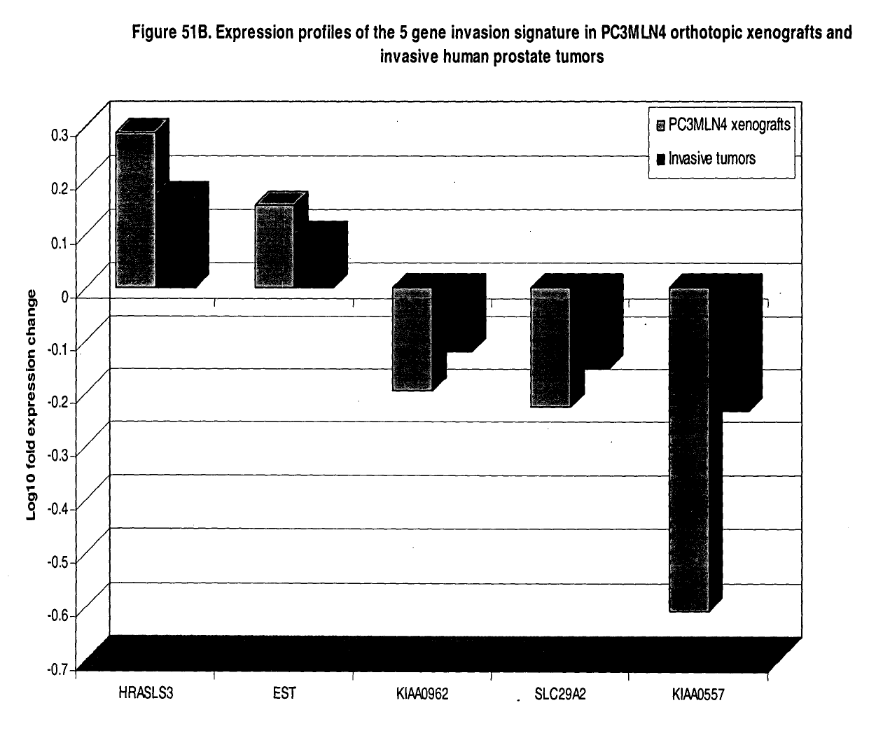

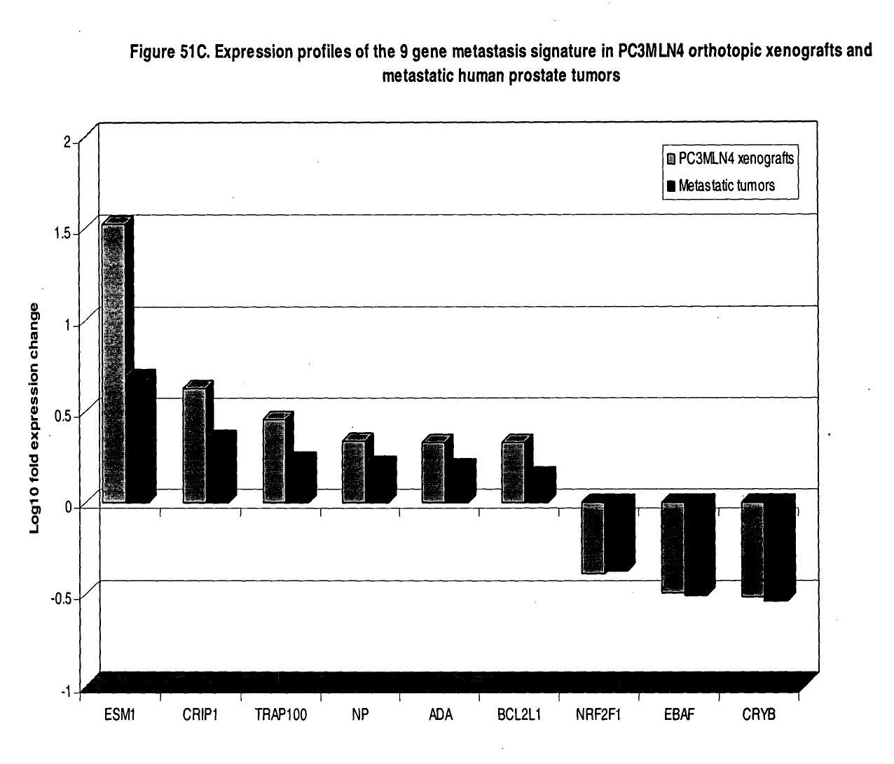

Applying the methods of the present invention, we selected from the set of 675 genes a concordant set of transcripts comprising 302 genes and exhibiting positive correlation (r=0.444) between cell lines data (NSCLC cell lines versus normal bronchial epithelial cells) and tissue samples data sets (poor prognosis samples versus good prognosis samples). We selected from the concordant set of 302 genes a set of 38 genes (lung adenocarcinoma poor prognosis predictor cluster 1—see Table 34) displaying high positive correlation (r=0.881) between the cell lines and tissue samples data sets (FIG. 44). This gene cluster exhibited a 64% success rate in clinical sample classification based on individual phenotype association indices (FIG. 45). As shown in FIG. 45, 16/16 of the lung adenocarcinoma samples of the good prognosis group had negative phenotype association indices, whereas 16/34 of lung adenocarcinoma specimens of the poor prognosis group displayed positive phenotype association indices.

TABLE 34
Lung adenocarcinoma poor prognosis predictor cluster 1.
38 genes (r = 0.881)

| Affymetrix Probe | |
|---|---|
| Set ID (U95Av2) | Description |
| 36990_at | ubiquitin carboxyl-terminal esterase L1 (ubiquitin thiolesterase) |
| 33998_at | neurotensin |
| 1481_at | matrix metalloproteinase 12 (macrophage elastase) |
| 36555_at | synuclein, gamma (breast cancer-specific protein 1) |
| 38389_at | 2′,5′-oligoadenylate synthetase 1 (40-46 kD) |
| 33128_s_at | Cluster Incl W68521: zd36f07.r1 Homo sapiens cDNA, 5 end/ |
| | clone = IMAGE-342757/clone_end = 5″/gb = W68521/gi = 1377410/ |
| | ug = Hs. 83393/len = 579″ |
| 40297_at | six transmembrane epithelial antigen of the prostate |
| 41531_at | Cluster Incl AI445461: tj34g07.x1 Homo sapiens cDNA, 3 end/ |
| | clone = IMAGE-2143452/clone_end = 3″/gb = AI445461/gi = 4288374/ |
| | ug = Hs. 3337/len = 775″ |
| 892_at | transmembrane 4 superfamily member 1 |
| 32821_at | Cluster Incl AI762213: wi54d04.x1 Homo sapiens cDNA, 3 end/ |
| | clone = IMAGE-2394055/clone_end = 3″/gb = AI762213/gi = 5177880/ |
| | ug = Hs. 204238/len = 677″ |
| 1651_at | ubiquitin carrier protein E2-C |
| 37921_at | neuronal pentraxin I |
| 36302_f_at | melanoma antigen, family A, 4 |
| 32426_f_at | melanoma antigen, family A, 1 (directs expression of antigen MZ2- |
| | E) |
| 32607_at | brain acid-soluble protein 1 |
| 41471_at | Cluster Incl W72424: zd66a09.s1 Homo sapiens cDNA, 3 end/ |
| | clone = IMAGE-345592/clone_end = 3″/gb = W72424/gi = 1382379/ |
| | ug = Hs. 112405/len = 604″ |
| 41758_at | chromosome 22 open reading frame 5 |
| 38354_at | CCAAT/enhancer binding protein (C/EBP), beta |
| 195_s_at | caspase 4, apoptosis-related cysteine protease |
| 33267_at | Cluster Incl AF035315: Homo sapiens clone 23664 and 23905 |
| | mRNA sequence/cds = UNKNOWN/gb = AF035315/gi = 2661077/ |
| | ug = Hs. 180737/len = 1331 |
| 39341_at | Cluster Incl AJ001902: Homo sapiens mRNA for TRIP6 (thyroid |
| | receptor interacting protein)/cds = (72,1502)/gb = AJ001902/ |
| | gi = 2558591/ug = Hs. 119498/len = 1653 |
| 34445_at | KIAA0471 gene product |
| 36201_at | glyoxalase I |
| 36736_f_at | phosphoserine phosphatase |
| 1057_at | cellular retinoic acid-binding protein 2 |
| 32072_at | mesothelin |
| 37811_at | calcium channel, voltage-dependent, alpha 2/delta subunit 2 |
| 41771_g_at | Cluster Incl AA420624: nc61c12.r1 Homo sapiens cDNA/ |
| | clone = IMAGE-745750/gb = AA420624/gi = 2094502/ |
| | ug = Hs. 183109/len = 533 |
| 41770_at | Cluster Incl AA420624: nc61c12.r1 Homo sapiens cDNA/ |
| | clone = IMAGE-745750/gb = AA420624/gi = 2094502/ |
| | ug = Hs. 183109/len = 533 |
| 41772_at | monoamine oxidase A |
| 40004_at | sine oculis homeobox (Drosophila) homolog 1 |
| 40367_at | bone morphogenetic protein 2 |
| 40508_at | glutathione S-transferase A4 |
| 33754_at | thyroid transcription factor 1 |
| 32154_at | transcription factor AP-2 alpha (activating enhancer-binding protein |
| | 2 alpha) |
| 37600_at | extracellular matrix protein 1 |
| 37874_at | flavin containing monooxygenase 5 |
| 37208_at | phosphoserine phosphatase-like |

Using the sample iteration and cluster reduction strategies described in the previous examples, we selected four additional sets of genes displaying high positive correlation between the cell lines (NSCLC cell lines versus normal bronchial epithelial cells) and tissue samples data sets (poor prognosis samples versus good prognosis samples) (see Tables 35-38) and thus having potential discriminating power in classification of lung adenocarcinoma samples.

TABLE 35
Lung adenocarcinoma poor prognosis predictor cluster 2.
19 genes (r = 0.938)

| Affymetrix Probe | |
|---|---|
| Set ID (U95Av2) | Description |
| 36555_at | synuclein, gamma (breast cancer-specific protein 1) |
| 41531_at | Cluster Incl AI445461: tj34g07.x1 Homo sapiens cDNA, 3 end/ |
| | clone = IMAGE-2143452/clone_end = 3″/gb = AI445461/ |
| | gi = 4288374/ug = Hs. 3337/len = 775″ |
| 1868_g_at | CASP8 and FADD-like apoptosis regulator |
| 37921_at | neuronal pentraxin I |
| 37918_at | integrin, beta 2 (antigen CD18 (p95), lymphocyte function-associated |
| | antigen 1; macrophage antigen 1 (mac-1) beta subunit) |
| 38422_s_at | four and a half LIM domains 2 |
| 39114_at | decidual protein induced by progesterone |
| 34375_at | small inducible cytokine A2 (monocyte chemotactic protein 1, |
| | homologous to mouse Sig-je) |
| 36495_at | fructose-1,6-bisphosphatase 1 |
| 37187_at | GRO2 oncogene |
| 37014_at | myxovirus (influenza) resistance 1, homolog of murine (interferon- |
| | inducible protein p78) |
| 925_at | interferon, gamma-inducible protein 30 |
| 39372_at | Cluster Incl W26480: 30b8 Homo sapiens cDNA/gb = W26480/ |
| | gi = 1307179/ug = Hs. 12214/len = 854 |
| 32072_at | mesothelin |
| 41771_g_at | Cluster Incl AA420624: nc61c12.r1 Homo sapiens cDNA/clone = |
| | IMAGE-745750/gb = AA420624/gi = 2094502/ |
| | ug = Hs. 183109/len = 533 |
| 40508_at | glutathione S-transferase A4 |
| 41772_at | monoamine oxidase A |
| 40004_at | sine oculis homeobox (Drosophila) homolog 1 |
| 37600_at | extracellular matrix protein 1 |

TABLE 36
Lung adenocarcinoma poor prognosis predictor cluster 3.
23 genes (r = 0.891)

| Affymetrix | |
|---|---|
| Probe | |
| Set ID | |
| (U95Av2) | Description |
| 41106_at | potassium intermediate/small conductance calcium- |
| | activated channel, subfamily N, member 4 |
| 1868_g_at | CASP8 and FADD-like apoptosis regulator |
| 41471_at | Cluster Incl W72424: zd66a09.s1 Homo sapiens cDNA, 3 |
| | end/clone = IMAGE-345592/clone_end = 3″/gb = |
| | W72424/gi = 1382379/ug = Hs. 112405/len = 604″ |
| 37921_at | neuronal pentraxin I |
| 38422_s_at | four and a half LIM domains 2 |
| 39114_at | decidual protein induced by progesterone |
| 34375_at | small inducible cytokine A2 (monocyte chemotactic protein |
| | 1, homologous to mouse Sig-je) |
| 36495_at | fructose-1,6-bisphosphatase 1 |
| 37187_at | GRO2 oncogene |
| 37014_at | myxovirus (influenza) resistance 1, homolog of murine |
| | (interferon-inducible protein p78) |
| 925_at | interferon, gamma-inducible protein 30 |
| 35766_at | keratin 18 |
| 39372_at | Cluster Incl W26480: 30b8 Homo sapiens cDNA/gb = |
| | W26480/gi = 1307179/ug = Hs. 12214/len = 854 |
| 32072_at | mesothelin |
| 40422_at | insulin-like growth factor binding protein 2 (36 kD) |
| 41771_g_at | Cluster Incl AA420624: nc61c12.r1 Homo sapiens cDNA/ |
| | clone = IMAGE-745750/gb = AA420624/gi = 2094502/ |
| | ug = Hs. 183109/len = 533 |
| 40508_at | glutathione S-transferase A4 |
| 1741_s_at | S37730/FEATURE = cds/DEFINITION = S37712S4 |
| | insulin-like growth factor binding protein-2 [human, |
| | placenta, Genomic, 1342 nt, segment 4 of 4] |
| 41772_at | monoamine oxidase A |
| 37874_at | flavin containing monooxygenase 5 |
| 37811_at | calcium channel, voltage-dependent, alpha 2/delta subunit 2 |
| 40004_at | sine oculis homeobox (Drosophila) homolog 1 |
| 37600_at | extracellular matrix protein 1 |

TABLE 37
Lung adenocarcinoma poor prognosis predictor cluster 4.
10 genes (r = 0.872)

| Affymetrix | |
|---|---|
| Probe Set ID | |
| (U95Av2) | Description |
| 34342_s_at | secreted phosphoprotein 1 (osteopontin, bone sialoprotein I, |
| | early T-lymphocyte activation 1) |
| 2092_s_at | secreted phosphoprotein 1 (osteopontin, bone sialoprotein I, |
| | early T-lymphocyte activation 1) |
| 37019_at | fibrinogen, B beta polypeptide |
| 38825_at | fibrinogen, A alpha polypeptide |
| 37233_at | oxidised low density lipoprotein (lectin-like) receptor 1 |
| 31512_at | immunoglobulin kappa variable 1-13 |
| 36736_f_at | phosphoserine phosphatase |
| 37811_at | calcium channel, voltage-dependent, alpha 2/delta subunit 2 |
| 40004_at | sine oculis homeobox (Drosophila) homolog 1 |
| 37874_at | flavin containing monooxygenase 5 |

TABLE 38
Lung adenocarcinoma poor prognosis predictor cluster 5.
6 genes (r = 0.918)

| Affymetrix | |
|---|---|
| Probe | |
| Set ID | Description |
| 34342_s_at | secreted phosphoprotein 1 (osteopontin, bone sialoprotein |
| | I, early T-lymphocyte activation 1) |
| 38825_at | fibrinogen, A alpha polypeptide |
| 31512_at | immunoglobulin kappa variable 1-13 |
| 37811_at | calcium channel, voltage-dependent, alpha 2/delta subunit 2 |
| 40004_at | sine oculis homeobox (Drosophila) homolog 1 |
| 37874_at | flavin containing monooxygenase 5 |

The scoring summary of the individual phenotype association indices calculated for each of the five poor prognosis predictor clusters are presented in Table 39 for the good prognosis patients and in Table 40 for the poor prognosis patients. Only a single patient in the good prognosis group had one positive association index. All the remaining 15 good prognosis patients had negative phenotype association indices for each of the five poor prognosis gene clusters (Table 39). In contrast, 30 of 34 poor prognosis patients had at least one positive association index and 27 of 34 poor prognosis patients scored at least two positive phenotype association indices (Table 40). Thus, applying the methods of the present invention and applying a criterion requiring at least 1 positive phenotype association index for poor prognosis classification, 45 of 50 (90%) adenocarcinoma patients in this data set could be correctly classified as having a good or a poor prognosis.

TABLE 39
Scoring summary of the lung adenocarcinoma poor
prognosis gene clusters for good prognosis patients.
Phenotype Association Indices

| | | | | | | Number of false |
|---|---|---|---|---|---|---|
| Sample | 38 genes | 19 genes | 23 genes | 10 genes | 6 genes | classifications |
| AD187 | −0.06452 | −0.04784 | 0.452696 | −0.00941 | −0.23775 | 1 |
| AD119 | −0.29927 | −0.33723 | −0.1148 | −0.28902 | −0.23916 | 0 |
| AD131 | −0.17964 | −0.48139 | −0.33392 | −0.401 | −0.17498 | 0 |
| AD163 | −0.12353 | −0.28925 | −0.0734 | −0.15033 | −0.01296 | 0 |
| AD170 | −0.17682 | −0.49435 | −0.34161 | −0.32239 | −0.64159 | 0 |
| AD186 | −0.34093 | −0.61548 | −0.28551 | −0.37547 | −0.19218 | 0 |
| AD203 | −0.50111 | −0.52408 | −0.06856 | −0.14395 | −0.21014 | 0 |
| AD250 | −0.27238 | −0.25103 | −0.12624 | −0.68264 | −0.68955 | 0 |
| AD305 | −0.17459 | −0.36628 | −0.29005 | −0.11941 | −0.39534 | 0 |
| AD308 | −0.6101 | −0.03024 | −0.02817 | −0.40192 | −0.34151 | 0 |
| AD317 | −0.276 | −0.56248 | −0.16234 | −0.57284 | −0.51591 | 0 |
| AD318 | −0.08142 | −0.60361 | −0.52572 | −0.30083 | −0.54905 | 0 |
| AD320 | −0.09336 | −0.40628 | −0.09197 | −0.16229 | −0.29432 | 0 |
| AD327 | −0.05072 | −0.11578 | −0.1069 | −0.11479 | −0.49102 | 0 |
| AD338 | −0.49705 | −0.45102 | −0.26864 | −0.803 | −0.84779 | 0 |
| AD367 | −0.03213 | −0.22574 | −0.30494 | −0.5605 | −0.39852 | 0 |

TABLE 40
Scoring summary of the lung adenocarcinoma poor prognosis
gene clusters for poor prognosis patients.
Phenotype Association Indices

| | | | | | | Number of correct |
|---|---|---|---|---|---|---|
| Sample | 38 genes | 19 genes | 23 genes | 10 genes | 6 genes | classification |
| AD277 | 0.234435 | 0.410067 | 0.736989 | 0.574246 | 0.712075 | 5 |
| AD330 | 0.413889 | 0.175061 | 0.101943 | 0.382497 | 0.242026 | 5 |
| AD374 | 0.055386 | 0.455203 | 0.549645 | 0.002916 | 0.052327 | 5 |
| AD177 | 0.304326 | 0.55951 | 0.423434 | −0.08411 | 0.479041 | 4 |
| AD258 | 0.43388 | −0.05816 | 0.293763 | 0.558311 | 0.70477 | 4 |
| AD276 | 0.171625 | −0.53343 | 0.415923 | 0.713297 | 0.80945 | 4 |
| AD287 | 0.233826 | −0.14383 | 0.281022 | 0.069933 | 0.221046 | 4 |
| AD323 | −0.1194 | 0.267964 | 0.027922 | 0.140244 | 0.399934 | 4 |
| AD352 | 0.115964 | 0.041747 | 0.196362 | 0.622718 | 0.802551 | 4 |
| AD157 | −0.08334 | 0.179166 | 0.102028 | −0.1272 | 0.294908 | 3 |
| AD164 | 0.281754 | 0.608169 | 0.31086 | −0.10786 | −0.4293 | 3 |
| AD208 | 0.236001 | 0.310463 | 0.230929 | −0.23772 | −0.70165 | 3 |
| AD221 | −0.23875 | −0.42763 | 0.261846 | 0.292941 | 0.749037 | 3 |
| AD236 | 0.172613 | −0.4351 | 0.155221 | −0.05824 | 0.650534 | 3 |
| AD275 | −0.04808 | 0.203627 | 0.111381 | 0.050702 | −0.17309 | 3 |
| AD296 | 0.438626 | 0.52086 | 0.084982 | −0.57093 | −0.9214 | 3 |
| AD301 | 0.048676 | −0.41297 | −0.27021 | 0.15905 | 0.049724 | 3 |
| AD043 | 0.047335 | −0.00851 | 0.357719 | −0.15053 | −0.23508 | 2 |
| AD127 | −0.07916 | −0.3513 | 0.273233 | −0.03922 | 0.184294 | 2 |
| AD262 | −0.05662 | 0.287899 | 0.423555 | −0.23891 | −0.12164 | 2 |
| AD304 | −0.21516 | 0.186401 | 0.076621 | −0.25509 | −0.18305 | 2 |
| AD332 | 0.241748 | 0.198359 | −0.20156 | −0.22034 | −0.06101 | 2 |
| AD334 | 0.234121 | −0.32246 | −0.47165 | 0.357084 | −0.03519 | 2 |
| AD346 | −0.54482 | −0.40513 | 0.228292 | −0.22006 | 0.355989 | 2 |
| AD361 | −0.46304 | 0.368086 | 0.071209 | −0.455 | −0.48077 | 2 |
| AD363 | −0.33631 | −0.1249 | −0.12018 | 0.161188 | 0.075687 | 2 |
| AD384 | −0.20144 | −0.3584 | 0.451957 | −0.13904 | 0.870275 | 2 |
| AD130 | −0.17359 | −0.26894 | 0.414704 | −0.2768 | −0.41716 | 1 |
| AD225 | −0.14786 | −0.2287 | 0.072267 | −0.0685 | −0.35463 | 1 |
| AD353 | −0.61406 | −0.52593 | 0.187469 | −0.89949 | −0.97919 | 1 |
| AD201 | −0.08499 | −0.4772 | −0.47199 | −0.23861 | −0.54777 | 0 |
| AD252 | −0.07534 | −0.4901 | −0.35684 | −0.23247 | −0.15586 | 0 |
| AD347 | −0.5658 | −0.52075 | −0.31889 | −0.60543 | −0.92335 | 0 |
| AD366 | −0.34494 | −0.56913 | −0.24398 | −0.14348 | −0.43697 | 0 |

### EXAMPLE 5

### Orthotopic Xenograft Gene Expression Profile as Predictive Reference of Expected Transcript Abundance Behavior in Clinical Samples and use to Identify Gene Clusters with Clinically Useful Properties

When human cancer cells derived from the metastatic tumors are injected into ectopic sites in nude mice most do not metastasize (1, 2). The host tissue environment influences metastatic ability of cancer cells in such a way that many human and animal tumors transplanted into nude mice metastasize only if placed in the orthotopic organ (3-8). Several orthotopic models of human cancer metastasis have been developed (9-15). The orthotopic model of human cancer metastasis in nude mice was utilized for in vivo selection of highly and poorly metastatic cell variants (6, 13-15). This approach was successfully applied for development of human prostate cancer cell variants with distinct metastatic potential (15). Experimental evidence indicates that enhancement of metastatic capability of human cancer cells transplanted orthotopically is associated with differential expression of several metastasis-associated genes that have been implicated earlier in certain key features of the metastatic phenotype (16). It is well established that even highly metastatic cells, when implanted ectopically, are not able to consistently produce metastasis.

Here we identified metastasis-associated gene expression signatures based on expression profiling human prostate carcinoma xenografts derived from the same highly metastatic variant implanted at orthotopic (metastasis promoting setting) and ectopic (metastasis suppressing setting) sites, demonstrating that distinct malignant behavior of highly metastatic cells associated with the site of inoculation in a nude mouse is dependent upon differential gene expression in prostate cancer cells implanted either orthotopically or ectopically. We utilized the Affymetrix GeneChip system to compare the expression profiles of 12,625 transcripts in highly metastatic variant PC-3MLN4 implanted at orthotopic (metastasis promoting setting) (“PC3MLN40R”) and ectopic (metastasis suppressing setting) (“PC3MLN4SC”) sites. PC-3MLN4 tumors growing in orthotopic metastasis-promoting setting appear to dramatically over-express a set of genes with well-established invasion-activation functions (FIG. 46). Changes in expression for each transcript are plotted as Log10Fold Change Average expression level in PC-3MLN40R versus Average expression level in less metastatic parental PC30R and PC3MOR (recurrence signatures) (FIG. 47A) or versus Average expression level in PC3PC-3MLN4SC (invasion signatures) (FIG. 47B) and Log10Fold Change Average expression level in aggressive (recurrent or invasive) versus Average expression level in corresponding non-aggressive (non-recurrent or non-invasive) clinical phenotypes. Expression profiling of the 12,625 transcripts in the orthotopic and s.c. xenografts derived from the cell variants of the PC-3 lineage was carried out. Transcripts differentially expressed at the statistically significant level (p<0.05; T-test) in the orthotopic PC-3M-LN4 tumors compared to the s.c. tumors of the same lineage as well as orthotopic tumors derived from the less metastatic parental PC-3M and PC-3 cell lines were identified using the Affymetrix MicroDB and Affymetrix DMT software. Similarly, transcripts differentially regulated in the 8 recurrent versus 13 non-recurrent (FIG. 47A) or 26 invasive versus 26 non-invasive (FIG. 47B) human prostate tumors at the statistically significant level (p<0.05; T-test) were identified. The small clusters of genes exhibiting highly concordant gene expression patterns in the xenograft model and clinical setting were identified using the methods of the invention. In the first example (FIG. 47A), comparisons of the average fold expression changes in highly metastatic PC3MLN4 orthotopic xenografts versus less metastatic parental PC3 and PC3M orthotopic xenografts and 8 recurrent versus 13 non-recurrent primary carcinomas were carried out and a Pearson correlation coefficient was calculated for set of transcripts exhibiting concordant expression changes (FIG. 47A). In the second example (FIG. 47B), comparisons of the average fold expression changes in orthotopic versus s.c. PC3MLN4 xenografts and 26 invasive versus 26 non-invasive primary carcinomas were carried out and a Pearson correlation coefficient was calculated for set of transcripts exhibiting concordant expression changes (FIG. 47B). The transcript abundance levels of several genes encoding matrix metalloproteinases (MMP9; MMP10; MMP1; MMP14 [FIG. 46A1-FIG. 46A4]) as well as components of plasminogen activator (PA)/PA receptor & plasminogen receptor system (uPA; tPA; uPA receptor; plasminogen receptor; PAI-1 [FIGS. 46B1-B4]) are substantially higher in PC-3MLN4 orthotopic tumors versus PC-3MLN4 s.c. (ectopic) tumors, reflecting a plausible mechanistic association of the induction of multiple invasion-activating enzymes with enhanced metastatic potential of PC-3MLN4 tumors in orthotopic setting. Consistent with this idea, the transcript abundance levels for these genes were uniformly lower in orthotopic tumors derived from less metastatic parental PC-3 (“PC30R”) and PC-3M (“PC3MOR”) cells compared to the PC-3MLN4 orthotopic tumors (FIGS. 46A & 46B). Decreased level of expression of protease and angiogenesis inhibitor Maspin in PC-3MLN4 orthotopic tumors (FIG. 46C4) provides an additional clinically relevant example of potential metastasis-promoting molecular alterations in this model since diminished level of Maspin was recently reported in clinical specimens of human prostate cancer (23, 24). Second, a functionally intriguing set of genes highlighted in this model is potentially relevant to metastatic affinity of human prostate carcinoma cells to the bone and represented by a constellation of adhesion molecules (FIG. 46D). Documented in this model is an increase in expression (in a metastasis-promoting setting) of non-epithelial cadherins such as osteoblast cadherins (OB-cadherin-1 and -2) as well as vascular endothelial cadherin (VE-cadherin) along with a concomitantly diminished level of expression of epithelial cadherin (E-cadherin) (FIG. 46D). These molecular aberrations identified in our model correlate with the clinical phenomenon described as a cadherin switching in human prostate carcinoma (25, 26). Interestingly, increased expression of the osteoblast cadherins in clinical prostate cancer specimens was associated with progression and metastasis of human prostate cancer (25, 26), supporting the notion that metastasis-associated molecular alterations identified in the model system are clinically relevant. Two other adhesion molecules expressed in PC-3MLN4 orthotopic tumors, MCAM and ALCAM (data not shown), share some common properties: they mediate both homotypic and heterotypic cell-cell adhesion crucial for metastasis of melanoma cells (27-30); they are expressed on activated leukocytes and on human endothelium (31-35). In addition, ALCAM expression was identified on bone marrow stromal and mesenchymal stem cells and implicated in bone marrow formation and hematopoiesis (31; 36-39). Interestingly, similarly to cadherins, ALCAM is capable to mediate cell-cell adhesion through homophilic ALCAM-ALCAM interactions (31, 40), thus, expression of ALCAM on human prostate carcinoma cells makes this molecule a viable candidate mediator of human prostate carcinoma homing to the bone. MCAM (MUC18) protein over-expression was reported recently in human prostate cancer cell lines, high-grade prostatic intraepithelial neoplasia (PIN), prostate carcinomas, and lymph node metastasis (41, 42).

Expression profiling experiments imply that human prostate carcinoma cells growing in orthotopic metastasis-promoting setting display many clinically relevant gene expression features. Highly aggressive clinically relevant biological behavior of human prostate cancer cells growing in the prostate of nude mice is particularly evident in a fluorescent orthotopic bone metastasis model recapitulating to a significant degree the clinical pattern of metastatic spread of advanced prostate cancer in men (12). Recent gene expression analysis experiments showed that molecular signatures of metastasis could be identified in primary solid tumors (43). We sought to determine whether human prostate carcinoma xenografts growing in the prostate of nude mice would carry the clinically relevant gene-expression signatures of metastasis. We compared the gene expression profiles of 9 metastatic and 23 primary human prostate tumors (the original clinical data were published in LaTulippe, E., Satagopan, J., Smith, A., Scher, H., Scardino, P., Reuter, V., Gerald, W. L. Comprehensive gene expression analysis of prostate cancer reveals distinct transcriptional programs associated with metastatic disease. Cancer Res., 62: 4499-4506, 2002) to identify a broad spectrum of transcripts differentially regulated at the statistically significant level (p<0.05) in metastatic human prostate cancer. Next, we compared a set of transcripts differentially regulated in clinical metastatic human prostate tumors with transcripts differentially regulated in orthotopic human prostate carcinoma xenografts versus subcutaneous (“s.c.”) (i.e., ectopic) tumors of the same lineage. This comparison identified a set of 131 genes that exhibited highly concordant behavior in clinical metastatic samples and orthotopic metastasis-promoting tumors (Pearson correlation coefficient, r=0.799; FIG. 48A; Table 41.0).

TABLE 41.0
Prostate cancer metastasis segregation cluster comprising 131 genes

| Affymetrix | Change | |
|---|---|---|
| Probe ID | direction in | |
| (U95Av2) | metastasis | Description |
| 33534_at | Up | Cluster Incl. X89426: H. sapiens mRNA for ESM-1 |
| | | protein/cds = (55,609)/gb = X89426/gi = 1150418/ |
| | | ug = Hs. 41716/len = 2006 |
| 33232_at | Up | Cluster Incl. AI017574: ou23f10.x1 Homo sapiens cDNA, |
| | | 3 end/clone = IMAGE-1627147/clone_end = 3/ |
| | | gb = AI017574/gi = 3231910/ug = Hs. 17409/len = 501 |
| 34289_f_at | Up | Cluster Incl. D50920: Human mRNA for KIAA0130 |
| | | gene, complete cds/cds = (73,3042)/gb = D50920/ |
| | | gi = 1469182/ug = Hs. 23106/len = 3468 |
| 38158_at | Up | Cluster Incl. D79987: Human mRNA for KIAA0165 |
| | | gene, complete cds/cds = (1113,6500)/gb = D79987/ |
| | | gi = 1136391/ug = Hs. 153479/len = 6662 |
| 430_at | Up | X00737/FEATURE = cds/DEFINITION = HSPNP Human |
| | | mRNA for purine nucleoside phosphorylase (PNP; EC |
| | | 2.4.2.1) |
| 907_at | Up | M13792/FEATURE = cds/DEFINITION = HUMADAG |
| | | Human adenosine deaminase (ADA) gene, complete cds |
| 34742_at | Up | Cluster Incl. Z23115: H. sapiens bcl-xL mRNA/ |
| | | cds = (134,835)/gb = Z23115/gi = 510900/ug = Hs. 239744/ |
| | | len = 926 |
| 1615_at | Up | Z23115/FEATURE = cds/DEFINITION = HSBCLXL |
| | | H. sapiens bcl-xL mRNA |
| 38110_at | Up | Cluster Incl. AF000652: Homo sapiens syntenin (sycl) |
| | | mRNA, complete cds/cds = (148,1044)/gb = AF000652/ |
| | | gi = 2795862/ug = Hs. 8180/len = 2162 |
| 38290_at | Up | Cluster Incl. AF037195: Homo sapiens regulator of G |
| | | protein signaling RGS14 mRNA, complete cds/ |
| | | cds = (73,1398)/gb = AF037195/gi = 2708809/ |
| | | ug = Hs. 9347/len = 1531 |
| 34642_at | Up | Cluster Incl. U28964: Homo sapiens 14-3-3 protein |
| | | mRNA, complete cds/cds = (126,863)/gb = U28964/ |
| | | gi = 899458/ug = Hs. 75103/len = 1030 |
| 36069_at | Up | Cluster Incl. AB007925: Homo sapiens mRNA for |
| | | KIAA0456 protein, partial cds/cds = (0,3287)/ |
| | | gb = AB007925/gi = 3413873/ug = Hs. 5003/len = 6305 |
| 1782_s_at | Up | M31303/FEATURE = mRNA/ |
| | | DEFINITION = HUMOP18A Human oncoprotein 18 |
| | | (Op18) gene, complete cds |
| 527_at | Up | U14518/FEATURE = /DEFINITION = HSU14518 Human |
| | | centromere protein-A (CENP-A) mRNA, complete cds |
| 1854_at | Up | X13293/FEATURE = cds/DEFINITION = HSBMYB |
| | | Human mRNA for B-myb gene |
| 40407_at | Up | Cluster Incl. U28386: Human nuclear localization |
| | | sequence receptor hSRP1alpha mRNA, complete cds/ |
| | | cds = (132,1721)/gb = U28386/gi = 899538/ug = Hs. 159557/ |
| | | len = 1976 |
| 36870_at | Up | Cluster Incl. AB018347: Homo sapiens mRNA for |
| | | KIAA0804 protein, partial cds/cds = (0,3636)/ |
| | | gb = AB018347/gi = 3882328/ug = Hs. 7316/len = 4216 |
| 1797_at | Up | U40343/FEATURE = /DEFINITION = HSU40343 Human |
| | | CDK inhibitor p19INK4d mRNA, complete cds |
| 1054_at | Up | M87339/FEATURE = /DEFINITION = HUMACT1A |
| | | Human replication factor C, 37-kDa subunit mRNA, |
| | | complete cds |
| 36922_at | Up | Cluster Incl. X59618: H. sapiens RR2 mRNA for small |
| | | subunit ribonucleotide reductase/cds = (194,1363)/ |
| | | gb = X59618/gi = 36154/ug = Hs. 75319/len = 2475 |
| 40726_at | Up | Cluster Incl. U37426: Human kinesin-like spindle protein |
| | | HKSP (HKSP) mRNA, complete cds/cds = (90,3260)/ |
| | | gb = U37426/gi = 1171152/ug = Hs. 8878/len = 4858 |
| 34879_at | Up | Cluster Incl. AF007875: Homo sapiens dolichol |
| | | monophosphate mannose synthase (DPM1) mRNA, |
| | | partial cds/cds = (0,761)/gb = AF007875/gi = 2258417/ |
| | | ug = Hs. 5085/len = 1054 |
| 39035_at | Up | Cluster Incl. AF006010: Human progestin induced protein |
| | | (DD5) mRNA, complete cds/cds = (33,8423)/ |
| | | gb = AF006010/gi = 4101694/ug = Hs. 11469/len = 8493 |
| 1624_at | Up | Stimulatory Gdp/Gtp Exchange Protein For C-Ki-Ras |
| | | P21 And Smg P21 |
| 34715_at | Up | Cluster Incl. U74612: Human hepatocyte nuclear factor- |
| | | 3/fork head homolog 11A (HFH-11A) mRNA complete |
| | | cds/cds = (114,2519)/gb = U74612/gi = 1842252/ |
| | | ug = Hs. 239/len = 3474 |
| 1235_at | Up | M86400/FEATURE = /DEFINITION = HUMPHPLA2 |
| | | Human phospholipase A2 mRNA, complete cds |
| 32683_at | Up | Cluster Incl. U18271: Human thymopoietin (TMPO) gene/ |
| | | cds = (313,2397)/gb = U18271/gi = 2182141/ |
| | | ug = Hs. 170225/len = 2796 |
| 41855_at | Up | Cluster Incl. AF030424: Homo sapiens histone |
| | | acetyltransferase 1 mRNA, complete cds/cds = (36,1295)/ |
| | | gb = AF030424/gi = 2623155/ug = Hs. 13340/len = 1568 |
| 981_at | Up | X74794/FEATURE = cds/DEFINITION = HSP1CDC21 |
| | | H. sapiens P1-Cdc21 mRNA |
| 39933_at | Up | Cluster Incl. X93921: H. sapiens mRNA for protein- |
| | | tyrosine-phosphatase (tissue type-testis)/cds = (0,968)/ |
| | | gb = X93921/gi = 1418935/ug = Hs. 3843/len = 1471 |
| 34855_at | Up | Cluster Incl. X76770: H. sapiens PAP mRNA/ |
| | | cds = UNKNOWN/gb = X76770/gi = 556782/ |
| | | ug = Hs. 49007/len = 1956 |
| 31597_r_at | Up | Cluster Incl. L36055: Human 4E-binding protein 1 |
| | | mRNA, complete cds/cds = (0,356)/gb = L36055/ |
| | | gi = 561629/ug = Hs. 198144/len = 357 |
| 182_at | Up | U01062/FEATURE = mRNA/ |
| | | DEFINITION = HUMIP3R3 Human type 3 inositol 1,4,5- |
| | | trisphosphate receptor (ITPR3) mRNA, complete cds |
| 40051_at | Up | Cluster Incl. D31762: Human mRNA for KIAA0057 |
| | | gene, complete cds/cds = (75,1187)/gb = D31762/ |
| | | gi = 498149/ug = Hs. 153954/len = 6974 |
| 1906_at | Up | Ras Inhibitor Inf |
| 38480_s_at | Up | Cluster Incl. U66867: Human ubiquitin conjugating |
| | | enzyme 9 (hUBC9) mRNA, complete cds/ |
| | | cds = (806,1282)/gb = U66867/gi = 1561758/ug = Hs. 84285/ |
| | | len = 1823 |
| 40786_at | Up | Cluster Incl. U37352: Human protein phosphatase 2A |
| | | Balpha1 regulatory subunit mRNA, complete cds/ |
| | | cds = (88,1632)/gb = U37352/gi = 1203811/ug = Hs. 171734/ |
| | | len = 4064 |
| 37729_at | Up | Cluster Incl. Y08614: Homo sapiens mRNA for CRM1 |
| | | protein/cds = (38,3253)/gb = Y08614/gi = 5541866/ |
| | | ug = Hs. 79090/len = 4148 |
| 38702_at | Up | Cluster Incl. AF070640: Homo sapiens clone 24781 |
| | | mRNA sequence/cds = UNKNOWN/gb = AF070640/ |
| | | gi = 3283913/ug = Hs. 108112/len = 1583 |
| 32578_at | Up | Cluster Incl. AW005997: wz91c01.x1 Homo sapiens |
| | | cDNA, 3 end/clone = IMAGE-2566176/clone_end = 3/ |
| | | gb = AW005997/gi = 5854775/ug = Hs. 78185/len = 702 |
| 890_at | Up | M74524/FEATURE = /DEFINITION = HUMHHR6A |
| | | Human HHR6A (yeast RAD 6 homologue) mRNA, |
| | | complete cds |
| 39337_at | Up | Cluster Incl. M37583: Human histone (H2A.Z) mRNA, |
| | | complete cds/cds = (106,492)/gb = M37583/gi = 184059/ |
| | | ug = Hs. 119192/len = 873 |
| 34484_at | Up | Cluster Incl. AI961669: wt65e11.x1 Homo sapiens |
| | | cDNA, 3 end/clone = IMAGE-2512364/clone_end = 3/ |
| | | gb = AI961669/gi = 5754382/ug = Hs. 118249/len = 565 |
| 41085_at | Up | Cluster Incl. AF025840: Homo sapiens DNA polymerase |
| | | epsilon subunit B (DPE2) mRNA, complete cds/ |
| | | cds = (130,1710)/gb = AF025840/gi = 2697122/ |
| | | ug = Hs. 99185/len = 1807 |
| 40690_at | Up | Cluster Incl. X54942: H. sapiens ckshs2 mRNA for Cks1 |
| | | protein homologue/cds = (95,334)/gb = X54942/gi = 29978/ |
| | | ug = Hs. 83758/len = 612 |
| 38818_at | Up | Cluster Incl. Y08685: H. sapiens mRNA for serine |
| | | palmitoyltransferase, subunit I/cds = (0,1421)/ |
| | | gb = Y08685/gi = 2564246/ug = Hs. 90458/len = 1621 |
| 34795_at | Up | Cluster Incl. U84573: Homo sapiens lysyl hydroxylase |
| | | isoform 2 (PLOD2) mRNA, complete cds/cds = (0,2213)/ |
| | | gb = U84573/gi = 2138313/ug = Hs. 41270/len = 3480 |
| 584_s_at | Up | M30938/FEATURE = mRNA#1/ |
| | | DEFINITION = HUMKUP Human Ku (p70/p80) subunit |
| | | mRNA, complete cds |
| 41823_at | Up | Cluster Incl. AJ132258: Homo sapiens mRNA for staufen |
| | | protein, partial/cds = (35,1525)/gb = AJ132258/ |
| | | gi = 4572587/ug = Hs. 6113/len = 3066 |
| 37445_at | Up | Cluster Incl. AB015633: Homo sapiens mRNA for type II |
| | | membrane protein, complete cds, clone-HP10481/ |
| | | cds = (104,1435)/gb = AB015633/gi = 4586843/ |
| | | ug = Hs. 112986/len = 1451 |
| 41569_at | Up | Cluster Incl. AI680675: tx40a08.x1 Homo sapiens cDNA, |
| | | 3 end/clone = IMAGE-2272022/clone_end = 3/ |
| | | gb = AI680675/gi = 4890857/ug = Hs. 44131/len = 554 |
| 1515_at | Up | Rad2 |
| 39724_s_at | Up | Cluster Incl. U58087: Human Hs-cul-1 mRNA, complete |
| | | cds/cds = (124,2382)/gb = U58087/gi = 1381141/ |
| | | ug = Hs. 14541/len = 2511 |
| 36492_at | Up | Cluster Incl. AI347155: tc04c11.x1 Homo sapiens cDNA, |
| | | 3 end/clone = IMAGE-2062868/clone_end = 3/ |
| | | gb = AI347155/gi = 4084361/ug = Hs. 5648/len = 750 |
| 33877_s_at | Up | Cluster Incl. AB028990: Homo sapiens mRNA for |
| | | KIAA1067 protein, partial cds/cds = (0,2072)/ |
| | | gb = AB028990/gi = 5689470/ug = Hs. 24375/len = 4704 |
| 35810_at | Up | Cluster Incl. AI525393: PT1.1_07_A11.r Homo sapiens |
| | | cDNA, 5 end/clone_end = 5/gb = AI525393/gi = 4439528/ |
| | | ug = Hs. 6895/len = 811 |
| 685_f_at | Up | K03460/FEATURE = cds/DEFINITION = HUMTUBA2H |
| | | Human alpha-tubulin isotype H2-alpha gene, last exon |
| 35165_at | Up | Cluster Incl. AF070582: Homo sapiens clone 24766 |
| | | mRNA sequence/cds = UNKNOWN/gb = AF070582/ |
| | | gi = 3387954/ug = Hs. 26118/len = 1744 |
| 36178_at | Up | Cluster Incl. U23143: Human mitochondrial serine |
| | | hydroxymethyltransferase gene, nuclear encoded |
| | | mitochondrion protein, complete cds/cds = (0,1451)/ |
| | | gb = U23143/gi = 746435/ug = Hs. 75069/len = 1452 |
| 32657_at | Up | Cluster Incl. D25278: Human mRNA for KIAA0036 |
| | | gene, complete cds/cds = (156,1952)/gb = D25278/ |
| | | gi = 434780/ug = Hs. 169387/len = 2535 |
| 38839_at | Up | Cluster Incl. AL096719: Homo sapiens mRNA; cDNA |
| | | DKFZp566N043 (from clone DKFZp566N043)/ |
| | | cds = UNKNOWN/gb = AL096719/gi = 5419854/ |
| | | ug = Hs. 91747/len = 2185 |
| 480_at | Up | U56816/FEATURE = /DEFINITION = HSU56816 Human |
| | | kinase Myt1 (Myt1) mRNA, complete cds |
| 982_at | Up | X74795/FEATURE = cds/DEFINITION = HSP1CDC46 |
| | | H. sapiens P1-Cdc46 mRNA |
| 38094_at | Up | Cluster Incl. M65028: Human hnRNP type A/B protein |
| | | mRNA, complete cds/cds = (142,996)/gb = M65028/ |
| | | gi = 337450/ug = Hs. 81361/len = 1537 |
| 37717_at | Up | Cluster Incl. L03532: Human M4 protein mRNA, |
| | | complete cds/cds = (11,2200)/gb = L03532/gi = 187280/ |
| | | ug = Hs. 79024/len = 2457 |
| 36994_at | Up | Cluster Incl. M62762: Human vacuolar H+ ATPase |
| | | proton channel subunit mRNA, complete cds/ |
| | | cds = (230,697)/gb = M62762/gi = 189675/ug = Hs. 76159/ |
| | | len = 1162 |
| 32573_at | Up | Cluster Incl. AL021546: Human DNA sequence from |
| | | BAC 15E1 on chromosome 12. Contains Cytochrome C |
| | | Oxidase Polypeptide VIa-liver precursor gene, 60S |
| | | ribosomal protein L31 pseudogene, pre-mRNA splicing |
| | | factor SRp30c gene, two putative genes, ESTs, STSs and |
| | | pu |
| 32236_at | Up | Cluster Incl. AF032456: Homo sapiens ubiquitin |
| | | conjugating enzyme G2 (UBE2G2) mRNA, complete cds/ |
| | | cds = (55,552)/gb = AF032456/gi = 3004908/ |
| | | ug = Hs. 192853/len = 2890 |
| 38385_at | Down | Cluster Incl. S65738: actin depolymerizing factor [human, |
| | | fetal brain, mRNA, 1452 nt]/cds = (72,569)/gb = S65738/ |
| | | gi = 415586/ug = Hs. 82306/len = 1452 |
| 38982_at | Down | Cluster Incl. W28865: 53g9 Homo sapiens cDNA/ |
| | | gb = W28865/gi = 1308876/ug = Hs. 109875/len = 926 |
| 36051_s_at | Down | Cluster Incl. X58199: Human mRNA for beta adducin/ |
| | | cds = (322,2502)/gb = X58199/gi = 29368/ug = Hs. 4852/ |
| | | len = 2597 |
| 37298_at | Down | Cluster Incl. AF044671: Homo sapiens MM46 mRNA, |
| | | complete cds/cds = (78,431)/gb = AF044671/gi = 4105274/ |
| | | ug = Hs. 7719/len = 859 |
| 34643_at | Down | Cluster Incl. M58458: Human ribosomal protein S4 |
| | | (RPS4X) isoform mRNA, complete cds/cds = (35,826)/ |
| | | gb = M58458/gi = 337509/ug = Hs. 75344/len = 888 |
| 32341_f_at | Down | Cluster Incl. U37230: Human ribosomal protein L23a |
| | | mRNA, complete cds/cds = (23,493)/gb = U37230/ |
| | | gi = 1574941/ug = Hs. 184776/len = 548 |
| 31956_f_at | Down | Cluster Incl. M17886: Human acidic ribosomal |
| | | phosphoprotein P1 mRNA, complete cds/cds = (129,473)/ |
| | | gb = M17886/gi = 190233/ug = Hs. 177592/len = 512 |
| 31957_r_at | Down | Cluster Incl. M17886: Human acidic ribosomal |
| | | phosphoprotein P1 mRNA, complete cds/cds = (129,473)/ |
| | | gb = M17886/gi = 190233/ug = Hs. 177592/len = 512 |
| 1488_at | Down | L77886/FEATURE = /DEFINITION = HUMPTPC |
| | | Human protein tyrosine phosphatase mRNA, complete |
| | | cds |
| 31861_at | Down | Cluster Incl. L14754: Human DNA-binding protein |
| | | (SMBP2) mRNA, complete cds/cds = (49,3030)/ |
| | | gb = L14754/gi = 401775/ug = Hs. 1521/len = 3892 |
| 31962_at | Down | Cluster Incl. L06499: Homo sapiens ribosomal protein |
| | | L37a (RPL37A) mRNA, complete cds/cds = (17,295)/ |
| | | gb = L06499/gi = 292438/ug = Hs. 184109/len = 357 |
| 34864_at | Down | Cluster Incl. AF070638: Homo sapiens clone 24448 |
| | | unknown mRNA, partial cds/cds = (0,659)/gb = AF070638/ |
| | | gi = 3283909/ug = Hs. 4973/len = 1348 |
| 32412_at | Down | Cluster Incl. M13934: Human ribosomal protein S14 |
| | | gene, complete cds/cds = (2,457)/gb = M13934/ |
| | | gi = 337498/ug = Hs. 3491/len = 503 |
| 36980_at | Down | Cluster Incl. U03105: Human B4-2 protein mRNA, |
| | | complete cds/cds = (113,1096)/gb = U03105/gi = 476094/ |
| | | ug = Hs. 75969/len = 2061 |
| 33116_f_at | Down | Cluster Incl. AA977163: oq25a04.s1 Homo sapiens |
| | | cDNA, 3 end/clone = IMAGE-1587342/clone_end = 3/ |
| | | gb = AA977163/gi = 3154609/ug = Hs. 82148/len = 524 |
| 35119_at | Down | Cluster Incl. X56932: H. sapiens mRNA for 23 kD highly |
| | | basic protein/cds = (17,628)/gb = X56932/gi = 23690/ |
| | | ug = Hs. 119122/len = 672 |
| 31509_at | Down | Cluster Incl. X64707: H. sapiens BBC1 mRNA/ |
| | | cds = (51,686)/gb = X64707/gi = 29382/ug = Hs. 180842/ |
| | | len = 942 |
| 31511_at | Down | Cluster Incl. U14971: Human ribosomal protein S9 |
| | | mRNA, complete cds/cds = (35,619)/gb = U14971/ |
| | | gi = 550022/ug = Hs. 180920/len = 692 |
| 41138_at | Down | Cluster Incl. M16279: Human MIC2 mRNA, complete |
| | | cds/cds = (177,734)/gb = M16279/gi = 188542/ |
| | | ug = Hs. 177543/len = 1238 |
| 33676_at | Down | Cluster Incl. X15940: Human mRNA for ribosomal |
| | | protein L31/cds = (7,384)/gb = X15940/gi = 36129/ |
| | | ug = Hs. 184014/len = 414 |
| 34592_at | Down | Cluster Incl. M13932: Human ribosomal protein S17 |
| | | mRNA, complete cds/cds = (25,432)/gb = M13932/ |
| | | gi = 337500/ug = Hs. 5174/len = 477 |
| 38060_at | Down | Cluster Incl. AI541336: pec1.2-7.A07.r Homo sapiens |
| | | cDNA, 5 end/clone_end = 5/gb = AI541336/gi = 4458709/ |
| | | ug = Hs. 80595/len = 717 |
| 32748_at | Down | Cluster Incl. AI557852: P6test.G05.r Homo sapiens |
| | | cDNA, 5 end/clone_end = 5/gb = AI557852/gi = 4490215/ |
| | | ug = Hs. 195453/len = 693 |
| 883_s_at | Down | M54915/FEATURE = /DEFINITION = HUMPIM1LE |
| | | Human h-pim-1 protein (h-pim-1) mRNA, complete cds |
| 829_s_at | Down | U21689/FEATURE = cds/DEFINITION = HSU21689 |
| | | Human glutathione S-transferase-P1c gene, complete cds |
| 37197_s_at | Down | Cluster Incl. AL050006: Homo sapiens mRNA; cDNA |
| | | DKFZp564A033 (from clone DKFZp564A033)/ |
| | | cds = (0,957)/gb = AL050006/gi = 4884074/ug = Hs. 7627/ |
| | | len = 1252 |
| 31527_at | Down | Cluster Incl. X17206: Human mRNA for LLRep3/ |
| | | cds = (240,905)/gb = X17206/gi = 34391/ug = Hs. 182426/ |
| | | len = 934 |
| 32276_at | Down | Cluster Incl. X03342: Human mRNA for ribosomal |
| | | protein L32/cds = (34,441)/gb = X03342/gi = 36131/ |
| | | ug = Hs. 169793/len = 505 |
| 683_at | Down | K02100/FEATURE = mRNA/DEFINITION = HUMOTC |
| | | Human ornithine transcarbamylase (OTC) mRNA, |
| | | complete coding sequence |
| 552_at | Down | U02570/FEATURE = /DEFINITION = HSU02570 Human |
| | | CDC42 GTPase-activating protein mRNA, partial cds |
| 1173_g_at | Down | Spermidine/Spermine N1-Acetyltransferase, Alt. Splice 2 |
| 31693_f_at | Down | Cluster Incl. Z80776: H. sapiens H2A/g gene/cds = (0,392)/ |
| | | gb = Z80776/gi = 1568542/ug = Hs. 239458/len = 393 |
| 39916_r_at | Down | Cluster Incl. J02984: Human insulinoma rig-analog |
| | | mRNA encoding DNA-binding protein, complete cds/ |
| | | cds = (29,466)/gb = J02984/gi = 184553/ug = Hs. 133230/ |
| | | len = 498 |
| 35852_at | Down | Cluster Incl. AB014558: Homo sapiens mRNA for |
| | | KIAA0658 protein, partial cds/cds = (0,1770)/ |
| | | gb = AB014558/gi = 3327129/ug = Hs. 7278/len = 4103 |
| 33619_at | Down | Cluster Incl. L01124: Human ribosomal protein S13 |
| | | (RPS13) mRNA, complete cds/cds = (32,487)/ |
| | | gb = L01124/gi = 307390/ug = Hs. 165590/len = 530 |
| 36355_at | Down | Cluster Incl. M13903: Human involucrin mRNA/ |
| | | cds = (0,1757)/gb = M13903/gi = 186520/ug = Hs. 157091/ |
| | | len = 1758 |
| 32436_at | Down | Cluster Incl. U14968: Human ribosomal protein L27a |
| | | mRNA, complete cds/cds = (16,462)/gb = U14968/ |
| | | gi = 550016/ug = Hs. 76064/len = 507 |
| 38639_at | Down | Cluster Incl. AF040963: Homo sapiens Mad4 homolog |
| | | (Mad4) mRNA, complete cds/cds = (13,642)/ |
| | | gb = AF040963/gi = 2792361/ug = Hs. 102402/len = 879 |
| 37009_at | Down | Cluster Incl. AL035079: dJ53C18.1 (Catalase)/ |
| | | cds = (74,1657)/gb = AL035079/gi = 4775614/ |
| | | ug = Hs. 76359/len = 2287 |
| 37027_at | Down | Cluster Incl. M80899: Human novel protein AHNAK |
| | | mRNA, partial sequence/cds = (0,3835)/gb = M80899/ |
| | | gi = 178282/ug = Hs. 76549/len = 4051 |
| 39294_at | Down | Cluster Incl. X16155: Human mRNA for chicken |
| | | ovalbumin upstream promoter transcription factor |
| | | (COUP-TF)/cds = (0,1256)/gb = X16155/gi = 30139/ |
| | | ug = Hs. 239468/len = 1513 |
| 39713_at | Down | Cluster Incl. AJ132440: Homo sapiens mRNA for PLU-1 |
| | | protein/cds = (89,4723)/gb = AJ132440/gi = 4902723/ |
| | | ug = Hs. 143323/len = 6355 |
| 32587_at | Down | Cluster Incl. U07802: Human Tis11d gene, complete cds/ |
| | | cds = (291,1739)/gb = U07802/gi = 984508/ug = Hs. 78909/ |
| | | len = 3655 |
| 41402_at | Down | Cluster Incl. AL080121: Homo sapiens mRNA; cDNA |
| | | DKFZp564O0823 (from clone DKFZp564O0823)/ |
| | | cds = (170,904)/gb = AL080121/gi = 5262554/ |
| | | ug = Hs. 105460/len = 2135 |
| 36899_at | Down | Cluster Incl. M97287: Human MAR/SAR DNA binding |
| | | protein (SATB1) mRNA, complete cds/cds = (214,2505)/ |
| | | gb = M97287/gi = 337810/ug = Hs. 74592/len = 2928 |
| 36039_s_at | Down | Cluster Incl. X93498: H. sapiens mRNA for 21-Glutamic |
| | | Acid-Rich Protein (21-GARP)/cds = UNKNOWN/ |
| | | gb = X93498/gi = 1673496/ug = Hs. 47438/len = 1160 |
| 33657_at | Down | Cluster Incl. L38941: Homo sapiens ribosomal protein |
| | | L34 (RPL34) mRNA, complete cds/cds = (20,373)/ |
| | | gb = L38941/gi = 1008855/ug = Hs. 179779/len = 392 |
| 41721_at | Down | Cluster Incl. AA658877: nt84c12.s1 Homo sapiens cDNA/ |
| | | clone = IMAGE-1205206/gb = AA658877/gi = 2595031/ |
| | | ug = Hs. 181350/len = 897 |
| 34775_at | Down | Cluster Incl. AF065388: Homo sapiens tetraspan NET-1 |
| | | mRNA, complete cds/cds = (121,846)/gb = AF065388/ |
| | | gi = 3152700/ug = Hs. 38972/len = 1278 |
| 1022_f_at | Down | V00542/FEATURE = mRNA/DEFINITION = HSIFR14 |
| | | Messenger RNA for human leukocyte (alpha) interferon |
| 35468_at | Down | Cluster Incl. AL050381: Homo sapiens mRNA; cDNA |
| | | DKFZp586B2023 (from clone DKFZp586B2023)/ |
| | | cds = UNKNOWN/gb = AL050381/gi = 4914611/ |
| | | ug = Hs. 172639/len = 1485 |
| 1147_at | Down | V-Erba Related Ear-3 Protein |
| 34365_at | Down | Cluster Incl. AF042386: Homo sapiens cyclophilin-33B |
| | | (CYP-33) mRNA, complete cds/cds = (60,950)/ |
| | | gb = AF042386/gi = 2828150/ug = Hs. 33251/len = 1099 |
| 39273_at | Down | Cluster Incl. AL022718: dJ1052M9.3 (mouse DOC4 |
| | | LIKE protein)/cds = (0,4094)/gb = AL022718/ |
| | | gi = 3763969/ug = Hs. 23796/len = 8728 |
| 33935_at | Down | Cluster Incl. AL035305: H. sapiens gene from PAC |
| | | 102G20/cds = (117,803)/gb = AL035305/gi = 4200223/ |
| | | ug = Hs. 27258/len = 2435 |
| 36040_at | Down | Cluster Incl. AI337192: qx88h10.x1 Homo sapiens |
| | | cDNA, 3 end/clone = IMAGE-2009635/clone_end = 3/ |
| | | gb = AI337192/gi = 4074119/ug = Hs. 47438/len = 925 |
| 39325_at | Down | Cluster Incl. U81523: Human endometrial bleeding |
| | | associated factor mRNA, complete cds/cds = (33,1145)/ |
| | | gb = U81523/gi = 2058537/ug = Hs. 25195/len = 1961 |
| 35546_at | Down | Cluster Incl. W28428: 49d8 Homo sapiens cDNA/ |
| | | gb = W28428 /gi = 13O8583 /ug = Hs.132153 /len = 812 |
| 32242_at | Down | Cluster Incl. AL038340: DKFZp566K192_s1 Homo |
| | | sapiens cDNA, 3 end/clone = DKFZp566K192/ |
| | | clone_end = 3/gb = AL038340/gi = 5407591/ug = Hs.1940/ |
| | | len = 746 |
| 762_f_at | Down | AB000905/FEATURE = cds JDEFINITION = AB000905 |
| | | Homo sapiens DNA for H4 histone, complete cds |
| 41106_at | Down | Cluster Incl. AF022797: Homo sapiens intermediate |
| | | conductance calcium-activated potassium channel |
| | | (hKCa4) mRNA, complete cds/cds = (396,1679)/ |
| | | gb = AF022797/gi = 2674355/ug = Hs.10082/len = 2238 |
| 38279_at | Down | Cluster Incl. D90150: Human Gx-alpha gene/ |
| | | cds = (619,1686)/gb = D90150/gi = 219668/ug = Hs.92002/ |
| | | len = 3289 |
| 1591_s_at | Down | J03242/FEATURE = /DEFINITION = HUMGFIL2 |
| | | Human insulin-lke growth factor II mRNA, complete cds |

Remarkably, when we compared the expression profiles of these 131 transcripts in orthotopic xenografts and individual clinical samples, we found that all metastatic prostate carcinomas have expression patterns highly similar to orthotopic xenografts as reflected in positive correlation of expression profiles, whereas all primary tumors displayed a negative correlation of expression profiles (FIG. 49A). We next attempted to refine the gene-expression signature associated with human prostate cancer metastasis to a small set of transcripts that would exhibit similar discrimination accuracy between metastatic and primary tumors. To achieve this we used the increase in correlation coefficient of gene expression profiles between orthotopic xenografts and clinical samples as a guide for reduction of transcripts number in a cluster (FIGS. 48B, C, and D). Using this strategy we were able to identify several smaller clusters of co-regulated genes exhibiting highly concordant behavior in the model system and clinical samples (FIGS. 48 A-D and Tables 41.1, 41.2, 41 & 42) and demonstrating highly accurate discrimination (at least 94%) between clinical samples of metastatic and primary human prostate carcinomas (FIGS. 49A-D and Table 42).

TABLE 41.1
Prostate cancer metastasis segregation cluster comprising 37 genes

| | Change | |
|---|---|---|
| Affymetrix | direction in | |
| ID (U95Av2) | metastasis | Description |
| 33534_at | Up | Cluster Incl. X89426: H. sapiens mRNA for ESM-1 |
| | | protein/cds = (55,609)/gb = X89426/gi = 1150418/ |
| | | ug = Hs. 41716/len = 2006 |
| 33232_at | Up | Cluster Incl. AI017574: ou23f10.x1 Homo sapiens |
| | | cDNA, 3 end/clone = IMAGE-1627147/clone_end = 3/ |
| | | gb = AI017574/gi = 3231910/ug = Hs. 17409/len = 501 |
| 34289_f_at | Up | Cluster Incl. D50920: Human mRNA for KIAA0130 |
| | | gene, complete cds/cds = (73,3042)/gb = D50920/ |
| | | gi = 1469182/ug = Hs. 23106/len = 3468 |
| 430_at | Up | X00737/FEATURE = cds/DEFINITION = HSPNP |
| | | Human mRNA for purine nucleoside phosphorylase |
| | | (PNP; EC 2.4.2.1) |
| 907_at | Up | M13792/FEATURE = cds/DEFINITION = HUMADAG |
| | | Human adenosine deaminase (ADA) gene, complete cds |
| 34742_at | Up | Cluster Incl. Z23115: H. sapiens bcl-xL mRNA/ |
| | | cds = (134,835)/gb = Z23115/gi = 510900/ug = Hs. 239744/ |
| | | len = 926 |
| 38110_at | Up | Cluster Incl. AF000652: Homo sapiens syntenin (sycl) |
| | | mRNA, complete cds/cds = (148,1044)/gb = AF000652/ |
| | | gi = 2795862/ug = Hs. 8180/len = 2162 |
| 38290_at | Up | Cluster Incl. AF037195: Homo sapiens regulator of G |
| | | protein signaling RGS14 mRNA, complete cds/ |
| | | cds = (73,1398)/gb = AF037195/gi = 2708809/ |
| | | ug = Hs. 9347/len = 1531 |
| 36870_at | Up | Cluster Incl. AB018347: Homo sapiens mRNA for |
| | | KIAA0804 protein, partial cds/cds = (0,3636)/ |
| | | gb = AB018347/gi = 3882328/ug = Hs. 7316/len = 4216 |
| 1624_at | Up | Stimulatory Gdp/Gtp Exchange Protein For C-Ki-Ras |
| | | P21 And Smg P21 |
| 41855_at | Up | Cluster Incl. AF030424: Homo sapiens histone |
| | | acetyltransferase 1 mRNA, complete cds/cds = (36,1295)/ |
| | | gb = AF030424/gi = 2623155/ug = Hs. 13340/len = 1568 |
| 36355_at | Down | Cluster Incl. M13903: Human involucrin mRNA/ |
| | | cds = (0,1757)/gb = M13903/gi = 186520/ug = Hs. 157091/ |
| | | len = 1758 |
| 32436_at | Down | Cluster Incl. U14968: Human ribosomal protein L27a |
| | | mRNA, complete cds/cds = (16,462)/gb = U14968/ |
| | | gi = 550016/ug = Hs. 76064/len = 507 |
| 38639_at | Down | Cluster Incl. AF040963: Homo sapiens Mad4 homolog |
| | | (Mad4) mRNA, complete cds/cds = (13,642)/ |
| | | gb = AF040963/gi = 2792361/ug = Hs. 102402/len = 879 |
| 37009_at | Down | Cluster Incl. AL035079: dJ53C18.1 (Catalase)/ |
| | | cds = (74,1657)/gb = AL035079/gi = 4775614/ |
| | | ug = Hs. 76359/len = 2287 |
| 37027_at | Down | Cluster Incl. M80899: Human novel protein AHNAK |
| | | mRNA, partial sequence/cds = (0,3835)/gb = M80899/ |
| | | gi = 178282/ug = Hs. 76549/len = 4051 |
| 39294_at | Down | Cluster Incl. X16155: Human mRNA for chicken |
| | | ovalbumin upstream promoter transcription factor |
| | | (COUP-TF)/cds = (0,1256)/gb = X16155/gi = 30139/ |
| | | ug = Hs. 239468/len = 1513 |
| 39713_at | Down | Cluster Incl. AJ132440: Homo sapiens mRNA for PLU-1 |
| | | protein/cds = (89,4723)/gb = AJ132440/gi = 4902723/ |
| | | ug = Hs. 143323/len = 6355 |
| 32587_at | Down | Cluster Incl. U07802: Human Tis11d gene, complete cds/ |
| | | cds = (291,1739)/gb = U07802/gi = 984508/ug = Hs. 78909/ |
| | | len = 3655 |
| 41402_at | Down | Cluster Incl. AL080121: Homo sapiens mRNA; cDNA |
| | | DKFZp564O0823 (from clone DKFZp564O0823)/ |
| | | cds = (170,904)/gb = AL080121/gi = 5262554/ |
| | | ug = Hs. 105460/len = 2135 |
| 36039_s_at | Down | Cluster Incl. X93498: H. sapiens mRNA for 21-Glutamic |
| | | Acid-Rich Protein (21-GARP)/cds = UNKNOWN/ |
| | | gb = X93498/gi = 1673496/ug = Hs. 47438/len = 1160 |
| 33657_at | Down | Cluster Incl. L38941: Homo sapiens ribosomal protein |
| | | L34 (RPL34) mRNA, complete cds/cds = (20,373)/ |
| | | gb = L38941/gi = 1008855/ug = Hs. 179779/len = 392 |
| 41721_at | Down | Cluster Incl. AA658877: nt84c12.s1 Homo sapiens |
| | | cDNA/clone = IMAGE-1205206/gb = AA658877/ |
| | | gi = 2595031/ug = Hs. 181350/len = 897 |
| 34775_at | Down | Cluster Incl. AF065388: Homo sapiens tetraspan NET-1 |
| | | mRNA, complete cds/cds = (121,846)/gb = AF065388/ |
| | | gi = 3152700/ug = Hs. 38972/len = 1278 |
| 1022_f_at | Down | V00542/FEATURE = mRNA/DEFINITION = HSIFR14 |
| | | Messenger RNA for human leukocyte (alpha) interferon |
| 35468_at | Down | Cluster Incl. AL050381: Homo sapiens mRNA; cDNA |
| | | DKFZp586B2023 (from clone DKFZp586B2023)/ |
| | | cds = UNKNOWN/gb = AL050381/gi = 4914611/ |
| | | ug = Hs. 172639/len = 1485 |
| 1147_at | Down | V-Erba Related Ear-3 Protein |
| 34365_at | Down | Cluster Incl. AF042386: Homo sapiens cyclophilin-33B |
| | | (CYP-33) mRNA, complete cds/cds = (60,950)/ |
| | | gb = AF042386/gi = 2828150/ug = Hs. 33251/len = 1099 |
| 33935_at | Down | Cluster Incl. AL035305: H. sapiens gene from PAC |
| | | 102G20/cds = (117,803)/gb = AL035305/gi = 4200223/ |
| | | ug = Hs. 27258/len = 2435 |
| 36040_at | Down | Cluster Incl. AI337192: qx88h10.x1 Homo sapiens |
| | | cDNA, 3 end/clone = IMAGE-2009635/clone_end = 3/ |
| | | gb = AI337192/gi = 4074119/ug = Hs. 47438/len = 925 |
| 39325_at | Down | Cluster Incl. U81523: Human endometrial bleeding |
| | | associated factor mRNA, complete cds/cds = (33,1145)/ |
| | | gb = U81523/gi = 2058537/ug = Hs. 25195/len = 1961 |
| 35546_at | Down | Cluster Incl. W28428: 49d8 Homo sapiens cDNA/ |
| | | gb = W28428/gi = 1308583/ug = Hs. 132153/len = 812 |
| 32242_at | Down | Cluster Incl. AL038340: DKFZp566K192_s1 Homo |
| | | sapiens cDNA, 3 end/clone = DKFZp566K192/ |
| | | clone_end = 3/gb = AL038340/gi = 5407591/ug = Hs. 1940/ |
| | | len = 746 |
| 762_f_at | Down | AB000905/FEATURE = cds/DEFINITION = AB000905 |
| | | Homo sapiens DNA for H4 histone, complete cds |
| 41106_at | Down | Cluster Incl. AF022797: Homo sapiens intermediate |
| | | conductance calcium-activated potassium channel |
| | | (hKCa4) mRNA, complete cds/cds = (396,1679)/ |
| | | gb = AF022797/gi = 2674355/ug = Hs. 10082/len = 2238 |
| 38279_at | Down | Cluster Incl. D90150: Human Gx-alpha gene/ |
| | | cds = (619,1686)/gb = D90150/gi = 219668/ug = Hs. 92002/ |
| | | len = 3289 |
| 1591_s_at | Down | J03242/FEATURE = /DEFINITION = HUMGFIL2 |
| | | Human insulin-lke growth factor II mRNA, complete cds |

TABLE 41.2
Prostate cancer metastasis segregation cluster comprising 12 genes

| | Change | |
|---|---|---|
| Affymetrix | direction in | |
| ID (U95Av2) | metastasis | Description |
| 33534_at | Up | Cluster Incl. X89426: H. sapiens mRNA for ESM-1 |
| | | protein/cds = (55,609)/gb = X89426/gi = 1150418/ |
| | | ug = Hs. 41716/len = 2006 |
| 33232_at | Up | Cluster Incl. AI017574: ou23f10.x1 Homo sapiens |
| | | cDNA, 3 end/clone = IMAGE-1627147/clone_end = 3/ |
| | | gb = AI017574/gi = 3231910/ug = Hs. 17409/len = 501 |
| 34289_f_at | Up | Cluster Incl. D50920: Human mRNA for KIAA0130 |
| | | gene, complete cds/cds = (73,3042)/gb = D50920/ |
| | | gi = 1469182/ug = Hs. 23106/len = 3468 |
| 430_at | Up | X00737/FEATURE = cds/DEFINITION = HSPNP |
| | | Human mRNA for purine nucleoside phosphorylase |
| | | (PNP; EC 2.4.2.1) |
| 907_at | Up | M13792/FEATURE = cds/DEFINITION = HUMADAG |
| | | Human adenosine deaminase (ADA) gene, complete cds |
| 34742_at | Up | Cluster Incl. Z23115: H. sapiens bcl-xL mRNA/ |
| | | cds = (134,835)/gb = Z23115/gi = 510900/ug = Hs. 239744/ |
| | | len = 926 |
| 36040_at | Down | Cluster Incl. AI337192: qx88h10.x1 Homo sapiens |
| | | cDNA, 3 end/clone = IMAGE-2009635/clone_end = 3/ |
| | | gb = AI337192/gi = 4074119/ug = Hs. 47438/len = 925 |
| 35546_at | Down | Cluster Incl. W28428: 49d8 Homo sapiens cDNA/ |
| | | gb = W28428/gi = 1308583/ug = Hs. 132153/len = 812 |
| 762_f_at | Down | AB000905/FEATURE = cds/DEFINITION = AB000905 |
| | | Homo sapiens DNA for H4 histone, complete cds |
| 41106_at | Down | Cluster Incl. AF022797: Homo sapiens intermediate |
| | | conductance calcium-activated potassium channel |
| | | (hKCa4) mRNA, complete cds/cds = (396,1679)/ |
| | | gb = AF022797/gi = 2674355/ug = Hs. 10082/len = 2238 |
| 38279_at | Down | Cluster Incl. D90150: Human Gx-alpha gene/ |
| | | cds = (619,1686)/gb = D90150/gi = 219668/ug = Hs. 92002/ |
| | | len = 3289 |
| 1591_s_at | Down | J03242/FEATURE = /DEFINITION = HUMGFIL2 |
| | | Human insulin-lke growth factor II mRNA, complete |
| | | cds |

Interestingly, the 9-gene molecular signature cluster (FIG. 48D; Tables 41&42) associated with human prostate cancer metastasis has several candidate markers and targets for mechanistic studies and/or drug development such as secreted proteins (ESM-1 and EBAF), transcription regulators (CRIP1, TRAP100, NRF2F1), two enzymes playing a key role in the purine salvage pathway (NP and ADA), an apoptosis inhibitor (BCL-X_{L}), and a molecular chaperone (CRYAB).

TABLE 41
The 9-gene molecular signature associated with metastatic prostate cancer

| | | GenBank | UniGene |
|---|---|---|---|
| Gene | Gene name | ID | ID |
| ESM1 | Endothelial cell-specific molecule 1 | X89426 | Hs. 41716 |
| CRIP1 | Cysteine-rich protein 1 | AI0175174 | Hs. 17409 |
| TRAP100 | Thyroid hormone receptor-associated protein | D50920 | Hs. 23106 |
| NP | Nucleoside phosphorylase | X00737 | Hs. 75514 |
| ADA | Adenosine deaminase | M13792 | Hs. 1217 |
| BCL2L1 | BCL2-like 1 | Z23115 | Hs. 305890 |
| NRF2F1 | Nuclear receptor subfamily 2, group F, member 1 | X16155 | Hs. 421993 |
| EBAF | Endometrial bleeding associated factor | U81523 | Hs. 25195 |
| CRYAB | Crystallin, alpha B | AL038340 | Hs. 391270 |

TABLE 42
Classification accuracy of metastasis segregation clusters

| Number of genes | Correlation | Performance | Performance | Overall |
|---|---|---|---|---|
| in cluster | coefficient | (metastases) | (primary tumors) | performance |
| 131 genes | r = 0.799 | 9 of 9 (100%) | 23 of 23 (100%) | 32 of 32 (100%) |
| 37 genes | r = 0.938 | 9 of 9 (100%) | 21 of 23 (91%) | 30 of 32 (94%) |
| 15 genes | r = 0.958 | 9 of 9 (100%) | 21 of 23 (91%) | 30 of 32 (94%) |
| 12 genes | r = 0.990 | 9 of 9 (100%) | 21 of 23 (91%) | 30 of 32 (94%) |
| 9 genes | r = 0.973 | 9 of 9 (100%) | 21 of 23 (91%) | 30 of 32 (94%) |
| 14 genes | r = 0.937 | 9 of 9 (100%) | 22 of 23 (96%) | 31 of 32 (97%) |

To further test the potential clinical relevance of the models, we attempted to utilize expression profiling of highly metastatic orthotopic human prostate carcinoma xenografts for identification of gene expression correlates of clinically significant phenotypes such as invasive behavior and recurrence propensity of human prostate tumors (the original clinical data utilized in these examples were recently published in Singh, D., Febbo, P. G., Ross, K., Jackson, D. G., Manola, C. L., Tamayo, P., Renshaw, A. A., D'Amico, A. V., Richie, J. P., Lander, E. S., Loda, M., Kantoff, P. W., Golub, T. R., Sellers, W. R. Gene expression correlates of clinical prostate cancer behavior. Cancer Cell, 1: 203-209, 2002). Using gene expression profiles of metastasis-promoting orthotopic xenografts as a predictive reference of expected transcript abundance behavior in clinical samples, we identified a five-gene cluster (Table 43) of co-regulated transcripts discriminating with 75% accuracy invasive versus non-invasive human prostate tumors (FIGS. 47B and 50A).

TABLE 43
The 5-gene molecular fingerprint associated with invasive
phenotype of human prostate cancer

| Gene | Gene name | GenBank ID | UniGene ID |
|---|---|---|---|
| HRASLS3 | HRAS-like suppressor 3 | X92814 | Hs. 37189 |
| | EST | AI986201 | Hs. 355812 |
| KIAA0962 | KIAA0962 protein | AB023179 | Hs. 9059 |
| SLC29A2 | Solute carrier family 29 | AF034102 | Hs. 32951 |
| KIAA0557 | KIAA0557 protein | AB011129 | Hs. 101414 |

20 of 26 samples (77%) obtained from the patients with invasive prostate cancer defined by histology as having positive surgical margins (“PSM”) and/or extra-capsular penetration (“PCP”) exhibited a positive correlation coefficient of expression of the five-gene cluster (Table 43) compared to orthotopic xenografts. In contrast, 19 of 26 samples (73%) from the patients with organ-confined disease showed a negative correlation coefficient of expression of the five-gene cluster (Table 43) compared to orthotopic xenografts (FIG. 50A). Furthermore, using this strategy we identified an eight-gene cluster (Table 44) of co-regulated transcripts discriminating with 90% accuracy human prostate tumors exhibiting recurrent or non-recurrent clinical behavior (FIGS. 47A& 50B).

TABLE 44
The 8-gene molecular fingerprint predicting recurrent
phenotype of human prostate cancer

| Gene | Gene name | GenBank ID | UniGene ID |
|---|---|---|---|
| MGC5466 | Hypothetical protein MGC5466 | U90904 | Hs. 83724 |
| CHAF1A | Chromatin assembly factor 1, | U20979 | Hs. 79018 |
| | subunit A | | |
| CDS2 | CDP-diacylglycerol synthase 2 | Y16521 | Hs. 24812 |
| STX7 | Syntaxin 7 | U77942 | Hs. 427065 |
| IER3 | Immediate early response 3 | S81914 | Hs. 76095 |
| GLUL | Glutamate-ammonia ligase | X59834 | Hs. 170171 |
| MYBPC1 | Myosin binding protein C | X73114 | Hs. 169849 |
| SOX9 | SRY-box 9 | Z46629 | Hs. 2316 |

In this example we compared a set of transcripts differentially regulated in recurrent versus non-recurrent human prostate tumors with transcripts differentially regulated in orthotopic human prostate carcinoma xenografts derived from highly metastatic PC3MLN4 cell variant versus orthotopic tumors of the less metastatic parental lineages, PC3 and PC3M. FIG. 50B illustrates application of the eight-gene cluster (Table 44) to characterize clinical prostate cancer samples according to their propensity for recurrence after therapy. The expression pattern of the genes in the recurrence predictor cluster was analyzed in each of twenty-one separate clinical samples. The analysis produces a quantitative phenotype association index (plotted on the Y-axis) for each of the twenty-one clinical prostate cancer samples. Tumors that are likely to recur are expected to have positive phenotype association indices reflecting positive correlation of gene expression with metastasis-promoting orthotopic xenografts, while those that are unlikely to recur are expected to have negative association indices.

FIG. 50B shows the phenotype association indices for eight samples from patients who later had recurrence as bars 1 through 8, while the association indices for thirteen samples from patients whose tumors did not recur is shown as bars 12 through 24. Eight of the eight samples (or 100%) from patients who later experienced recurrence had positive phenotype association indices and so were properly classified. Eleven of the thirteen samples (or 84.6%) from patients whose tumors did not recur had negative phenotype association indices and so were properly classified as non-recurrent tumors. Thus, overall, nineteen of the twenty-one samples (or 90.5%) were properly classified using an eight-gene recurrence predictor cluster.

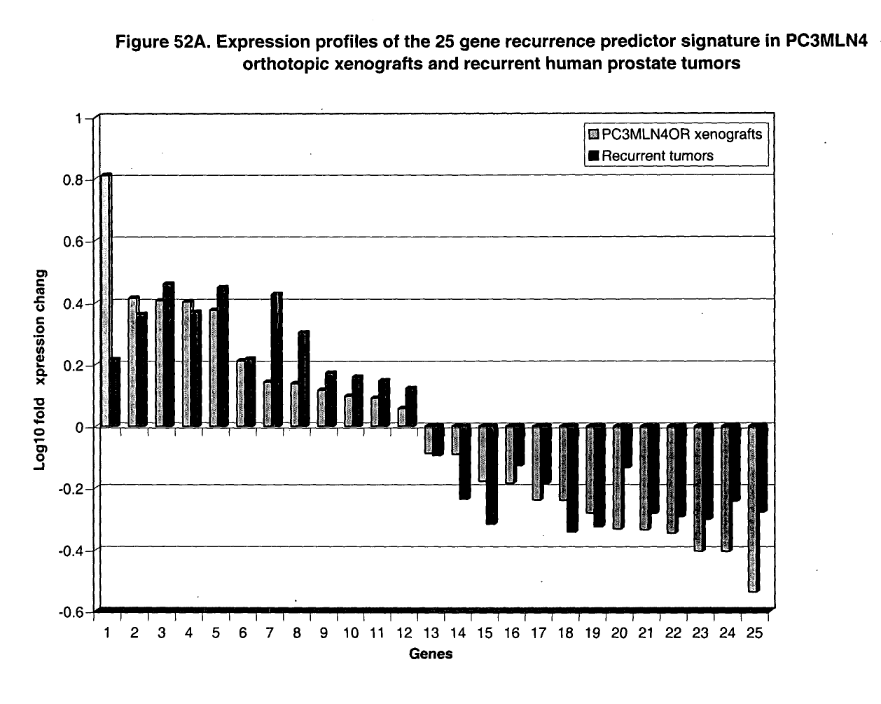

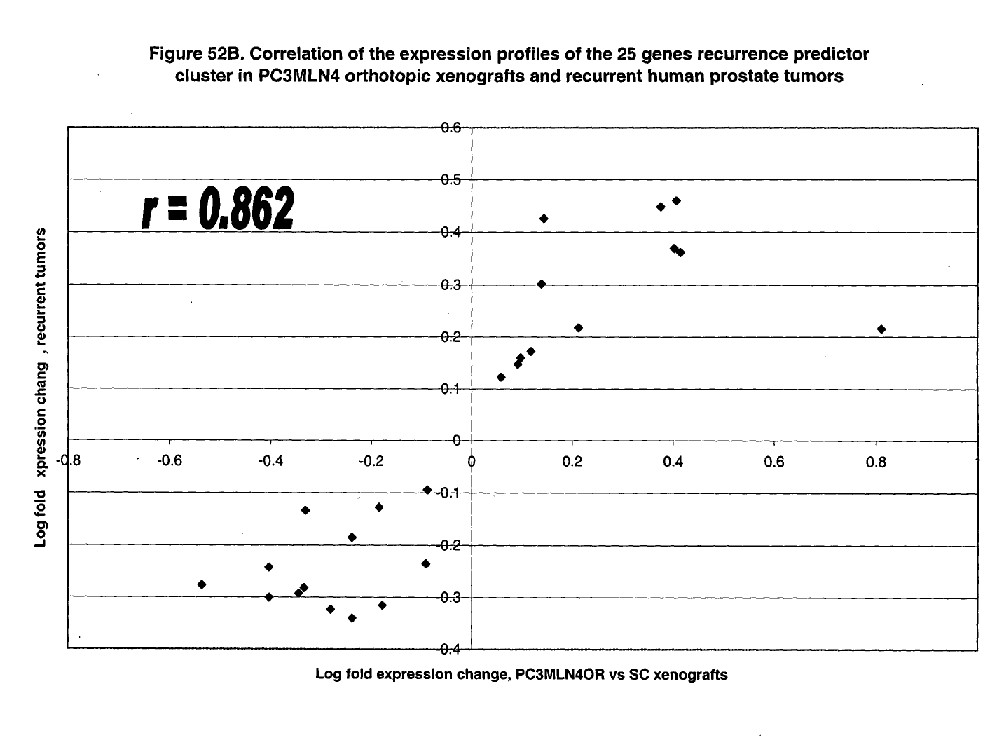

Next we compared a set of transcripts differentially regulated in recurrent versus non-recurrent human prostate tumors with transcripts differentially regulated in orthotopic human prostate carcinoma xenografts derived from highly metastatic PC3MLN4 cell variant versus subcutaneous (“s.c.”) ectopic tumors of the same lineage. This comparison identified a set of 25 genes (FIGS. 52A & B & Table 45) that exhibited highly concordant behavior in clinical recurrent samples and orthotopic metastasis-promoting tumors (Pearson correlation coefficient, r=0.862; FIG. 52B).

TABLE 45
The 25-gene molecular signature predicting recurrent prostate cancer

| Gene | Gene name | GenBank ID | UniGene ID |
|---|---|---|---|
| ETS1 | v-ets erythroblastosis virus E26 oncogene | X14798 | Hs. 18063 |
| | homolog 1 | | |
| MGC5466 | Hypothetical protein MGC5466 | U90904 | Hs. 83724 |
| CA2 | carbonic anhydrase II | J03037 | Hs. 155097 |
| LRP2 | Megalin | U33837 | Hs. 153595 |
| EPHA3 | receptor tyrosine kinase HEK | M83941 | Hs. 123642 |
| Wnt5A | proto-oncogene Wnt5A | L20861 | Hs. 152213 |
| ADRA1A | adrenergic, alpha-1A-, receptor | D32202 | Hs. 52931 |
| | EST | R38263 | Hs. 375190 |
| CDS2 | CDP-diacylglycerol synthase | Y16521 | Hs. 24812 |
| | EST | AL050002 | Hs. 94795 |
| STX7 | syntaxin 7 | U77942 | Hs. 427065 |
| RANBP3 | RAN binding protein 3 | Y08698 | Hs. 176657 |
| FSTL1 | follistatin-like 1 | U06863 | Hs. 433622 |
| ZFP36L2 | zinc finger protein 36 | U07802 | Hs. 78909 |
| GGT2 | gamma-glutamyltransferase 2 | M30474 | Hs. 289098 |
| KIAA0476 | KIAA0476 protein | AB007945 | Hs. 6684 |
| ITPR1 | inositol 1,4,5-trisphosphate receptor, type 1 | D26070 | Hs. 198443 |
| ITCH | Itchy homolog E3 ubiquitin protein ligase | AF038564 | Hs. 98074 |
| CD44 | CD44 antigen | L05424 | Hs. 169610 |
| TNRC15 | Trinucleotide repeat containing 15 | AB014542 | Hs. 323317 |
| MXI1 | MAX interacting protein 1 | L07648 | Hs. 118630 |
| TCF2 | transcription factor 2, hepatic | X58840 | Hs. 169853 |
| KCNN4 | intermediate conductance calcium- | AF022797 | Hs. 10082 |
| | activated potassium channel | | |
| APS | Adaptor protein | AB000520 | Hs. 105052 |
| SOX9 | SRY-box 9 | Z46629 | Hs. 2316 |

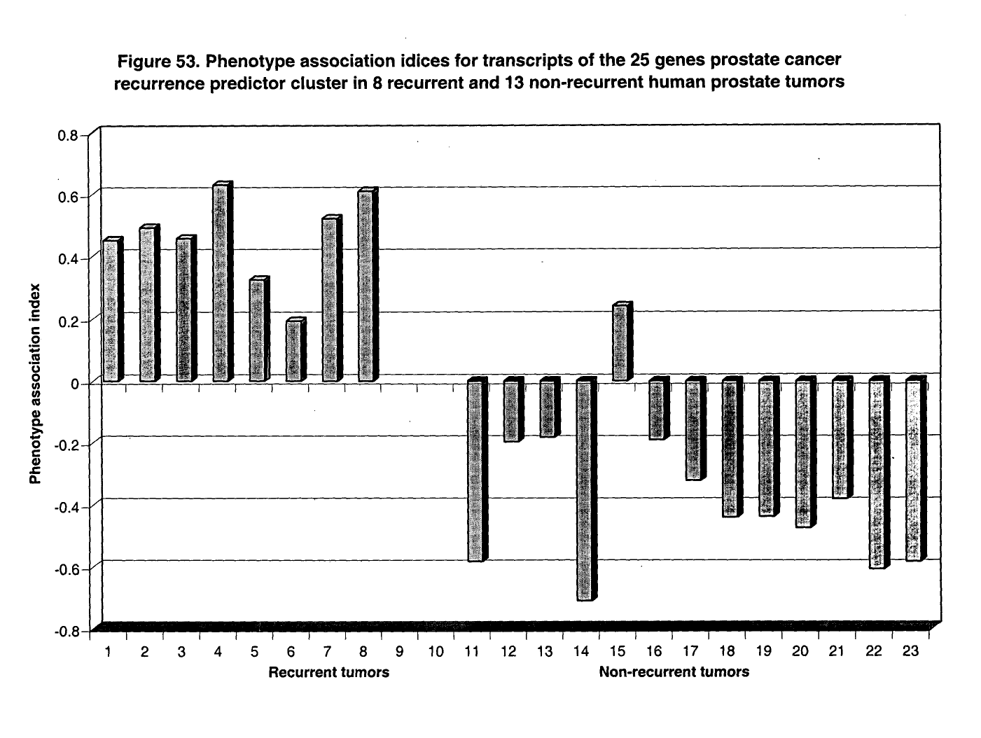

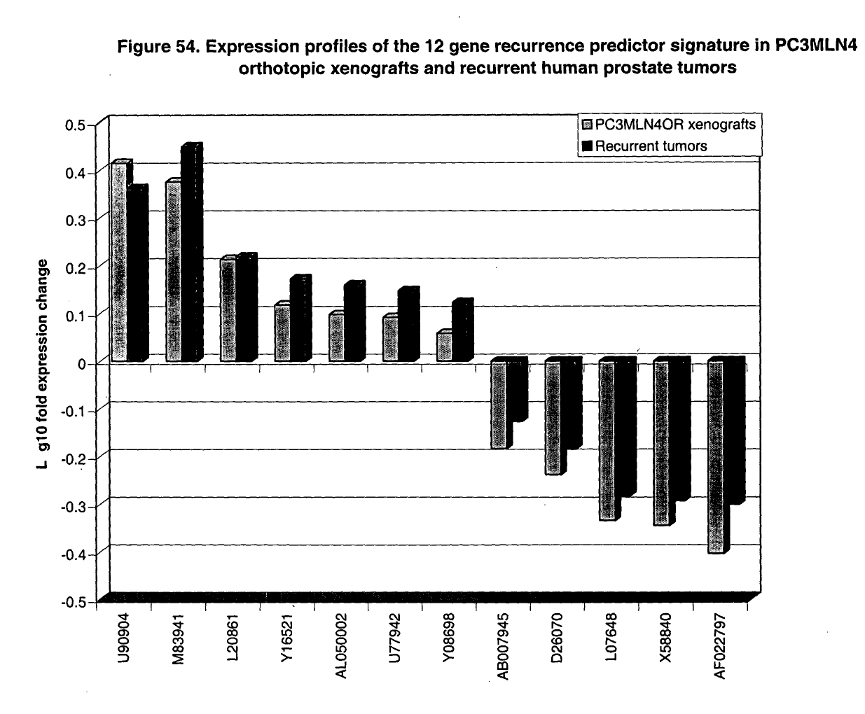

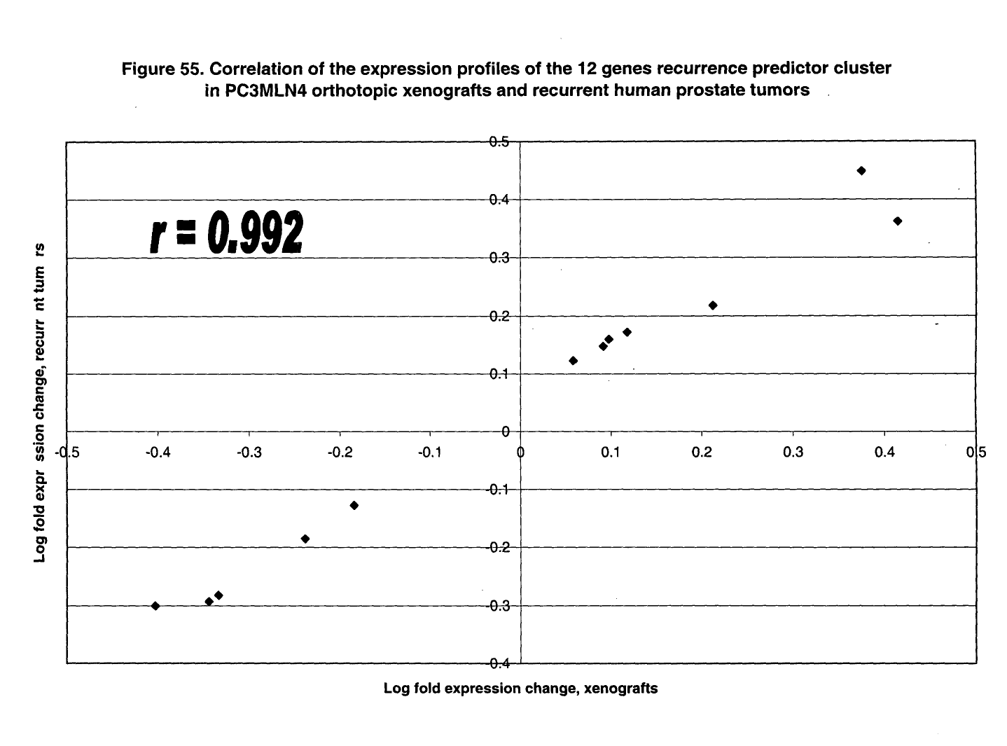

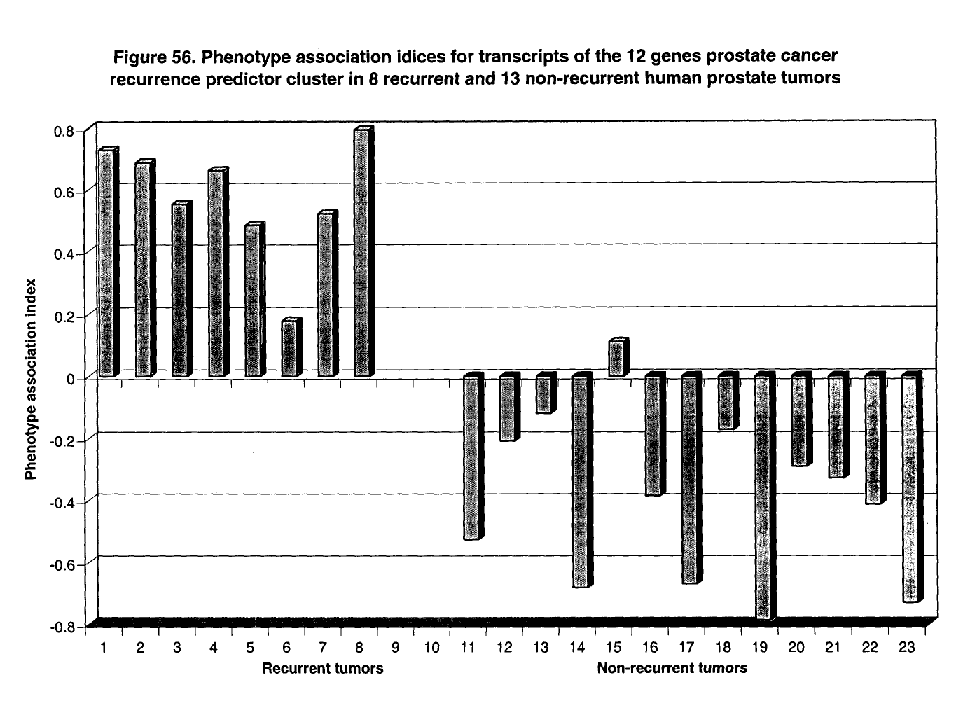

When we compared the expression profiles of these 25 transcripts in orthotopic xenografts and individual clinical samples, we found that all recurrent prostate carcinomas have expression patterns highly similar to orthotopic xenografts as reflected in positive correlation of expression profiles, whereas 12 of 13 non-recurrent tumors displayed a negative correlation of expression profiles (FIG. 53). We next attempted to refine the gene-expression signature associated with human prostate cancer metastasis to a smaller set of transcripts that would exhibit similar discrimination accuracy between recurrent and non-recurrent tumors. To achieve this we used the increase in correlation coefficient of gene expression profiles between orthotopic xenografts and clinical samples as a guide for reducing the number of genes in the cluster (cf FIGS. 52B & 55). Using this strategy we identified a smaller cluster of 12 co-regulated genes (FIG. 54 & Table 46) exhibiting highly concordant behavior in the model system and clinical samples (r=0.992; FIG. 55) and demonstrating highly accurate discrimination (20 of 21 samples, or 95% were correctly classified) between clinical samples of recurrent and non-recurrent human prostate carcinomas (FIG. 56).

TABLE 46
The 12-gene molecular signature predicting recurrent prostate cancer

| | | GenBank | UniGene |
|---|---|---|---|
| Gene | Gene name | ID | ID |
| MGC5466 | Hypothetical protein MGC5466 | U90904 | Hs. 83724 |
| EPHA3 | Receptor tyrosine kinase HEK | M83941 | Hs. 123642 |
| Wnt5A | Proto-oncogene Wnt5A | L20861 | Hs. 152213 |
| CDS2 | CDP-diacylglycerol synthase | Y16521 | Hs. 24812 |
| | EST | AL050002 | Hs. 94795 |
| STX7 | Syntaxin 7 | U77942 | Hs. 427065 |
| RANBP3 | RAN binding protein 3 | Y08698 | Hs. 176657 |
| KIAA0476 | KIAA0476 protein | AB007945 | Hs. 6684 |
| ITPR1 | Inositol 1,4,5-trisphosphate | D26070 | Hs. 198443 |
| | receptor, type 1 | | |
| MXI1 | MAX interacting protein 1 | L07648 | Hs. 118630 |
| TCF2 | Transcription factor 2, hepatic | X58840 | Hs. 169853 |
| KCNN4 | Intermediate conductance | AF022797 | Hs. 10082 |
| | calcium-activated potassium | | |
| | channel | | |

In conclusion, using gene expression profiles of metastasis-promoting orthotopic xenografts as a predictive reference of expected transcript abundance behavior in clinical samples, we identified clusters of co-regulated genes discriminating with 75-100% accuracy among metastatic versus primary, invasive versus non-invasive, and recurrent versus non-recurrent human prostate tumors. Our data indicate that human prostate cancer cells derived from metastatic lesions have stable “genetic memory” of metastatic behavior and that genetic signatures associated with metastatic phenotype could be revived by growth in a metastasis-promoting orthotopic environment. The genetic signatures of metastatic prostate cancer have the ability to be used as nucleic acid-based and/or protein-based clinical prognostic and diagnostic tests useful in clinical management of prostate cancer patients, and as a source of targets for novel therapeutic approaches for disease management.

### EXAMPLE 6

### Selection of the Gene Clusters with Clinically Useful Properties Using the Best-Fit Sample(S) as a Reference Standard

Application of the present invention for identification of gene clusters with useful clinical properties was not limited by the availability of the suitable reference standard such as the appropriate cell lines and/or in vivo model systems. When a suitable reference standard was not readily available an algorithm utilizing the expression profile(s) of the best-fit sample(s) as a reference standard was applied for selection of the minimum segregation set of genes. As the first step of such analysis we compared the gene expression profiles of two distinct sets of samples that are subjects of classification (for example, metastatic and non-metastatic human breast tumors) to identify a broad spectrum of transcripts differentially regulated at a statistically significant level (p<0.05) in metastatic human breast cancer. If desirable, further criteria such as a particular cut-off based on fold expression changes (e.g., 2-fold, 3-fold, etc.) can be applied for selecting differentially expressed genes. Next, we calculated the average expression values for each transcript of the differentially expressed genes in the metastatic and non-metastatic tumors and determined the average fold expression change in metastatic versus non-metastatic tumors (“average” metastatic expression profile). We then determined the individual expression profiles for each sample within the two classification groups by calculating fold expression change for each transcript of the differentially expressed class of genes in a given sample by dividing an individual expression value of a gene by the average expression value for a particular gene across the entire data set. At the next step, we determined the individual phenotype association indices across the entire data set by calculating the Pearson correlation coefficient between the “average” metastatic expression profile and individual expression profiles. Next, the selection of the best-fit sample(s) was performed based on a highest positive and/or negative value(s) of the individual phenotype association index. The expression profile(s) of the best-fit sample(s) was utilized to refine the gene-expression signature associated with a particular phenotype to a small set of transcripts that would exhibit high discrimination accuracy between metastatic and non-metastatic tumors. To achieve this we used the increase in correlation coefficient of gene expression profiles between the “average” metastatic expression profile and an expression profile(s) of the best-fit sample(s) as a guide for reducing the number of members within a cluster.

### EXAMPLE 7

### Selection of the Gene Clusters Discriminating Between Invasive and Non-Invasive Human Prostate Cancer

The methods of the invention were used along with the data reported by Singh, et al. (2002) to identify gene clusters associated with an invasive phenotype. These data were the supplemental data reported in Singh, D., Febbo, P. G., et al., “Gene Expression Correlates of Clinical Prostate Cancer Behavior,” Cancer Cell March 2002 1:203-209, incorporated herein by reference. The clinical human prostate tumor samples were divided into two groups, invasive and non-invasive, as reported in Singh, et al. (2002). Invasive phenotype was assessed by determining the presence or absence of positive surgical margins (“PSM”) and positive or negative capsular penetration (“PCP”). The reference set was obtained following the procedures described above in part B, using the supplemental data reported in Singh, et al. (2002) for 26 invasive (identified as having positive surgical margins and/or positive capsular penetration) and 26 non-invasive (identified as having no evidence of positive surgical margins and/or positive capsular penetration) human prostate tumors. Thus, the first reference set was obtained by using the Affymetrix MicroDB (version 3.0) and Affymetrix Data Mining Tools (DMT) (version 3.0) data analysis software to identify genes that were differentially regulated in invasive group compared to non-invasive group of patients at the statistically significant level (p<0.05; Student T-test). Candidate genes were included in the first reference set if they were identified by the DMT software as having p values of 0.05 or less both for up-regulated and down-regulated genes. 114 genes were identified as being members of the reference set (Table 47).

TABLE 47
114 genes differentially regulated in 26 invasive
versus 26 non-invasive human prostate tumors.

| Affymetrix | |
|---|---|
| Probe Set ID | |
| (U95Av2) | Description |
| 40635_at | Cluster Incl. AF089750: Homo sapiens flotillin-1 mRNA, complete cds/ |
| | cds = (164,1447)/gb = AF089750/gi = 3599572/ug = Hs. 179986/ |
| | len = 1796 |
| 36993_at | Cluster Incl. M33210: Human colony stimulating factor 1 receptor |
| | (CSF1R) gene/cds = (0,283)/gb = M33210/gi = 532592/ug = Hs. 76144/ |
| | len = 2206 |
| 38682_at | Cluster Incl. AF045581: Homo sapiens BRCA1 associated protein 1 |
| | (BAP1) mRNA, complete cds/cds = (39,2228)/gb = AF045581/ |
| | gi = 2854120/ug = Hs. 106674/len = 3506 |
| 38260_at | Cluster Incl. AL050306: Human DNA sequence from clone 475B7 on |
| | chromosome Xq12.1-13. Contains the 3 part of the gene for a novel |
| | KIAA0615 and KIAA0323 LIKE protein, the gene for a novel protein, |
| | ESTs, STSs, GSSs and two putative CpG islands/cds = (48,2201)/ |
| | gb = AL050306/gi = 5419784/ug = Hs. 90625/len = 2395 |
| 41725_at | Cluster Incl. U89896: Homo sapiens casein kinase I gamma 2 mRNA, |
| | complete cds/cds = (239,1486)/gb = U89896/gi = 1890117/ |
| | ug = Hs. 181390/len = 1749 |
| 34880_at | Cluster Incl. AC002115: Human DNA from overlapping chromosome |
| | 19 cosmids R31396, F25451, and R31076 containing COX6B and |
| | UPKA, genomic sequence/cds = (336,1355)/gb = AC002115/ |
| | gi = 2098573/ug = Hs. 5086/len = 1473 |
| 32140_at | Cluster Incl. Y08110: H. sapiens mRNA for mosaic protein LR11/ |
| | cds = (80,6724)/gb = Y08110/gi = 1552323/ug = Hs. 166294/len = 6840 |
| 35704_at | Cluster Incl. X92814: H. sapiens mRNA for rat HREV107-like protein/ |
| | cds = (407,895)/gb = X92814/gi = 1054751/ug = Hs. 37189/len = 1070 |
| 32212_at | Cluster Incl. AL049703: Human gene from PAC 179D3, chromosome |
| | X, isoform of mitochondrial apoptosis inducing factor, AIF, AF100928/ |
| | cds = (96,1925)/gb = AL049703/gi = 4678806/ug = Hs. 18720/len = 2121 |
| 1385_at | M77349/FEATURE = /DEFINITION = HUMTGFBIG Human |
| | transforming growth factor-beta induced gene product (BIGH3) |
| | mRNA, complete cds |
| 37585_at | Cluster Incl. X13482: Human mRNA for U2 snRNP-specific A protein/ |
| | cds = (56,823)/gb = X13482/gi = 37546/ug = Hs. 80506/len = 1033 |
| 41869_at | Cluster Incl. U78310: Homo sapiens pescadillo mRNA, complete cds/ |
| | cds = (58,1824)/gb = U78310/gi = 2194202/ug = Hs. 13501/len = 2235 |
| 33833_at | Cluster Incl. J05243: Human nonerythroid alpha-spectrin (SPTAN1) |
| | mRNA, complete cds/cds = (102,7520)/gb = J05243/gi = 179105/ |
| | ug = Hs. 237180/len = 7787 |
| 38794_at | Cluster Incl. X53390: Human mRNA for upstream binding factor |
| | (hUBF)/cds = (147,2441)/gb = X53390/gi = 509240/ug = Hs. 89781/ |
| | len = 3097 |
| 33915_at | Cluster Incl. W22655: 71B9 Homo sapiens cDNA/clone = (not- |
| | directional)/gb = W22655/gi = 1299488/ug = Hs. 26070/len = 761 |
| 35905_s_at | Cluster Incl. U34995: Human normal keratinocyte subtraction library |
| | mRNA, clone H22a, complete sequence/cds = UNKNOWN/ |
| | gb = U34995/gi = 1497857/ug = Hs. 195188/len = 1626 |
| 39798_at | Cluster Incl. R87876: yo45h01.r1 Homo sapiens cDNA, 5 end/ |
| | clone = IMAGE-180913/clone_end = 5/gb = R87876/gi = 946689/ |
| | ug = Hs. 153177/len = 483 |
| 1878_g_at | M13194/FEATURE = mRNA/DEFINITION = HUMERCC1 Human |
| | excision repair protein (ERCC1) mRNA, complete cds, clone pcDE |
| 41116_at | Cluster Incl. AI799802: wc43d09.x1 Homo sapiens cDNA, 3 end/ |
| | clone = IMAGE-2321393/clone_end = 3/gb = AI799802/gi = 5365274/ |
| | ug = Hs. 101516/len = 688 |
| 35961_at | Cluster Incl. AL049390: Homo sapiens mRNA; cDNA |
| | DKFZp586O1318 (from clone DKFZp586O1318)/cds = UNKNOWN/ |
| | gb = AL049390/gi = 4500184/ug = Hs. 22689/len = 2322 |
| 37390_at | Cluster Incl. D86977: Human mRNA for KIAA0224 gene, complete cds/ |
| | cds = (136,3819)/gb = D86977/gi = 1504027/ug = Hs. 78054/len = 4226 |
| 38841_at | Cluster Incl. AF068195: Homo sapiens putative glialblastoma cell |
| | differentiation-related protein (GBDR1) mRNA, complete cds/ |
| | cds = (58,1062)/gb = AF068195/gi = 3192872/ug = Hs. 9194/len = 1493 |
| 35787_at | Cluster Incl. AI986201: wr81a01.x1 Homo sapiens cDNA, 3 end/ |
| | clone = IMAGE-2494056/clone_end = 3/gb = AI986201/gi = 5813478/ |
| | ug = Hs. 66881/len = 814 |
| 39379_at | Cluster Incl. AL049397: Homo sapiens mRNA; cDNA |
| | DKFZp586C1019 (from clone DKFZp586C1019)/cds = UNKNOWN/ |
| | gb = AL049397/gi = 4500188/ug = Hs. 12314/len = 1720 |
| 928_at | L02785/FEATURE = /DEFINITION = HUMDRA Homo sapiens colon |
| | mucosa-associated (DRA) mRNA, complete cds |
| 37349_r_at | Cluster Incl. AI817618: wk39f01.x1 Homo sapiens cDNA, 3 end/ |
| | clone = IMAGE-2417785/clone_end = 3/gb = AI817618/gi = 5436697/ |
| | ug = Hs. 77558/len = 734 |
| 32933_r_at | Cluster Incl. AL050122: Homo sapiens mRNA; cDNA DKFZp586E121 |
| | (from clone DKFZp586E121)/cds = UNKNOWN/gb = AL050122/ |
| | gi = 4884330/ug = Hs. 227742/len = 1843 |
| 34909_at | Cluster Incl. AC004990: Homo sapiens PAC clone DJ1185I07 from |
| | 7q11.23-q21/cds = (0,1766)/gb = AC004990/gi = 3924668/ |
| | ug = Hs. 128653/len = 1767 |
| AFFX- | U18530 SGD: YEL018W Yeast S. cerevisiae Protein of unknown |
| YEL018w/_at | function |
| 37054_at | Cluster Incl. J04739: Human bactericidal permeability increasing |
| | protein (BPI) mRNA, complete cds/cds = (30,1493)/gb = J04739/ |
| | gi = 179528/ug = Hs. 89535/len = 1813 |
| 38871_at | Cluster Incl. AJ006288: Homo sapiens mRNA for bcl-10 protein/ |
| | cds = (690,1391)/gb = AJ006288/gi = 4049459/ug = Hs. 193516 |
| | len = 1877 |
| 37800_r_at | Cluster Incl. AI263099: qz35b09.x1 Homo sapiens cDNA, 3 end/ |
| | clone = IMAGE-2028857/clone_end = 3/gb = AI263099/gi = 3871302/ |
| | ug = Hs. 126261/len = 838 |
| 37236_at | Cluster Incl. M11437: Human kininogen gene/cds = (0,1934)/ |
| | gb = M11437/gi = 186752/ug = Hs. 77741/len = 1935 |
| 38198_at | Cluster Incl. AL079275: Homo sapiens mRNA full length insert cDNA |
| | clone EUROIMAGE 566443/cds = UNKNOWN/gb = AL079275/ |
| | gi = 5102578/ug = Hs. 157078/len = 2082 |
| 35640_at | Cluster Incl. D14822: Human chimeric mRNA derived from AML1 |
| | gene and MTG8(ETO) gene, partial sequence/cds = (0,597)/ |
| | gb = D14822/gi = 467498/ug = Hs. 31551/len = 799 |
| 39828_at | Cluster Incl. AA477714: zu44e09.s1 Homo sapiens cDNA, 3 end/ |
| | clone = IMAGE-740872/clone_end = 3/gb = AA477714/gi = 2206348/ |
| | ug = Hs. 111554/len = 588 |
| 38938_at | Cluster Incl. AI816413: au47f05.x1 Homo sapiens cDNA, 3 end/ |
| | clone = IMAGE-2517921/clone_end = 3/gb = AI816413/gi = 5431959/ |
| | ug = Hs. 210862/len = 586 |
| 39654_at | Cluster Incl. S67156: ASP = aspartoacylase [human, kidney, mRNA, |
| | 1435 nt]/cds = (158,1099)/gb = S67156/gi = 455833/ug = Hs. 32042/ |
| | len = 1417 |
| 1393_at | L20348/FEATURE = expanded_cds/DEFINITION = HUMOMDLN04 |
| | Homo sapiens oncomodulin gene, exon 5 |
| 35920_at | Cluster Incl. N55205: yv44g05.s1 Homo sapiens cDNA, 3 end/ |
| | clone = IMAGE-245624/clone_end = 3/gb = N55205/gi = 1198084/ |
| | ug = Hs. 20205/len = 458 |
| 31368_at | Cluster Incl. W27967: 40b10 Homo sapiens cDNA/gb = W27967/ |
| | gi = 1307915/ug = Hs. 136154/len = 755 |
| 39912_at | Cluster Incl. AB006179: Homo sapiens mRNA for heparin-sulfate 6- |
| | sulfotransferase, complete cds/cds = (111,1343)/gb = AB006179/ |
| | gi = 3073774/ug = Hs. 132884/len = 2051 |
| 35489_at | Cluster Incl. M82962: Human N-benzoyl-L-tyrosyl-p-amino-benzoic |
| | acid hydrolase alpha subunit (PPH alpha) mRNA, complete cds/ |
| | cds = (9,2249)/gb = M82962/gi = 535474/ug = Hs. 179704/len = 2902 |
| 34486_at | Cluster Incl. U88897: Human endogenous retroviral H D2 leader region, |
| | protease region, and integrase/envelope region mRNA sequence/ |
| | cds = UNKNOWN/gb = U88897/gi = 2104917/ug = Hs. 11828/len = 1004 |
| 32596_at | Cluster Incl. W25828: 13g2 Homo sapiens cDNA/gb = W25828/ |
| | gi = 1305951/ug = Hs. 79362/len = 744 |
| 34057_at | Cluster Incl. U84392: Human Na+-dependent purine specific transporter |
| | mRNA, complete cds/cds = (59,2035)/gb = U84392/gi = 2731438/ |
| | ug = Hs. 193665/len = 2459 |
| 31759_at | Cluster Incl. W26220: 22d9 Homo sapiens cDNA/gb = W26220/ |
| | gi = 1306631/ug = Hs. 136089/len = 687 |
| 1485_at | L36642/FEATURE = mRNA/DEFINITION = HUMRPTK Homo |
| | sapiens receptor protein-tyrosine kinase (HEK11) mRNA, complete cds |
| 39475_at | Cluster Incl. L37199: Homo sapiens (clone cD24-1) Huntingtons |
| | disease candidate region mRNA fragment/cds = UNKNOWN/ |
| | gb = L37199/gi = 600520/ug = Hs. 117487/len = 1356 |
| 33012_at | Cluster Incl. L09753: Homo sapiens CD30 ligand mRNA, complete cds/ |
| | cds = (114,818)/gb = L09753/gi = 349277/ug = Hs. 1313/len = 1906 |
| 1321_s_at | U43916/FEATURE = /DEFINITION = HSU43916 Human tumor- |
| | associated membrane protein homolog (TMP) mRNA, complete cds |
| 32387_at | Cluster Incl. AB017494: Homo sapiens mRNA for LCAT-like |
| | lysophospholipase (LLPL), complete cds/cds = (32,1270)/ |
| | gb = AB017494/gi = 4589719/ug = Hs. 227221/len = 1400 |
| 35565_at | Cluster Incl. U79301: Human clone 23842 mRNA sequence/ |
| | cds = UNKNOWN/gb = U79301/gi = 1710286/ug = Hs. 135617/ |
| | len = 1582 |
| 1832_at | M62397/FEATURE = /DEFINITION = HUMCRCMUT Human |
| | colorectal mutant cancer protein mRNA, complete cds |
| 39924_at | Cluster Incl. AB020660: Homo sapiens mRNA for KIAA0853 protein, |
| | partial cds/cds = (0,2905)/gb = AB020660/gi = 4240194/ug = Hs. 136102/ |
| | len = 4363 |
| 39281_at | Cluster Incl. AB002378: Human mRNA for KIAA0380 gene, complete |
| | cds/cds = (745,5313)/gb = AB002378/gi = 2224700/ug = Hs. 239022/ |
| | len = 5790 |
| 34976_at | Cluster Incl. M60052: Human histidine-rich calcium binding protein |
| | (HRC) mRNA, complete cds/cds = (170,2269)/gb = M60052/gi = 183918/ |
| | ug = Hs. 1480/len = 2365 |
| 39642_at | Cluster Incl. AL080199: Homo sapiens mRNA; cDNA DKFZp434E082 |
| | (from clone DKFZp434E082)/cds = UNKNOWN/gb = AL080199/ |
| | gi = 5262682/ug = Hs. 30504/len = 1034 |
| 33615_at | Cluster Incl. X64994: H. sapiens HGMP07I gene for olfactory receptor/ |
| | cds = (0,944)/gb = X64994/gi = 32085/ug = Hs. 163670/len = 945 |
| 32054_at | Cluster Incl. AF048732: Homo sapiens cyclin T2b mRNA, complete cds/ |
| | cds = (0,2192)/gb = AF048732/gi = 2981199/ug = Hs. 155478/len = 2193 |
| 36383_at | Cluster Incl. M17254: Human erg2 gene encoding erg2 protein, |
| | complete cds/cds = (0,1388)/gb = M17254/gi = 182186/ug = Hs. 159432/ |
| | len = 1389 |
| 154_at | X07024/FEATURE = cds/DEFINITION = HSCCG1 Human X |
| | chromosome mRNA for CCG1 protein inv. in cell proliferation |
| 39882_at | Cluster Incl. U66035: Human X-linked deafness dystonia protein (DDP) |
| | mRNA, complete cds/cds = (35,328)/gb = U66035/gi = 3123842/ |
| | ug = Hs. 125565/len = 1169 |
| 35452_at | Cluster Incl. AL109690: Homo sapiens mRNA full length insert cDNA |
| | clone EUROIMAGE 190711/cds = UNKNOWN/gb = AL109690/ |
| | gi = 5689787/ug = Hs. 169950/len = 2031 |
| 39926_at | Cluster Incl. U59913: Human chromosome 5 Mad homolog Smad5 |
| | mRNA, complete cds/cds = (130,1527)/gb = U59913/gi = 1654324/ |
| | ug = Hs. 37501/len = 2205 |
| 39246_at | Cluster Incl. Z75330: H. sapiens mRNA for nuclear protein SA-1/ |
| | cds = (400,4176)/gb = Z75330/gi = 2204212/ug = Hs. 234435/len = 4337 |
| 40248_at | Cluster Incl. AL022165: dJ71L16.5 (KIAA0267 LIKE putative |
| | Na(+)/H(+) exchanger)/cds = (0,1852)/gb = AL022165/gi = 3281985/ |
| | ug = Hs. 154353/len = 3487 |
| 31446_s_at | Cluster Incl. D89501: Human PBI gene, complete cds/cds = (14,418)/ |
| | gb = D89501/gi = 1854451/ug = Hs. 166099/len = 576 |
| 37937_at | Cluster Incl. AJ005257: Homo sapiens partial mRNA for beta- |
| | transducin family protein (putative)/cds = (0,262)/gb = AJ005257/ |
| | gi = 3043442/ug = Hs. 85570/len = 1349 |
| 39914_r_at | Cluster Incl. W28976: 54e5 Homo sapiens cDNA/gb = W28976/ |
| | gi = 1308924/ug = Hs. 133151/len = 903 |
| 37514_s_at | Cluster Incl. AB008047: Homo sapiens sMAP mRNA for small MBL- |
| | associated protein, complete cds/cds = (26,583)/gb = AB008047/ |
| | gi = 5002493/ug = Hs. 119983/len = 725 |
| 971_s_at | Y00083/FEATURE = cds/DEFINITION = HSGTSF Human mRNA for |
| | glioblastoma-derived T-cell suppressor factor G-TsF (transforming |
| | growth factor-beta2, TGF-beta2) |
| 41863_at | Cluster Incl. AF070623: Homo sapiens clone 24468 mRNA sequence/ |
| | cds = UNKNOWN/gb = AF070623/gi = 3283889/ug = Hs. 13423/ |
| | len = 1226 |
| 39304_g_at | Cluster Incl. Y14153: Homo sapiens mRNA for beta-transducin repeat |
| | containing protein/cds = (69,1778)/gb = Y14153/gi = 2995193/ |
| | ug = Hs. 239742/len = 2141 |
| 35003_at | Cluster Incl. AA534868: nf82b01.s1 Homo sapiens cDNA, 3 end/ |
| | clone = IMAGE-926377/clone_end = 3/gb = AA534868/gi = 2279121/ |
| | ug = Hs. 152400/len = 595 |
| 34059_at | Cluster Incl. AA586695: nn42h06.s1 Homo sapiens cDNA, 3 end/ |
| | clone = IMAGE-1086587/clone_end = 3/gb = AA586695/gi = 2397509/ |
| | ug = Hs. 193956/len = 522 |
| 41112_at | Cluster Incl. AB011129: Homo sapiens mRNA for KIAA0557 protein, |
| | partial cds/cds = (0,1482)/gb = AB011129/gi = 3043637/ug = Hs. 101414/ |
| | len = 5627 |
| 31922_i_at | Cluster Incl. U60269: Human endogenous retrovirus HER V-K(HML6) |
| | proviral clone HML6.17 putative polymerase and envelope genes, |
| | partial cds, and 3LTR/cds = (0,491)/gb = U60269/gi = 1408208/ |
| | ug = Hs. 159902/len = 492 |
| 2023_g_at | M77198/FEATURE = /DEFINITION = HUMRPKB Human rac protein |
| | kinase beta mRNA, complete cds |
| 40919_at | Cluster Incl. M81830: Human somatostatin receptor isoform 2 (SSTR2) |
| | gene, complete cds/cds = (0,1109)/gb = M81830/gi = 307435/ |
| | ug = Hs. 184841/len = 1110 |
| 677_s_at | J04430/FEATURE = mRNA/DEFINITION = HUMACP5 Human |
| | tartrate-resistant acid phosphatase type 5 mRNA, complete cds |
| 41291_at | Cluster Incl. AC004528: Homo sapiens chromosome 19, cosmid |
| | R32184/cds = (0,1589)/gb = AC004528/gi = 3025444/ug = Hs. 238519/ |
| | len = 1590 |
| 32746_at | Cluster Incl. AF015451: Homo sapiens Usurpin-beta mRNA, complete |
| | cds/cds = (0,1388)/gb = AF015451/gi = 3133282/ug = Hs. 195175/ |
| | len = 1389 |
| 39364_s_at | Cluster Incl. Y18207: Homo sapiens mRNA for protein phosphatase 1 |
| | (PPP1R5)/cds = (91,1044)/gb = Y18207/gi = 3805818/ug = Hs. 12112/ |
| | len = 1158 |
| 135_g_at | X95632/FEATURE = cds/DEFINITION = HSARGBPIA H.sapiens |
| | mRNA for Arg protein tyrosine kinase-binding protein |
| 37785_at | Cluster Incl. U69563: U69563 Homo sapiens cDNA/clone = 25050/ |
| | gb = U69563/gi = 2731394/ug = Hs. 124940/len = 1657 |
| 39190_s_at | Cluster Incl. AC002126: Homo sapiens DNA from chromosome 19- |
| | cosmids R30102-R29350-R27740 containing MEF2B, genomic |
| | sequence/cds = (0,307)/gb = AC002126/gi = 2329908/ug = Hs. 125220/ |
| | len = 308 |
| 41550_at | Cluster Incl. AF091071: Homo sapiens clone 192 Rer1 mRNA, |
| | complete cds/cds = (76,696)/gb = AF091071/gi = 3859979/ug = Hs. 40500/ |
| | len = 1400 |
| 40240_at | Cluster Incl. AC004131: Homo sapiens Chromosome 16 BAC clone |
| | CIT987SK-A-69G12/cds = (0,1211)/gb = AC004131/gi = 3342217/ |
| | ug = Hs. 154050/len = 1887 |
| 38224_at | Cluster Incl. U71300: Human snRNA activating protein complex 50 kD |
| | subunit (SNAP50) mRNA, complete cds/cds = (14,1249)/gb = U71300/ |
| | gi = 1619945/ug = Hs. 164915/len = 1848 |
| 41534_at | Cluster Incl. AB006755: Homo sapiens mRNA for PCDH7 (BH-Pcdh)a, |
| | complete cds/cds = (1010,4219)/gb = AB006755/gi = 2979417/ |
| | ug = Hs. 34073/len = 4648 |
| 1569_r_at | L42243/FEATURE = exon#3/DEFINITION = HUMIFNAM08 Homo |
| | sapiens (clone 51H8) alternatively spliced interferon receptor |
| | (IFNAR2) gene, exon 9 and complete cds s |
| 35960_at | Cluster Incl. AF031416: Homo sapiens IkB kinase beta subunit mRNA, |
| | complete cds/cds = (0,2270)/gb = AF031416/gi = 3213216/ |
| | ug = Hs. 226573/len = 2271 |
| 32149_at | Cluster Incl. AA532495: nj54a10.s1 Homo sapiens cDNA/ |
| | clone = IMAGE-996282/gb = AA532495/gi = 2276749/ug = Hs. 183752/ |
| | len = 549 |
| 1668_s_at | L15409/FEATURE = /DEFINITION = HUMHIPLIND Homo sapiens |
| | (clone g7) von Hippel-Lindau disease tumor suppressor mRNA |
| | sequence |
| 32877_i_at | Cluster Incl. AA524802: nh33h11.s1 Homo sapiens cDNA/ |
| | clone = IMAGE-954213/gb = AA524802/gi = 2265730/ug = Hs. 203907/ |
| | len = 500 |
| 37152_at | Cluster Incl. L07592: Human peroxisome proliferator activated receptor |
| | mRNA, complete cds/cds = (337,1662)/gb = L07592/gi = 190229/ |
| | ug = Hs. 106415/len = 3301 |
| 33155_at | Cluster Incl. M95740: Human alpha-L-iduronidase gene/cds = (0,1961)/ |
| | gb = M95740/gi = 178412/ug = Hs. 89560/len = 2234 |
| 34031_i_at | Cluster Incl. U90268: Human Krit1 mRNA, complete cds/ |
| | cds = (25,1614)/gb = U90268/gi = 2149601/ug = Hs. 93810/len = 1986 |
| 39504_at | Cluster Incl. AF014643: Homo sapiens connexin46.6 (Cx46.6) gene, |
| | complete cds/cds = (28,1338)/gb = AF014643/gi = 2738576/ |
| | ug = Hs. 100072/len = 2087 |
| 40975_s_at | Cluster Incl. AL050258: Novel human mRNA similar to mouse tuftelin- |
| | interacting protein 10 mRNA, AF097181/cds = (263,2776)/ |
| | gb = AL050258/gi = 4886426/ug = Hs. 20225/len = 3565 |
| 40241_at | Cluster Incl. U09850: Human zinc finger protein (ZNF143) mRNA, |
| | complete cds/cds = (37,1917)/gb = U09850/gi = 495571/ug = Hs. 154095/ |
| | len = 3908 |
| 33723_at | Cluster Incl. AL049346: Homo sapiens mRNA; cDNA DKFZp566B213 |
| | (from clone DKFZp566B213)/cds = UNKNOWN/gb = AL049346/ |
| | gi = 4500130/ug = Hs. 194051/len = 1554 |
| 1459_at | M68941/FEATURE = mRNA/DEFINITION = HUMPTYPH Human |
| | protein-tyrosine phosphatase mRNA, complete cds |
| 40033_at | Cluster Incl. AL022328: Human DNA sequence from clone 402G11 on |
| | chromosome 22q13.31-13.33 Contains genes for SAPK3 (stress- |
| | activated protein kinase 3), PRKM11 (protein kinase mitogen-activated |
| | 11), KIAA0315, ESTs, GSSs and CpG islands/cds = (11,1105)/ |
| | gb = AL022328/gi = 5263010/ug = Hs. 57732/len = 2341 |
| 39661_s_at | Cluster Incl. AF034102: Homo sapiens NBMPR-insensitive nucleoside |
| | transporter ei (ENT2) mRNA, complete cds/cds = (237,1607)/ |
| | gb = AF034102/gi = 2811136/ug = Hs. 32951/len = 2522 |
| 37629_at | Cluster Incl. M55268: Human casein kinase II alpha subunit mRNA, |
| | complete cds/cds = (163,1215)/gb = M55268/gi = 177837/ug = Hs. 82201/ |
| | len = 1677 |
| 1624_at | Stimulatory Gdp/Gtp Exchange Protein For C-Ki-Ras P21 And Smg |
| | P21 |
| 1903_at | Ras-Related Protein Rap1b |
| 33170_at | Cluster Incl. AB023179: Homo sapiens mRNA for KIAA0962 protein, |
| | partial cds/cds = (0,1893)/gb = AB023179/gi = 4589567/ug = Hs. 9059/ |
| | len = 5460 |
| 33175_at | Cluster Incl. AA156237: z150c09.s1 Homo sapiens cDNA, 3 end/ |
| | clone = IMAGE-505360/clone_end = 3/gb = AA156237/gi = 1727855/ |
| | ug = Hs. 90804/len = 644 |
| 38044_at | Cluster Incl. AF035283: Homo sapiens clone 23916 mRNA sequence/ |
| | cds = UNKNOWN/gb = AF035283/gi = 2661034/ug = Hs. 8022/ |
| | len = 2022 |
| 40440_at | Cluster Incl. AL080119: Homo sapiens mRNA; cDNA |
| | DKFZp564M2423 (from clone DKFZp564M2423)/cds = (85,1248)/ |
| | gb = AL080119/gi = 5262550/ug = Hs. 165998/len = 2183 |
| 35254_at | Cluster Incl. AB007447: Homo sapiens mRNA for Fln29, complete cds/ |
| | cds = (54,1802)/gb = AB007447/gi = 2463530/ug = Hs. 5148/len = 2618 |

Next, we calculated phenotype association indices for all 52 samples and determined that this gene cluster exhibited a 77% success rate in clinical sample classification based on individual phenotype association indices (Table 48). As shown in Table 48, 22/26 (or 85%) of the invasive prostate cancer samples had positive phenotype association indices, whereas 18/26 (or 69%) of non-invasive prostate cancer samples displayed negative phenotype association indices. Overall, 40 of 52 samples (or 77%) were correctly classified.

TABLE 48
Classification accuracy of the prostate cancer invasion clusters

| | r value | | | |
|---|---|---|---|---|
| | (Phenotype | | | |
| | Association | Invasive | Non-invasive | |
| Cluster | Index) | tumors | tumors | Overall |
| 114 genes | 0.704 | 22/26 (85%) | 18/26 (69%) | 40/52 (77%) |
| 53 genes | 0.893 | 22/26 (85%) | 17/26 (65%) | 39/52 (75%) |
| 39 genes | 0.972 | 22/26 (85%) | 18/26 (69%) | 40/52 (77%) |
| 26 genes | 0.994 | 23/26 (88%) | 17/26 (65%) | 40/52 (77%) |
| 24 genes | 0.997 | 21/26 (81%) | 17/26 (65%) | 38/52 (73%) |
| 22 genes | 0.995 | 21/26 (81%) | 18/26 (69%) | 39/52 (75%) |

Next, we identified a single best-fit invasive prostate cancer sample displaying the correlation coefficient of 0.704 to the average expression profile of the 26 invasive prostate cancer samples. The expression profile of this single best-fit invasive prostate cancer sample was utilized as a second reference set.

The concordance set was obtained by selecting only those genes having a consistent direction of the differential expression in both the first and the second reference sets (i.e., greater gene expression difference in the invasive cf. the non-invasive samples and greater gene expression in the best-fit tumor sample cf. the average expression value across the entire data set or vice-versa). The concordance set comprised of 107 genes (r=0.721). A minimum segregation set was selected following the procedures described in above. Scatter plots were generated of the log₁₀ transformed average −fold expression change in the first reference set and average −fold expression change in the second reference set (in case of a single best-fit tumor it was the log₁₀ t transformed ratio of the expression value for a gene to the average expression value across the entire data set). For the samples of the first reference set, <expression>₁ corresponds to the average expression value for gene x over all samples from patients who had invasive tumors and <expression>₂ corresponds to the average expression value for gene x over all samples from patients who had non-invasive tumors. A minimum segregation set was identified by selecting a subset of the highly correlated genes between two reference sets from the invasiveness concordance set. Using this approach we identified five gene clusters discriminating with high accuracy between invasive and non-invasive human prostate tumors. The members of these invasion predictors or invasion minimum segregation sets (invasion minimum segregation gene clusters) are listed in Tables 49-54. The classification performance for each of these gene clusters is presented in the Table 48.

TABLE 49
53-gene signature of invasive prostate cancer

| Affymetrix | |
|---|---|
| Probe Set ID | |
| (U95Av2) | Description |
| 1878_g_at | M13194/FEATURE = mRNA/DEFINITION = HUMERCC1 Human |
| | excision repair protein (ERCC1) mRNA, complete cds, clone pcDE |
| 33833_at | Cluster Incl. J05243: Human nonerythroid alpha-spectrin (SPTAN1) |
| | mRNA, complete cds/cds = (102,7520)/gb = J05243/gi = 179105/ |
| | ug = Hs. 237180/len = 7787 |
| 33915_at | Cluster Incl. W22655: 71B9 Homo sapiens cDNA/clone = (not- |
| | directional)/gb = W22655/gi = 1299488/ug = Hs. 26070/len = 761 |
| 35787_at | Cluster Incl. AI986201: wr81a01.x1 Homo sapiens cDNA, 3 end/ |
| | clone = IMAGE-2494056/clone_end = 3/gb = AI986201/gi = 5813478/ |
| | ug = Hs. 66881/len = 814 |
| 37390_at | Cluster Incl. D86977: Human mRNA for KIAA0224 gene, complete cds/ |
| | cds = (136,3819)/gb = D86977/gi = 1504027/ug = Hs. 78054/len = 4226 |
| 38260_at | Cluster Incl. AL050306: Human DNA sequence from clone 475B7 on |
| | chromosome Xq12.1-13. Contains the 3 part of the gene for a novel |
| | KIAA0615 and KIAA0323 LIKE protein, the gene for a novel protein, |
| | ESTs, STSs, GSSs and two putative CpG islands/cds = (48,2201)/ |
| | gb = AL050306/gi = 5419784/ug = Hs. 90625/len = 2395 |
| 38794_at | Cluster Incl. X53390: Human mRNA for upstream binding factor (hUBF)/ |
| | cds = (147,2441)/gb = X53390/gi = 509240/ug = Hs. 89781/len = 3097 |
| 38841_at | Cluster Incl. AF068195: Homo sapiens putative glialblastoma cell |
| | differentiation-related protein (GBDR1) mRNA, complete cds/ |
| | cds = (58,1062)/gb = AF068195/gi = 3192872/ug = Hs. 9194/len = 1493 |
| 39379_at | Cluster Incl. AL049397: Homo sapiens mRNA; cDNA DKFZp586C1019 |
| | (from clone DKFZp586C1019)/cds = UNKNOWN/gb = AL049397/ |
| | gi = 4500188/ug = Hs. 12314/len = 1720 |
| 40635_at | Cluster Incl. AF089750: Homo sapiens flotillin-1 mRNA, complete cds/ |
| | cds = (164,1447)/gb = AF089750/gi = 3599572/ug = Hs. 179986/len = 1796 |
| 41116_at | Cluster Incl. AI799802: wc43d09.x1 Homo sapiens cDNA, 3 end/ |
| | clone = IMAGE-2321393/clone_end = 3/gb = AI799802/gi = 5365274/ |
| | ug = Hs. 101516/len = 688 |
| 41869_at | Cluster Incl. U78310: Homo sapiens pescadillo mRNA, complete cds/ |
| | cds = (58,1824)/gb = U78310/gi = 2194202/ug = Hs. 13501/len = 2235 |
| 1321_s_at | U43916/FEATURE = /DEFINITION = HSU43916 Human tumor- |
| | associated membrane protein homolog (TMP) mRNA, complete cds |
| 154_at | X07024/FEATURE = cds/DEFINITION = HSCCG1 Human X |
| | chromosome mRNA for CCG1 protein inv. in cell proliferation |
| 1569_r_at | L42243/FEATURE = exon #3/DEFINITION = HUMIFNAM08 Homo |
| | sapiens (clone 51H8) alternatively spliced interferon receptor (IFNAR2) |
| | gene, exon 9 and complete cds s |
| 1668_s_at | L15409/FEATURE = /DEFINITION = HUMHIPLIND Homo sapiens |
| | (clone g7) von Hippel-Lindau disease tumor suppressor mRNA sequence |
| 1832_at | M62397/FEATURE = /DEFINITION = HUMCRCMUT Human |
| | colorectal mutant cancer protein mRNA, complete cds |
| 1903_at | Ras-Related Protein Rap1b |
| 31368_at | Cluster Incl. W27967: 40b10 Homo sapiens cDNA/gb = W27967/ |
| | gi = 1307915/ug = Hs. 136154/len = 755 |
| 31446_s_at | Cluster Incl. D89501: Human PBI gene, complete cds/cds = (14,418)/ |
| | gb = D89501/gi = 1854451/ug = Hs. 166099/len = 576 |
| 31922_i_at | Cluster Incl. U60269: Human endogenous retrovirus HERV-K(HML6) |
| | proviral clone HML6.17 putative polymerase and envelope genes, partial |
| | cds, and 3LTR/cds = (0,491)/gb = U60269/gi = 1408208/ug = Hs. 159902/ |
| | len = 492 |
| 32054_at | Cluster Incl. AF048732: Homo sapiens cyclin T2b mRNA, complete cds/ |
| | cds = (0,2192)/gb = AF048732/gi = 2981199/ug = Hs. 155478/len = 2193 |
| 32149_at | Cluster Incl. AA532495: nj54a10.s1 Homo sapiens cDNA/ |
| | clone = IMAGE-996282/gb = AA532495/gi = 2276749/ug = Hs. 183752/ |
| | len = 549 |
| 32596_at | Cluster Incl. W25828: 13g2 Homo sapiens cDNA/gb = W25828/ |
| | gi = 1305951/ug = Hs. 79362/len = 744 |
| 33615_at | Cluster Incl. X64994: H. sapiens HGMP07I gene for olfactory receptor/ |
| | cds = (0,944)/gb = X64994/gi = 32085/ug = Hs. 163670/len = 945 |
| 33723_at | Cluster Incl. AL049346: Homo sapiens mRNA; cDNA DKFZp566B213 |
| | (from clone DKFZp566B213)/cds = UNKNOWN/gb = AL049346/ |
| | gi = 4500130/ug = Hs. 194051/len = 1554 |
| 34057_at | Cluster Incl. U84392: Human Na+-dependent purine specific transporter |
| | mRNA, complete cds/cds = (59,2035)/gb = U84392/gi = 2731438/ |
| | ug = Hs. 193665/len = 2459 |
| 34059_at | Cluster Incl. AA586695: nn42h06.s1 Homo sapiens cDNA, 3 end/ |
| | clone = IMAGE-1086587/clone_end = 3/gb = AA586695/gi = 2397509/ |
| | ug = Hs. 193956/len = 522 |
| 34486_at | Cluster Incl. U88897: Human endogenous retroviral H D2 leader region, |
| | protease region, and integrase/envelope region mRNA sequence/ |
| | cds = UNKNOWN/gb = U88897/gi = 2104917/ug = Hs. 11828/len = 1004 |
| 34909_at | Cluster Incl. AC004990: Homo sapiens PAC clone DJ1185I07 from |
| | 7q11.23-q21/cds = (0,1766)/gb = AC004990/gi = 3924668/ug = Hs. 128653/ |
| | len = 1767 |
| 35489_at | Cluster Incl. M82962: Human N-benzoyl-L-tyrosyl-p-amino-benzoic acid |
| | hydrolase alpha subunit (PPH alpha) mRNA, complete cds/cds = (9,2249)/ |
| | gb = M82962/gi = 535474/ug = Hs. 179704/len = 2902 |
| 35565_at | Cluster Incl. U79301: Human clone 23842 mRNA sequence/ |
| | cds = UNKNOWN/gb = U79301/gi = 1710286/ug = Hs. 135617/len = 1582 |
| 35640_at | Cluster Incl. D14822: Human chimeric mRNA derived from AML1 gene |
| | and MTG8(ETO) gene, partial sequence/cds = (0,597)/gb = D14822/ |
| | gi = 467498/ug = Hs. 31551/len = 799 |
| 35960_at | Cluster Incl. AF031416: Homo sapiens IkB kinase beta subunit mRNA, |
| | complete cds/cds = (0,2270)/gb = AF031416/gi = 3213216/ug = Hs. 226573/ |
| | len = 2271 |
| 37054_at | Cluster Incl. J04739: Human bactericidal permeability increasing protein |
| | (BPI) mRNA, complete cds/cds = (30,1493)/gb = J04739/gi = 179528/ |
| | ug = Hs. 89535/len = 1813 |
| 37785_at | Cluster Incl. U69563: U69563 Homo sapiens cDNA/clone = 25050/ |
| | gb = U69563/gi = 2731394/ug = Hs. 124940/len = 1657 |
| 38198_at | Cluster Incl. AL079275: Homo sapiens mRNA full length insert cDNA |
| | clone EUROIMAGE 566443/cds = UNKNOWN/gb = AL079275/ |
| | gi = 5102578/ug = Hs. 157078/len = 2082 |
| 38871_at | Cluster Incl. AJ006288: Homo sapiens mRNA for bcl-10 protein/ |
| | cds = (690,1391)/gb = AJ006288/gi = 4049459/ug = Hs. 193516/len = 1877 |
| 38938_at | Cluster Incl. AI816413: au47f05.x1 Homo sapiens cDNA, 3 end/ |
| | clone = IMAGE-2517921/clone_end = 3/gb = AI816413/gi = 5431959/ |
| | ug = Hs. 210862/len = 586 |
| 39304_g_at | Cluster Incl. Y14153: Homo sapiens mRNA for beta-transducin repeat |
| | containing protein/cds = (69,1778)/gb = Y14153/gi = 2995193/ |
| | ug = Hs. 239742/len = 2141 |
| 39364_s_at | Cluster Incl. Y18207: Homo sapiens mRNA for protein phosphatase 1 |
| | (PPP1R5)/cds = (91,1044)/gb = Y18207/gi = 3805818/ug = Hs. 12112/ |
| | len = 1158 |
| 39475_at | Cluster Incl. L37199: Homo sapiens (clone cD24-1) Huntingtons disease |
| | candidate region mRNA fragment/cds = UNKNOWN/gb = L37199/ |
| | gi = 600520/ug = Hs. 117487/len = 1356 |
| 39661_s_at | Cluster Incl. AF034102: Homo sapiens NBMPR-insensitive nucleoside |
| | transporter ei (ENT2) mRNA, complete cds/cds = (237,1607)/ |
| | gb = AF034102/gi = 2811136/ug = Hs. 32951/len = 2522 |
| 39882_at | Cluster Incl. U66035: Human X-linked deafness dystonia protein (DDP) |
| | mRNA, complete cds/cds = (35,328)/gb = U66035/gi = 3123842/ |
| | ug = Hs. 125565/len = 1169 |
| 39912_at | Cluster Incl. AB006179: Homo sapiens mRNA for heparan-sulfate 6- |
| | sulfotransferase, complete cds/cds = (111,1343)/gb = AB006179/ |
| | gi = 3073774/ug = Hs. 132884/len = 2051 |
| 39924_at | Cluster Incl. AB020660: Homo sapiens mRNA for KIAA0853 protein, |
| | partial cds/cds = (0,2905)/gb = AB020660/gi = 4240194/ug = Hs. 136102/ |
| | len = 4363 |
| 39926_at | Cluster Incl. U59913: Human chromosome 5 Mad homolog Smad5 |
| | mRNA, complete cds/cds = (130,1527)/gb = U59913/gi = 1654324/ |
| | ug = Hs. 37501/len = 2205 |
| 40241_at | Cluster Incl. U09850: Human zinc finger protein (ZNF143) mRNA, |
| | complete cds/cds = (37,1917)/gb = U09850/gi = 495571/ug = Hs. 154095/ |
| | len = 3908 |
| 40975_s_at | Cluster Incl. AL050258: Novel human mRNA similar to mouse tuftelin- |
| | interacting protein 10 mRNA, AF097181/cds = (263,2776)/ |
| | gb = AL050258/gi = 4886426/ug = Hs. 20225/len = 3565 |
| 41112_at | Cluster Incl. AB011129: Homo sapiens mRNA for KIAA0557 protein, |
| | partial cds/cds = (0,1482)/gb = AB011129/gi = 3043637/ug = Hs. 101414/ |
| | len = 5627 |
| 41550_at | Cluster Incl. AF091071: Homo sapiens clone 192 Rer1 mRNA, complete |
| | cds/cds = (76,696)/gb = AF091071/gi = 3859979/ug = Hs. 40500/len = 1400 |
| 677_s_at | J04430/FEATURE = mRNA/DEFINITION = HUMACP5 Human tartrate- |
| | resistant acid phosphatase type 5 mRNA, complete cds |
| 971_s_at | Y00083/FEATURE = cds/DEFINITION = HSGTSF Human mRNA for |
| | glioblastoma-derived T-cell suppressor factor G-TsF (transforming |
| | growth factor-beta2, TGF-beta2) |

TABLE 50
39-gene signature of invasive prostate cancer

| Affymetrix | |
|---|---|
| Probe Set ID | |
| (U95Av2) | Description |
| 1878_g_at | M13194/FEATURE = mRNA/DEFINITION = HUMERCC1 Human |
| | excision repair protein (ERCC1) mRNA, complete cds, clone pcDE |
| 33833_at | Cluster Incl. J05243: Human nonerythroid alpha-spectrin (SPTAN1) |
| | mRNA, complete cds/cds = (102,7520)/gb = J05243/gi = 179105/ |
| | ug = Hs. 237180/len = 7787 |
| 33915_at | Cluster Incl. W22655: 71B9 Homo sapiens cDNA/clone = (not-directional)/ |
| | gb = W22655/gi = 1299488/ug = Hs. 26070/len = 761 |
| 35787_at | Cluster Incl. AI986201: wr81a01.x1 Homo sapiens cDNA, 3 end/ |
| | clone = IMAGE-2494056/clone_end = 3/gb = AI986201/gi = 5813478/ |
| | ug = Hs. 66881/len = 814 |
| 37390_at | Cluster Incl. D86977: Human mRNA for KIAA0224 gene, complete cds/ |
| | cds = (136,3819)/gb = D86977/gi = 1504027/ug = Hs. 78054/len = 4226 |
| 38260_at | Cluster Incl. AL050306: Human DNA sequence from clone 475B7 on |
| | chromosome Xq12.1-13. Contains the 3 part of the gene for a novel |
| | KIAA0615 and KIAA0323 LIKE protein, the gene for a novel protein, |
| | ESTs, STSs, GSSs and two putative CpG islands/cds = (48,2201)/ |
| | gb = AL050306/gi = 5419784/ug = Hs. 90625/len = 2395 |
| 38794_at | Cluster Incl. X53390: Human mRNA for upstream binding factor (hUBF)/ |
| | cds = (147,2441)/gb = X53390/gi = 509240/ug = Hs. 89781/len = 3097 |
| 38841_at | Cluster Incl. AF068195: Homo sapiens putative glialblastoma cell |
| | differentiation-related protein (GBDR1) mRNA, complete cds/ |
| | cds = (58,1062)/gb = AF068195/gi = 3192872/ug = Hs. 9194/len = 1493 |
| 39379_at | Cluster Incl. AL049397: Homo sapiens mRNA; cDNA DKFZp586C1019 |
| | (from clone DKFZp586C1019)/cds = UNKNOWN/gb = AL049397/ |
| | gi = 4500188/ug = Hs. 12314/len = 1720 |
| 40635_at | Cluster Incl. AF089750: Homo sapiens flotillin-1 mRNA, complete cds/ |
| | cds = (164,1447)/gb = AF089750/gi = 3599572/ug = Hs. 179986/len = 1796 |
| 41116_at | Cluster Incl. AI799802: wc43d09.x1 Homo sapiens cDNA, 3 end/ |
| | clone = IMAGE-2321393/clone_end = 3/gb = AI799802/gi = 5365274/ |
| | ug = Hs. 101516/len = 688 |
| 41869_at | Cluster Incl. U78310: Homo sapiens pescadillo mRNA, complete cds/ |
| | cds = (58,1824)/gb = U78310/gi = 2194202/ug = Hs. 13501/len = 2235 |
| 1321_s_at | U43916/FEATURE = /DEFINITION = HSU43916 Human tumor- |
| | associated membrane protein homolog (TMP) mRNA, complete cds |
| 1668_s_at | L15409/FEATURE = /DEFINITION = HUMHIPLIND Homo sapiens |
| | (clone g7) von Hippel-Lindau disease tumor suppressor mRNA sequence |
| 1832_at | M62397/FEATURE = /DEFINITION = HUMCRCMUT Human colorectal |
| | mutant cancer protein mRNA, complete cds |
| 1903_at | Ras-Related Protein Rap1b |
| 31368_at | Cluster Incl. W27967: 40b10 Homo sapiens cDNA/gb = W27967/ |
| | gi = 1307915/ug = Hs. 136154/len = 755 |
| 31446_s_at | Cluster Incl. D89501: Human PBI gene, complete cds/cds = (14,418)/ |
| | gb = D89501/gi = 1854451/ug = Hs. 166099/len = 576 |
| 31922_i_at | Cluster Incl. U60269: Human endogenous retrovirus HER V-K(HML6) |
| | proviral clone HML6.17 putative polymerase and envelope genes, partial |
| | cds, and 3LTR/cds = (0,491)/gb = U60269/gi = 1408208/ug = Hs. 159902/ |
| | len = 492 |
| 32054_at | Cluster Incl. AF048732: Homo sapiens cyclin T2b mRNA, complete cds/ |
| | cds = (0,2192)/gb = AF048732/gi = 2981199/ug = Hs. 155478/len = 2193 |
| 32149_at | Cluster Incl. AA532495: nj54a10.s1 Homo sapiens cDNA/clone = IMAGE- |
| | 996282/gb = AA532495/gi = 2276749/ug = Hs. 183752/len = 549 |
| 33723_at | Cluster Incl. AL049346: Homo sapiens mRNA; cDNA DKFZp566B213 |
| | (from clone DKFZp566B213)/cds = UNKNOWN/gb = AL049346/ |
| | gi = 4500130/ug = Hs. 194051/len = 1554 |
| 34059_at | Cluster Incl. AA586695: nn42h06.s1 Homo sapiens cDNA, 3 end/ |
| | clone = IMAGE-1086587/clone_end = 3/gb = AA586695/gi = 2397509/ |
| | ug = Hs. 193956/len = 522 |
| 34909_at | Cluster Incl. AC004990: Homo sapiens PAC clone DJ1185I07 from |
| | 7q11.23-q21/cds = (0,1766)/gb = AC004990/gi = 3924668/ug = Hs. 128653/ |
| | len = 1767 |
| 35489_at | Cluster Incl. M82962: Human N-benzoyl-L-tyrosyl-p-amino-benzoic acid |
| | hydrolase alpha subunit (PPH alpha) mRNA, complete cds/cds = (9,2249)/ |
| | gb = M82962/gi = 535474/ug = Hs. 179704/len = 2902 |
| 35640_at | Cluster Incl. D14822: Human chimeric mRNA derived from AML1 gene |
| | and MTG8(ETO) gene, partial sequence/cds = (0,597)/gb = D14822/ |
| | gi = 467498/ug = Hs. 31551/len = 799 |
| 37054_at | Cluster Incl. J04739: Human bactericidal permeability increasing protein |
| | (BPI) mRNA, complete cds/cds = (30,1493)/gb = J04739/gi = 179528/ |
| | ug = Hs. 89535/len = 1813 |
| 37785_at | Cluster Incl. U69563: U69563 Homo sapiens cDNA/clone = 25050/ |
| | gb = U69563/gi = 2731394/ug = Hs. 124940/len = 1657 |
| 38198_at | Cluster Incl. AL079275: Homo sapiens mRNA full length insert cDNA |
| | clone EUROIMAGE 566443/cds = UNKNOWN/gb = AL079275/ |
| | gi = 5102578/ug = Hs. 157078/len = 2082 |
| 38871_at | Cluster Incl. AJ006288: Homo sapiens mRNA for bcl-10 protein/ |
| | cds = (690,1391)/gb = AJ006288/gi = 4049459/ug = Hs. 193516/len = 1877 |
| 39475_at | Cluster Incl. L37199: Homo sapiens (clone cD24-1) Huntingtons disease |
| | candidate region mRNA fragment/cds = UNKNOWN/gb = L37199/ |
| | gi = 600520/ug = Hs. 117487/len = 1356 |
| 39661_s_at | Cluster Incl. AF034102: Homo sapiens NBMPR-insensitive nucleoside |
| | transporter ei (ENT2) mRNA, complete cds/cds = (237,1607)/ |
| | gb = AF034102/gi = 2811136/ug = Hs. 32951/len = 2522 |
| 39882_at | Cluster Incl. U66035: Human X-linked deafness dystonia protein (DDP) |
| | mRNA, complete cds/cds = (35,328)/gb = U66035/gi = 3123842/ |
| | ug = Hs. 125565/len = 1169 |
| 39912_at | Cluster Incl. AB006179: Homo sapiens mRNA for heparan-sulfate 6- |
| | sulfotransferase, complete cds/cds = (111,1343)/gb = AB006179/ |
| | gi = 3073774/ug = Hs. 132884/len = 2051 |
| 40241_at | Cluster Incl. U09850: Human zinc finger protein (ZNF143) mRNA, |
| | complete cds/cds = (37,1917)/gb = U09850/gi = 495571/ug = Hs. 154095/ |
| | len = 3908 |
| 40975_s_at | Cluster Incl. AL050258: Novel human mRNA similar to mouse tuftelin- |
| | interacting protein 10 mRNA, AF097181/cds = (263,2776)/gb = AL050258/ |
| | gi = 4886426/ug = Hs. 20225/len = 3565 |
| 41550_at | Cluster Incl. AF091071: Homo sapiens clone 192 Rer1 mRNA, complete |
| | cds/cds = (76,696)/gb = AF091071/gi = 3859979/ug = Hs. 40500/len = 1400 |
| 677_s_at | J04430/FEATURE = mRNA/DEFINITION = HUMACP5 Human tartrate- |
| | resistant acid phosphatase type 5 mRNA, complete cds |
| 971_s_at | Y00083/FEATURE = cds/DEFINITION = HSGTSF Human mRNA for |
| | glioblastoma-derived T-cell suppressor factor G-TsF (transforming growth |
| | factor-beta2, TGF-beta2) |

TABLE 51
26-gene signature of invasive prostate cancer

| Affymetrix | |
|---|---|
| Probe Set ID | |
| (U95Av2) | Description |
| 36993_at | Cluster Incl. M33210: Human colony stimulating factor 1 receptor |
| | (CSF1R) gene/cds = (0,283)/gb = M33210/gi = 532592/ug = Hs. 76144/ |
| | len = 2206 |
| 38682_at | Cluster Incl. AF045581: Homo sapiens BRCA1 associated protein 1 |
| | (BAP1) mRNA, complete cds/cds = (39,2228)/gb = AF045581/ |
| | gi = 2854120/ug = Hs. 106674/len = 3506 |
| 41725_at | Cluster Incl. U89896: Homo sapiens casein kinase I gamma 2 mRNA, |
| | complete cds/cds = (239,1486)/gb = U89896/gi = 1890117/ |
| | ug = Hs. 181390/len = 1749 |
| 32212_at | Cluster Incl. AL049703: Human gene from PAC 179D3, chromosome |
| | X, isoform of mitochondrial apoptosis inducing factor, AIF, AF100928/ |
| | cds = (96,1925)/gb = AL049703/gi = 4678806/ug = Hs. 18720/len = 2121 |
| 1385_at | M77349/FEATURE = /DEFINITION = HUMTGFBIG Human |
| | transforming growth factor-beta induced gene product (BIGH3) |
| | mRNA, complete cds |
| 37585_at | Cluster Incl. X13482: Human mRNA for U2 snRNP-specific A protein/ |
| | cds = (56,823)/gb = X13482/gi = 37546/ug = Hs. 80506/len = 1033 |
| 1903_at | Ras-Related Protein Rap1b |
| 39661_s_at | Cluster Incl. AF034102: Homo sapiens NBMPR-insensitive nucleoside |
| | transporter ei (ENT2) mRNA, complete cds/cds = (237,1607)/ |
| | gb = AF034102/gi = 2811136/ug = Hs. 32951/len = 2522 |
| 40241_at | Cluster Incl. U09850: Human zinc finger protein (ZNF143) mRNA, |
| | complete cds/cds = (37,1917)/gb = U09850/gi = 495571/ug = Hs. 154095/ |
| | len = 3908 |
| 40975_s_at | Cluster Incl. AL050258: Novel human mRNA similar to mouse tuftelin- |
| | interacting protein 10 mRNA, AF097181/cds = (263,2776)/ |
| | gb = AL050258/gi = 4886426/ug = Hs. 20225/len = 3565 |
| 32149_at | Cluster Incl. AA532495: nj54a10.s1 Homo sapiens cDNA/ |
| | clone = IMAGE-996282/gb = AA532495/gi = 2276749/ug = Hs. 183752/ |
| | len = 549 |
| 39190_s_at | Cluster Incl. AC002126: Homo sapiens DNA from chromosome 19- |
| | cosmids R30102-R29350-R27740 containing MEF2B, genomic |
| | sequence/cds = (0,307)/gb = AC002126/gi = 2329908/ug = Hs. 125220/ |
| | len = 308 |
| 32746_at | Cluster Incl. AF015451: Homo sapiens Usurpin-beta mRNA, complete |
| | cds/cds = (0,1388)/gb = AF015451/gi = 3133282/ug = Hs. 195175/ |
| | len = 1389 |
| 34059_at | Cluster Incl. AA586695: nn42h06.s1 Homo sapiens cDNA, 3 end/ |
| | clone = IMAGE-1086587/clone_end = 3/gb = AA586695/gi = 2397509/ |
| | ug = Hs. 193956/len = 522 |
| 39914_r_at | Cluster Incl. W28976: 54e5 Homo sapiens cDNA/gb = W28976/ |
| | gi = 1308924/ug = Hs. 133151/len = 903 |
| 32054_at | Cluster Incl. AF048732: Homo sapiens cyclin T2b mRNA, complete |
| | cds/cds = (0,2192)/gb = AF048732/gi = 2981199/ug = Hs. 155478/ |
| | len = 2193 |
| 1832_at | M62397/FEATURE = /DEFINITION = HUMCRCMUT Human |
| | colorectal mutant cancer protein mRNA, complete cds |
| 1321_s_at | U43916/FEATURE = /DEFINITION = HSU43916 Human tumor- |
| | associated membrane protein homolog (TMP) mRNA, complete cds |
| 35489_at | Cluster Incl. M82962: Human N-benzoyl-L-tyrosyl-p-amino-benzoic |
| | acid hydrolase alpha subunit (PPH alpha) mRNA, complete cds/ |
| | cds = (9,2249)/gb = M82962/gi = 535474/ug = Hs. 179704/len = 2902 |
| 39912_at | Cluster Incl. AB006179: Homo sapiens mRNA for heparan-sulfate 6- |
| | sulfotransferase, complete cds/cds = (111,1343)/gb = AB006179/ |
| | gi = 3073774/ug = Hs. 132884/len = 2051 |
| 31368_at | Cluster Incl. W27967: 40b10 Homo sapiens cDNA/gb = W27967/ |
| | gi = 1307915/ug = Hs. 136154/len = 755 |
| 35640_at | Cluster Incl. D14822: Human chimeric mRNA derived from AML1 |
| | gene and MTG8(ETO) gene, partial sequence/cds = (0,597)/ |
| | gb = D14822/gi = 467498/ug = Hs. 31551/len = 799 |
| 38198_at | Cluster Incl. AL079275: Homo sapiens mRNA full length insert cDNA |
| | clone EUROIMAGE 566443/cds = UNKNOWN/gb = AL079275/ |
| | gi = 5102578/ug = Hs. 157078/len = 2082 |
| 38871_at | Cluster Incl. AJ006288: Homo sapiens mRNA for bcl-10 protein/ |
| | cds = (690,1391)/gb = AJ006288/gi = 4049459/ug = Hs. 193516/ |
| | len = 1877 |
| 37054_at | Cluster Incl. J04739: Human bactericidal permeability increasing |
| | protein (BPI) mRNA, complete cds/cds = (30,1493)/gb = J04739/ |
| | gi = 179528/ug = Hs. 89535/len = 1813 |
| 34909_at | Cluster Incl. AC004990: Homo sapiens PAC clone DJ1185I07 from |
| | 7q11.23-q21/cds = (0,1766)/gb = AC004990/gi = 3924668/ |
| | ug = Hs. 128653/len = 1767 |

TABLE 52
24-gene signature of invasive prostate cancer

| Affymetrix | |
|---|---|
| Probe Set ID | |
| (U95Av2) | Description |
| 40635_at | Cluster Incl. AF089750: Homo sapiens flotillin-1 mRNA, complete cds/ |
| | cds = (164,1447)/gb = AF089750/gi = 3599572/ug = Hs. 179986/len = 1796 |
| 38260_at | Cluster Incl. AL050306: Human DNA sequence from clone 475B7 on |
| | chromosome Xq12.1-13. Contains the 3 part of the gene for a novel |
| | KIAA0615 and KIAA0323 LIKE protein, the gene for a novel protein, |
| | ESTs, STSs, GSSs and two putative CpG islands/cds = (48,2201)/ |
| | gb = AL050306/gi = 5419784/ug = Hs. 90625/len = 2395 |
| 41869_at | Cluster Incl. U78310: Homo sapiens pescadillo mRNA, complete cds/ |
| | cds = (58,1824)/gb = U78310/gi = 2194202/ug = Hs. 13501/len = 2235 |
| 1878_g_at | M13194/FEATURE = mRNA/DEFINITION = HUMERCC1 Human |
| | excision repair protein (ERCC1) mRNA, complete cds, clone pcDE |
| 41116_at | Cluster Incl. AI799802: wc43d09.x1 Homo sapiens cDNA, 3 end/ |
| | clone = IMAGE-2321393/clone_end = 3/gb = AI799802/gi = 5365274/ |
| | ug = Hs. 101516/len = 688 |
| 37390_at | Cluster Incl. D86977: Human mRNA for KIAA0224 gene, complete cds/ |
| | cds = (136,3819)/gb = D86977/gi = 1504027/ug = Hs. 78054/len = 4226 |
| 38841_at | Cluster Incl. AF068195: Homo sapiens putative glialblastoma cell |
| | differentiation-related protein (GBDR1) mRNA, complete cds/ |
| | cds = (58,1062)/gb = AF068195/gi = 3192872/ug = Hs. 9194/len = 1493 |
| 35787_at | Cluster Incl. AI986201: wr81a01.x1 Homo sapiens cDNA, 3 end/ |
| | clone = IMAGE-2494056/clone_end = 3/gb = AI986201/gi = 5813478/ |
| | ug = Hs. 66881/len = 814 |
| 1903_at | Ras-Related Protein Rap1b |
| 39661_s_at | Cluster Incl. AF034102: Homo sapiens NBMPR-insensitive nucleoside |
| | transporter ei (ENT2) mRNA, complete cds/cds = (237,1607)/ |
| | gb = AF034102/gi = 2811136/ug = Hs. 32951/len = 2522 |
| 40241_at | Cluster Incl. U09850: Human zinc finger protein (ZNF143) mRNA, |
| | complete cds/cds = (37,1917)/gb = U09850/gi = 495571/ug = Hs. 154095/ |
| | len = 3908 |
| 40975_s_at | Cluster Incl. AL050258: Novel human mRNA similar to mouse tuftelin- |
| | interacting protein 10 mRNA, AF097181/cds = (263,2776)/ |
| | gb = AL050258/gi = 4886426/ug = Hs. 20225/len = 3565 |
| 32149_at | Cluster Incl. AA532495: nj54a10.s1 Homo sapiens cDNA/ |
| | clone = IMAGE-996282/gb = AA532495/gi = 2276749/ug = Hs. 183752/ |
| | len = 549 |
| 34059_at | Cluster Incl. AA586695: nn42h06.s1 Homo sapiens cDNA, 3 end/ |
| | clone = IMAGE-1086587/clone_end = 3/gb = AA586695/gi = 2397509/ |
| | ug = Hs. 193956/len = 522 |
| 1832_at | M62397/FEATURE=/DEFINITION = HUMCRCMUT Human |
| | colorectal mutant cancer protein mRNA, complete cds |
| 1321_s_at | U43916/FEATURE=/DEFINITION = HSU43916 Human tumor- |
| | associated membrane protein homolog (TMP) mRNA, complete cds |
| 35489_at | Cluster Incl. M82962: Human N-benzoyl-L-tyrosyl-p-amino-benzoic acid |
| | hydrolase alpha subunit (PPH alpha) mRNA, complete cds/cds = (9,2249)/ |
| | gb = M82962/gi = 535474/ug = Hs. 179704/len = 2902 |
| 39912_at | Cluster Incl. AB006179: Homo sapiens mRNA for heparan-sulfate 6- |
| | sulfotransferase, complete cds/cds = (111,1343)/gb = AB006179/ |
| | gi = 3073774/ug = Hs. 132884/len = 2051 |
| 31368_at | Cluster Incl. W27967: 40b10 Homo sapiens cDNA/gb = W27967/ |
| | gi = 1307915/ug = Hs. 136154/len = 755 |
| 35640_at | Cluster Incl. D14822: Human chimeric mRNA derived from AML1 gene |
| | and MTG8(ETO) gene, partial sequence/cds = (0,597)/gb = D14822/ |
| | gi = 467498/ug = Hs. 31551/len = 799 |
| 38198_at | Cluster Incl. AL079275: Homo sapiens mRNA full length insert cDNA |
| | clone EUROIMAGE 566443/cds = UNKNOWN/gb = AL079275/ |
| | gi = 5102578/ug = Hs. 157078/len = 2082 |
| 38871_at | Cluster Incl. AJ006288: Homo sapiens mRNA for bcl-10 protein/ |
| | cds = (690,1391)/gb = AJ006288/gi = 4049459/ug = Hs. 193516/len = 1877 |
| 37054_at | Cluster Incl. J04739: Human bactericidal permeability increasing protein |
| | (BPI) mRNA, complete cds/cds = (30,1493)/gb = J04739/gi = 179528/ |
| | ug = Hs. 89535/len = 1813 |
| 34909_at | Cluster Incl. AC004990: Homo sapiens PAC clone DJ1185I07 from |
| | 7q11.23-q21/cds = (0,1766)/gb = AC004990/gi = 3924668/ug = Hs. 128653/ |
| | len = 1767 |

TABLE 53
22-gene-signature of invasive prostate cancer

| Affymetrix | |
|---|---|
| Probe Set ID | |
| (U95Av2) | Description |
| 40635_at | Cluster Incl. AF089750: Homo sapiens flotillin-1 mRNA, complete cds/ |
| | cds = (164,1447)/gb = AF089750/gi = 3599572/ug = Hs. 179986/len = 1796 |
| 38260_at | Cluster Incl. AL050306: Human DNA sequence from clone 475B7 on |
| | chromosome Xq12.1-13. Contains the 3 part of the gene for a novel |
| | KIAA0615 and KIAA0323 LIKE protein, the gene for a novel protein, |
| | ESTs, STSs, GSSs and two putative CpG islands/cds = (48,2201)/ |
| | gb = AL050306/gi = 5419784/ug = Hs. 90625/len = 2395 |
| 33833_at | Cluster Incl. J05243: Human nonerythroid alpha-spectrin (SPTAN1) |
| | mRNA, complete cds/cds = (102,7520)/gb = J05243/gi = 179105/ |
| | ug = Hs. 237180/len = 7787 |
| 38794_at | Cluster Incl. X53390: Human mRNA for upstream binding factor (hUBF)/ |
| | cds = (147,2441)/gb = X53390/gi = 509240/ug = Hs. 89781/len = 3097 |
| 33915_at | Cluster Incl. W22655: 71B9 Homo sapiens cDNA/clone = (not- |
| | directional)/gb = W22655/gi = 1299488/ug = Hs. 26070/len = 761 |
| 39379_at | Cluster Incl. AL049397: Homo sapiens mRNA; cDNA DKFZp586C1019 |
| | (from clone DKFZp586C1019)/cds = UNKNOWN/gb = AL049397/ |
| | gi = 4500188/ug = Hs. 12314/len = 1720 |
| 1903_at | Ras-Related Protein Rap1b |
| 39661_s_at | Cluster Incl. AF034102: Homo sapiens NBMPR-insensitive nucleoside |
| | transporter ei (ENT2) mRNA, complete cds/cds = (237,1607)/ |
| | gb = AF034102/gi = 2811136/ug = Hs. 32951/len = 2522 |
| 40241_at | Cluster Incl. U09850: Human zinc finger protein (ZNF143) mRNA, |
| | complete cds/cds = (37,1917)/gb = U09850/gi = 495571/ug = Hs. 154095/ |
| | len = 3908 |
| 40975_s_at | Cluster Incl. AL050258: Novel human mRNA similar to mouse tuftelin- |
| | interacting protein 10 mRNA, AF097181/cds = (263,2776)/ |
| | gb = AL050258/gi = 4886426/ug = Hs. 20225/len = 3565 |
| 32149_at | Cluster Incl. AA532495: nj54a10.s1 Homo sapiens cDNA/ |
| | clone = IMAGE-996282/gb = AA532495/gi = 2276749/ug = Hs. 183752/ |
| | len = 549 |
| 34059_at | Cluster Incl. AA586695: nn42h06.s1 Homo sapiens cDNA, 3 end/ |
| | clone = IMAGE-1086587/clone_end = 3/gb = AA586695/gi = 2397509/ |
| | ug = Hs. 193956/len = 522 |
| 1832_at | M62397/FEATURE=/DEFINITION = HUMCRCMUT Human |
| | colorectal mutant cancer protein mRNA, complete cds |
| 1321_s_at | U43916/FEATURE=/DEFINITION = HSU43916 Human tumor- |
| | associated membrane protein homolog (TMP) mRNA, complete cds |
| 35489_at | Cluster Incl. M82962: Human N-benzoyl-L-tyrosyl-p-amino-benzoic acid |
| | hydrolase alpha subunit (PPH alpha) mRNA, complete cds/cds = (9,2249)/ |
| | gb = M82962/gi = 535474/ug = Hs. 179704/len = 2902 |
| 39912_at | Cluster Incl. AB006179: Homo sapiens mRNA for heparan-sulfate 6- |
| | sulfotransferase, complete cds/cds = (111,1343)/gb = AB006179/ |
| | gi = 3073774/ug = Hs. 132884/len = 2051 |
| 31368_at | Cluster Incl. W27967: 40b10 Homo sapiens cDNA/gb = W27967/ |
| | gi = 1307915/ug = Hs. 136154/len = 755 |
| 35640_at | Cluster Incl. D14822: Human chimeric mRNA derived from AML1 gene |
| | and MTG8(ETO) gene, partial sequence/cds = (0,597)/gb = D14822/ |
| | gi = 467498/ug = Hs. 31551/len = 799 |
| 38198_at | Cluster Incl. AL079275: Homo sapiens mRNA full length insert cDNA |
| | clone EUROIMAGE 566443/cds = UNKNOWN/gb = AL079275/ |
| | gi = 5102578/ug = Hs. 157078/len = 2082 |
| 38871_at | Cluster Incl. AJ006288: Homo sapiens mRNA for bcl-10 protein/ |
| | cds = (690,1391)/gb = AJ006288/gi = 4049459/ug = Hs. 193516/len = 1877 |
| 37054_at | Cluster Incl. J04739: Human bactericidal permeability increasing protein |
| | (BPI) mRNA, complete cds/cds = (30,1493)/gb = J04739/gi = 179528/ |
| | ug = Hs. 89535/len = 1813 |
| 34909_at | Cluster Incl. AC004990: Homo sapiens PAC clone DJ1185I07 from |
| | 7q11.23-q21/cds = (0,1766)/gb = AC004990/gi = 3924668/ug = Hs. 128653/ |
| | len = 1767 |

### EXAMPLE 8

### Selection of the Gene Clusters Discriminating Between Metastatic and Non-Metastatic Human Breast Cancer

In this example we utilized gene expression data and associated clinical information published in the recent study on gene expression profiling of breast cancer (van't Veer, L. J., et al., “Gene expression profiling predicts clinical outcome of breast cancer,” Nature, 415: 530-536, 2002, incorporated herein by reference). This study identifies 70 genes whose expression pattern is strongly predictive of a short post-diagnosis and treatment interval to distant metastases (van't Veer, L. J., et al., 2002). The expression pattern of these 70 genes discriminate with 81% (optimized sensitivity threshold) or 83% (optimal accuracy threshold) accuracy the patient's prognosis in the group of 78 young women diagnosed with sporadic lymph-node-negative breast cancer (this group comprises of 34 patients who developed distant metastases within 5 years and 44 patients who continued to be disease-free after a period of at least 5 years; they constitute a poor prognosis and good prognosis group, correspondingly). The authors described in this paper the second independent groups of breast cancer patients comprising 11 patients who developed distant metastases within 5 years and 8 patients who continued to be disease-free after a period of at least 5 years. We applied the method of the present invention to further reduce the number of genes whose expression patterns represent genetic signatures of breast cancer with “poor prognosis” or “good prognosis.” In our example we utilized the data derived from a group of 19 patients as a training set of samples, and the data derived from a group of 78 patients as a test set of samples.

Using the methods of present invention, we calculated the phenotype association indices for 19 samples of the training set and determined that this gene cluster exhibited a 84% success rate in clinical sample classification based on individual phenotype association indices (Table 54). As shown in Table 54, 7/8 (or 88%) of the good prognosis breast cancer samples had negative phenotype association indices, whereas 9/11 (or 82%) of poor prognosis breast cancer samples displayed negative phenotype association indices. Overall, 16 of 19 samples (or 84%) were correctly classified.

TABLE 54
Classification accuracy of the breast cancer
prognosis predictor gene clusters

| Cluster | r value | Good prognosis | Poor prognosis | Overall |
|---|---|---|---|---|
| 70 genes | | 7/8 (88%) | 9/11 (82%) | 16/19 (84%) |
| 19 genes | 0.984 | 7/8 (88%) | 9/11 (82%) | 16/19 (84%) |
| 19 genes | 0.984 | 29/44 (66%) | 28/34 (82%) | 57/78 (73%) |
| 9 genes | 0.984 | 7/8 (88%) | 10/11 (91%) | 17/19 (89%) |
| 9 genes | 0.984 | 32/44 (73%) | 28/34 (82%) | 60/78 (77%) |
| 22 genes | 0.975 | 7/8 (88%) | 10/11 (91%) | 17/19 (89%) |
| 22 genes | 0.975 | 29/44 (66%) | 29/34 (85%) | 58/78 (74%) |
| 12 genes | 0.989 | 7/8 (88%) | 10/11 (91%) | 17/19 (89%) |
| 12 genes | 0.989 | 31/44 (70%) | 28/34 (82%) | 59/78 (76%) |

Next, we identified two best-fit poor prognosis breast cancer samples displaying the correlation coefficient of 0.751 and 0.832 to the average expression profile of the 11 poor prognosis breast cancer samples. The average expression profile of the 11 poor prognosis breast cancer samples was utilized as a first reference set. The average expression profile of these two best-fit poor prognosis breast cancer samples was utilized as a second reference set.

The concordance set was obtained by selecting only those genes having a consistent direction of the differential expression in both the first and the second reference sets (i.e., greater gene expression difference in the poor prognosis cf. the good prognosis samples and greater gene expression in the best-fit tumor sample cf. the average expression value across the entire data set or vice-versa). The concordance set comprised of 44 genes (r=0.950). A minimum segregation set was selected following the procedures described above. Scatter plots were generated of the log₁₀ transformed average −fold expression change in the first reference set and average −fold expression change in the second reference set (in case of a single best-fit tumor it was the log₁₀ transformed ratio of the expression value for a gene to the average expression value across the entire data set). For the samples of the first reference set, <expression>₁ corresponds to the average expression value for gene x over all samples from patients who had invasive tumors and <expression>₂ corresponds to the average expression value for gene x over all samples from patients who had non-invasive tumors. A minimum segregation set was identified by selecting a subset of the highly correlated genes between two reference sets from the concordance set. Using this approach we identified two gene clusters (19-gene cluster and 9-gene cluster) discriminating with high accuracy between poor prognosis and good prognosis human breast tumors in both training and test sets of clinical samples. These two breast cancer metastasis predictors or poor prognosis minimum segregation sets are listed in Tables 55 & 56. The classification performance for each of these gene clusters is presented in the Table 54.

TABLE 55
19-gene signature of breast cancer prognosis predictor (r = 0.984)

| Gene ID (Chip identified in van't Veer, | |
|---|---|
| L. J., et al., 2002) | Sequence Name |
| Contig55725_RC | EST |
| NM_005915 | MCM6 |
| Contig46218_RC | EST |
| NM_001809 | CENPA |
| NM_016359 | LOC51203 |
| NM_002073 | GNAZ |
| NM_014321 | ORC6L |
| NM_016448 | L2DTL |
| NM_002916 | RFC4 |
| NM_003875 | GMPS |
| NM_014791 | KIAA0175 |
| Contig28552_RC | EST |
| NM_003981 | PRC1 |
| AL137718 | DIAPH3 |
| NM_000849 | GSTM3 |
| NM_003862 | FGF18 |
| NM_004994 | MMP9 |
| NM_003239 | TGFB3 |
| NM_020974 | CEGP1 |

TABLE 56
9-gene signature of breast cancer prognosis predictor (r = 0.984)

| Gene ID (Chip identified in van't Veer, | |
|---|---|
| L. J., et al., 2002) | Sequence Name |
| Contig55725_RC | EST |
| NM_005915 | MCM6 |
| Contig46218_RC | EST |
| NM_003875 | GMPS |
| NM_000849 | GSTM3 |
| NM_003862 | FGF18 |
| NM_004994 | MMP9 |
| NM_003239 | TGFB3 |
| NM_020974 | CEGP1 |

In the next example, the average expression profile of all 19 breast cancer samples obtained from 11 patients with poor prognosis and 8 patients with good prognosis was utilized as a first reference set. Next, we calculated the individual phenotype association indices and identified a single best-fit poor prognosis breast cancer sample displaying the correlation coefficient of 0.677 to the average expression profile of the 19 breast cancer samples. The average expression profile of this single best-fit poor prognosis breast cancer sample was utilized as a second reference set.

The concordance set was obtained by selecting only those genes having a consistent direction of the differential expression in both the first and the second reference sets (i.e., greater gene expression difference in the poor prognosis cf. the good prognosis samples and greater gene expression in the best-fit tumor sample cf. the average expression value across the entire data set or vice-versa). The concordance set comprised of 47 genes (r=0.822). A minimum segregation set was selected following the procedures described in the introduction to the Detailed Description of the Preferred Embodiments and the Materials & Methods sections. Scatter plots were generated of the log₁₀ transformed average −fold expression change in the first reference set and average −fold expression change in the second reference set (in case of a single best-fit tumor it was the log₁₀ transformed ratio of the expression value for a gene to the average expression value across the entire data set). For the samples of the first reference set, <expression>₁ corresponds to the average expression value for gene x over all samples from patients who had invasive tumors and <expression>₂ corresponds to the average expression value for gene x over all samples from patients who had non-invasive tumors. A minimum segregation set was identified by selecting a subset of the highly correlated genes between two reference sets from the concordance set. Using this approach we identified two gene clusters (22-gene cluster and 12-gene cluster) discriminating with high accuracy between poor prognosis and good prognosis human breast tumors in both training and test sets of clinical samples. These two breast cancer metastasis predictors or poor prognosis minimum segregation sets are listed in Tables 57 & 58. The classification performance for each of these gene clusters is presented in the Table 54.

TABLE 57
22-gene signature of breast cancer prognosis predictor (r = 0.975)

| Gene ID (Chip identified in van't Veer, | |
|---|---|
| L. J., et al., 2002) | Sequence Name |
| NM_005915 | MCM6 |
| Contig46218_RC | EST |
| AA555029_RC | EST |
| NM_016359 | LOC51203 |
| Contig56457_RC | TMEFF1 |
| NM_007036 | ESM1 |
| NM_007203 | AKAP2 |
| AF073519 | SERF1A |
| NM_015984 | UCH37 |
| NM_014321 | ORC6L |
| U82987 | BBC3 |
| Contig2399_RC | SM-20 |
| NM_003882 | WISP1 |
| AB037863 | KIAA1442 |
| Contig63649_RC | EST |
| Contig20217_RC | EST |
| AF055033 | IGFBP5 |
| NM_003862 | FGF18 |
| NM_003239 | TGFB3 |
| NM_000849 | GSTM3 |
| NM_000599 | IGFBP5 |
| NM_020974 | CEGP1 |

TABLE 58
12-gene signature of breast cancer prognosis predictor (r = 0.989)

| Gene ID (Chip identified in van't Veer, | |
|---|---|
| L. J., et al., 2002) | Sequence Name |
| NM_005915 | MCM6 |
| NM_007036 | ESM1 |
| NM_007203 | AKAP2 |
| AF073519 | SERF1A |
| NM_015984 | UCH37 |
| NM_014321 | ORC6L |
| AF055033 | IGFBP5 |
| NM_003862 | FGF18 |
| NM_003239 | TGFB3 |
| NM_000849 | GSTM3 |
| NM_000599 | IGFBP5 |
| NM_020974 | CEGP1 |

### EXAMPLE 9

### Selection of the Gene Clusters Predicting Good and Poor Prognosis of Human Lung Carcinoma.

We applied the methods of the present invention to identify gene expression profiles distinguishing lung adenocarcinoma samples from normal lung specimens as well as highly malignant phenotype of lung adenocarcinoma, associated with short survival after diagnosis and therapy, from less aggressive lung cancers, associated with longer patient's survival. Clinical data set utilized in this example was published (Bhattacharjee, A., Richards, W. G., Staunton, J., Li, C., Monti, S., Vasa, P., Ladd, C., Beheshti, J., Bueno, R., Gillette, M., Loda, M., Weber, G., Mark, E. J., Lander, E. S., Wong, W., Johnson, B. E., Golub, T. R., Sugarbaker, D. J., Meyerson, M. Classification of human lung carcinomas by mRNA expression profiling reveals distinct adenocarcinoma subclasses. PNAS, 98: 13790-13795, 2001; incorporated herein by reference).

Using the clinical data set and associated clinical history (Bhattacharje et al., 2001), we selected two groups of adenocarcinoma patients having markedly distinct survival after diagnosis and therapy: poor prognosis group 1 comprising 34 patients with the median survival of 8.5 month (range 0.1-17.3 month) and good prognosis group 2 comprising 16 patients with the median survival of 84 month (range 75.4-106.1 month). As a starting point, we utilized a set of the 675 transcripts selected based on a statistical analysis of the quality of the dataset and variability of gene expression across the dataset (Bhattacharje et al., 2001). Applying methods of the present invention, we identified a set of 38 genes displaying at least a 2-fold difference in the average values of the mRNA expression levels between 34 poor prognosis samples versus 16 good prognosis samples (Table 59).

TABLE 59
38 genes differentially regulated in human lung adenocarcinomas
exhibiting poor and good clinical outcomes after the therapy.

| Affymetrix Probe | |
|---|---|
| Set ID (U95Av2) | Description |
| 1665_s_at | Endothelial Cell Growth Factor 1 |
| 38428_at | matrix metalloproteinase 1 (interstitial collagenase) |
| 40544_g_at | achaete-scute complex (Drosophila) homolog-like 1 |
| 34898_at | amphiregulin (schwannoma-derived growth factor) |
| 1482_g_at | matrix metalloproteinase 12 (macrophage elastase) |
| 35175_f_at | eukaryotic translation elongation factor 1 alpha 2 |
| 1481_at | matrix metalloproteinase 12 (macrophage elastase) |
| 38389_at | 2′,5′-oligoadenylate synthetase 1 (40-46 kD) |
| 40543_at | achaete-scute complex (Drosophila) homolog-like 1 |
| 408_at | GRO1 oncogene (melanoma growth stimulating activity, alpha) |
| 40004_at | sine oculis homeobox (Drosophila) homolog 1 |
| 35938_at | phospholipase A2, group IVA (cytosolic, calcium-dependent) |
| 37874_at | flavin containing monooxygenase 5 |
| 33754_at | thyroid transcription factor 1 |
| 38790_at | epoxide hydrolase 1, microsomal (xenobiotic) |
| 32275_at | secretory leukocyte protease inhibitor (antileukoproteinase) |
| 32081_at | citron (rho-interacting, serine/threonine kinase 21) |
| 32154_at | transcription factor AP-2 alpha (activating enhancer-binding protein |
| | 2 alpha) |
| 206_at | cathepsin E |
| 36623_at | Cluster Incl AB011406: Homo sapiens mRNA for alkalin |
| | phosphatase, complete cds/cds = (176,1750)/gb = AB011406/ |
| | gi = 3401944/ug = Hs. 75431 /len = 2510 |
| 37576_at | Purkinje cell protein 4 |
| 37811_at | calcium channel, voltage-dependent, alpha 2/delta subunit 2 |
| 39681_at | zinc finger protein 145 (Kruppel-like, expressed in promyelocytic |
| | leukemia) |
| 1270_at | RAP1, GTPase activating protein 1 |
| 32570_at | hydroxyprostaglandin dehydrogenase 15-(NAD) |
| 37600_at | extracellular matrix protein 1 |
| 31844_at | homogentisate 1,2-dioxygenase (homogentisate oxidase) |
| 35834_at | alpha-2-glycoprotein 1, zinc |
| 36681_at | apolipoprotein D |
| 37430_at | arachidonate 15-lipoxygenase, second type |
| 36680_at | amylase, alpha 2B; pancreatic |
| 40031_at | aldehyde dehydrogenase 3 |
| 38773_at | carbonyl reductase 1 |
| 765_s_at | lectin, galactoside-binding, soluble, 4 (galectin 4) |
| 37209_g_at | phosphoserine phosphatase-like |
| 36736_f_at | phosphoserine phosphatase |
| 41069_at | chondromodulin I precursor |
| 37208_at | phosphoserine phosphatase-like |

Next, we calculated the phenotype association indices for all 50 samples and determined that this gene cluster exhibited a 72% success rate in clinical sample classification based on individual phenotype association indices (Table 60). As shown in Table 60, 12/16 (or 75%) of the lung adenocarcinoma samples of the good prognosis group had negative phenotype association indices, whereas 24/34 (or 71%) of lung adenocarcinoma specimens of the poor prognosis group displayed positive phenotype association indices. Overall, 36 of 50 samples (or 72%) were correctly classified.

TABLE 60
Classification accuracy of lung adenocarcinoma
prognosis predictor clusters

| Cluster | r value | Poor prognosis | Good Prognosis | Overall |
|---|---|---|---|---|
| 38 genes | 0.771 | 24/34 (71%) | 12/16 (75%) | 36/50 (72%) |
| 26 genes | 0.938 | 13/34 (38%) | 15/16 (94%) | 28/50 (56%) |
| 15 genes | 0.942 | 28/34 (82%) | 11/16 (69%) | 39/50 (78%) |

Next, we identified 8 best-fit poor prognosis samples displaying the correlation coefficient of 0.3 or higher to the average expression profile of the 34 poor prognosis samples. We calculated the average expression profile for these 8 best-fit poor prognosis samples by dividing the average expression value for each gene in the 8 samples of the best-fir set by the average expression value across the entire data set.

Next, we selected from an initial set of 38 genes a set of 26 genes (lung adenocarcinoma poor prognosis predictor cluster 1—see Table 61) displaying high positive correlation (r=0.938) between the best-fit tumors and poor prognosis samples data sets. This gene cluster exhibited a 56% success rate in clinical sample classification based on individual phenotype association indices (Table 60). As shown in Table 60, 15/16 (or 94%) of the lung adenocarcinoma samples of the good prognosis group had negative phenotype association indices, whereas 13/34 of lung adenocarcinoma specimens of the poor prognosis group displayed positive phenotype association indices. Overall, 28 of 50 samples (or 56%) were correctly classified.

TABLE 61
26 genes of the lung adenocarcinoma poor
prognosis predictor cluster 1.

| Affymetrix Probe | |
|---|---|
| Set ID (U95Av2) | Description |
| 1665_s_at | Endothelial Cell Growth Factor 1 |
| 38428_at | matrix metalloproteinase 1 (interstitial collagenase) |
| 40544_g_at | achaete-scute complex (Drosophila) homolog-like 1 |
| 1482_g_at | matrix metalloproteinase 12 (macrophage elastase) |
| 1481_at | matrix metalloproteinase 12 (macrophage elastase) |
| 38389_at | 2′,5′-oligoadenylate synthetase 1 (40-46 kD) |
| 40543_at | achaete-scute complex (Drosophila) homolog-like 1 |
| 408_at | GRO1 oncogene (melanoma growth |
| | stimulating activity, alpha) |
| 35938_at | phospholipase A2, group IVA |
| | (cytosolic, calcium-dependent) |
| 37874_at | flavin containing monooxygenase 5 |
| 33754_at | thyroid transcription factor 1 |
| 38790_at | epoxide hydrolase 1, microsomal (xenobiotic) |
| 32275_at | secretory leukocyte protease inhibitor |
| | (antileukoproteinase) |
| 32081_at | citron (rho-interacting, serine/threonine kinase 21) |
| 206_at | cathepsin E |
| 36623_at | Cluster Incl AB011406: Homo sapiens |
| | mRNA for alkalin |
| | phosphatase, complete cds/cds = (176,1750)/gb = |
| | AB011406/gi = 3401944/ug = Hs. 75431/len = 2510 |
| 37576_at | Purkinje cell protein 4 |
| 37811_at | calcium channel, voltage-dependent, |
| | alpha 2/delta subunit 2 |
| 32570_at | hydroxyprostaglandin dehydrogenase 15-(NAD) |
| 37600_at | extracellular matrix protein 1 |
| 31844_at | homogentisate 1,2-dioxygenase |
| | (homogentisate oxidase) |
| 36681_at | apolipoprotein D |
| 36680_at | amylase, alpha 2B; pancreatic |
| 38773_at | carbonyl reductase 1 |
| 37209_g_at | phosphoserine phosphatase-like |
| 36736_f_at | phosphoserine phosphatase |

To improve the classification accuracy, we selected from an initial set of 38 genes a set of 15 genes (lung adenocarcinoma poor prognosis predictor cluster 2—see Table 62) displaying high positive correlation (r=0.942) between the best-fit tumors and poor prognosis samples data sets.

TABLE 62
15 genes of the lung adenocarcinoma
poor prognosis predictor cluster 2.

| Affymetrix | |
|---|---|
| Probe Set ID | |
| (U95Av2) | Description |
| 1665_s_at | Endothelial Cell Growth Factor 1 |
| 38428_at | matrix metalloproteinase 1 (interstitial collagenase) |
| 40544_g_at | achaete-scute complex (Drosophila) homolog-like 1 |
| 1482_g_at | matrix metalloproteinase 12 (macrophage elastase) |
| 1481_at | matrix metalloproteinase 12 (macrophage elastase) |
| 38389_at | 2′,5′-oligoadenylate synthetase 1 (40-46 kD) |
| 40543_at | achaete-scute complex (Drosophila) homolog-like 1 |
| 408_at | GRO1 oncogene (melanoma growth stimulating activity, |
| | alpha) |
| 35938_at | phospholipase A2, group IVA (cytosolic, calcium- |
| | dependent) |
| 39681_at | zinc finger protein 145 (Kruppel-like, expressed in |
| | promyelocytic leukemia) |
| 35834_at | alpha-2-glycoprotein 1, zinc |
| 40031_at | aldehyde dehydrogenase 3 |
| 765_s_at | lectin, galactoside-binding, soluble, 4 (galectin 4) |
| 41069_at | chondromodulin I precursor |
| 37208_at | phosphoserine phosphatase-like |

This gene cluster exhibited a 78% success rate in clinical sample classification based on individual phenotype association indices (Table 60). As shown in Table 60, 11/16 (or 69%) of the lung adenocarcinoma samples of the good prognosis group had negative phenotype association indices, whereas 28/34 (or 82%) of lung adenocarcinoma specimens of the poor prognosis group displayed positive phenotype association indices. Overall, 39 of 50 samples (or 78%) were correctly classified.

### EXAMPLE 10

### Selection of the Gene Clusters Associated with Metastatic Cancer

The methods of the present invention were used along with the data reported by Ramaswamy et al. (2003) to identify gene clusters distinguishing between the human primary adenocarcinomas of diverse origin and metastatic adenocarcinoma lesions. These data were the supplemental data reported in Ramaswamy, S., Ross, K. N., Lander, E. S., Golub, T. R. “A molecular signature of metastasis in primary solid tumors,” Nature Genetics, January 2003, 33: 49-54, incorporated herein by reference. Ramaswamy et al. (2003) identified the 17-gene cluster expression profile of which distinguishes 12 metastatic adenocarcinoma nodules of diverse origin and 64 human primary adenocarcinomas of diverse origin (lung, breast, prostate, colorectal, uterus, ovary). Both metastatic lesions and primary adenocarcinomas were representing the same diverse spectrum of tumor types obtained from different individuals (Ramaswamy et al., 2003).

The expression profile of the 17-gene cluster in metastatic versus primary tumors was utilized as a first reference set.

Next, we calculated the phenotype association indices for all 76 samples and determined that this gene cluster exhibited a 45% success rate in clinical sample classification based on individual phenotype association indices (Table 63). As shown in Table 63, 12/12 (or 100%) of the metastatic samples had positive phenotype association indices, whereas 22/64 (or 34%) of primary tumor samples displayed negative phenotype association indices. Overall, 34 of 76 samples (or 45%) were correctly classified.

TABLE 63
Classification accuracy of the metastases segregation gene clusters
(r = 0.000 discrimination threshold)

| | r | Primary tumors | | | | | | Primary | | |
|---|---|---|---|---|---|---|---|---|---|---|
| Cluster | value | Breast | Colon | Lung | Prostate | Uterus | Ovary | tumors | Metastases | Overall |
| 17 genes | 0.964 | 2 of | 4 of | 3 of | 8 of 10 | 5 of 10 | 0 of | 22/64 | 12/12 | 34/76 |
| | | 11 | 11 | 11 | | | 11 | (34%) | (100%) | (45%) |
| 12 genes | 0.991 | 3 of | 5 of | 0 of | 8 of 10 | 6 of 10 | 0 of | 22/64 | 12/12 | 34/76 |
| | | 11 | 11 | 11 | | | 11 | (34%) | (100%) | (45%) |
| 11 genes | 0.992 | 8 of | 6 of | 6 of | 4 of 10 | 6 of 10 | 2 of | 32/64 | 12/12 | 44/76 |
| | | 11 | 11 | 11 | | | 11 | (50%) | (100%) | (58%) |
| 8 genes | 0.989 | 3 of | 7 of | 1 of | 8 of 10 | 6 of 10 | 1 of | 26/64 | 12/12 | 38/76 |
| | | 11 | 11 | 11 | | | 11 | (41%) | (100%) | (50%) |
| 7 genes | 0.993 | 7 of | 6 of | 7 of | 6 of 10 | 7 of 10 | 2 of | 35/64 | 12/12 | 47/76 |
| | | 11 | 11 | 11 | | | 11 | (55%) | (100%) | (62%) |

The classification accuracy of the 17-gene cluster was much improved when the discrimination threshold was set at the level of 0.400 of a correlation coefficient. As shown in Table 64, 12/12 (or 100%) of the metastatic samples had phenotype association indices higher than 0.400, whereas 48/64 (or 75%) of primary tumor samples displayed phenotype association indices lower than 0.400. Overall, 60 of 76 samples (or 79%) were correctly classified.

TABLE 64
Classification accuracy of the metastases segregation gene clusters
(r = 0.400 discrimination threshold)

| | r | Primary tumors | | | | | | Primary | | |
|---|---|---|---|---|---|---|---|---|---|---|
| Cluster | value | Breast | Colon | Lung | Prostate | Uterus | Ovary | tumors | Metastases | Overall |
| 17 genes | 0.964 | 9 of | 7 of | 8 of | 8 of 10 | 8 of 10 | 8 of | 48/64 | 12/12 | 60/76 |
| | | 11 | 11 | 11 | | | 11 | (75%) | (100%) | (79%) |
| 12 genes | 0.991 | 10 of | 7 of | 7 of | 8 of 10 | 8 of 10 | 3 of | 43/64 | 12/12 | 55/76 |
| | | 11 | 11 | 11 | | | 11 | (67%) | (100%) | (72%) |
| 11 genes | 0.992 | 11 of | 7 of | 8 of | 8 of 10 | 8 of 10 | 8 of | 50/64 | 12/12 | 62/76 |
| | | 11 | 11 | 11 | | | 11 | (78%) | (100%) | (82%) |
| 8 genes | 0.989 | 8 of | 7 of | 7 of | 8 of 10 | 7 of 10 | 5 of | 42/64 | 12/12 | 54/76 |
| | | 11 | 11 | 11 | | | 11 | (66%) | (100%) | (71%) |
| 7 genes | 0.993 | 11 of | 7 of | 8 of | 8 of 10 | 7 of 10 | 7 of | 49/64 | 12/12 | 61/76 |
| | | 11 | 11 | 11 | | | 11 | (77%) | (100%) | (80%) |

Next, we identified three best-fit metastatic samples displaying the correlation coefficient of 0.870, 0.923, and 0.874 to the average expression profile of the 12 metastatic samples. The average expression profile of these three best-fit metastatic samples was utilized as a second reference set.

The expression profile of the best-fit samples was utilized to refine the gene-expression signature associated with a metastatic phenotype to a small set of transcripts that would exhibit high discrimination accuracy between metastatic lesions and primary tumors. Thus, selecting a subset of the highly correlated genes between two reference sets identified a minimum segregation set suitable for clinical samples classification. Using this approach we identified four gene clusters discriminating with high accuracy between metastatic lesions and primary tumors. The members of these metastases minimum segregation sets (metastases minimum segregation gene clusters) are listed in Tables 65-68. The classification performance for each of these gene clusters is presented in the Tables 63 and 64.

TABLE 65
12-gene signature of metastases
Affymetrix Probe ID (U95Av2)

J03464_s_at
L37747_s_at
RC_AA430032_at
X85372_at
RC_AA608850_at
HG110-HT110_s_at
Z74615_at
U23946_at
D43968_at
U48959_at
D17408_s_at
D00654_at

TABLE 66
11-gene signature of metastases
Affymetrix Probe ID (U95Av2)

J03464_s_at
L37747_s_at
RC_AA430032_at
X85372_at
RC_AA608850_at
HG110-HT110_s_at
Z74615_at
U23946_at
D43968_at
M83664_at
AF001548_rna1_at

TABLE 67
8-gene signature of metastases
Affymetrix Probe ID (U95Av2)

J03464_s_at
L37747_s_at
RC_AA430032_at
U23946_at
D43968_at
U48959_at
D17408_s_at
D00654_at

TABLE 68
7-gene signature of metastases
Gene ID (Chip identified in van't Veer

J03464_s_at
L37747_s_at
RC_AA430032_at
U23946_at
D43968_at
M83664_at
AF001548_rna1_at

References

1. Fidler, I. J. The nude mouse model for studies of human cancer metastasis. In: V. Schirrmacher and R. Schwartz-Abliez (eds.). pp. 11-17. Berlin: Springer-Verlag, 1989.

2. Fidler, I. J. Critical factors in the biology of human cancer metastasis. Cancer Res., 50, 6130-6138, 1990.

3. Fidler, I. J., Naito, S., Pathak, S. Orhtotopic implantation is essential for the selection, growth and metastasis of human renal cell cancer in nude mice. Cancer Metastasis Rev., 9, 149-165, 1990.

4. Giavazzi, R., Campbell, D. E., Jessup, J. M., Cleary, K., and Fidler, I. J. Metastatic behavior of tumor cells isolated from primary and metastatic human colorectal carcinomas implanted into different sites in nude mice. Cancer Res., 46: 1928-1948, 1986.

5. Naito, S., von Eschenbach, A. C., Giavazzi, R., and Fidler, I. J. Growth and metastasis of tumor cells isolated from a renal cell carcinoma implanted into different organs of nude mice. Cancer Res., 46: 4109-4115, 1986.

6. McLemore, T. L., et al. Novel intrapulmonary model for orthotopic propagation of human lung cancer in athymic nude mice. Cancer Res., 47: 5132-5140, 1987.

7. Fu, X., Herrera, H., and Hoffman, R. M. Orthotopic growth and metastasis of human prostate carcinoma in nude mice after transplantation of histologically intact tissue. Int. J. Cancer, 52: 987-990, 1992.

8. Stephenson, R. A., Dinney, C.P.N., Gohji, K., Ordonez, N. G., Killion, J. J., and Fidler, I. J. Metastatic model for human prostate cancer using orthotopic implantation in nude mice. J. Natl. Cancer Inst., 84: 951-957, 1992.

9. Pettaway, C. A., Stephenson, R. A., and Fidler, I. J. Development of orthotopic models of metastatic human prostate cancer. Cancer Bull. (Houst.), 45: 424-429, 1993.

10. An, Z., Wang, X., Geller, J., Moossa, A. R., and Hoffman, R. M. Surgical orthotopic implantation allows high lung and lymph node metastasis expression of human prostate carcinoma cell line PC-3 in nude mice. The Prostate, 34: 169-174, 1998.

11. Wang, X., An, Z., Geller, J., and Hoffman, R. M. High-malignancy orthotopic mouse model of human prostate cancer LNCaP. The Prostate, 39: 182-186, 1999.

12. Yang, M., Jiang, P., Sun, F.-X., Hasegawa, S., Baranov, E., Chishima, T., Shimada, H., Moosa, A. R., and Hofman, R. M. A fluorescent orthotopic bone metastasis model of human prostate cancer. Cancer Res., 59: 781-786, 1999.

13. Morikawa, K., Walker, S. M., Jessup, J. M., Cleary, K., and Fidler, I. J. In vivo selection of highly metastatic cells from surgical specimens of different primary human colon carcinoma implanted in nude mice. Cancer Res., 48: 1943-1948, 1988.

14. Dinney, C. P. N. et al. Isolation and characterization of metastatic variants from human transitional cell carcinoma passaged by orthotopic implantation in athymic nude mice. J. Urol., 154: 1532-1538, 1995.

15. Pettaway, C. A., Pathak, S., Greene, G., Ramirez, E., Wilson, M. R., Killion, J. J., and Fidler, I. J. Selection of highly metastatic variants of different human prostatic carcinomas using orthotopic implantation in nude mice. Clinical Cancer Res., 2: 1627-1636, 1996.

16. Greene, G. F., Kitadai, Y., Pettaway, C. A., von Eschenbach, A. C., Bucana, C. D., Fidler, I. J. Correlation of metastasis-related gene expression with metastatic potential in human prostate carcinoma cells implanted in nude mice using an in situ messenger RNA hybridization technique. American J. Pathology, 150: 1571-1582, 1997.

17. Glinsky, G. V. and Glinsky, V. V. Apoptosis and metastasis: a superior resistance of metastatic cancer cells to programmed cell death. Cancer Lett. 101:43-51, 1996.

18. Glinsky, G. V., Price, J. E., Glinsky, V. V., Mossine, V. V., Kiriakova, G. and Metcalf, J. B. Inhibition of human breast cancer metastasis in nude mice by synthetic glycoamines. Cancer Res. 56:5319-24, 1996.

19. Glinsky, G. V., Glinsky, V. V., Ivanova, A. B. and Hueser, C. J. Apoptosis and metastasis: increased apoptosis resistance of metastatic cancer cells is associated with the profound deficiency of apoptosis execution mechanisms. Cancer Lett. 115:185-93, 1997.

20. Lockhart, D. J., Dong, H., Byrne, M. C., Follettie, M. T., Gallo, M. V., Chee, M. S., Mittmann, M., Wang, C., Kobayashi, M., Horton, H. and Brown, E. L. Expression monitoring by hybridization to high-density oligonucleotide arrays [see comments]. Nat. Biotechnol., 14:1675-80, 1996.

21. Singh, D., Febbo, P. G., Ross, K., Jackson, D. G., Manola, C. L., Tamayo, P., Renshaw, A. A., D'Amico, A. V., Richie, J. P., Lander, E. S., Loda, M., Kantoff, P. W., Golub, T. R., Sellers, W. R. Gene expression correlates of clinical prostate cancer behavior. Cancer Cell, 1: 203-209, 2002.

22. Gorgani, N. N., Smith, B. A., Kono, D. H., Theofilopoulos, A. N. Histidine-rich glycoprotein binds DNA and Fc□RI and potentiates the ingestion of apoptotic cells by macrophages. J. Immunol., 169: 4745-4751, 2002.

23. Machtens, S., Serth, J., Bokemeyer, C., Bathke, W., Minssen, A., Kollmannsberger, C., Hartmann, J., Knuchel, R., Kondo, M., Jonas, U., Kuczyk, M. Expression of the p53 and Maspin protein in primary prostate cancer: correlation with clinical features. Int J Cancer, 95: 337-342, 2001.

24. Zou, Z., Zhang, W., Young, D., Gleave, M. G., Rennie, P., Connell, T., Connelly, R., Moul, J., Srivastava, S., Sesterhenn, I. Maspin expression profile in human prostate cancer (CaP) and in vitro induction of Maspin expression by androgen ablation. Clin Cancer Res, 8: 1172-1177, 2002.

25. Bussemakers, M J, Van Bokhoven, A, Tomita, K, Jansen, C F, Schalken, J A. Complex cadherin expression in human prostate cancer cells. Int. J. Cancer, 85:446-450, 2000.

26. Tomita, K, Van Bokhoven, A, Van Leenders, G J, Ruijter, E T, Jansen, C F, Bussemakers, M J, Schalken, J A. Cadherin switching in human prostate cancer progression. Cancer Res., 60: 3650-3654, 2000.

27. Mills L, Tellez C, Huang S, Baker C, McCarty M, Green L, Gudas J M, Feng X, Bar-Eli M. Fully human antibodies to MCAM/MUC18 inhibit tumor growth and metastasis of human melanoma. Cancer Res., 62:5106-5114, 2002.

28. Johnson J P, Bar-Eli M, Jansen B, Markhof E. Melanoma progression-associated glycoprotein MUC18/MCAM mediates homotypic cell adhesion through interaction with a heterophilic ligand. Int J Cancer, 73:769-774, 1997.

29. Van Kempen L C, van den Oord J J, Van Muijen G N, Weidle U H, Bloemers H P, Swart G W. Activated leukocyte cell adhesion molecule/CD 166, a marker of tumor progression in primary malignant melanoma of the skin. Am J Pathol., 156:769-774, 2000.

30. Degen W G, Van Kempen L C, Gijzen E G, Van Groningen J J, Can Kooyk Y, Bloemers H O, Swart G W. MEMD, a new cell adhesion molecule in metastasizing human melanoma cell lines, is identical to ALCAM (activated leukocyte cell adhesion molecule). Am J Pathol., 152:805-813, 1998.

31. Swart G W. Activated leukocyte cell adhesion molecule (CD166/ALCAM): developmental and mechanistic aspects of cell clustering and cell migration. Eur J Cell Biol., 81:313-321, 2002.

32. Ohneda O, Ohneda K, Arai F, Lee J, Miyamoto T, Fukushima Y, Dowbenko D, Lasky L A, Suda T. ALCAM (CD166): its role in hematopoietic and endothelial development. Blood, 98:2134-2142, 2001.

33. Bowen M A, Patel D D, Li X, Modrell B, Malacko A R, Wang W C, Marquardt H, Neubauer M, Pesando J M, Francke U, et al. Cloning, mapping, and characterization of activated leukocyte-cell adhesion molecule (ALCAM), a CD6 ligand. J Exp Med., 181:2213-2220, 1995.

34. Bardin N, Anfossa F, Masse J M, Cramer E, Sabatier F, Le Bivic A, Sampol J, Dignat-George F. Identification of CD146 as a component of the endothelial junction involved in the control of cell-cell cohesion. Blood, 98:3677-3736, 2001.

35. Pickl W F, Majdic O, Fischer G F, Petzelbauer P, Fae I, Waclavicek M, Stockl J, Scheinecker C, Vidicki T, Aschauer H, Johnson J P, Knapp W. MUC18/MCAM (CD 146), an activation antigen of human T lymphocytes. J Immunol., 158:2107-2115, 1997.

36. Arai F, Ohneda O, Miyamoto T, Zhang X Q, Suda T. Mesenchymal stem cells in perichondrium express activated leukocyte cell adhesion molecule and participate in bone marrow formation. J Exp Med., 195:1549-1563, 2002.

37. Seshi B, Kumar S, Sellers D. Human bone marrow stromal cell: coexpression of markers specific for multiple mesenchymal cell lineages. Blood Cells Mol Dis., 26:234-246, 2000.

38. Guo Z, Yang J, Liu X, Li X, Hou C, Tang P H, Mao N. Biological features of mesenchymal stem cells from human bone marrow. Chin Med J (Engl), 114:950-953, 2001.

39. Bruder S P, Ricalton N S, Boynton R E, Connolly T J, Jaiswal N, Zaia J, Barry F P. Mesenchymal stem cell surface antigen SB-10 corresponds to activated leukocyte cell adhesion molecule and is involved in osteogenic differentiation. J Bone Miner Res., 13:655-663, 1998.

40. Léon C. L. T. van Kempen, Judith M. D. T. Nelissen, Winfried G. J. Degen, Ruurd Torensma, Ulrich H. Weidle, Henri P. J. Bloemers, Carl G. Figdor, and Guido W. M. Molecular Basis for the Homophilic Activated Leukocyte Cell Adhesion Molecule (ALCAM)-ALCAM Interaction. J. Biol. Chem., 276: 25783-25790, 2001.

41. Wu G J, Wu M W, Wang S W, Liu Z, Qu P, Peng Q, Yang H, Varma V A, Sun Q C, Petros J A, Lim S D, Amin M B. Isolation and characterization of the major-form of human MUC18 cDNA gene and correlation of MUC18 over-expression in prostate cancer cell lines and tissues with malignant progression. Gene, 279:17-31, 2001.

42. Wu G J, Varma V A, Wu M W, Wang S W, Qu P, Yang H, Petros J A, Lim S D, Amin M B. Expression of a human cell adhesion molecule, MUC18, in prostate cancer cell lines and tissues. Prostate, 48:305-315, 2001.

43. Ramaswamy, S., Ross, K. N., Lander, E. S., Golub, T. R. A molecular signature of metastasis in primary solid tumors. Nature Genetics, 33: 49-54, 2003.

44. LaTulippe, E., Satagopan, J., Smith, A., Scher, H., Scardino, P., Reuter, V., Gerald, W. L. Comprehensive gene expression analysis of prostate cancer reveals distinct transcriptional programs associated with metastatic disease. Cancer Res., 62: 4499-4506, 2002.

### EXAMPLE 11

### Use of Expression Data with Other Metrics to Predict Prostate Cancer Patient Survival

Introduction

Critical clinical need in development of reliable prognostic markers suitable for stratification of prostate cancer patients is clearly demonstrated by the results of a recent randomized study of the therapeutic efficacy of surgery versus watch and wait strategy demonstrating only modest 6.6% absolute reduction in mortality after prostatectomy compared to observation, despite the association of surgery with a 50% reduction in hazard ration of death from prostate cancer (1). It appears that a measurable clinical benefit of surgery is limited to poorly defined sub-population of prostate cancer patients. Therefore, an improved ability to identify a sub-group of prostate cancer patients who would benefit from therapy should have a significant immediate positive clinical and socio-economic impact.

Widely used biochemical, histopathological, and clinical criteria such as PSA level, Gleason score, the clinical tumor stage and molecular genetic approaches assaying loss of tumor suppressors or gain of oncogenes (2) had only limited success with respect to prostate cancer patients stratification and demonstrated a significant variability in predictive value among different clinical laboratories and hospitals. Furthermore, best existing markers cannot reliably identify at the time of diagnosis a poor prognosis group of prostate cancer patients that ultimately would fail therapy (3). Classification nomograms that incorporate measurements of several individual pre- and postoperative parameters are generally recognized as most efficient clinically useful models currently available for prediction of the probability of relapse-free survival after therapy of individual prostate cancer patients (4-7). However, one of the significant deficiencies of these classification systems is that they have only limited utility in predicting the differences in outcomes readily observed between patients diagnosed with prostate cancers exhibiting similar clinical, histopathological, and biochemical features. Therefore, a critical clinical need exists to improve the classification accuracy of prostate cancer patients with respect to clinical outcome after therapy.

Expression profiling of prostate tumor samples using oligonucleotide or cDNA microarray technology revealed gene expression signatures associated with human prostate cancer (8-19), including potential prostate cancer prognosis markers (9, 14, 16, 17). However, one of the major limitations of these studies was that the same clinical data set was utilized for both signature discovery and validation. Furthermore, usually only a single or few hits were validated using independent methods and independent clinical data sets, thus diminishing the potential advantage of the use of a panel of markers over a single marker in diagnostic and/or prognostic applications.

Here we applied a microarray-based gene expression profiling approach to identify molecular signatures distinguishing sub-groups of patients with differing outcome and develop a stratification algorithm demonstrating high discrimination accuracy between sub-groups of prostate cancer patients with distinct clinical outcome after therapy in a training set of 21 prostate cancer patients. To validate a potential clinical utility of discovered genetic signatures, we confirmed the discrimination power of proposed prostate cancer prognosis stratification algorithm using an independent set of 79 clinical tumor samples.

Our data indicate that identified molecular signatures provide the bases for developing clinical prognostic tests suitable for stratification of prostate cancer patients at the time of diagnosis with respect to likelihood of negative or positive clinical outcome after therapy. Our results provide experimental evidence of a transcriptional resemblance between metastatic human prostate carcinoma xenografts in nude mice and primary prostate tumors from patients subsequently developing relapse after therapy. These data suggest that genetically defined metastasis-promoting features of primary tumors are one of the major contributing factors of aggressive clinical behavior and unfavorable prognosis in prostate cancer patients.

Materials and Methods

Clinical Samples.

We utilized in our experiments two independent sets of clinical samples for signature discovery (training outcome set of 21 samples) and validation (validation outcome set of 79 samples). Original gene expression profiles of the training set of 21 clinical samples analyzed in this study were recently reported (14). Primary gene expression data files of clinical samples as well as associated clinical information were provided by Dr. W. Sellers and can be found at http://www-genome.wi.mit.edu/cancer/.

Prostate tumor tissues comprising validation data set were obtained from 79 prostate cancer patients undergoing therapeutic or diagnostic procedures performed as part routine clinical management at MSKCC. Clinical and pathological features of 79 prostate cancer cases comprising validation outcome set are presented in the Table 70. Median follow-up after therapy in this cohort of patients was 70 months. Samples were snap-frozen in liquid nitrogen and stored at −80° C. Each sample was examined histologically using H&E-stained cryostat sections. Care was taken to remove nonneoplastic tissues from tumor samples. Cells of interest were manually dissected from the frozen block, trimming away other tissues. All of the studies were conducted under MSKCC Institutional Review Board-approved protocols.

Cell Culture.

Cell lines used in this study were previously described (19). The LNCap- and PC-3-derived cell lines were developed by consecutive serial orthotopic implantation, either from metastases to the lymph node (for the LN series), or reimplanted from the prostate (Pro series). This procedure generated cell variants with differing tumorigenicity, frequency and latency of regional lymph node metastasis (19). Except where noted, cell lines were grown in RPMI1640 supplemented with 10% FBS and gentamycin (Gibco BRL) to 70-80% confluence and subjected to serum starvation as described (19), or maintained in fresh complete media, supplemented with 10% FBS.

Orthotopic Xenografts. Orthotopic xenografts of human prostate PC-3 cells and sublines used in this study were developed by surgical orthotopic implantation as previously described (19). Briefly, 2×10⁶ cultured PC3 cells, PC3M or PC3MLN4 sublines were injected subcutaneously into male athymic mice, and allowed to develop into firm palpable and visible tumors over the course of 2-4 weeks. Intact tissue was harvested from a single subcutaneous tumor and surgically implanted in the ventral lateral lobes of the prostate gland in a series of six athymic mice per cell line subtype. The mice were examined periodically for suprapubic masses, which appeared for all subline cell types, in the order PC3MLN4>PC3M>>PC3. Tumor-bearing mice were sacrificed by CO₂ inhalation over dry ice and necropsy was carried out in a 2-4° C. cold room. Typically, bilaterally symmetric prostate gland tumors in the shape of greatly distended prostate glands were apparent. Prostate tumor tissue was excised and snap frozen in liquid nitrogen. The elapsed time from sacrifice to snap freezing was <5 min. A systematic gross and microscopic post mortem examination was carried out.

Tissue Processing for mRNA and RNA Isolation.

Fresh frozen orthotopic tumor was examined by use of hematoxylin and eosin stained frozen sections. Orthotopic tumors of all sublines exhibited similar morphology consisting of sheets of monotonous closely packed tumor cells with little evidence of differentiation interrupted by only occasional zones of largely stromal components, vascular lakes, or lymphocytic infiltrates. Fragments of tumor judged free of these non-epithelial clusters were used for mRNA preparation. Frozen tissue (1-3 mm×1-3 mm) was submerged in liquid nitrogen in a ceramic mortar and ground to powder. The frozen tissue powder was dissolved and immediately processed for mRNA isolation using a Fast Tract kit for mRNA extraction (Invitrogen, Carlsbad, Calif., see above) according to the manufacturers instructions.

RNA and mRNA Extraction.

For gene expression analysis, cells were harvested in lysis buffer 2 hrs after the last media change at 70-80% confluence and total RNA or mRNA was extracted using the RNeasy (Qiagen, Chatsworth, Calif.) or FastTract kits (Invitrogen, Carlsbad, Calif.). Cell lines were not split more than 5 times prior to RNA extraction, except where noted.

Affymetrix Arrays.

The protocol for mRNA quality control and gene expression analysis was that recommended by Affymetrix (http://www.affvmetrix.com). In brief, approximately one microgram of mRNA was reverse transcribed with an oligo(dT) primer that has a T7 RNA polymerase promoter at the 5′ end. Second strand synthesis was followed by cRNA production incorporating a biotinylated base. Hybridization to Affymetrix U95Av2 arrays representing 12,625 transcripts overnight for 16 h was followed by washing and labeling using a fluorescently labeled antibody. The arrays were read and data processed using Affymetrix equipment and software as reported previously (18, 19).

Data Analysis.

Detailed protocols for data analysis and documentation of the sensitivity, reproducibility and other aspects of the quantitative statistical microarray analysis using Affymetrix technology have been reported (18, 19). 40-50% of the surveyed genes were called present by the Affymetrix Microarray Suite 5.0 software in these experiments. The concordance analysis of differential gene expression across the data sets was performed using Affymetrix MicroDB v.3.0 and DMT v.3.0 software as described earlier (18, 19). We processed the microarray data using the Affymetrix Microarray Suite v.5.0 software and performed statistical analysis of expression data set using the Affymetrix MicroDB and Affymetrix DMT software. This analysis identified a set of 218 genes (91 up-regulated and 127 down-regulated transcripts) differentially regulated in tumors from patients with recurrent versus non-recurrent prostate cancer at the statistically significant level (p<0.05) defined by both T-test and Mann-Whitney test (Table 69). The concordance analysis of differential gene expression across the clinical and experimental data sets was performed using Affymetrix MicroDB v.3.0 and DMT v.3.0 software as described earlier (19). The Pearson correlation coefficient for individual test samples and appropriate reference standard was determined using the Microsoft Excel software as described in the signature discovery protocol.

Survival Analysis.

The Kaplan-Meier survival analysis was carried out using the Prism 4.0 software. Statistical significance of the difference between the survival curves for different groups of patients was assessed using Chi square and Logrank tests.

Discovery and validation of the prostate cancer recurrence predictor algorithm. According to the present invention, clinically relevant genetic signatures can be found by searching for clusters of co-regulated genes that display highly concordant transcript abundance behavior across multiple experimental models and clinical settings that model or represent malignant phenotypes of interest (Glinsky, G. V., Krones-Herzig, A., Glinskii, A. B., Gebauer, G. Microarray analysis of xenograft-derived cancer cell lines representing multiple experimental models of human prostate cancer. Molecular Carcinogenesis, 37: 209-221, 2003; Example 5, supra; Glinsky, G. V., Krones-Herzig, A., Glinskii, A. B. Malignancy-associated regions of transcriptional activation: gene expression profiling identifies common chromosomal regions of a recurrent transcriptional activation in human prostate, breast, ovarian, and colon cancers. Neoplasia, 5: 21-228; Glinsky, G. V., Ivanova, Y. A., Glinskii, A. B. Common malignancy-associated regions of transcriptional activation (MARTA) in human prostate, breast, ovarian, and colon cancers are targets for DNA amplification. Cancer Letters, in press, 2003). Thus, a primary criterion in selecting genes for inclusion within the cluster is the concordance of changes in expression rather than a magnitude of changes (e.g., fold change). Accordingly, transcripts of interest are expected to have a tightly controlled “rank order” of expression within a cluster of co-regulated genes reflecting a balance of up- and down-regulation as a desired regulatory end-point in a cell. A degree of resemblance of the transcript abundance rank order within a gene cluster between a test sample and reference standard is measured by a Pearson correlation coefficient and designated as a phenotype association index (PAI), as described fully in the introduction of the Detailed Description of Preferred Embodiments section. To identify genes with consistently concordant expression patterns across multiple data sets and various experimental conditions, we compared the expression profile of 218 genes (test samples) to the expression profiles of transcripts differentially regulated in multiple experimental models (reference standard) of human prostate cancer (Glinsky, G. V., Krones-Herzig, A., Glinskii, A. B., Gebauer, G. Microarray analysis of xenograft-derived cancer cell lines representing multiple experimental models of human prostate cancer. Molecular Carcinogenesis, 37: 209-221, 2003).

The transcripts comprising each signature were selected based on Pearson correlation coefficients (r>0.95) reflecting a degree of similarity of expression profiles in clinical tumor samples (recurrent versus non-recurrent tumors) and experimental samples using the following protocol.

Step 1. Sets of differentially regulated transcripts were independently identified for each experimental conditions (see below) and clinical samples using the Affymetrix microarray processing and statistical analysis software package as described in this examples's Materials and Methods section.

Step 2. Sub-sets of transcripts exhibiting concordant expression changes in clinical and experimental samples were identified using the Affymetrix MicroDB and DMT software. Sub-sets of transcripts were identified with concordant changes of transcript abundance behavior in recurrent versus non-recurrent clinical tumor samples (218 transcripts) and experimental conditions independently defined for each signature (Signature 1: PC-3MLN4 orthotopic versus s.c. xenografts; Signature 2: PC-3MLN4 versus PC-3M & PC-3 orthotopic xenografts; Signature 3: PC-3/LNCap consensus class, Glinsky, G. V., Krones-Herzig, A., Glinskii, A. B., Gebauer, G. Microarray analysis of xenograft-derived cancer cell lines representing multiple experimental models of human prostate cancer. Molecular Carcinogenesis, 37: 209-221, 2003). Thus, from a set of 218 transcripts three concordant sub-sets of transcripts were identified corresponding to each binary comparison of clinical and experimental samples.

Step 3. Small gene clusters were selected as sub-sets of genes exhibiting concordant changes of transcript abundance behavior in recurrent versus non-recurrent clinical tumor samples (218 transcripts) and experimental conditions defined for each signature (Signature 1: PC-3MLN4 orthotopic versus s.c. xenografts; Signature 2: PC-3MLN4 versus PC-3M & PC-3 orthotopic xenografts; Signature 3: PC-3/LNCap consensus class, Glinsky, G. V., Krones-Herzig, A., Glinskii, A. B., Gebauer, G.. Microarray analysis of xenograft-derived cancer cell lines representing multiple experimental models of human prostate cancer. Molecular Carcinogenesis, 37: 209-221, 2003). Expression profiles were presented as log10 average fold changes for each transcript and processed for visualization and Pearson correlation analysis using Microsoft Excel software. The cut-off criterion for cluster formation was set to exceed a Pearson correlation coefficient 0.95 among the log10 transformed average expression values in the compared groups.

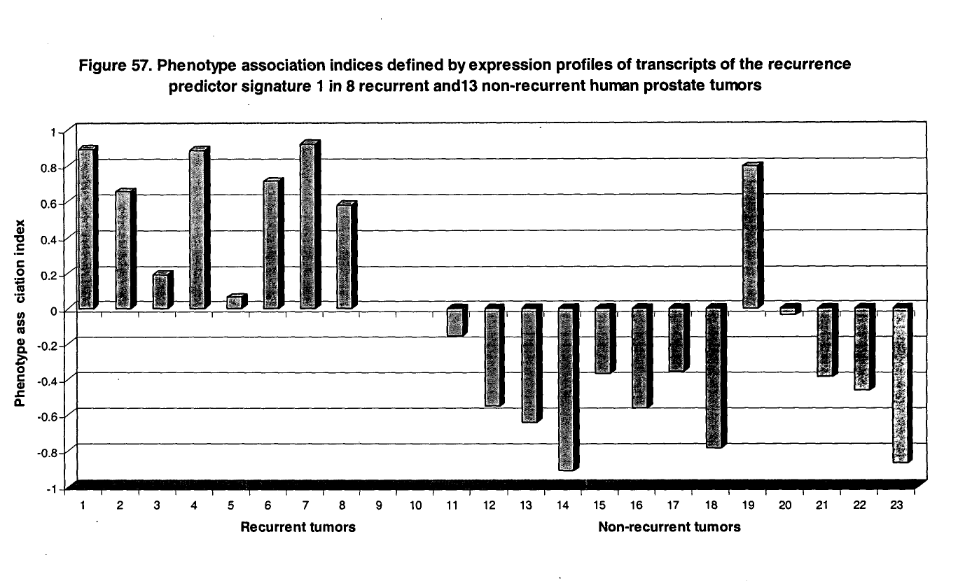

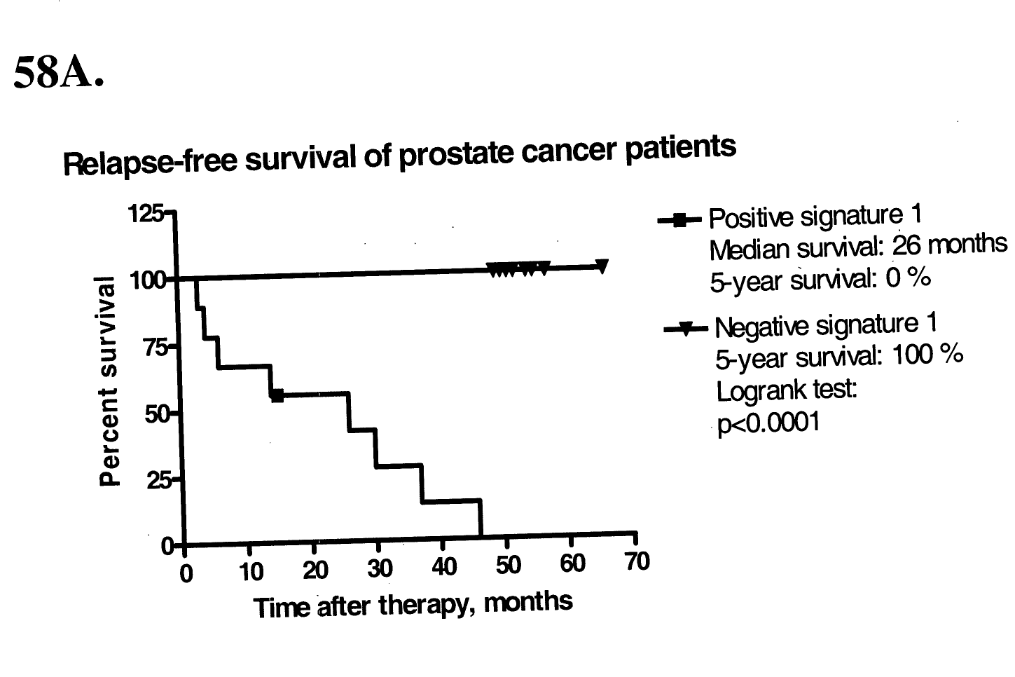

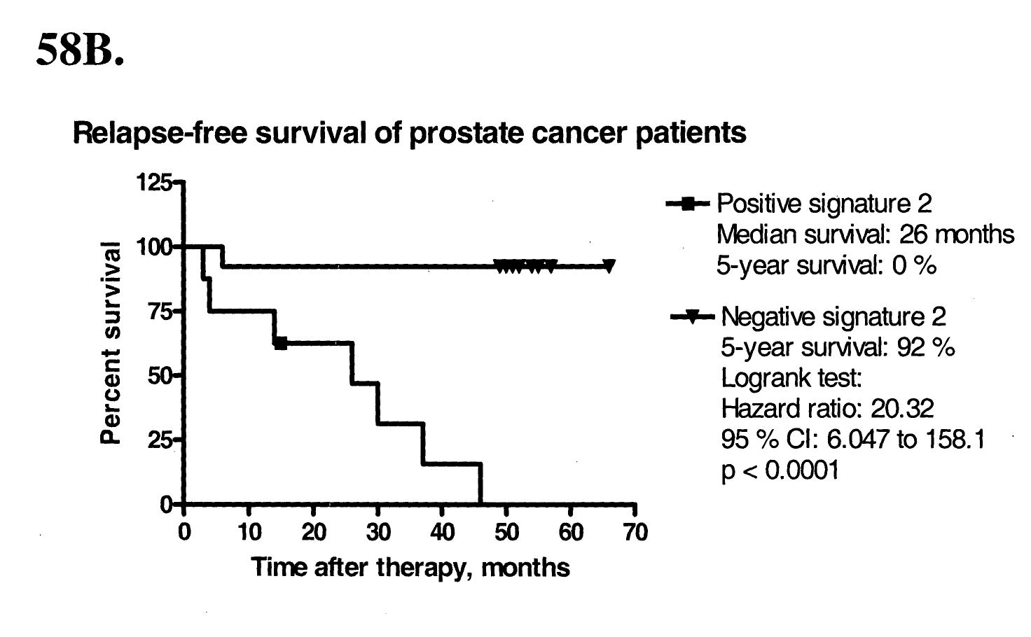

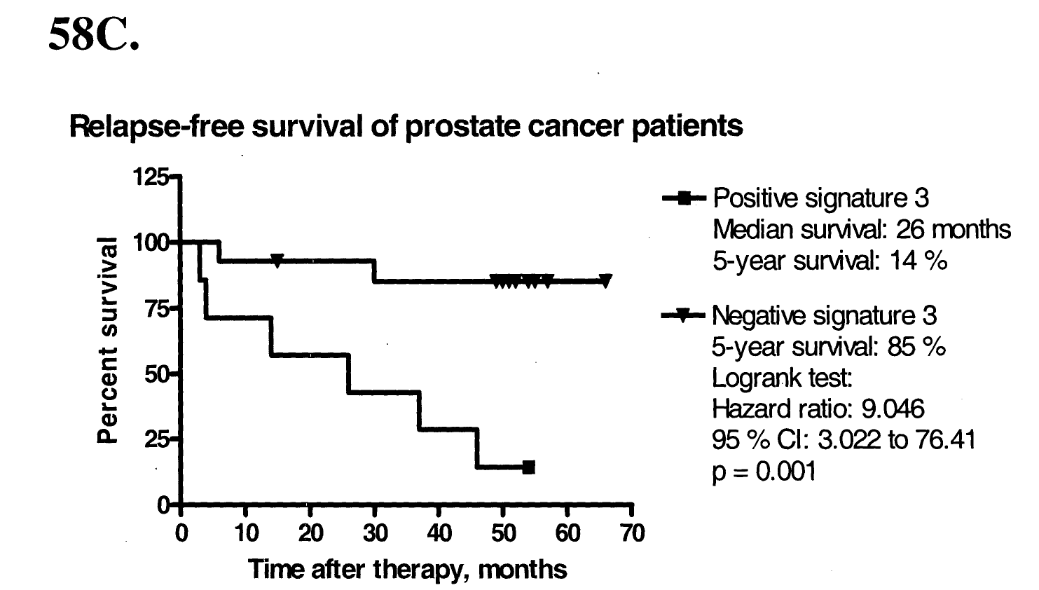

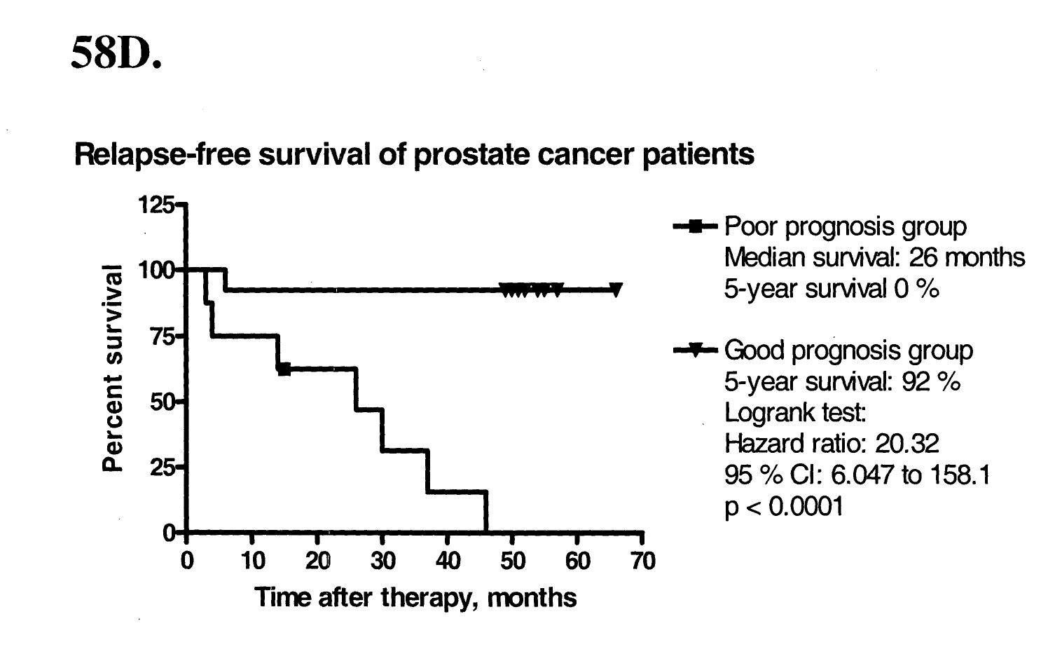

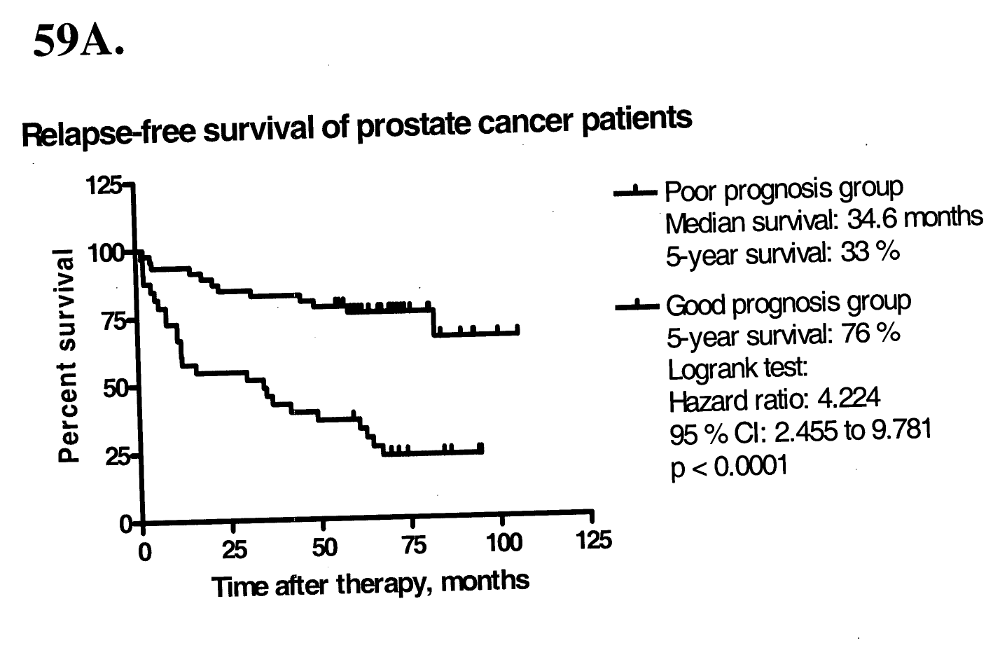

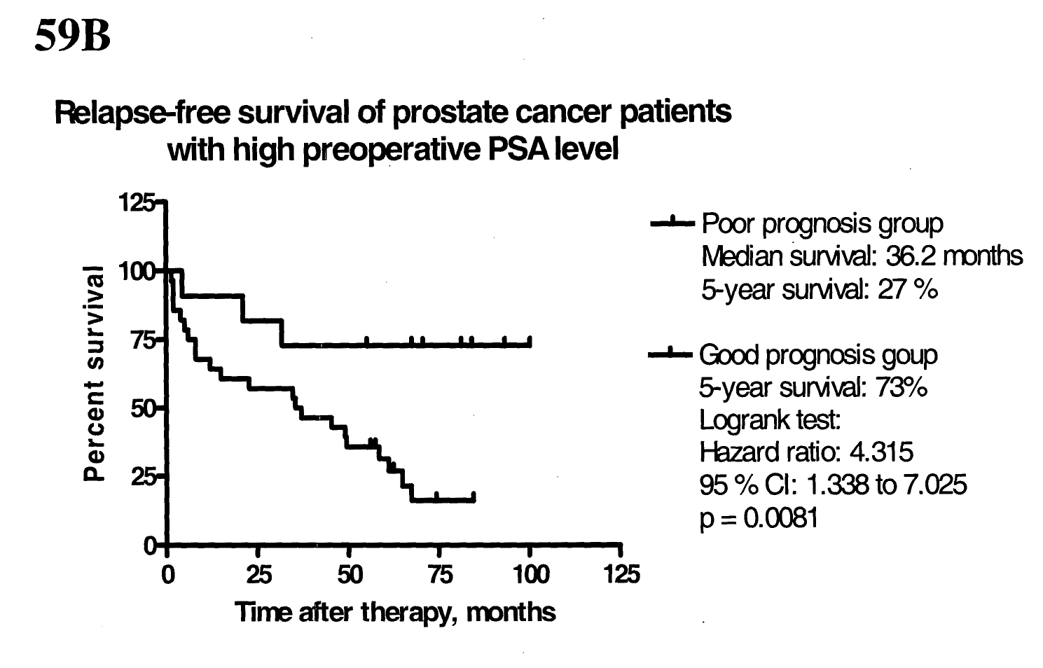

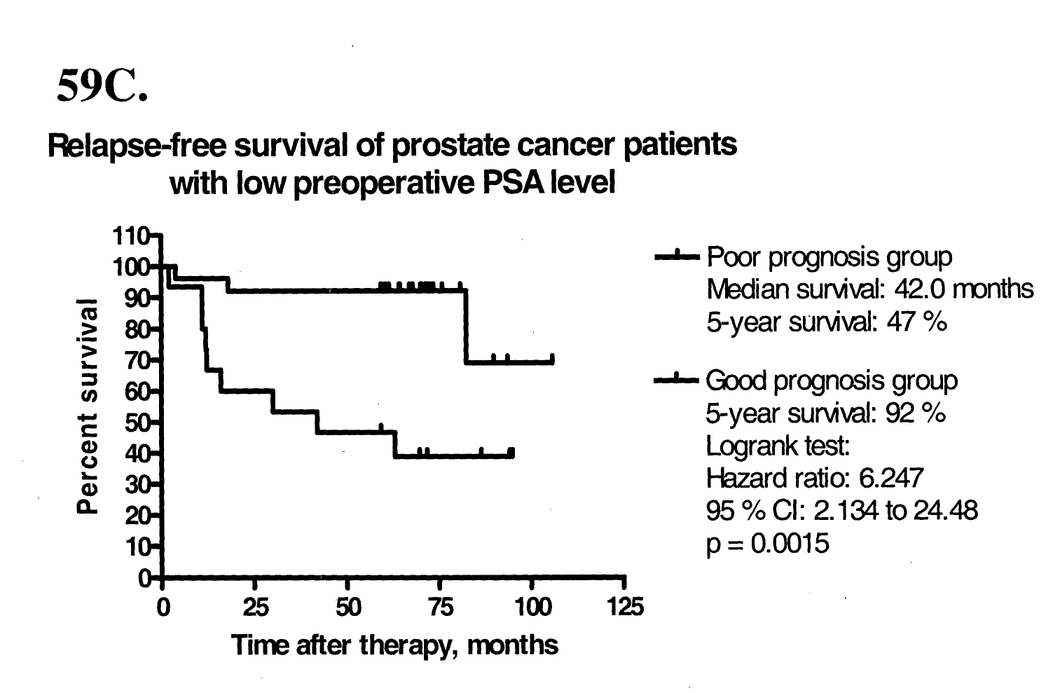

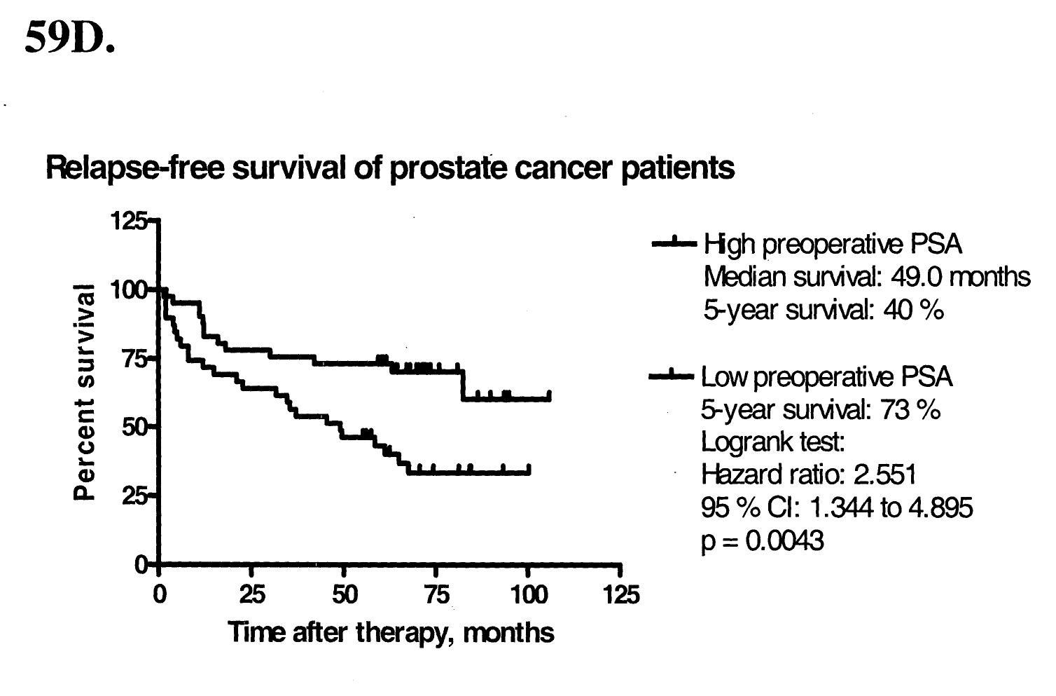

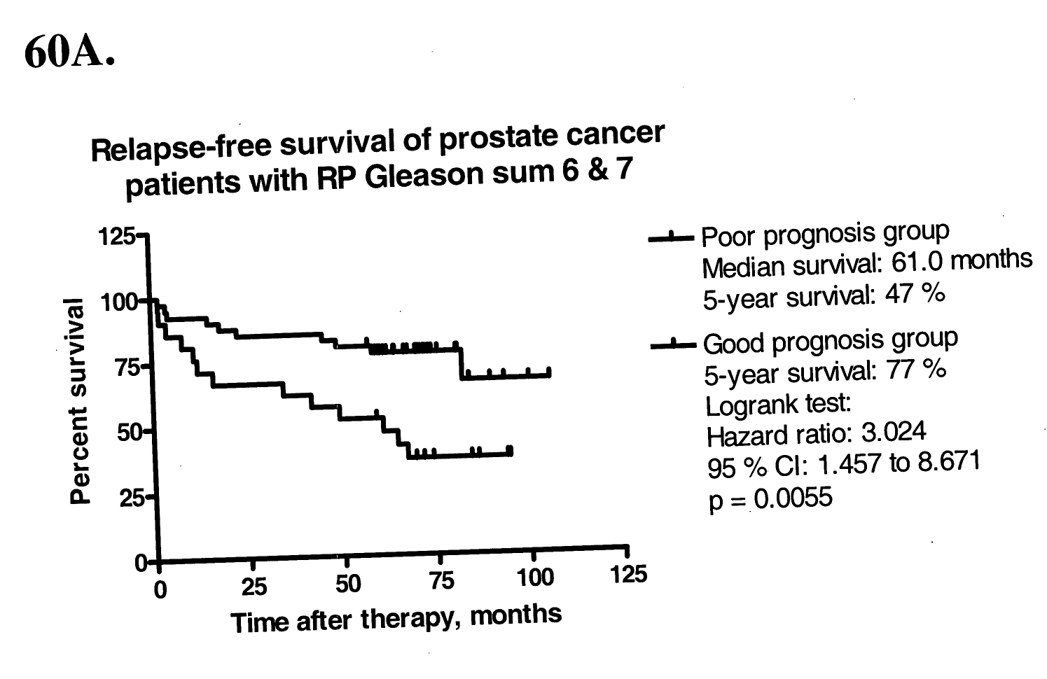

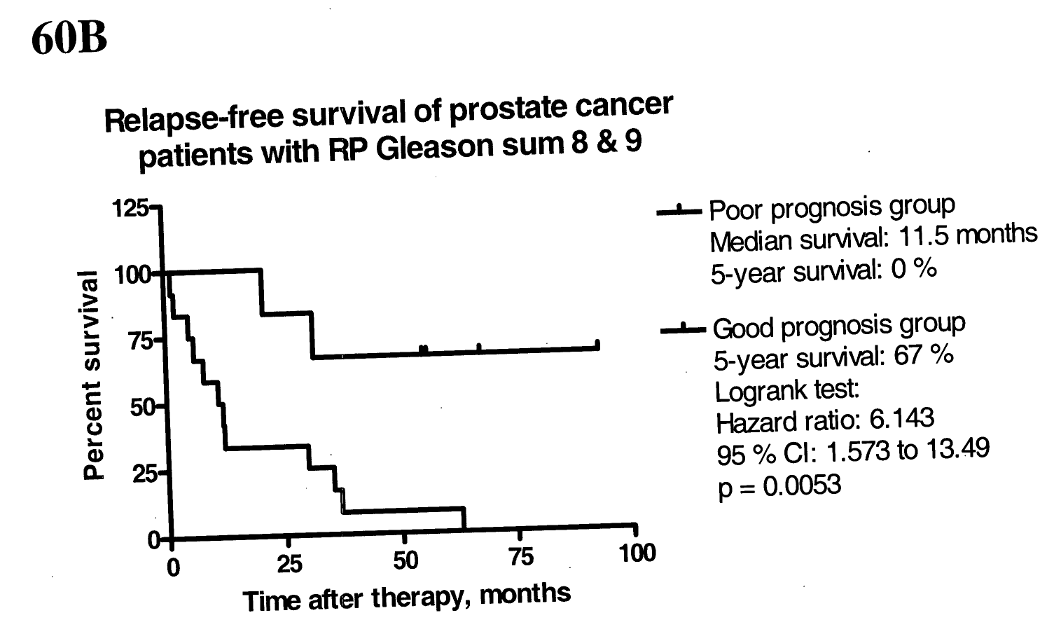

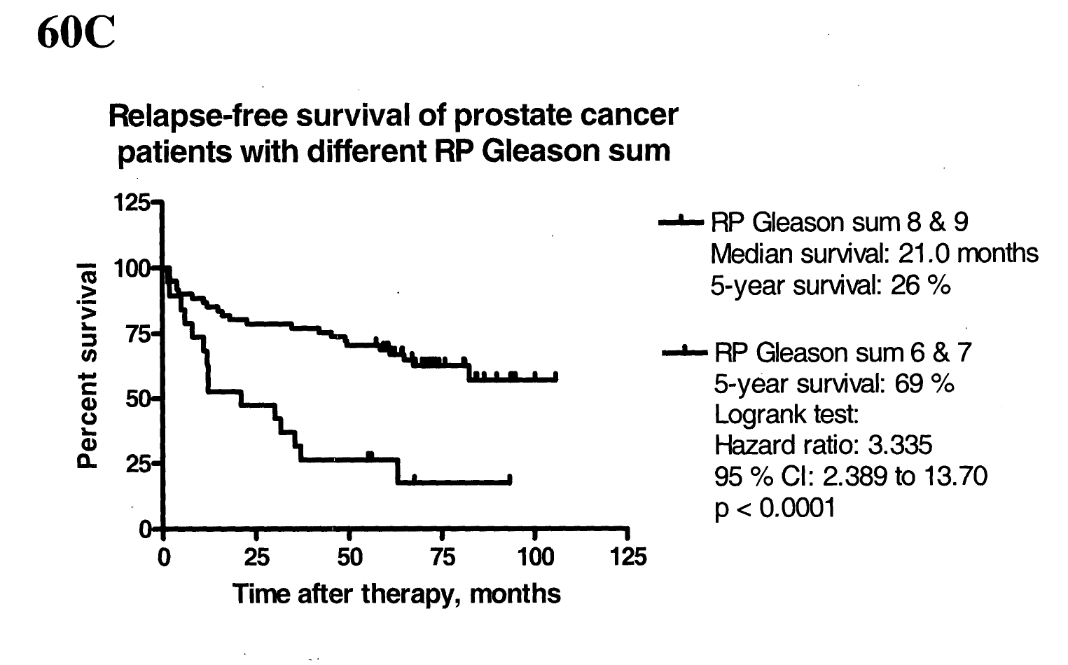

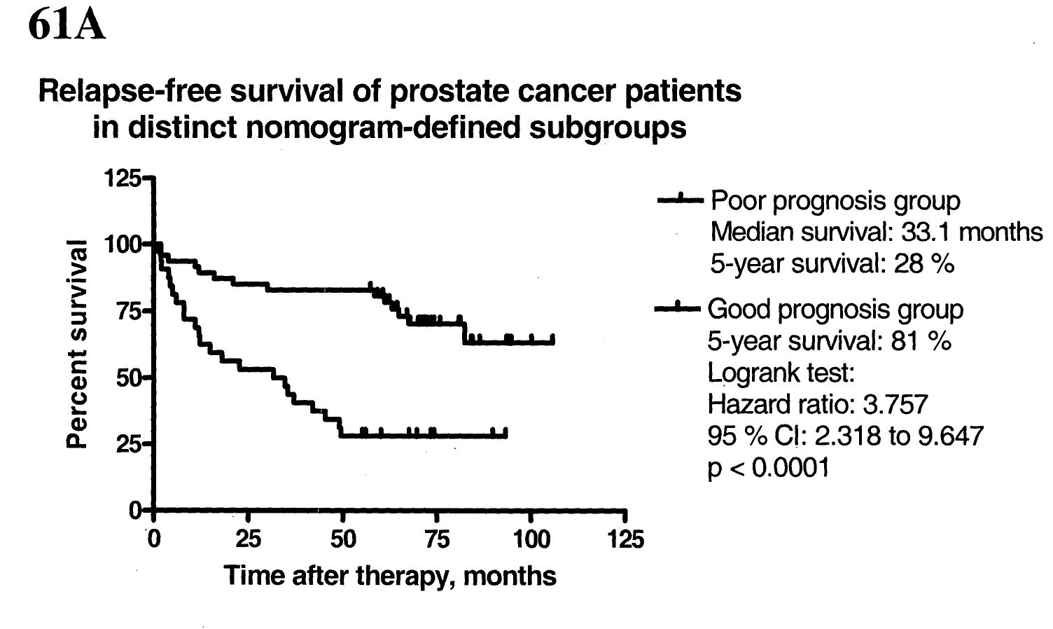

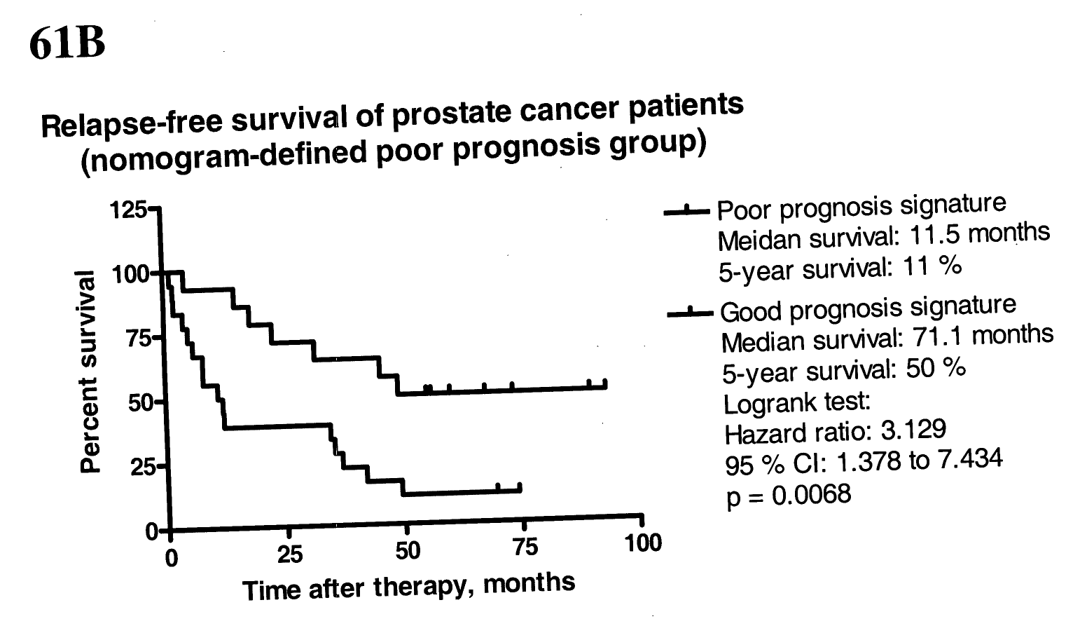

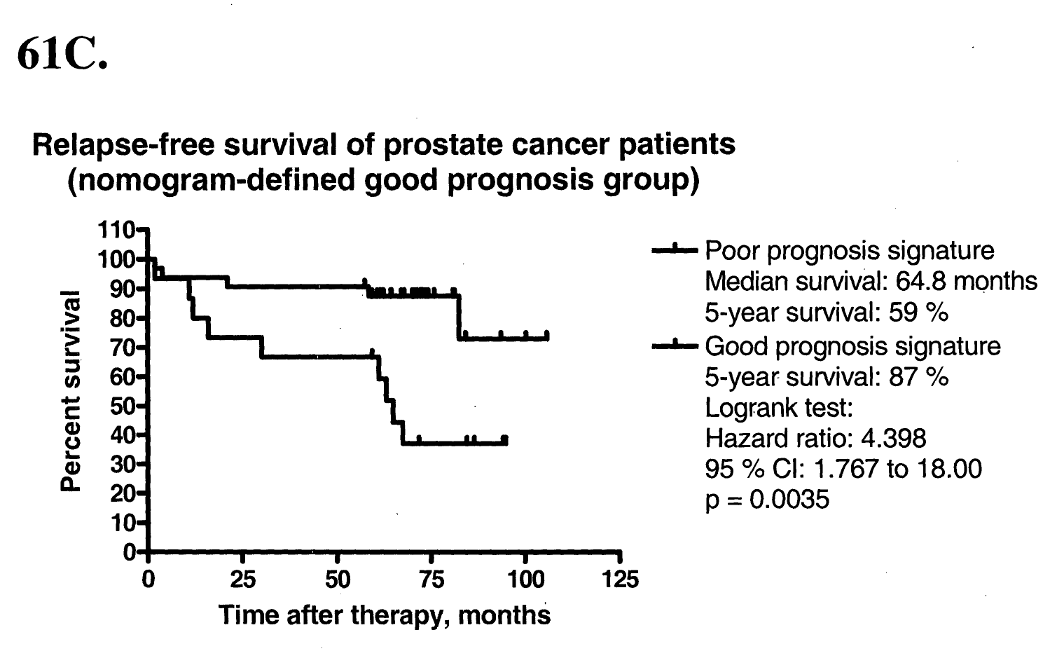

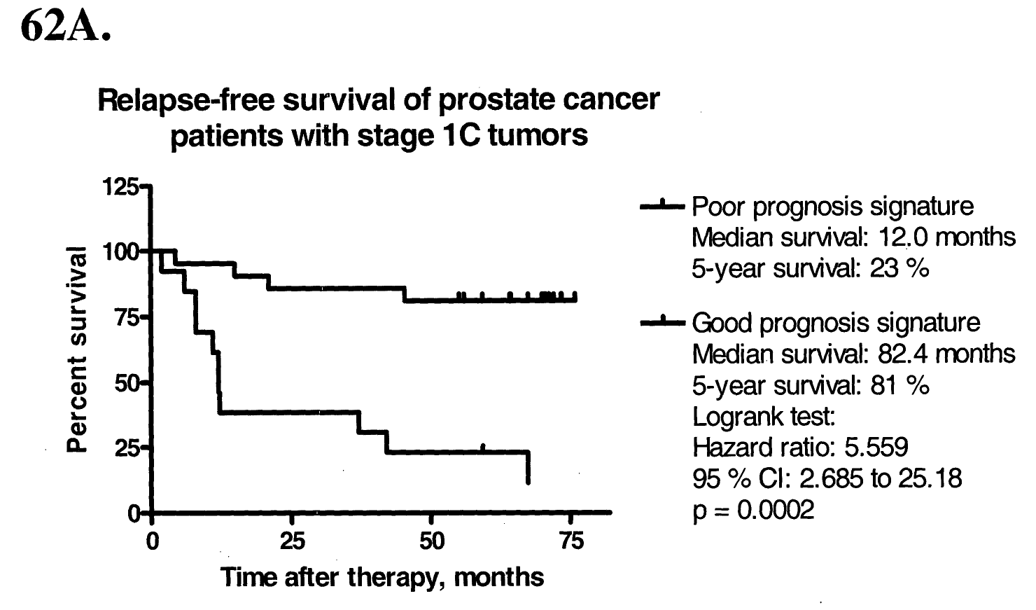

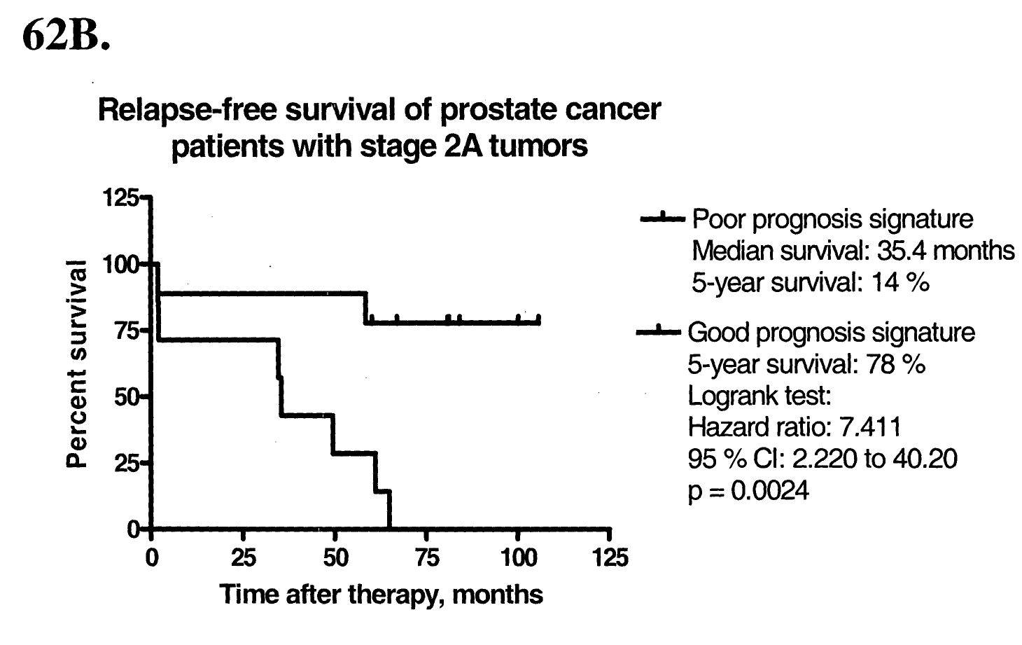

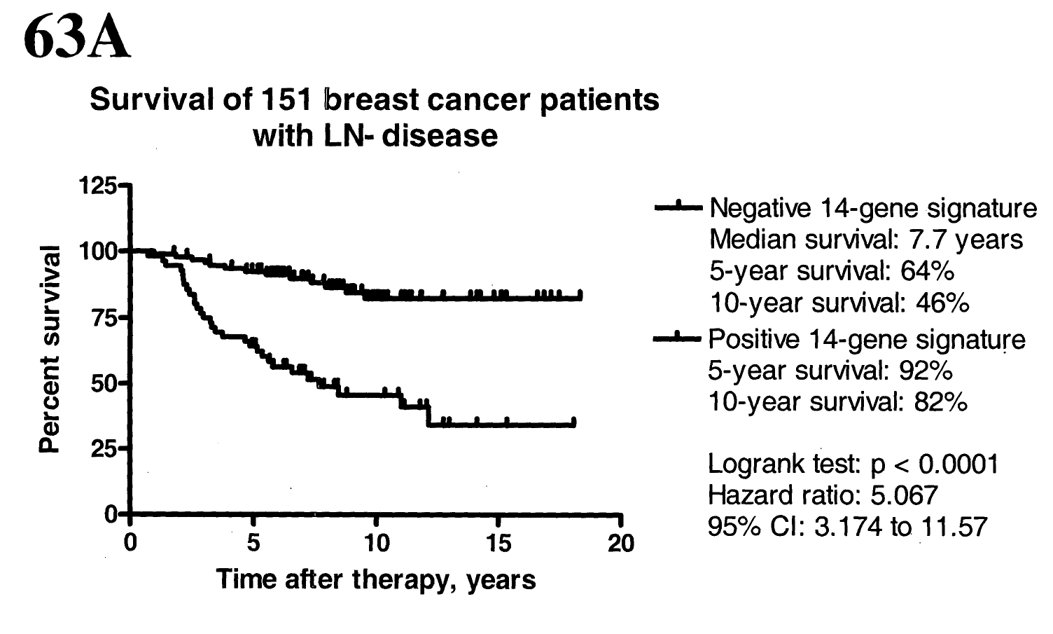

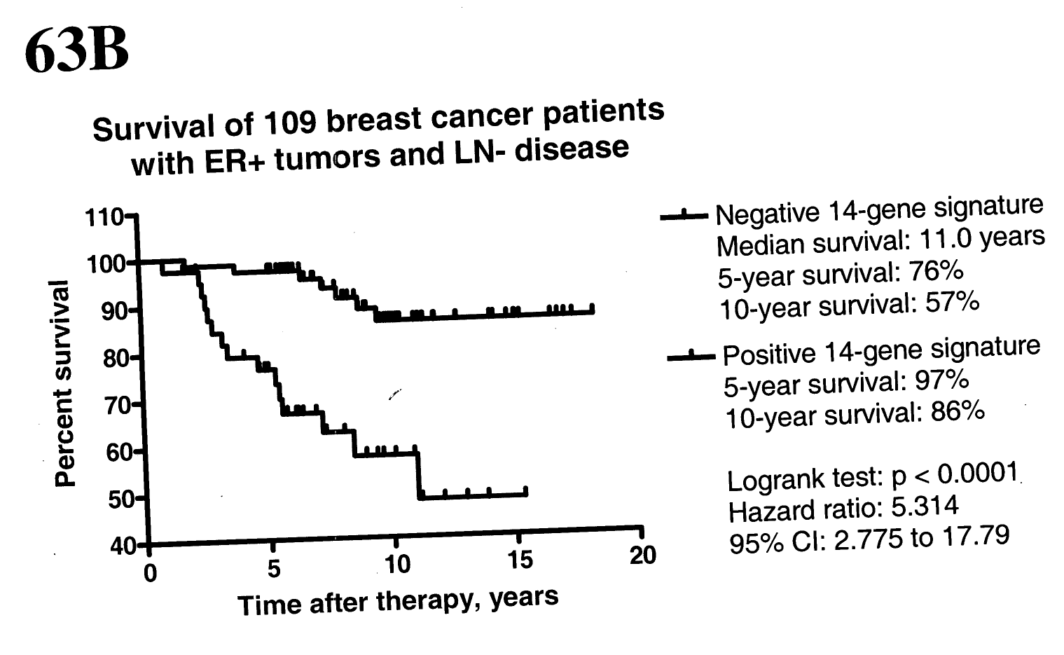

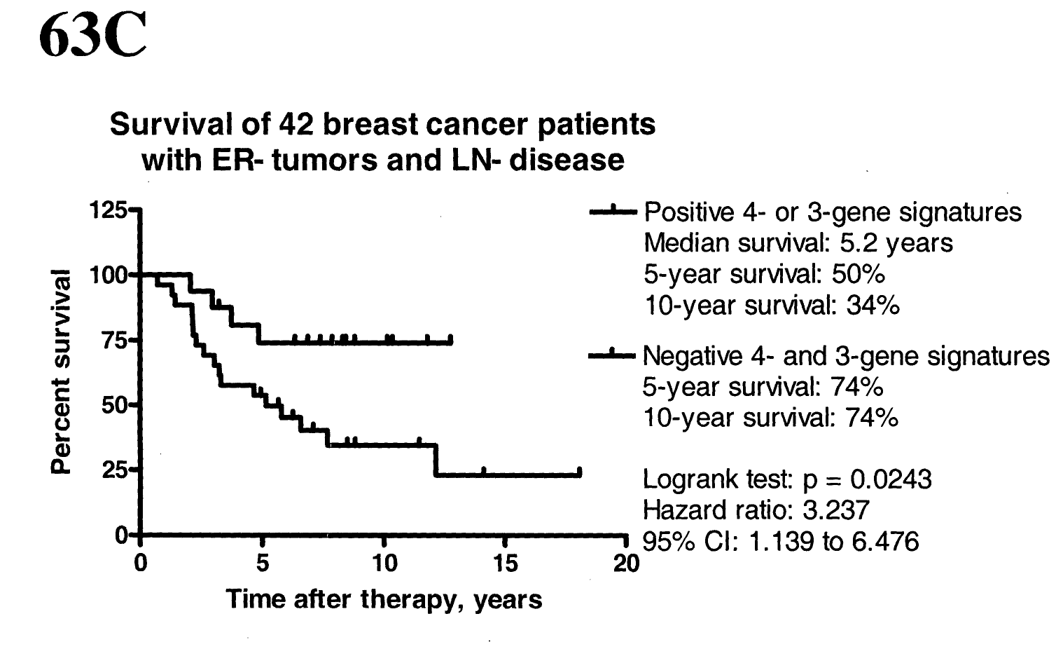

Step 4. Small gene clusters exhibiting highly concordant pattern of expression (Pearson correlation coefficient, r>0.95) in clinical and experimental samples (identified in step 3) were evaluated for their ability to discriminate clinical samples with distinct outcomes after the therapy. To assess a potential prognostic relevance of individual gene clusters, we calculated a Pearson correlation coefficient for each of 21 tumor samples (training data set) by comparing the expression profiles of individual samples to the reference expression profiles of relevant experimental samples defined for each signature and an “average” expression profile of recurrent versus non-recurrent tumors. As explained above, we named the corresponding correlation coefficients calculated for individual samples the phenotype association indices (PAIs). We evaluated the prognostic power of identified clusters of co-regulated transcripts based on their ability to segregate the patients with recurrent and non-recurrent prostate tumors into distinct sub-groups and selected a single best performing cluster for each binary condition (FIG. 57; Tables 69 & 70).

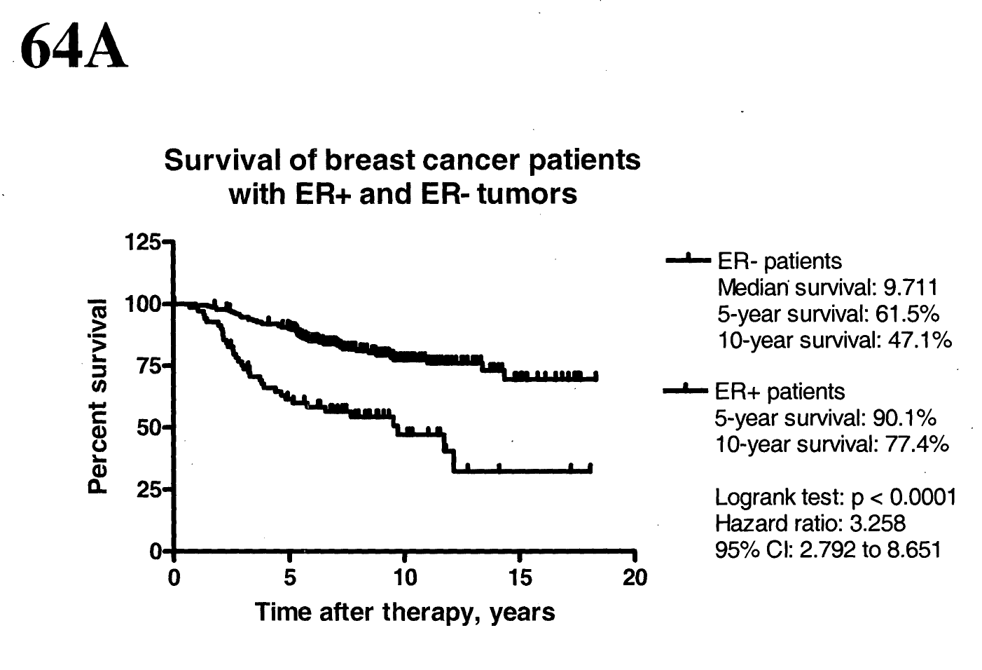

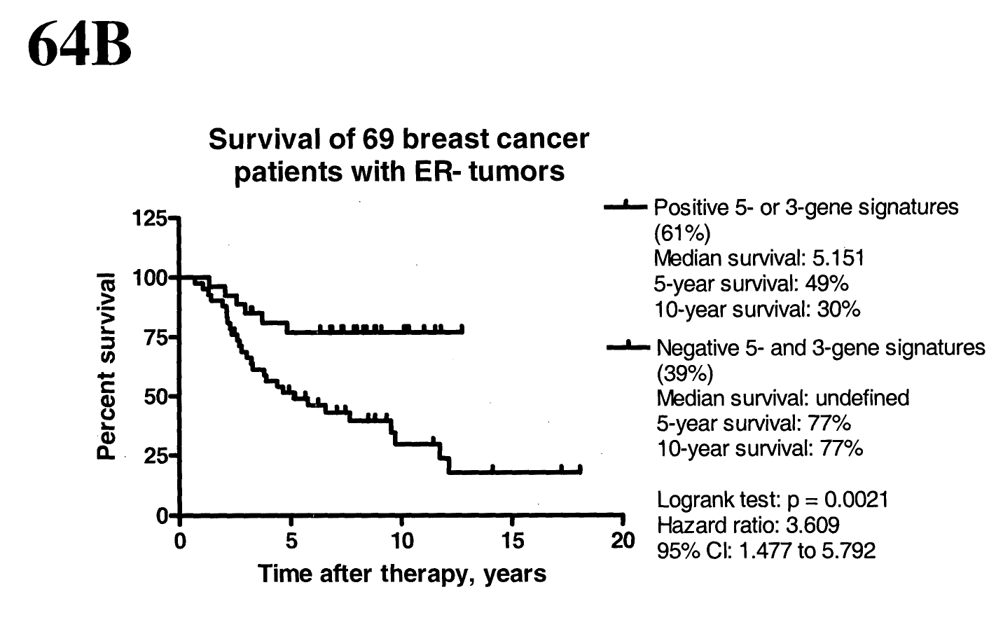

Step 5. We used Kaplan-Meier survival analysis to assess the prognostic power of each best-performing cluster in predicting the probability that patients would remain disease-free after therapy (FIG. 58-62). We selected the prognosis discrimination cut-off value for each signature based on highest level of statistical significance in patient's stratification into poor and good prognosis groups as determined by the log-rank test (lowest P value and highest hazard ratio; Table 70 & FIGS. 58-62). Clinical samples having the Pearson correlation coefficient at or higher than the cut-off value were identified as having the poor prognosis signature. Clinical samples with the Pearson correlation coefficient lower the cut-off value were identified as having the good prognosis signature.

Step 6. We developed a prostate cancer recurrence predictor algorithm taking into account calls from all three individual signatures. We selected the common prognosis discrimination cut-off value for all three signatures based on highest level of statistical significance in patient's stratification into poor and good prognosis groups as determined by Kaplan-Meier survival analysis (lowest P value and highest hazard ratio defined by the log-rank test; Table 70 & FIG. 58-62). Clinical samples having the Pearson correlation coefficient at or higher the cut-off value defined by at least two signatures were identified as having the poor prognosis signature. Clinical samples with the Pearson correlation coefficient lower than the cut-off value defined by at least two signatures were identified as having the good prognosis signature. We found that the cut-off value of PAIs>0.2 scored in two of three individual clusters allowed to achieve the 90% recurrence prediction accuracy (Table 70).

Step 7. We validated the prognostic power of prostate cancer recurrence predictor algorithm alone and in combination with the established markers of outcome using an independent clinical set of 79 prostate cancer patients (FIGS. 58-6269 & 71).

Results

Identification of Molecular Signatures Distinguishing Sub-Groups of Prostate Cancer Patients with Distinct Clinical Outcomes After Therapy.

To identify the outcome predictor signatures, we utilized as a training data set the expression analysis of 12,625 transcripts in 21 prostate tumor samples obtained from prostate cancer patients with distinct clinical outcomes after therapy. Using biochemical evidence of relapse after therapy as a criterion of treatment failure, 21 patients were divided into two sub-groups, representing prostate cancer with recurrent (8 patients) and non-recurrent (13 patients) clinical behavior (14). We processed the original U95Av2 GeneChip CEL files using the Affymetrix Microarray Suite 5.0 software and performed statistical analysis of expression data set using the Affymetrix MicroDB and Affymetrix DMT software. This analysis identified a set of 218 genes (91 up-regulated and 127 down-regulated transcripts) differentially regulated in tumors from patients with recurrent versus non-recurrent prostate cancer at the statistically significant level (p<0.05) defined by both T-test and Mann-Whitney test (Table 70).

To reduce the number of hits in potential outcome predictor clusters and identify transcripts of potential biological relevance, we compared the expression profile of 218 genes to the expression profiles of transcripts differentially regulated in multiple experimental models of human prostate cancer (Glinsky, G. V., Krones-Herzig, A., Glinskii, A. B., Gebauer, G. Microarray analysis of xenograft-derived cancer cell lines representing multiple experimental models of human prostate cancer. Molecular Carcinogenesis, 37: 209-221, 2003, and Example 5, supra) in search for genes with consistently concordant expression patterns across multiple data sets and various experimental conditions. We identified several small gene clusters exhibiting highly concordant pattern of expression (Pearson correlation coefficient, r>0.95) in clinical and experimental samples. We evaluated the prognostic power of each identified cluster of co-regulated transcripts based on ability to segregate the patients with recurrent and non-recurrent prostate tumors into distinct sub-groups. To assess a potential prognostic relevance of individual gene clusters, we calculated a Pearson correlation coefficient for each of 21 tumor samples by comparing the expression profiles of individual samples to the “average” expression profile of recurrent versus non-recurrent tumors and expression profiles of relevant experimental samples (Table 69 and FIG. 57). Based on expected correlation of expression profiles of identified gene clusters with recurrent clinical behavior of prostate cancer, we named the corresponding correlation coefficients calculated for individual samples the phenotype association indices (PAIs).

Using this strategy we identified several gene clusters (Tables 69 & 70) discriminating with 86-95% accuracy human prostate tumors exhibiting recurrent or non-recurrent clinical behavior (FIG. 57 and Tables 69 & 70). The transcripts comprising each signature in Table 69 were selected based on Pearson correlation coefficients (r>0.95) reflecting a degree of similarity of expression profiles in clinical tumor samples (recurrent versus non-recurrent tumors) and experimental samples. Selection of transcripts was performed from sets of genes exhibiting concordant changes of transcript abundance behavior in recurrent versus non-recurrent clinical tumor samples (218 transcripts) and experimental conditions independently defined for each signature (Signature 1: PC-3MLN4 orthotopic versus s.c. xenografts; Signature 2: PC-3MLN4 versus PC-3M & PC-3 orthotopic xenografts; Signature 3: PC-3/LNCap consensus class, Glinsky, G. V., Krones-Herzig, A., Glinskii, A. B., Gebauer, G. Microarray analysis of xenograft-derived cancer cell lines representing multiple experimental models of human prostate cancer. Molecular Carcinogenesis, 37: 209-221, 2003, and Example 5, supra). The expression profiles were presented as log10 average fold changes for each transcript.

TABLE 69
Gene expression signatures associated with recurrent prostate cancer.

| | | |
|---|---|---|
| | GenBank | UniGene |
| Gene Name | ID | ID |

14/19 Signature 1

| | | | |
|---|---|---|---|
| LocusLink | | | |
| Name | | | |
| MGC5466 | Hypothetical protein MGC5466 | U90904 | Hs. 83724 |
| Wnt5A | proto-oncogene Wnt5A | L20861 | Hs. 152213 |
| KIAA0476 | KIAA0476 protein | AB007945 | Hs. 6684 |
| ITPR1 | inositol 1,4,5-trisphosphate | D26070 | Hs. 198443 |
| | receptor, type 1 | | |
| TCF2 | transcription factor 2, hepatic | X58840 | Hs. 169853 |

14/19 Signature 2

| | | | |
|---|---|---|---|
| Gene | | | |
| MGC5466 | Hypothetical protein MGC5466 | U90904 | Hs. 83724 |
| CHAF1A | Chromatin assembly factor 1, | U20979 | Hs. 79018 |
| | subunit A | | |
| CDS2 | CDP-diacylglycerol synthase 2 | Y16521 | Hs. 24812 |
| IER3 | Immediate early response 3 | S81914 | Hs. 76090 |

14/19 Signature 3

| | | | |
|---|---|---|---|
| LocusLink | | | |
| Name | | | |
| PPFIA3 | Protein tyrosine phosphatase, | AB014554 | Hs. 109299 |
| | receptor type, f polypeptide | | |
| COPEB | Core promoter element | AF001461 | Hs. 285313 |
| | binding protein | | |
| FOS | V-fos oncogene homolog | V01512 | Hs. 25647 |
| JUNB | Jun B proto-oncogene | X51345 | Hs. 400124 |
| ZFP36 | zinc finger protein 36, C3H type | M92843 | Hs. 343586 |

Table 70 illustrates data from 21 prostate cancer patients who provided tumor samples comprising a signature discovery (training) data set that were classified according to whether they had a good-prognosis signature or poor-prognosis signature based on PAI values defined by either individual recurrence predictor signatures or a recurrence predictor algorithm that takes into account calls from all three signatures. The number of correct predictions in the poor-prognosis and good-prognosis groups is shown as a fraction of patients with the observed clinical outcome after therapy (8 patients developed relapse and 13 patients remained disease-free). Correlation coefficients reflect a degree of similarity of expression profiles in clinical tumor samples (recurrent versus non-recurrent tumors) and experimental samples (Signature 1: PC-3MLN4 orthotopic versus s.c. xenografts; Signature 2: PC-3MLN4 versus PC-3M & PC-3 orthotopic xenografts; Signature 3: PC-3/LNCap consensus class, Glinsky, G. V., Krones-Herzig, A., Glinskii, A. B., Gebauer, G. Microarray analysis of xenograft-derived cancer cell lines representing multiple experimental models of human prostate cancer. Molecular Carcinogenesis, 37: 209-221, 2003; and Example 5, supra). P values were calculated with use of the log-rank test and reflect the statistically significant difference in the probability that patients would remain disease-free between poor-prognosis and good-prognosis sub-groups.

TABLE 70
Prostate cancer recurrence prediction accuracy in a good-prognosis
and a poor-prognosis sub-group of patients defined according to
whether they had a good-prognosis or a poor-prognosis signature.

| Recurrence | Correlation | Recurrent | Non-recurrent | | |
|---|---|---|---|---|---|
| signature | coefficient | cancer | cancer | Overall | P value |
| Signature 1 | r = 0.983 | 100% (8 of 8) | 92% (12 of 13) | 95% (20 of 21) | <0.0001 |
| Signature 2 | r = 0.963 | 88% (7 of 8) | 92% (12 of 13) | 90% (19 of 21) | <0.0001 |
| Signature 3 | r = 0.996 | 75% (6 of 8) | 92% (12 of 13) | 86% (18 of 21) | 0.001 |
| Algorithm | NA | 88% (7 of 8) | 92% (12 of 13) | 90% (19 of 21) | <0.0001 |

FIG. 57 illustrates application of the five-gene cluster (Table 69, signature 1) to characterize clinical prostate cancer samples according to their propensity for recurrence after therapy. The expression pattern of the genes in the recurrence predictor cluster was analyzed in each of twenty-one separate clinical samples. The analysis produces a quantitative phenotype association index (plotted on the Y-axis) for each of the twenty-one clinical prostate cancer samples. Tumors that are likely to recur are expected to have positive phenotype association indices reflecting positive correlation of gene expression with metastasis-promoting orthotopic xenografts, while those that are unlikely to recur are expected to have negative association indices.

The figure shows the phenotype association indices for eight samples from patients who later had recurrence as bars 1 through 8, while the association indices for thirteen samples from patients whose tumors did not recur is shown as bars 11 through 23. Eight of the eight samples (or 100%) from patients who later experienced recurrence had positive phenotype association indices and so were properly classified. Twelve of the thirteen samples (or 92.3%) from patients whose tumors did not recur had negative phenotype association indices and so were properly classified as non-recurrent tumors. Thus, overall, twenty of the twenty-one samples (or 95.2%) were properly classified using a five-gene recurrence predictor signature. Two alternative clusters identified using this strategy showed similar sample classification performance (Tables 69 & 70).

To further evaluate the prognostic power of the identified gene expression signatures, we performed Kaplan-Meier survival analysis using as a clinical end-point disease-free interval (“DFI”) after therapy in prostate cancer patients with positive and negative PAIs. The Kaplan-Meier survival curves showed a highly significant difference in the probability that prostate cancer patients would remain disease-free after therapy between the groups with positive and negative PAIs defined by the signatures (FIGS. 58A-C), suggesting that patients with positive PAIs exhibit a poor outcome signature whereas patients with negative PAIs manifest a good outcome signature. The estimated hazard ration for disease recurrence after therapy in the group of patients with positive PAIs as compared with the group of patients with negative PAIs defined by the recurrence predictor signature 3 (Table 69) was 9.046 (FIG. 58 C) (95% confidence interval of ratio, 3.022 to 76.41; P=0.001). 86% of patients with the positive PAIs had a disease recurrence within 5 years after therapy, whereas 85% of patients with the negative PAIs remained relapse-free at least 5 years (FIG. 58C). Based on this analysis, we identified the group of prostate cancer patients with positive PAIs as a poor prognosis group and the group of prostate cancer patients with negative PAIs as a good prognosis group.

Theoretically, the recurrence predictor algorithm based on a combination of signatures should be more robust than a single predictor signature, particularly during the validation analysis using an independent test cohort of patients. Next we analyzed whether a combination of the three signatures would perform in the patient's classification test with similar accuracy as the individual signatures. We found that the cut-off value of PAIs>0.2 scored in two of three individual clusters allowed to achieve the 90% recurrence prediction accuracy (Table 70). This recurrence predictor algorithm correctly identified 88% of patients with recurrent and 92% of patients with non-recurrent disease (Table 70). The Kaplan-Meier survival analysis (FIG. 58D) showed that the median relapse-free survival after therapy of patients in the poor prognosis group was 26 months. All patients in the poor prognosis group had a disease recurrence within 5 years after therapy, whereas 92% of patients in the good prognosis group remained relapse-free at least 5 years. The estimated hazard ration for disease recurrence after therapy in the poor prognosis group of patients as compared with the good prognosis group of patients defined by the recurrence predictor algorithm was 20.32 (95% confidence interval of ratio, 6.047 to 158.1; P<0.0001).

Validation of the Outcome Predictor Signatures using Independent Clinical Data Set.

To validate the potential clinical utility of identified molecular signatures, we evaluated the prognostic power of signatures applied to an independent set of 79 clinical samples obtained from 37 prostate cancer patients who developed recurrence after the therapy and 42 patients who remained disease-free. The Kaplan-Meier survival analysis demonstrated that all three recurrence predictor signatures (Table 69) segregate prostate cancer patients into sub-groups with statistically significant differences in the probability of remaining relapse-free after therapy (Table 71). Interestingly, application of the recurrence predictor algorithm (requiring a cut-off value of PAIs>0.2 scored in two of three individual clusters) appears to perform better than individual signatures in patient's stratification test using an independent data set (Table 71).

Table 71 summarizes classification of 79 prostate cancer patients who provided tumor samples. These samples comprise a signature validation (test) data set and were classified according to whether they had a good-prognosis signature or poor-prognosis signature based on PAI values defined by either individual recurrence predictor signatures or recurrence predictor algorithm that takes into account calls from all three signatures. Kaplan-Meier analysis was performed to evaluate the probability that patients would remain disease free according to whether they had a poor-prognosis or a good-prognosis signature and determine the proportion of patients who would remain disease-free at least 5 years after therapy in a poor-prognosis and a good-prognosis sub-groups. Hazard ratios, 95% confidence intervals, and P values were calculated with use of the log-rank test.

TABLE 71
Stratification of 79 prostate cancer patients into poor
and good prognosis groups at time of diagnosis
based on recurrence predictor signatures.

| | Poor | Good | | | |
|---|---|---|---|---|---|
| | prognosis, | prognosis, | | 95% | |
| Recurrence | 5-year | 5-year | Hazard | Confidence | |
| signature | survival | survival | ratio | interval of ratio | P value |
| Signature 1 | 41% | 78% | 2.858 | 1.405 to 5.143 | 0.0028 |
| Signature 2 | 44% | 79% | 3.473 | 1.584 to 5.806 | 0.0008 |
| Signature 3 | 41% | 76% | 3.351 | 1.810 to 6.907 | 0.0002 |
| Algorithm | 33% | 76% | 4.224 | 2.455 to 9.781 | <0.0001 |

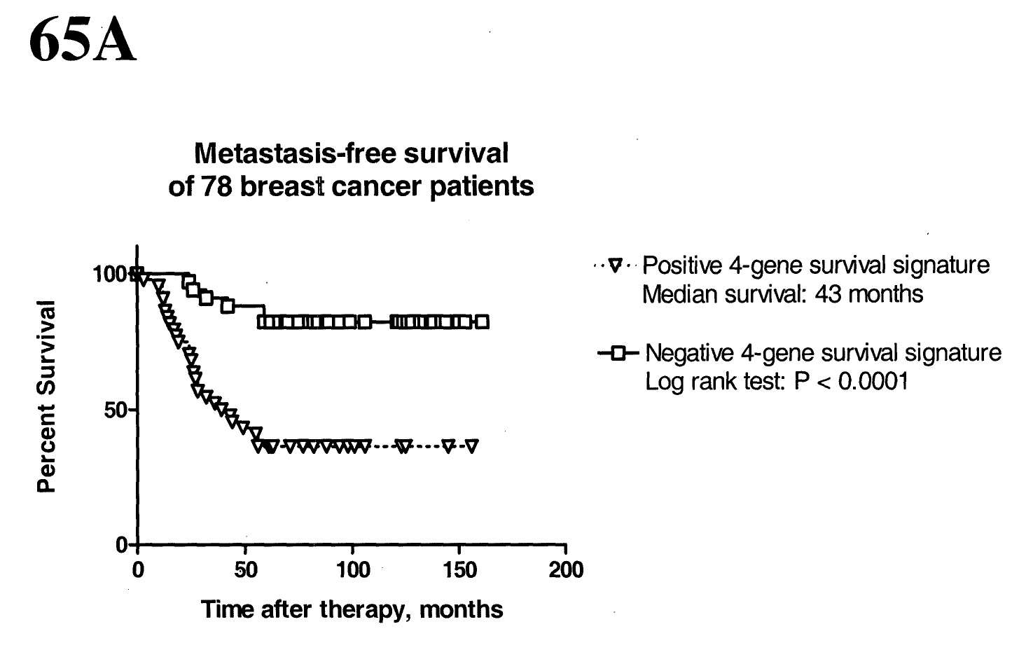

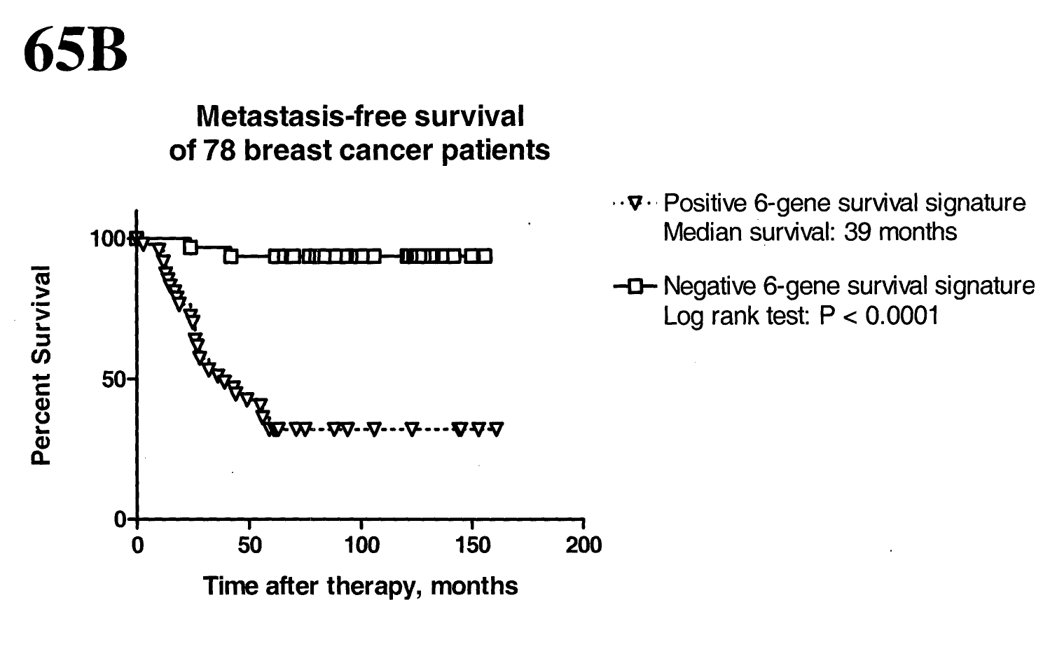

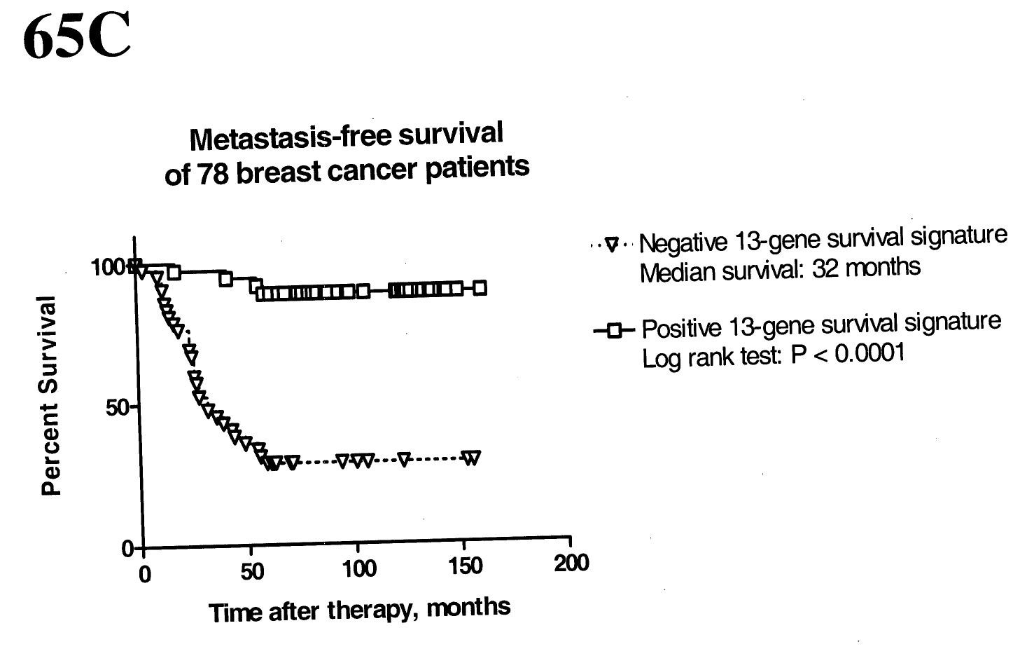

Kaplan-Meier survival analysis (FIG. 59A) showed that the median relapse-free survival after therapy of patients classified within the poor prognosis group (defined by the recurrence predictor algorithm) was 34.6 months. 67% of patients in the poor prognosis group had a disease recurrence within 5 years after therapy, whereas 76% of patients in the good prognosis group remained relapse-free at least 5 years. The estimated hazard ration for disease recurrence after therapy in the poor prognosis group as compared with the good prognosis group of patients defined by the recurrence predictor algorithm was 4.224 (95% confidence interval of ratio, 2.455 to 9.781; P<0.0001). Overall, the application of the recurrence predictor algorithm allowed accurate stratification into poor prognosis group 82% of patients who failed the therapy within one year after prostatectomy. The recurrence predictor algorithm seems to demonstrate more accurate performance in patient's classification compared to the conventional markers of outcome such as preoperative PSA level or RP Gleason sum (FIGS. 59-60 and Table 72).

Recurrence Predictor Signatures Provide Additional Predictive Value over Conventional Markers of Outcome.

Next we determined that application of the recurrence predictor signatures provides additional predictive value when combined with conventional markers of outcome such as preoperative PSA level and Gleason score. Both preoperative PSA level and RP Gleason sum were significant predictors of prostate cancer recurrence after therapy in the validation cohort of 79 patients (FIGS. 59D and 60C).

Kaplan-Meier survival analysis (FIG. 59D) showed that the median relapse-free survival after therapy of patients in the poor prognosis group defined by the high preoperative PSA level was 49.0 months. 60% of patients in the poor prognosis group had a disease recurrence within 5 years after therapy, whereas 73% of patients in the good prognosis group remained relapse-free at least 5 years. The estimated hazard ration for disease recurrence after therapy in the poor prognosis group as compared with the good prognosis group of patients defined by the preoperative PSA level was 2.551 (95% confidence interval of ratio, 1.344 to 4.895; P=0.0043). However, prediction of the outcome after therapy based on preoperative PSA level accurately stratified into the poor prognosis group only 65% of patients who failed the therapy within one year after prostatectomy (Table 72).

Table 72 shows the number of correct predictions in poor-prognosis and good-prognosis groups as a fraction of patients with the observed clinical outcome after therapy (37 patients developed relapse and 42 patients remained disease-free). PSA and Gleason sum cut-off values for segregation of poor-prognosis and good-prognosis sub-groups were defined to achieve the most accurate and statistically significant recurrence prediction in this cohort of patients. Multiparameter nomogram-based prognosis predictor was defined as described in this example's Materials & Methods using 50% relapse-free survival probability as a cut-off for patient's stratification into poor and good prognosis subgroups.

TABLE 72
Prostate cancer recurrence prediction accuracy in poor-prognosis
and good-prognosis sub-groups of patients defined by a gene
expression-based recurrence predictor algorithm alone or in
combination with established biochemical and
histopathological markers of outcome.

| Recurrence | | Non-recurrent | Year one | |
|---|---|---|---|---|
| predictor | Recurrent cancer | cancer | recurrence | Overall |
| Recurrence | 68% (25 of 37) | 81% (34 of 42) | 82% (14 of 17) | 75% (59 of |
| Algorithm | | | | 79) |
| PSA | 68% (25 of 37) | 67% (28 of 42) | 65% (11 of 17) | 67% (53 of |
| | | | | 79) |
| PSA & Algorithm | 84% (31 of 37) | 71% (30 of 42) | 88% (15 of 17) | 77% (61 of |
| | | | | 79) |
| RP Gleason sum | 38% (14 of 37) | 90% (38 of 42) | 47% (8 of 17) | 66% (52 of |
| | | | | 79) |
| RP Gleason sum & | 68% (25 of 37) | 81% (34 of 42) | 82% (14 of 17) | 75% (59 of |
| Algorithm | | | | 79) |
| PSA & RP Gleason | 81% (30 of 37) | 67% (28 of 42) | 82% (14 of 17) | 73% (58 of |
| | | | | 79) |
| Nomogram | 62% (23 of 37) | 79% (33 of 42) | 71% (12 of 17) | 71% (56 of |
| | | | | 79) |
| Nomogram & | 68% (25 of 37) | 81% (34 of 42) | 82% (14 of 17) | 75% (59 of |
| Algorithm | | | | 79) |

We next determined that application of the recurrence predictor algorithm identifies sub-groups of patients with distinct clinical outcome after therapy in both high and low PSA-expressing groups, thus adding additional predictive value to the therapy outcome classification based on preoperative PSA level alone.

In the group of patients with high preoperative PSA level (FIG. 59B), the median relapse-free survival after therapy of patients in the poor prognosis sub-group defined by the recurrence predictor algorithm was 36.2 months. 73% of patients in the poor prognosis sub-group had a disease recurrence within 5 years after therapy. Conversely, 73% of patients in the good prognosis sub-group remained relapse-free at least 5 years. The estimated hazard ration for disease recurrence after therapy in the poor prognosis sub-group as compared with the good prognosis sub-group of patients defined by the recurrence predictor algorithm was 4.315 (95% confidence interval of ratio, 1.338 to 7.025; P=0.0081).

In the group of patients with low preoperative PSA level (FIG. 59C), the median relapse-free survival after therapy of patients in the poor prognosis sub-group defined by the recurrence predictor algorithm was 42.0 months. 53% of patients in the poor prognosis sub-group had a disease recurrence within 5 years after therapy, whereas 92% of patients in the good prognosis sub-group remained relapse-free at least 5 years. The estimated hazard ration for disease recurrence after therapy in the poor prognosis sub-group as compared with the good prognosis sub-group of patients defined by the recurrence predictor algorithm was 6.247 (95% confidence interval of ratio, 2.134 to 24.48; P=0.0015). Overall, combining information from the recurrence predictor algorithm with preoperative PSA level measurement allowed 88% of patients who failed the therapy within one year after prostatectomy to be accurately classified within the poor prognosis group (Table 72).

Radical prostatectomy (“RP”) Gleason sum is a significant predictor of relapse-free survival in the validation cohort of 79 prostate cancer patients (FIG. 60C). Kaplan-Meier survival analysis (FIG. 60C) demonstrated that the median relapse-free survival after therapy of patients with the RP Gleason sum 8 & 9 was 21.0 months, thus defining the poor prognosis group based on histopathological criteria. 74% of patients in the poor prognosis group had a disease recurrence within 5 years after therapy, whereas 69% of patients in the good prognosis group (RP Gleason sum 6 & 7) remained relapse-free at least 5 years. The estimated hazard ration for disease recurrence after therapy in the poor prognosis group as compared with the good prognosis group of patients defined by the RP Gleason sum criteria was 3.335 (95% confidence interval of ratio, 2.389 to 13.70; P<0.0001). RP Gleason sum-based outcome classification accurately stratified into poor prognosis group only 47% of patients who failed the therapy within one year after prostatectomy (Table 72).

In the group of patients with RP Gleason sum 6 & 7 (FIG. 60A), the median relapse-free survival after therapy of patients in the poor prognosis sub-group defined by the recurrence predictor algorithm was 61.0 months. 53% of patients in the poor prognosis sub-group had a disease recurrence within 5 years after therapy, whereas 77% of patients in the good prognosis sub-group remained relapse-free at least 5 years. The estimated hazard ration for disease recurrence after therapy in the poor prognosis sub-group as compared with the good prognosis sub-group of patients defined by the recurrence predictor algorithm was 3.024 (95% confidence interval of ratio, 1.457 to 8.671; P=0.0055).

In the group of patients with RP Gleason sum 8 & 9 (FIG. 60B), the median relapse-free survival after therapy in the poor prognosis sub-group defined by the recurrence predictor algorithm was 11.5 months. 100% of patients in the poor prognosis sub-group had a disease recurrence within 5 years after therapy, whereas 67% of patients in the good prognosis sub-group remained relapse-free at least 5 years. The estimated hazard ration for disease recurrence after therapy in the poor prognosis sub-group as compared with the good prognosis sub-group of patients defined by the recurrence predictor algorithm was 6.143 (95% confidence interval of ratio, 1.573 to 13.49; P=0.0053). Overall, patient's classification using a combination of the recurrence predictor algorithm and RP Gleason sum allowed 82% of patients who failed the therapy within one year after prostatectomy to be accurately classified as members of the poor prognosis group (Table 72). Based on this analysis we concluded that application of the recurrence predictor algorithm provides an additional predictive value to the therapy outcome classification based on established markers of outcome.

Recurrence Predictor Signatures Provide Additional Predictive Value over Outcome Prediction Based on Multiparameter Nomogram.

Classification nomograms are generally recognized most efficient clinically useful models currently available for prediction of the probability of relapse-free survival after therapy of individual prostate cancer patients (Kattan M. W., Eastham J. A., Stapleton A. M., Wheeler T. M., Scardino P. T. A preoperative nomogram for disease recurrence following radical prostatectomy for prostate cancer. J. Natl. Cancer Inst., 90: 766-771, 1998; D'Amico A. V., Whittington R., Malkowicz S. B., Fondurulia J., Chen M-H, Kaplan I., Beard C. J., Tomaszewski J. E., Renshaw A. A., Wein A., Coleman C. N. Pretreatment nomogram for prostate-specific antigen recurrence after radical prostatectomy or external-beam radiation therapy for clinically localised prostate cancer. J. Clin. Oncol., 17: 168-172, 1999; Graefen M., Noldus J., Pichlmeier A., Haese P., Hammerer S., Fernandez S., Conrad R., Henke E., Huland E., Huland H. Early prostate-specific antigen relapse after radical retropubic prostatectomy: prediction on the basis of preoperative and postoperative tumor characteristics. Eur. Urol., 36: 21-30, 1999; Kattan M. W., Wheeler T. M., Scardino P. T. Postoperative nomogram for disease recurrence after radical prostatectomy for prostate cancer. J. Clin. Oncol., 17: 1499-1507, 1999.). We applied the Kattan nomogram utilizing multiple postoperative parameters (Kattan, et al. (1999)) for prognosis prediction classification in the test group of 79 prostate cancer patients.

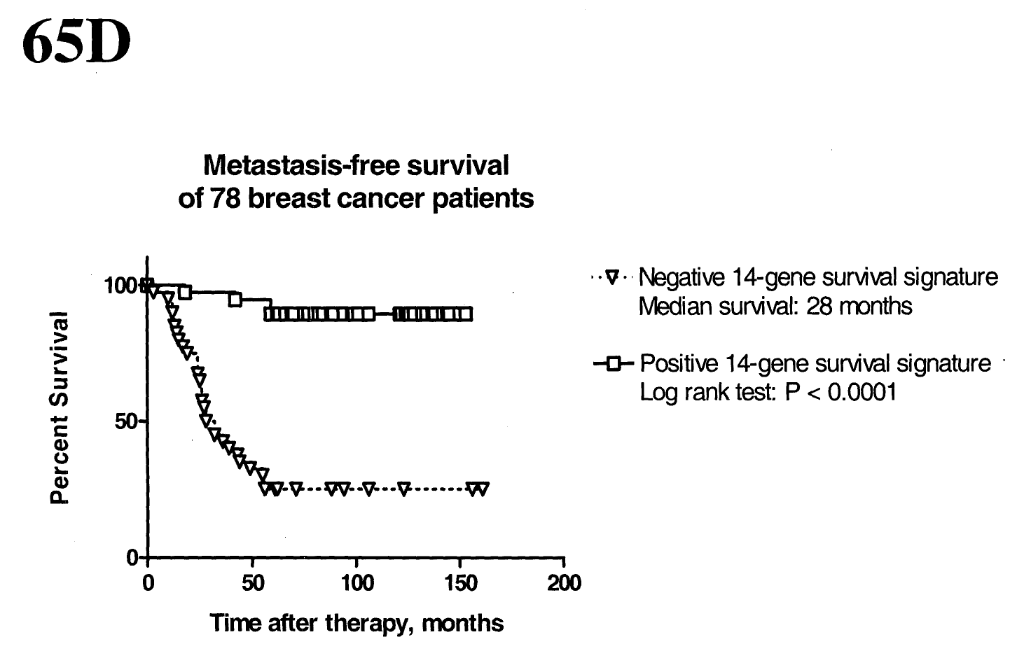

Kaplan-Meier survival analysis (FIG. 61A) showed that the median relapse-free survival after therapy of patients in the poor prognosis group defined by the Kattan nomogram was 33.1 months. 72% of patients in the poor prognosis group had a disease recurrence within 5 years after therapy, whereas 81% of patients in the good prognosis group remained relapse-free at least 5 years. The estimated hazard ration for disease recurrence after therapy in the poor prognosis group as compared with the good prognosis group of patients defined by the Kattan nomogram was 3.757 (95% confidence interval of ratio, 2.318 to 9.647; P<0.0001). Prediction of the outcome after therapy based on Kattan nomogram accurately stratified into poor prognosis group 71% of patients who failed the therapy within one year after prostatectomy (Table 72).

Application of the recurrence predictor algorithm identified sub-groups of patients with distinct clinical outcome after therapy in both poor and good prognosis groups defined by the Kattan nomogram, thus adding additional predictive value to the therapy outcome classification based on nomogram alone.

In the poor prognosis group of patients defined by the Kattan nomogram the application of the recurrence predictor algorithm appears to identify two sub-groups of patients with statistically significant difference in the probability to remain relapse-free after therapy (FIG. 61B). Median relapse-free survival after therapy of patients in the poor prognosis sub-group defined by the recurrence predictor algorithm was 11.5 months compared to median relapse-free survival of 71.1 months in the good prognosis sub-group (FIG. 61B). 89% of patients in the poor prognosis sub-group had a disease recurrence within 5 years after therapy. Conversely, 50% of patients in the good prognosis sub-group remained relapse-free at least 5 years. The estimated hazard ration for disease recurrence after therapy in the poor prognosis sub-group as compared with the good prognosis sub-group of patients defined by the recurrence predictor algorithm was 3.129 (95% confidence interval of ratio, 1.378 to 7.434; P=0.0068).

Similarly, in the good prognosis group of patients identified based on application of the Kattan nomogram, the recurrence predictor algorithm seems to define two sub-groups of patients with statistically significant difference in the probability to remain relapse-free after therapy (FIG. 61C). Median relapse-free survival after therapy of patients in the poor prognosis sub-group defined by the recurrence predictor algorithm was 64.8 months. 41% of patients in the poor prognosis sub-group had a disease recurrence within 5 years after therapy. Conversely, 87% of patients in the good prognosis sub-group remained relapse-free at least 5 years. The estimated hazard ration for disease recurrence after therapy in the poor prognosis sub-group as compared with the good prognosis sub-group of patients defined by the recurrence predictor algorithm was 4.398 (95% confidence interval of ratio, 1.767 to 18.00; P=0.0035). Overall, combination of the recurrence predictor algorithm and Kattan nomogram allowed accurate stratification into poor prognosis group 82% of patients who failed the therapy within one year after prostatectomy (Table 72).

Recurrence Predictor Algorithm Defines Poor and Good Prognosis Sub-Groups of Patients Diagnosed with the Early Stage Prostate Cancer.

Identification of sub-groups of patients with distinct clinical outcome after therapy would be particularly desirable in a cohort of patients diagnosed with the early stage prostate cancer. Next we determined that recurrence predictor signatures are useful in defining sub-groups of patients diagnosed with early stage prostate cancer and having a statistically significant difference in the likelihood of disease relapse after therapy.

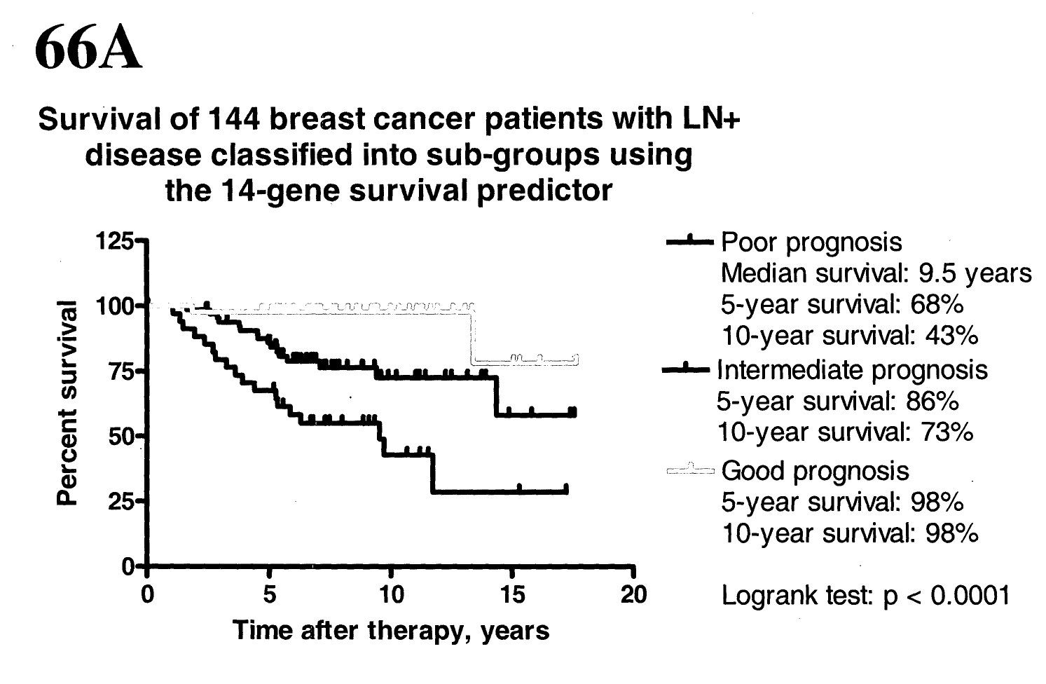

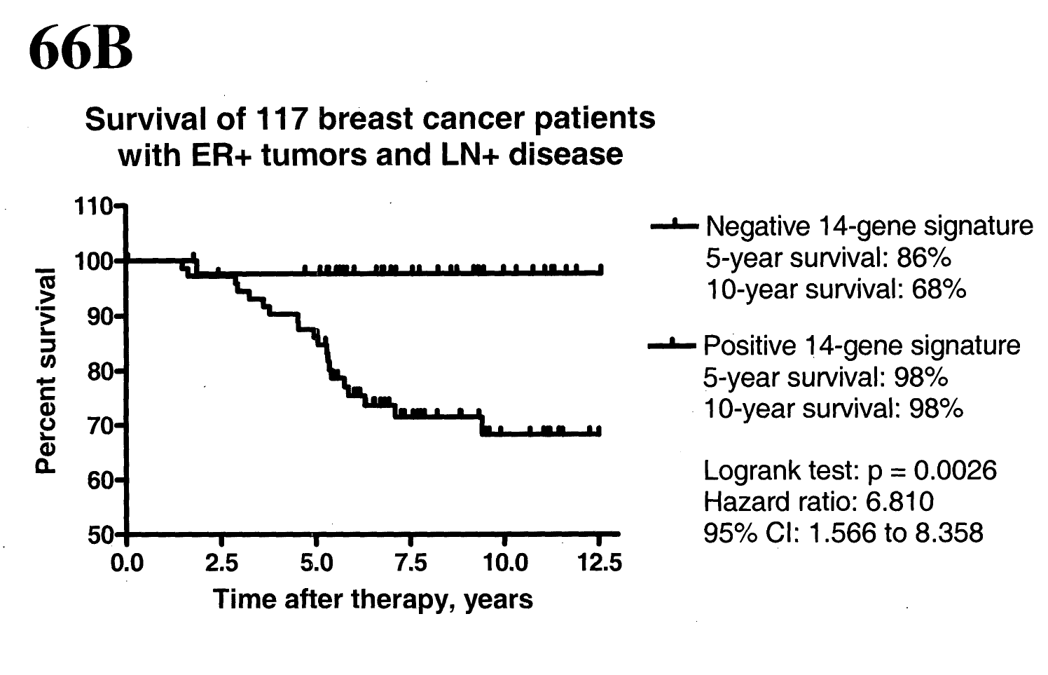

In the group of patients diagnosed with the stage 1C prostate cancer (FIG. 62A), the median relapse-free survival after therapy in the poor prognosis sub-group defined by the recurrence predictor algorithm was 12 months. In contrast, the median relapse-free survival after therapy in the good prognosis group was 82.4 months. 77% of patients in the poor prognosis sub-group had a disease recurrence within 5 years after therapy. Conversely, 81% of patients in the good prognosis sub-group remained relapse-free at least 5 years. The estimated hazard ration for disease recurrence after therapy in the poor prognosis sub-group as compared with the good prognosis sub-group of patients defined by the recurrence predictor algorithm was 5.559 (95% confidence interval of ratio, 2.685 to 25.18; P=0.0002).

In the group of patients diagnosed with the stage 2A prostate cancer (FIG. 62B), the median relapse-free survival after therapy in the poor prognosis sub-group defined by the recurrence predictor algorithm was 35.4 months. 86% of patients in the poor prognosis sub-group had a disease recurrence within 5 years after therapy, whereas 78% of patients in the good prognosis sub-group remained relapse-free at least 5 years. The estimated hazard ratio for disease recurrence after therapy in the poor prognosis sub-group as compared with the good prognosis sub-group of patients defined by the recurrence predictor algorithm was 7.411 (95% confidence interval of ratio, 2.220 to 40.20; P=0.0024). Based on this analysis we concluded that application of the recurrence predictor algorithm seems to provide potentially useful clinical information in stratification of patients diagnosed with the early stage prostate cancer into sub-groups with statistically significant difference in the likelihood of disease recurrence after therapy.

Discussion

As a result of the broad application of measurements of PSA level in the blood for early detection of prostate cancer in the United States, an increasing proportion of prostate cancer patients are diagnosed with early-stage tumors that apparently confined to the prostate gland and many patients have seemingly indolent disease not affecting individual's survival (Potosky, A., Feuer, E., Levin, D. Impact of screening on incidence and mortality of prostate cancer in the United States. Epidemiol. Rev., 23: 181-186, 2001). The considerable clinical heterogeneity of the early stage prostate cancer represents a highly significant health care and socio-economic challenge because prostate cancer is expected to be diagnosed in ˜200,000 individuals every year (Greenlee, R. T., Hill-Hamon, M. B., Murray, T., Thun, M. Cancer statistics, 2001. CA Cancer J. Clin., 51: 15-36, 2001). Consequently, it can be argued that, unlike other types of cancer, development of efficient prognostic tests rather than early detection is critical for improvement of clinical decision-making and management of prostate cancer.

We hypothesized that clinically relevant genetic signatures can be found by searching for clusters of co-regulated genes that display highly concordant transcript abundance behavior across multiple experimental models and clinical settings that model or represent malignant phenotypes of interest (Glinsky, G. V., Krones-Herzig, A., Glinskii, A. B., Gebauer, G. Microarray analysis of xenograft-derived cancer cell lines representing multiple experimental models of human prostate cancer. Molecular Carcinogenesis, 37: 209-221, 2003; Glinsky, G. V., Krones-Herzig, A., Glinskii, A. B. Malignancy-associated regions of transcriptional activation: gene expression profiling identifies common chromosomal regions of a recurrent transcriptional activation in human prostate, breast, ovarian, and colon cancers. Neoplasia, 5: 21-228; Glinsky, G. V., Ivanova, Y. A., Glinskii, A. B. Common malignancy-associated regions of transcriptional activation (MARTA) in human prostate, breast, ovarian, and colon cancers are targets for DNA amplification. Cancer Letters, in press, 2003). Thus, according to this model the primary criterion in a transcript selection process should be the concordance of changes in expression rather the magnitude of changes (e.g., fold change). One of the predictions of this model is that transcripts of interest are expected to have a tightly controlled “rank order” of expression within a cluster of co-regulated genes reflecting a balance of up- and down-regulated mRNAs as a desired regulatory end-point in a cell. A degree of resemblance of the transcript abundance rank order within a gene cluster between a test sample and reference standard is measured by a Pearson correlation coefficient and designated a phenotype association index (“PAI”).

Using this strategy we discovered and validated a prostate cancer recurrence predictor algorithm that is suitable for stratifying patients at the time of diagnosis into poor and good prognosis sub-groups with statistically significant differences in the disease-free survival after therapy. The algorithm is based on application of gene expression signatures associated with biochemical recurrence of prostate cancer. The signatures (Table 69) were defined using clusters of co-regulated genes exhibiting highly concordant expression profiles (r>0.95) in metastatic nude mouse models of human prostate carcinoma and tumor samples from patients with recurrent prostate cancer (see Example 5).

A few previous studies have applied oligonucleotide or cDNA microarrays for identification of gene expression signatures associated with biochemical recurrence of human prostate cancer (Dhanasekaran, S. M., Barrette, T. R., Ghosh, D., Shah, R., Varambally, S., Kurachi, K., Pienta, K. J., Rubin, M. A., Chinnalyan, A. M. Delineation of prognostic biomarkers in prostate cancer. Nature, 412:822-826, 2001; Singh, D., Febbo, P. G., Ross, K., Jackson, D. G., Manola, C. L., Tamayo, P., Renshaw, A. A., D'Amico, A. V., Richie, J. P., Lander, E. S., Loda, M., Kantoff, P. W., Golub, T. R., Sellers, W. R. Gene expression correlates of clinical prostate cancer behavior. Cancer Cell, 1: 203-209, 2002; Varambally, S., Dhanasekaran, S. M., Zhou, M., Barrette, T. R., Kumar-Sinha, C., Sanda, M. G., Ghosh, D., Pineta, K. J., Sewalt, R. G., Otte, A. P., Rubin, M. A., Chinnalyan, A. M. The polycomb group protein EZH2 is involved in progression of prostate cancer. Nature, 419: 624-629, 2002; Henshall, S. M., Afar, D. E., Hiller, J., Horvath, L. G., Quinn, D. I., Rasiah, K. K., Gish, K., Willhite, D., Kench, J. G., Gardiner-Garden, M., Stricker, P. D., Scher, H. I., Grygiel, J. J., Agus, D. B., Mack, D. H., Sutherland, R. L. Survival analysis of genome-wide gene expression profiles of prostate cancers identifies new prognostic targets of disease relapse. Cancer Res., 63: 4196-4203, 2003). One of the major deficiencies of these studies that somewhat limited their significance was that a single clinical data set was utilized for both signature discovery and validation. To our knowledge, the work reported here is the first genome-wide expression profiling study of human prostate cancer that utilizes one clinical data set for signature discovery and algorithm development, and a second independent data set for validation of the prostate cancer recurrence predictor algorithm.

One of the interesting features of described here prostate cancer recurrence predictor algorithm is that it provides additional predictive value over conventional markers of outcome such as pre-operative PSA level and Gleason sum. Another important feature of identified recurrence predictor algorithm is its ability to stratify patients diagnosed with the early stage prostate cancer into sub-groups with statistically-distinct likelihoods of biochemical relapse after therapy. Importantly, the recurrence predictor algorithm segregates into poor prognosis group 88% of patients who subsequently developed disease recurrence within one year after prostatectomy. Based on this analysis we concluded that identified in this study genetic signatures (as well as others that can be determined using the methods of the invention) have a significant potential for developing highly accurate clinical prognostic tests suitable for stratifying prostate cancer patients at the time of diagnosis with respect to likelihood of negative or positive clinical outcome after therapy.

The causal genetic, molecular, and biological distinctions between prostate tumors with recurrent and indolent clinical behavior remain largely unknown. The results reported in this example and in Example 5 provide the first experimental evidence of a clinically relevant transcriptional resemblance between metastatic human prostate carcinoma xenografts growing orthotopically in nude mice and primary prostate tumors from patients that subsequently developed a biochemical relapse after therapy. This work provides a model for investigation of the potential functional relevance of identified transcriptional aberrations and suggests that genetically defined metastasis-promoting features of primary tumors seem to be one of the major contributing factors of aggressive clinical behavior and unfavorable prognosis in prostate cancer patients. This conclusion is consistent with results of the several recent studies aimed at definition of metastasis predictor signatures in the primary human tumors representing multiple types of epithelial cancers (van't Veer, L. J., Dai, H., van de Vijver, M. J., et al. Gene expression profiling predicts clinical outcome of breast cancer. Nature, 415: 530-536, 2002; van de Vijver, M. J., He, Y. D., van't Veer, L. J., et al. A gene expression signature as a predictor of survival in breast cancer. N. Engl. J. Med., 347: 1999-2009, 2002; Ramaswamy, S., Ross, K. N., Lander, E. S., Golub, T. R. A molecular signature of metastasis in primary solid tumors. Nature Genetics, 33: 49-54, 2003). Our results indicate that sub-groups of prostate cancer patients with poor and good prognosis gene expression signatures reflect the presence of two genetically defined sub-types of human prostate carcinoma manifesting dramatic statistically significant differences in response to therapy and clinically distinct courses of disease progression.

One of the dominant views on prostate cancer pathogenesis is the concept of progression from hormone-dependent early stage prostate cancer to hormone-refractory metastatic late stage disease with the apparent implication of increased proportion of patients with poor prognosis at the advanced stage of progression. However, in our validation data set of 79 samples the actual frequency of recurrence remains relatively constant among the patients with different stages of prostate cancer: 47% (16 of 34) in stage 1C; 56% (9 of 16) in stage 2A; and 41% (12 of 29) in stages 2B/2C/3A. These data suggest that progression of the disease occurs only in a sub-group of patients. Interestingly, in a sub-group of patients with good prognosis signatures the frequency of recurrence appears to increase in the patients with the late-stage prostate cancer: 24% (5 of 21) in stage 1C; 22% (2 of 9) in stage 2A; 33% (3 of 9) in stage 2B; 40% (2 of 5) in stage 2C/3A. These results seem to imply that patients with the good prognosis signatures may represent a sub-group undergoing a classical prostate cancer progression with a gradual increase in malignant potential. The patients with poor prognosis signatures may represent a genetically and biologically distinct sub-type of prostate cancer exhibiting highly malignant behavior at the early stage of disease with the frequency of recurrence 85% (11 of 13) in stage 1C and 100% (7 of 7) in stage 2A patients.

In summary, using expression profiles of highly metastatic models of human prostate cancer in nude mice as a predictive reference of expected transcript abundance behavior in recurrent prostate tumors, we identified and validated recurrence predictor signatures of human prostate cancer. Prostate cancer recurrence predictor signatures provide additional predictive value to the conventional markers of outcome and will be clinically useful in stratifying prostate cancer patients into sub-groups with distinct clinical manifestation of disease and different response to therapy.

References

1. Holmberg, L., Bill-Axelson, A., Helgesen, F., Salo, J. O., Folmerz, P., Haggman, M., Andersson, S. O., Sapngberg, A., Busch, C., Nording, S., et al. 2002. N. Engl. J. Med. 347, 781-789.

2. Thomas, G. V., and Loda, M. 2002. Molecular staging of prostate cancer. In Prostate Cancer Principles & Practice. P. W. Kantoff, P. R. Carroll, and A. V. D'Amico, eds. (Philadelphia: Lippincott Williams & Wilkins), pp. 287-303.

3. DeMarzo, A. M., Nelson, W. G., Isaacs, W. B., Epstein, J. I. 2003. Pathological and molecular aspects of prostate cancer. Lancet, 361: 955-964.

4. Kattan M. W., Eastham J. A., Stapleton A. M., Wheeler T. M., Scardino P. T. A preoperative nomogram for disease recurrence following radical prostatectomy for prostate cancer. J. Natl. Cancer Inst., 90: 766-771, 1998.

5. D'Amico A. V., Whittington R., Malkowicz S. B., Fondurulia J., Chen M-H, Kaplan I., Beard C. J., Tomaszewski J. E., Renshaw A. A., Wein A., Coleman C. N. Pretreatment nomogram for prostate-specific antigen recurrence after radical prostatectomy or external-beam radiation therapy for clinically localised prostate cancer. J. Clin. Oncol., 17: 168-172, 1999.

6. Graefen M., Noldus J., Pichlmeier A., Haese P., Hammerer S., Fernandez S., Conrad R., Henke E., Huland E., Huland H. Early prostate-specific antigen relapse after radical retropubic prostatectomy: prediction on the basis of preoperative and postoperative tumor characteristics. Eur. Urol., 36: 21-30, 1999.

7. Kattan M. W., Wheeler T. M., Scardino P. T. Postoperative nomogram for disease recurrence after radical prostatectomy for prostate cancer. J. Clin. Oncol., 17: 1499-1507, 1999.

8. Magee, J. A., Araki, T., Patil, S., Ehrig, T., True, L., Humphrey, P. A., Catalona, W. J., Watson, M. A., Milbrandt, J. Expression profiling reveals hepsin overexpression in prostate cancer. Cancer Res., 61: 5692-5696, 2001.

9. Dhanasekaran, S. M., Barrette, T. R., Ghosh, D., Shah, R., Varambally, S., Kurachi, K., Pienta, K. J., Rubin, M. A., Chinnalyan, A. M. Delineation of prognostic biomarkers in prostate cancer. Nature, 412:822-826, 2001.

10. Welsh, J. B., Sapinoso, L. M., Su, A. I., Kern, S. G., Wang-Rodriguez, J., Moskaluk, C. A., Frierson, H. F., Jr., Hampton, G. M. Analysis of gene expression identifies candidate markers and pharmacological targets in prostate cancer. Cancer Res., 61: 5974-5978, 2001.

11. Luo, J., Duggan, D. J., Chen, Y., Sauvageot, J., Ewing, C. M., Bittner, M. L., Trent, J. M., Isaacs, W. B. Human prostate cancer and benign prostatic hyperplasia: molecular dissection by gene expression profiling. Cancer Res., 61: 4683-4688, 2001.

12. Stamey, T A, Warrington, J A, Caldwell, M C, Chen, Z, Fan, Z, Mahadevappa, M, McNeal, J E, Nolley, R, Zhang, Z. Molecular genetic profiling of Gleason grade 4/5 prostate cancers compared to benign prostatic hyperplasia. J. Urol., 166: 2171-2177, 2001.

13. Luo, J., Dunn, T, Ewing, C, Sauvageot, J., Chen, Y, Trent, J, Isaacs, W. Gene expression signature of benign prostatic hyperplasia revealed by cDNA microarray analysis. Prostate, 51: 189-200, 2002.

14. Singh, D., Febbo, P. G., Ross, K., Jackson, D. G., Manola, C. L., Tamayo, P., Renshaw, A. A., D'Amico, A. V., Richie, J. P., Lander, E. S., Loda, M., Kantoff, P. W., Golub, T. R., Sellers, W. R. Gene expression correlates of clinical prostate cancer behavior. Cancer Cell, 1: 203-209, 2002.

15. Rhodes, D. R., Barrette, T. R., Rubin, M. A., Ghosh, D., Chinnaiyan, A. M. Meta-analysis of microarrays: interstudy validation of gene expression profiles reveals pathways dysregulation in prostate cancer. Cancer Res., 62: 4427-4433, 2002.

16. Varambally, S., Dhanasekaran, S. M., Zhou, M., Barrette, T. R., Kumar-Sinha, C., Sanda, M. G., Ghosh, D., Pineta, K. J., Sewalt, R. G., Otte, A. P., Rubin, M. A., Chinnalyan, A. M. The polycomb group protein EZH2 is involved in progression of prostate cancer. Nature, 419: 624-629, 2002.

17. Henshall, S. M., Afar, D. E., Hiller, J., Horvath, L. G., Quinn, D. I., Rasiah, K. K., Gish, K., Willhite, D., Kench, J. G., Gardiner-Garden, M., Stricker, P. D., Scher, H. I., Grygiel, J. J., Agus, D. B., Mack, D. H., Sutherland, R. L. Survival analysis of genome-wide gene expression profiles of prostate cancers identifies new prognostic targets of disease relapse. Cancer Res., 63: 4196-4203, 2003.

18. LaTulippe, E., Satagopan, J., Smith, A., Scher, H., Scardino, P., Reuter, V., Gerald, W. L. Comprehensive gene expression analysis of prostate cancer reveals distinct transcriptional programs associated with metastasis. Cancer Res., 62: 4499-4506, 2002.

19. Glinsky, G. V., Krones-Herzig, A., Glinskii, A. B., Gebauer, G. Microarray analysis of xenograft-derived cancer cell lines representing multiple experimental models of human prostate cancer. Molecular Carcinogenesis, 37: 209-221, 2003.

20. Potosky, A., Feuer, E., Levin, D. Impact of screening on incidence and mortality of prostate cancer in the United States. Epidemiol. Rev., 23: 181-186, 2001.

21. Greenlee, R. T., Hill-Hamon, M. B., Murray, T., Thun, M. Cancer statistics, 2001. CA Cancer J. Clin., 51: 15-36, 2001.

22. Glinsky, G. V., Krones-Herzig, A., Glinskii, A. B. Malignancy-associated regions of transcriptional activation: gene expression profiling identifies common chromosomal regions of a recurrent transcriptional activation in human prostate, breast, ovarian, and colon cancers. Neoplasia, 5: 21-228.

23. Glinsky, G. V., Ivanova, Y. A., Glinskii, A. B. Common malignancy-associated regions of transcriptional activation (MARTA) in human prostate, breast, ovarian, and colon cancers are targets for DNA amplification. Cancer Letters, in press, 2003.

24. van't Veer, L. J., Dai, H., van de Vijver, M. J., et al. Gene expression profiling predicts clinical outcome of breast cancer. Nature, 415: 530-536, 2002

25. van de Vijver, M. J., He, Y. D., van't Veer, L. J., et al. A gene expression signature as a predictor of survival in breast cancer. N. Engl. J. Med., 347: 1999-2009, 2002.

26. Ramaswamy, S., Ross, K. N., Lander, E. S., Golub, T. R. A molecular signature of metastasis in primary solid tumors. Nature Genetics, 33: 49-54, 2003.

### EXAMPLE 12

### Use of Expression Data with Other Metrics to Predict Breast Cancer Patient Survival

Introduction

Highly accurate prognostic tests are essential for individualized decision-making process during clinical management of cancer patients leading to rational and more efficient selection of appropriate therapeutic interventions and improved outcome after therapy. In breast cancer, patients are classified into broad subgroups with poor and good prognosis reflecting a different probability of disease recurrence and survival after therapy. Distinct prognostic subgroups are identified using a combination of clinical and pathological criteria: age, primary tumor size, status of axillary lymph nodes, histologic type and pathologic grade of tumor, and hormone receptor status (Goldhirsch, A., Glick, J. H., Gelber, R. D., Coates, A. S., Seen, H. J. Meeting highlights: International Consensus Panel on the Treatment of Primary Breast Cancer: Seventh International Conference on Adjuvant Therapy of Primary Breast Cancer. J. Clin. Oncol., 19: 3817-3827, 2001; Eifel, P., Axelson, J. A., Costa, J., et al. National Institute of Health Consensus Development Conference Summary: adjuvant therapy for breast cancer, Nov. 1-3, 2000. J. Natl. Cancer Inst., 93: 979-989, 2001.)

One of the most critical treatment decisions during the clinical management of breast cancer patients is the use of adjuvant systemic therapy. Adjuvant systemic therapy significantly improves disease-free and overall survival in breast cancer patients with both lymph-node negative and lymph-node positive disease (Early Breast Cancer Trialists' Collaborative Group. Polychemotherapy for early breast cancer: an overview of the randomized trials. Lancet, 352: 930-942, 1998; Early Breast Cancer Trialists' Collaborative Group. Tamoxifen for early breast cancer: an overview of the randomized trials. Lancet, 351: 1451-1467, 1998). It is generally accepted that breast cancer patients with poor prognosis would gain the most benefits from the adjuvant systemic therapy (Goldhirsch, et al., 2001; Eifel et al., 2001).

Diagnosis of lymph-node status is important in therapeutic decision-making, prediction of disease outcome, and probability of breast cancer recurrence. Invasion into axillary lymph nodes is recognized as one of the most important prognostic factors (Krag, D., Weaver, D., Ashikaga, T., et al. The sentinel node in breast cancer—a multicenter validation study. N. Engl. J. Med., 339: 941-946, 1998; Singletary, S. E., Allred, C., Ashley, P., et al. Revision of the American Joint Committee on cancer staging system for breast cancer. J. Clin. Oncol., 20: 3628-3636, 2002; Jatoli, I., Hilsenbeck, S. G., Clark, G. M., Osborne, C. K. Significance of axillary lymph node metastasis in primary breast cancer. J. Clin. Oncol., 17: 2334-2340, 1999). Most patients diagnosed with lymph-node negative breast cancer can be effectively treated with surgery and local radiation therapy. However, results of several studies show that 22-33% of breast cancer patients with no detectable lymph-node involvement and classified into a good prognosis subgroup develop recurrence of disease after a 10-year follow-up (Early Breast Cancer Trialists' Collaborative Group. Tamoxifen for early breast cancer: an overview of the randomized trials. Lancet, 351: 1451-1467, 1998). Therefore, accurate identification of breast cancer patients with lymph-node negative tumors who are at high risk of recurrence is critically important for rational treatment decision and improved clinical outcome in the individual patient.

Microarray-based gene expression profiling of human cancers rapidly emerged as a new powerful screening technique generating hundreds of novel diagnostic, prognostic, and therapeutic targets (Golub, T. R., Slonim, D. K., Tamayo, P., et al. Molecular classification of cancer: class discovery and class prediction by gene expression monitoring. Science, 286: 531-537, 1999; Alizadeh, A. A., Eisen, M. B., Davis, R. E., et al. Distinct types of diffuse large B-cell lymphoma identified by gene expression profiling. Nature, 403: 503-511, 2000; Alizadeh, A. A., Ross, D. T., Perou, C. M., van de Rijn, M. Towards a novel classification of human malignancies based on gene expression patterns. J. Pathol., 195: 41-52, 2001; Battacharjee, A., Richards, W. G., Staunton, J., et al. Classification of human lung carcinomas by mRNA expression profiling reveals distinct adenocarcinoma subclasses. Proc. Natl. Acad. Sci. USA, 98: 13790-13795, 2001; Yeoh, E.-J., Ross, M. E., Shurtleff, S. A., et al. Classification, subtype discovery, and prediction of outcome in pediatric acute lymphoblastic leukemia by gene expression profiling. Cancer Cell, 1: 133-143, 2002; Dyrskot, L., Thykjaer, T., Kruhoffer, M., Jensen, J. L., Marcussen, N., Hamilton-Dutoit, S., Wolf, H., Orntoft, T. Identifying distinct classes of bladder carcinoma using microarrays. Nature Genetics, 33: 90-96, 2003). Recently breast cancer gene expression signatures have been identified that are associated with the estrogen receptor and lymph node status of patients and can aid in classification of breast cancer patients into subgroups with different clinical outcome after therapy (Perou, C. M., Sorlie, T., Eisen, M. B., et al. Molecular portrait of human breast tumors. Nature, 406: 747-752, 2000; Gruvberger, S., Ringner, M., Chen, Y., et al. Estrogen receptor status in breast cancer is associated with remarkably distinct gene expression patterns. Cancer Res., 61: 5979-5984, 2001; West, M., Blanchette, C., Dressman, H., et al. Predicting the clinical status of human breast cancer by using gene expression profiles. Proc. Natl. Acad. Sci. USA, 98: 11462-11467, 2001; Ahr, A., Karn, T., Sollbach, C., et al. Identification of high risk breast cancer patients by gene expression profiling. Lancet, 359: 131-132, 2002; van't Veer, L. J., dai, H., van de Vijver, M. J., et al. Gene expression profiling predicts clinical outcome of breast cancer. Nature, 415: 530-536, 2002; Sorlie, T., Perou, C. M., Tibshirani, R., et al. Gene expression patterns of breast carcinomas distinguish tumor subclasses with clinical implications. Proc. Natl. Acad. Sci. USA, 98: 10869-10874, 2001; Heedenfalk, I., Duggan, D., Chen, Y., et al. Gene-expression profiles in hereditary breast cancer. N. Engl. J. Med., 344: 539-548, 2001; van de Vijver, M. J., He, Y. D., van't Veer, L. J., et al. A gene expression signature as a predictor of survival in breast cancer. N. Engl. J. Med., 347: 1999-2009, 2002; Huang, E., Cheng, S. H., Dressman, H., Pittman, J., Tsou, M. H., Horng, C. F., Bild, A., Iversen, E. S., Liao, M., Chen, C. M., West, M., Nevins, J. R., Huang, A. T. Gene expression predictors of breast cancer outcome. Lancet, 361: 1590-1596, 2003).

One of the significant limitations of these array-based studies is that they generated vast data sets comprising many attractive targets with diagnostic and prognostic potential. Design and performance of meaningful follow-up experiments such as translation of the array-generated hits into quantitative RT-PCR-based analytical assays would require a significant data reduction. Furthermore, clinical implementation of novel prognostic tests would require integration of genomic data and best-established conventional markers of the outcome.

Here, we translate a large microarray-based breast cancer outcome predictor signature into quantitative RT-PCR-based assays of mRNA abundance levels of small gene clusters performing with similar classification accuracy. We demonstrate that identified molecular signatures provide additional predictive values over well-established conventional prognostic markers for breast cancer such as hormone receptor status and lymph node involvement. These data indicate that quantitative laboratory tests measuring expression profiles of identified small gene clusters are useful for stratifying breast cancer patients into sub-groups with distinct likelihood of positive outcome after therapy and assisting in selection of optimal treatment strategies.

Materials and Methods

The same general methods as described in Example 11 were used to carry out the work reported in this example.

Results and Discussion

The 70-gene Breast Cancer Metastasis and Survival Predictor Signature Represents a Heterogeneous Set of Small Gene Clusters Independently Performing with High Therapy Outcome Prediction Accuracy.

Recent study on gene expression profiling of breast cancer identifies 70 genes whose expression pattern is strongly predictive of a short post-diagnosis and treatment interval to distant metastases (van't Veer, et al., 2002). The expression pattern of these 70 genes discriminates with 81% (optimized sensitivity threshold) or 83% (optimal accuracy threshold) accuracy the patient's prognosis in the group of 78 young women diagnosed with sporadic lymph-node-negative breast cancer (this group comprises of 34 patients who developed distant metastases within 5 years and 44 patients who continued to be disease-free at least 5 years after therapy; they constitute clinically defined poor prognosis and good prognosis groups, correspondingly). We reduced the number of genes whose expression patterns represent genetic signatures of breast cancer with “poor prognosis” or “good prognosis.” Measurements of mRNA expression levels of 70 genes in established human breast carcinoma cell lines (MCF7; MDA-MB-435; MDA-MB-468; MDA-MB-231; MDA-MB-435Br1; MDA-MB-435BL3) and primary cultures of normal human breast epithelial cells were performed utilizing Q-RT-PCR method, which is generally accepted as the most reliable method of gene expression analysis and unambiguous confirmation of a gene identity. For each breast cancer cell line concordant sets of genes were identified exhibiting both positive and negative correlation between fold expression changes in cancer cell lines versus control cell line and poor prognosis group versus good prognosis group patient samples. Minimum segregation sets were selected from corresponding concordance sets and individual phenotype association indices were calculated. The four top-performing breast cancer metastasis predictor gene clusters are listed in Table 73.

A breast cancer poor prognosis predictor cluster comprising 6 genes was identified (r=0.981) using MDA-MB-468 cell line gene expression profile as a reference standard. 32 of 34 samples from the poor prognosis group had positive phenotype association indices, whereas 29 of 44 samples from the good prognosis group had negative phenotype association indices yielding 78% overall accuracy in sample classification. Another breast cancer poor prognosis predictor cluster comprising 4 genes was identified (r=0.944) using MDA-MB-435BL3 cell line gene expression profile as a reference standard. Using this 4-gene cluster, 28 of 34 samples from the poor prognosis group had positive phenotype association indices, whereas 28 of 44 samples from the good prognosis group had negative phenotype association indices overall yielding 72% accuracy in sample classification.

A breast cancer good prognosis predictor cluster comprising 14 genes was identified (r=−0.952) using MDA-MB-435Br1 cell line gene expression profile as a reference standard. 30 of 34 samples from the poor prognosis group had negative phenotype association indices, whereas 34 of 44 samples from the good prognosis group had positive phenotype association indices yielding 82% overall accuracy in sample classification. Another breast cancer good prognosis predictor cluster comprising 13 genes (r=−0.992) was identified using MCF7 cell line gene expression profile as a reference standard. 30 of 34 samples from the poor prognosis group had negative phenotype association indices, whereas 32 of 44 samples from the good prognosis group had positive phenotype association indices yielding 79% overall accuracy in sample classification.

The transcripts comprising each signature listed in Table 73 were selected based on Pearson correlation coefficients (r>0.95) reflecting a degree of similarity of expression profiles in clinical tumor samples (34 recurrent versus 44 non-recurrent tumors) and experimental cell line samples. Selection of transcripts was performed from sets of genes exhibiting concordant changes of transcript abundance behavior in recurrent versus non-recurrent clinical tumor samples (70 transcripts) and experimental conditions independently defined for each signature (6-gene signature: MDA-MB468 cells versus control; 4-gene signature: MDA-MB-435BL3 cells versus control; 13-gene signature: MCF7 cells versus control; 14-gene signature: MDA-MB-435Br1 cells versus control) (see also Example 2). mRNA expression levels of 70 genes comprising parent microarray-defined signature (van't Veer, L. J., et al., 2002; van de Vijver, M. J., et al., 2002) were measured by standard quantitative RT-PCR method in multiple established human breast cancer cell lines using GAPDH expression for normalization and compared to the expression in a control cell line. Control cells were primary cultures of normal human breast epithelial cells. Expression profiles were presented as log10 average fold changes for each transcript.

TABLE 73
Gene expression signatures predicting survival of breast cancer patients.

| | | | |
|---|---|---|---|
| LocusLink | | Gene ID (Chip identified in | UniGene |
| Name | Description | van't Veer, L. J., et al., 2002) | ID |

6-gene signature (same as Table 27)

| | | | |
|---|---|---|---|
| FLT1 | Fms-related tyrosine | NM_002019 | Hs. 381093 |
| | kinase 1 | | |
| BBC3 | BCL2 binding component 3 | U82987 | Hs. 87246 |
| TGFB3 | Transforming growth | NM_003239 | Hs. 2025 |
| | factor, beta 3 | | |
| MS4A7 | Membrane-spanning 4- | AF201951 | Hs. 11090 |
| | domains | | |
| GSTM3 | Glutathione S-transferase | NM_000849 | Hs. 2006 |
| | M3 | | |
| FGF18 | Fibroblast growth factor | NM_003862 | Hs. 49585 |
| | 18 | | |

4-gene signature

| | | | |
|---|---|---|---|
| HEC | Highly expressed in | NM_006101 | Hs. 58169 |
| | cancer | | |
| MCM6 | Minichromosome | NM_005915 | Hs. 155462 |
| | maintenance deficient 6 | | |
| GSTM3 | Glutathione S-transferase | NM_000849 | Hs. 2006 |
| | M3 | | |
| FGF18 | Fibroblast growth factor | NM_003862 | Hs. 49585 |
| | 18 | | |

13-gene signature (same as Table 29)

| | | | |
|---|---|---|---|
| CEGP1 | SCUBE2 signal peptide, | NM_020974 | Hs. 222399 |
| | CUB domain | | |
| FGF18 | Fibroblast growth factor | NM_003862 | Hs. 49585 |
| | 18 | | |
| GSTM3 | Glutathione S-transferase | NM_000849 | Hs. 2006 |
| | M3 | | |
| TGFB3 | Transforming growth | NM_003239 | Hs. 2025 |
| | factor, beta 3 | | |
| MS4A7 | Membrane-spanning 4- | AF201951 | Hs. 11090 |
| | domains | | |
| EST | Hypothetical protein | Contig55377_RC | Hs. 218182 |
| AP2B1 | Adaptor-related protein | NM_001282 | Hs. 74626 |
| | complex 2 | | |
| CCNE2 | Cyclin E2 | NM_004702 | Hs. 30464 |
| KIAA0175 | Maternal embryonic | NM_014791 | Hs. 184339 |
| | leucine zipper kinase | | |
| EXT1 | Exostoses (multiple) 1 | NM_000127 | Hs. 184161 |
| LOC341692 | Similar to Diap3 protein | Contig46218_RC | Hs. 283127 |
| PK428 | CDC42 binding protein | NM_003607 | Hs. 18586 |
| | kinase alpha | | |

14-gene signature (same as Table 28)

| | | | |
|---|---|---|---|
| MS4A7 | Membrane-spanning 4- | AF201951 | Hs. 11090 |
| | domains | | |
| TGFB3 | Transforming growth | NM_003239 | Hs. 2025 |
| | factor, beta 3 | | |
| BBC3 | BCL2 binding component 3 | U82987 | Hs. 87246 |
| AP2B1 | Adaptor-related protein | NM_001282 | Hs. 74626 |
| | complex 2 | | |
| ALDH4A1 | Aldehyde dehydrogenase | NM_003748 | Hs. 77448 |
| | 4 family, member A1 | | |
| FLJ11190 | Chromosome 20, open | NM_018354 | Hs. 155071 |
| | reading frame 46 | | |
| DC13 | DC13 protein | NM_020188 | Hs. 6879 |
| GMPS | Guanine monophosphate | NM_003875 | Hs. 5398 |
| | synthetase | | |
| AKAP2 | A kinase (PRKA) anchor | Contig57258_RC | Hs. 42322 |
| | protein | | |
| DCK | Deoxycytidine kinase | NM_000788 | Hs. 709 |
| ECT2 | Epithelial cell | Contig25991 | Hs. 122579 |
| | transforming sequence 2 | | |
| EST | ESTs, weakly similar to | Contig38288_RC | |
| | quiescin | | |
| OXCT | 3-oxoacid CoA transferase | NM_000436 | Hs. 177584 |
| EXT1 | Exostoses (multiple) 1 | NM_000127 | Hs. 184161 |

To demonstrate the ability to reduce the number of genes in the cluster, while maintaining predictive power, we selected subsets of genes within a minimum segregation set so as to raise the correlation coefficient, and tested the performance of the cluster as the set was reduced from 9 to 2 genes. Specifically, classification was performed in a cohort of 78 breast cancer patients. The outcome predictor clusters were identified using MDA-MB-435BL3 human breast carcinoma cell line as a reference standard. These results are shown in Tables 73.1 and 73.2.

TABLE 73.1
Classification accuracy of breast cancer outcome predictor algorithm
based on 9-gene parent cluster and smaller gene clusters derived from
the parent 9-gene cluster.

| Number of genes | Correlation | Poor | Good | |
|---|---|---|---|---|
| in cluster | coefficient | prognosis | prognosis | Overall |
| 9 genes | 0.945 | 31 of 34 | 27 of 44 | 58 of 78 |
| | | (91%) | (61%) | (74%) |
| 5 genes | 0.900 | 20 of 34 | 36 of 44 | 56 of 44 |
| | | (59%) | (82%) | (72%) |
| 4 genes | 0.956 | 28 of 34 | 28 of 44 | 56 of 44 |
| | | (82%) | (64%) | (72%) |
| 2 genes | 1.000 | 27 of 34 | 30 of 44 | 57 of 44 |
| | | (79%) | ((68%) | (73%) |

TABLE 73.2
Genes contained within reduced clusters

| 9-gene cluster | 5-gene cluster | 4-gene cluster | 2-gene cluster |
|---|---|---|---|
| HEC | HEC | HEC | HEC |
| AI377418 | MCM6 | MCM6 | FGF18 |
| MCM6 | BBC3 | GSTM3 | |
| BBC3 | ALDH4 | FGF18 | |
| ALDH4 | AP2B1 | | |
| AP2B1 | | | |
| PECI | | | |
| GSTM3 | | | |
| FGF18 | | | |

As described in Example 2, we validated the classification accuracy using an independent data set, and tested performance of the 13 genes good prognosis predictor cluster on a set of 19 samples obtained from 11 breast cancer patients who developed distant metastases within five years after diagnosis and treatment and 8 patients who remained disease free for at least five years (van't Veer, L. J., et al., 2002). 9 of 11 samples from the poor prognosis group had negative phenotype association indices, whereas 6 of 8 samples from the good prognosis group had positive phenotype association indices yielding 79% overall accuracy in sample classification.

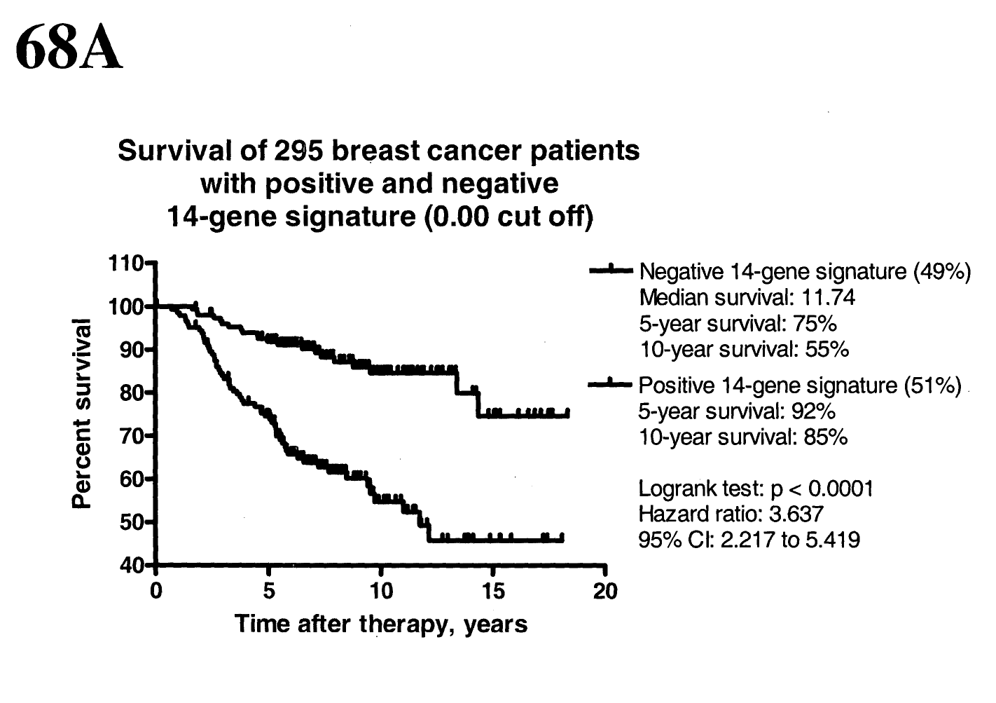

Kaplan-Meier analysis showed that metastasis-free survival after therapy was significantly different in breast cancer patients segregated into good and poor prognosis groups based on relative values of expression signatures defined by all four small gene clusters (FIG. 65A). These data indicate that quantitative laboratory tests measuring expression profiles of identified small gene clusters are useful in stratifying breast cancer patients into sub-groups with statistically distinct probabilities of remaining disease-free after therapy.

Small Gene Clusters and a Large Parent Signature Perform with Similar Therapy Outcome Prediction Accuracy in an Independent Cohort of 295 Breast Cancer Patients.

Recently the breast cancer prognosis prediction accuracy of the 70-gene signature was validated in a large cohort of 295 patients with either lymph node-negative or lymph node-positive breast cancer (van de Vijver, M. J., et al., 2002). The expression profile of the 70-gene breast cancer outcome predictor signature was highly informative in forecasting the probability of remaining free of distant metastasis and predicting the overall survival after therapy (id.). We compared the classification accuracy of small gene clusters and a large 70-gene parent signature applied to a cohort of 295 patients.

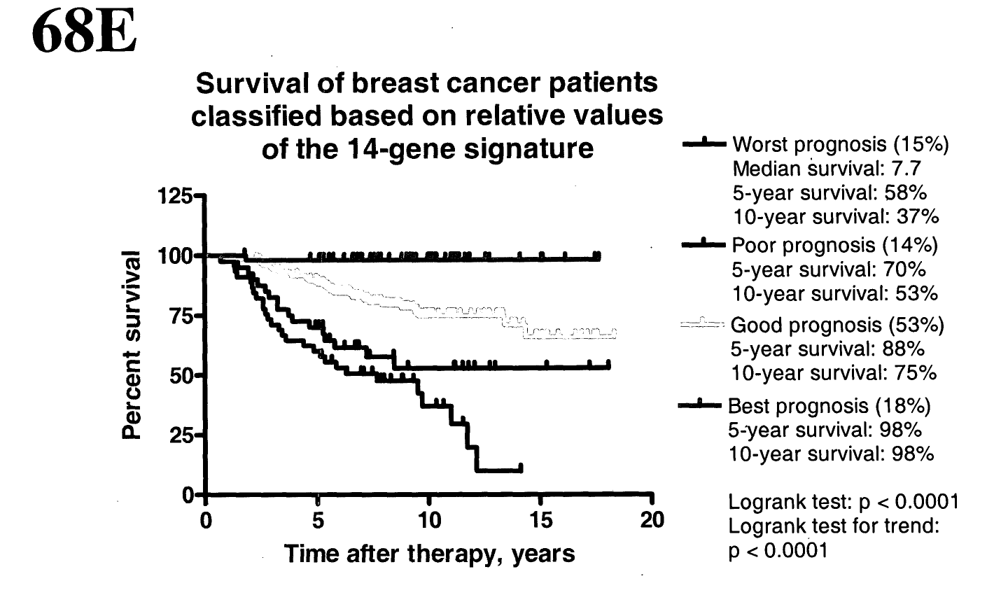

As shown in the Table 74, identified small gene clusters and a large parent signature perform similarly in identifying sub-groups of breast cancer patients with poor and good prognosis defined by differences in the probability of the overall survival after therapy. At the several classification threshold levels small gene clusters fully recapitulate or even outperform the 70-gene parent signature in classification accuracy of the 295 breast cancer patients (Table 74). Taken together these data are consistent with the idea that the 70-gene breast cancer prognosis signature represents a heterogeneous set of small gene clusters with high therapy outcome prediction potential. Consistent with this idea, the application of the 14-gene survival predictor signature was highly informative in classification of breast cancer patients into sub-groups with statistically significant difference in the probability of survival after therapy (FIG. 68). Interestingly, the highly significant difference (p<0.0001) in the survival probability between poor and good prognosis groups defined by the 14-gene signature was achieved using multiple classification threshold levels providing additional flexibility in selection of a desirable 5- or 10-year survival level defining good prognosis group (FIG. 68B).

To generate the data in Table 74, 295 breast cancer patients were classified according to whether they had a good-prognosis signature or poor-prognosis signature defined by individual therapy outcome predictor signatures. Kaplan-Meier analysis was performed to evaluate the probability that patients would survive according to whether they had a poor-prognosis or a good-prognosis signature and determine the proportion of patients who would survive at least 5 or 10 years after therapy in poor-prognosis and good-prognosis sub-groups. Hazard ratios, 95% confidence intervals, and P values were calculated with use of the log-rank test. The number of correct predictions in poor-prognosis prognosis and good-prognosis groups is shown as a fraction of patients with the observed clinical outcome after therapy (79 patients died and 216 patients remained alive). The classification performance of different signatures were evaluated using one common threshold level (0.00) and optimized threshold levels adjusted for each gene cluster to achieve the most statistically significant (highest hazard ratio and lowest P value) discrimination in survival probability between patients assigned to poor and good prognosis groups.

TABLE 74
Stratification of 295 breast cancer patients at the time of diagnosis into poor and
good prognosis groups using different therapy outcome predictor signatures

| | Poor | Good | | | | | |
|---|---|---|---|---|---|---|---|
| Outcome | prognosis, | prognosis, | Correct | Correct | | | |
| Signature | 5-(10)- | 5-(10)- | predictions, | predictions, | | 95% | |
| (cut off | year | year | poor | good | Hazard | Confidence | |
| value) | survival | survival | outcome | outcome | ratio | interval | P value |
| 70-gene | 75% | 97% | 70 of 79 | 106 of 216 | 6.327 | 2.498 to | <0.0001 |
| (0.45) | (56%) | (92%) | (89%) | (49%) | | 6.077 | |
| 70-gene | 64% | 91% | 42 of 79 | 174 of 216 | 3.867 | 3.405 to | <0.0001 |
| (0.00) | (46%) | (80%) | (53%) | (81%) | | 9.809 | |
| 13-gene | 73% | 98% | 71 of 79 | 106 of 216 | 7.005 | 2.560 to | <0.0001 |
| (0.12) | (56%) | (93%) | (90%) | (49%) | | 6.237 | |
| 13-gene | 73% | 97% | 69 of 79 | 115 of 216 | 6.519 | 2.728 to | <0.0001 |
| (0.04) | (54%) | (92%) | (87%) | (53%) | | 6.610 | |
| 13-gene | 73% | 96% | 67 of 79 | 118 of 216 | 5.698 | 2.663 to | <0.0001 |
| (0.00) | (54%) | (90%) | (85%) | (55%) | | 6.450 | |
| 14-gene | 77% | 96% | 72 of 79 | 79 of 216 | 5.220 | 1.912 to | <0.0001 |
| (0.37) | (62%) | (91%) | (91%) | (37%) | | 4.874 | |
| 14-gene | 76% | 95% | 69 of 79 | 95 of 216 | 4.701 | 2.038 to | <0.0001 |
| (0.28) | (59%) | (89%) | (87%) | (44%) | | 5.016 | |
| 14-gene | 75% | 92% | 58 of 79 | 130 of 216 | 3.637 | 2.217 to | <0.0001 |
| (0.00) | (55%) | (85%) | (73%) | (60%) | | 5.419 | |
| 14-gene | 65% | 91% | 45 of 79 | 176 of 216 | 4.171 | 3.632 to | <0.0001 |
| (−0.55) | (45%) | (81%) | (57%) | (81%) | | 10.21 | |
| 6-gene | 78% | 96% | 70 of 79 | 85 of 216 | 4.543 | 1.901 to | <0.0001 |
| (−0.12) | (62%) | (88%) | (89%) | (39%) | | 4.756 | |
| 6-gene | 78% | 92% | 64 of 79 | 101 of 216 | 3.314 | 1.757 to | <0.0001 |
| (0.00) | (60%) | (86%) | (81%) | (47%) | | 4.282 | |
| 4-gene | 73% | 93% | 60 of 79 | 136 of 216 | 4.389 | 2.723 to | <0.0001 |
| (0.20) | (53%) | (85%) | (76%) | (63%) | | 6.735 | |
| 4-gene | 75% | 93% | 60 of 79 | 119 of 216 | 3.519 | 2.050 to | <0.0001 |
| (0.00) | (58%) | (84%) | (76%) | (55%) | | 4.983 | |

The 70-gene Signature, in Contrast to Small Gene Clusters, is Not Suitable for Breast Cancer Outcome Prediction in Patients with Estrogen Receptor Negative Tumors.

Consistent with well-established prognostic value of the estrogen receptor status of breast tumors (see Introduction), 97 percent of patients in the good prognosis group defined by the 70-gene signature had estrogen receptor positive (ER+) tumors (van de Vijver, M. J., et al., 2002). Conversely, ninety six percent of breast cancer patients with the estrogen receptor negative (ER−) tumors (66 of 69 patients at the cut off level <0.45) had expression profile of the 70 genes predictive of a poor outcome after therapy. Two important conclusions can be drawn from this association. First, breast cancer patients with ER+ tumors and poor prognosis expression profile of the 70 genes may have yet unidentified functional defect within an ER-response pathway. Second, a 70-gene signature appears to assign rather uniformly a vast majority of the patients with ER− tumors into poor prognosis category and, therefore, is not suitable for prognosis prediction in this group of breast cancer patients.

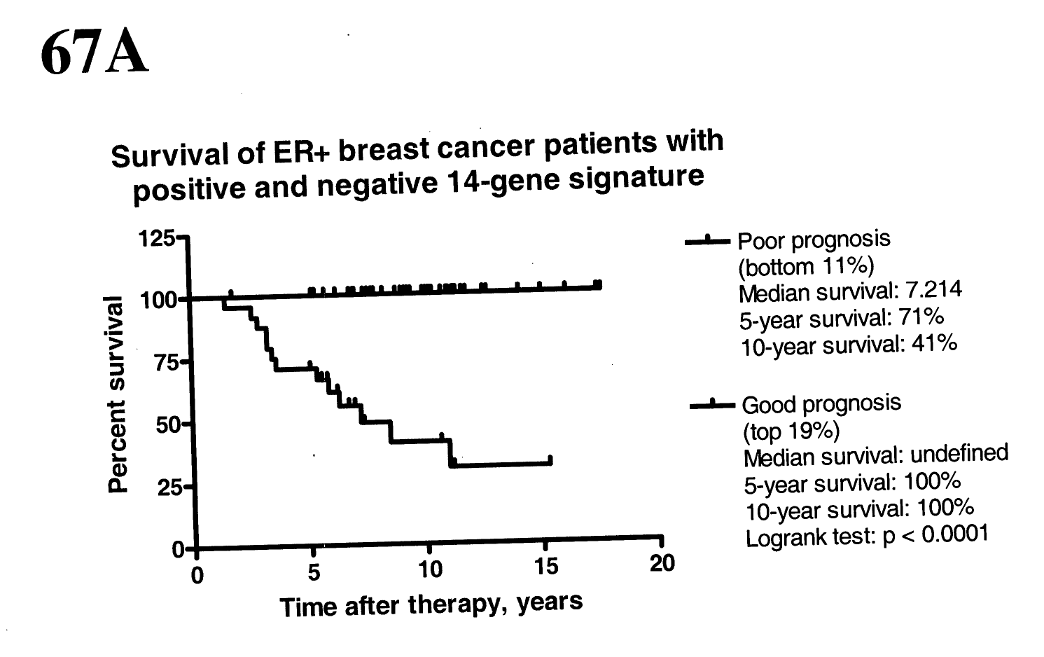

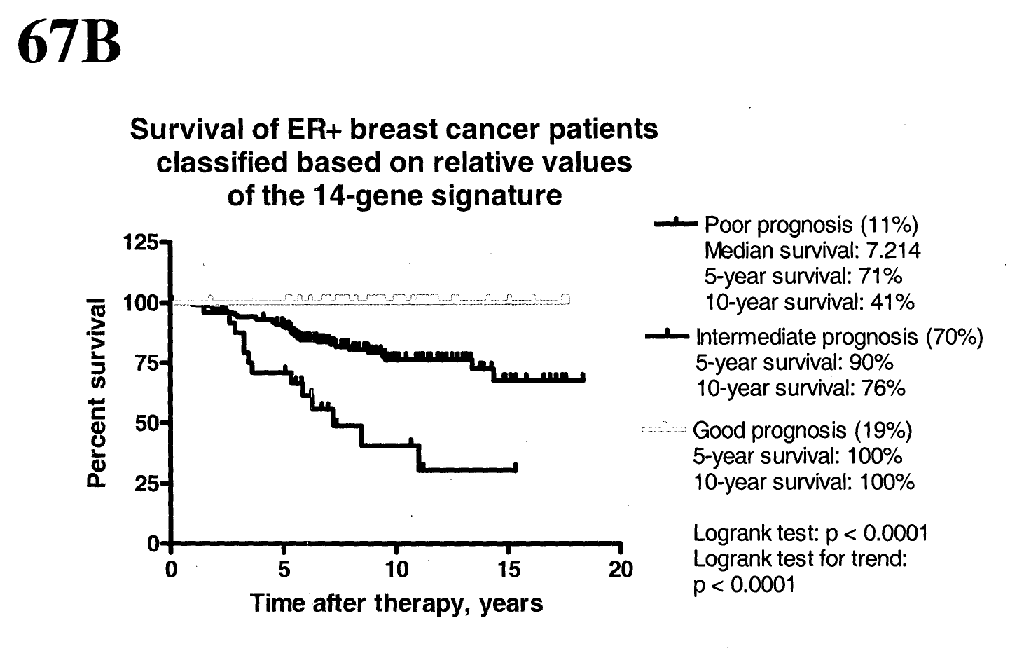

In agreement with many previous observations, patients with ER− tumors had significantly worst survival after therapy compared to the patients with ER+ tumors in the cohort of 295 breast cancer patients (FIG. 64A). The Kaplan-Meier survival analysis (FIG. 64A) showed that the median relapse-free survival after therapy of patients with the ER− tumors was 9.7 years. Only 47.1% of patients with ER-negative tumors survived 10 years after therapy compared to 77.4% patients with ER+ tumors. The estimated hazard ration for survival after therapy in the poor prognosis group as compared with the good prognosis group of patients defined by the ER status was 3.258 (95% confidence interval of ratio, 2.792 to 8.651; P<0.0001).

Next we determined that application of a survival predictor algorithm would identify sub-groups of patients with distinct clinical outcome after therapy in breast cancer patients with ER-negative tumors, thus providing additional predictive value to the therapy outcome classification based on ER status alone. We were unable to generate statistically meaningful prognostic stratification of ER-negative breast cancer patients using a parent 70-gene signature (data not shown). However, we were able to identify two small gene clusters comprising 5 and 3 genes (Table 75) that appear highly informative in classifying breast cancer patients with ER-negative tumors into good and poor prognosis sub-groups with statistically distinct probability of survival after therapy (FIG. 64B).

TABLE 75
Gene expression signatures predicting survival of breast cancer patients
with estrogen receptor-negative tumors.

| | | | |
|---|---|---|---|
| | | Gene ID (Chip | |
| | | identified in van't | |
| | | Veer, L. J., et | UniGene |
| Gene | Description | al., 2002) | ID |

5-gene signature

| | | | |
|---|---|---|---|
| EST | Unknown | Contig63649_RC | |
| L2DTL | RA-regulated nuclear | NM_016448 | Hs.126774 |
| | matrix-associated | | |
| | protein | | |
| DCK | Deoxycytidine kinase | NM_000788 | Hs.709 |
| DKFZP564- | G protein-coupled | AL080079 | Hs.44197 |
| D0462 | receptor 126 | | |
| LOC286052 | Hypothetical protein | AA555029_RC | Hs.100691 |
| | LOC286052 | | |

3-gene signature

| | | | |
|---|---|---|---|
| GNAZ | Guanine nucleotide | NM_002073 | Hs.92002 |
| | binding protein | | |
| PK428 | CDC42 binding protein | NM_003607 | Hs.18586 |
| | kinase alpha | | |
| LYRIC | LYRIC/3D3 | AK000745 | Hs.243901 |

In the group of 69 breast cancer patients with ER-negative tumors (FIG. 64B), the median survival after therapy of patients in the poor prognosis sub-group defined by the survival predictor algorithm was 5.2 years. Only 30% of patients in the poor prognosis sub-group survived 10 years after therapy compared to 77% patients in the good prognosis sub-group. The estimated hazard ration for survival after therapy in the poor prognosis sub-group as compared with the good prognosis sub-group of patients defined by the survival predictor algorithm was 3.609 (95% confidence interval of ratio, 1.477 to 5.792; P=0.0021).

Outcome Classification of Breast Cancer Patients with ER-positive Tumors Using a 14-Gene Survival Predictor Signature.

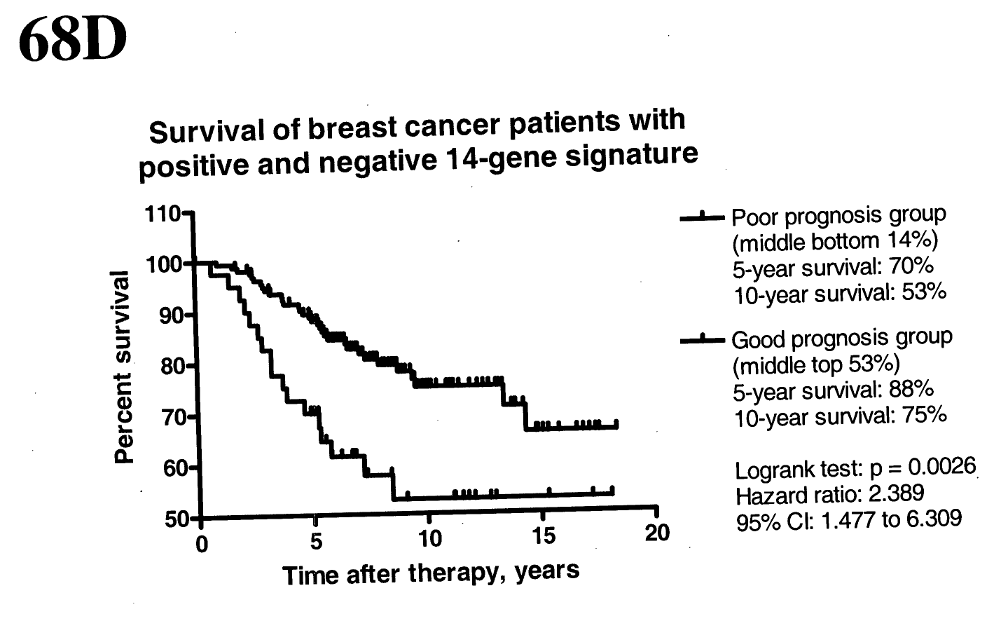

To further validate the clinical utility of identified signatures, we determined that application of a 14-gene survival predictor cluster is informative in classifying breast cancer patients with ER-positive tumors. Kaplan-Meier analysis showed that application of the 14-gene survival predictor signature identified three sub-groups of patients with statistically distinct probabilities of survival after therapy in the cohort of 226 breast cancer patients with ER-positive tumors (FIGS. 67A&B). The median survival after therapy of patients in the poor prognosis sub-group defined by the 14-gene survival predictor signature was 7.2 years (FIG. 67A). Only 41% of patients in the poor prognosis sub-group survived 10 years after therapy compared to 100% patients in the good prognosis sub-group (P<0.0001). A large, statistically distinct sub-group of patients with an intermediate expression pattern of the 14-gene signature and an intermediate prognosis was identified by Kaplan-Meier survival analysis (FIG. 67B). The patients in the sub-group with an intermediate prognosis had 90% 5-year survival and 76% 10-year survival after therapy (FIG. 67B). Thus, the 14-gene survival predictor signature is highly informative in classifying breast cancer patients with ER-positive tumors into good, intermediate, and poor prognosis sub-groups with statistically significant differences in the probability of survival after therapy (FIGS. 67A&B).

Therapy Outcome Prediction in Breast Cancer Patients with Lymph Node-Negative Disease Using Survival Predictor Signatures.

Invasion into axillary lymph nodes is considered as one of the most important negative prognostic factors in breast cancer and patients with no detectable lymph node involvement are classified as having good prognosis (Krag, et al., 1998; Singletary, et al., 2002; Jatoli, et al., 1999). Breast cancer patients with lymph node negative disease typically would not be selected for adjuvant systemic therapy and usually treated with surgery and radiation. Recent data demonstrated that up to 33% of these patients would fail therapy and develop recurrence of the disease after a 10-year follow-up (Early Breast Cancer Trialists' Collaborative Group. Tamoxifen for early breast cancer: an overview of the randomized trials. Lancet, 351: 1451-1467, 1998). Therefore, we tested whether application of the 14-gene survival predictor signature would aid in identifying breast cancer patients with lymph-node negative tumors that are at high risk of treatment failure.

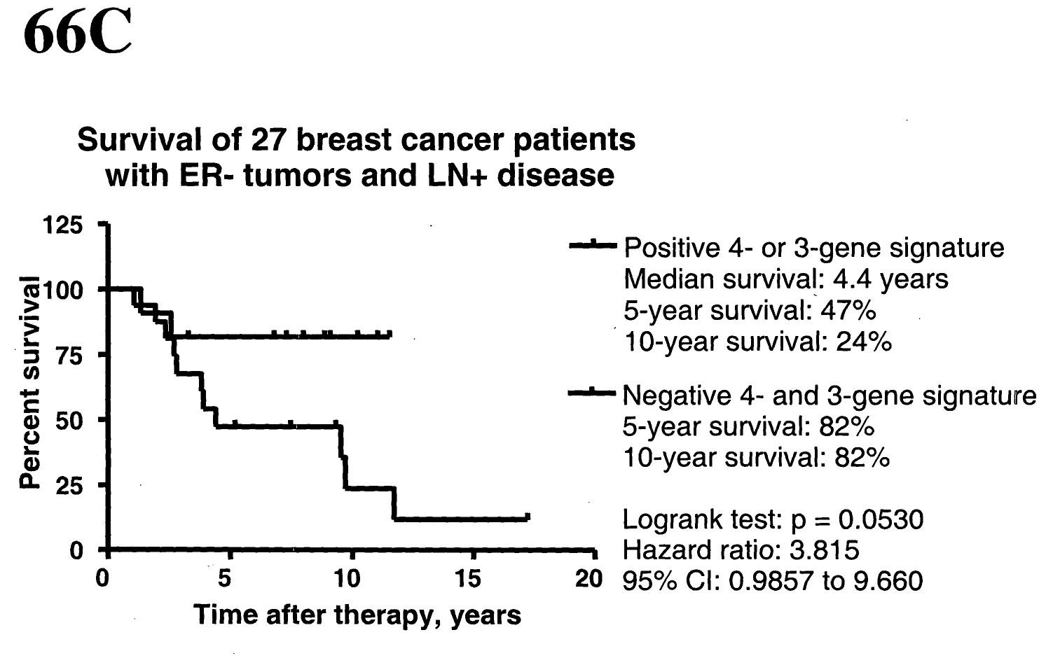

Kaplan-Meier analysis showed that the 14-gene survival predictor signature (Tables 29 and 73) identified two sub-groups of patients with statistically distinct probability of survival after therapy in the cohort of 151 breast cancer patients with lymph node negative disease (FIG. 63A). The median survival after therapy of patients in the poor prognosis sub-group defined by the 14-gene survival predictor signature was 7.7 years (FIG. 63A). Only 46% of patients in the poor prognosis sub-group survived 10 years after therapy compared to 82% patients in the good prognosis sub-group (P<0.0001). The estimated hazard ration for survival after therapy in the poor prognosis sub-group as compared with the good prognosis sub-group of patients defined by the 14-gene survival predictor signature was 5.067 (95% confidence interval of ratio, 3.174 to 11.57; P<0.0001).

Kaplan-Meier analysis also demonstrated that the 14-gene survival predictor signature identified two sub-groups of patients with statistically distinct probability of survival after therapy in the cohort of 109 breast cancer patients with ER-positive tumors and lymph node negative disease (FIG. 63B). The median survival after therapy of patients in the poor prognosis sub-group defined by the 14-gene survival predictor signature was 11.0 years (FIG. 63B). 10-year survival after therapy in the poor prognosis sub-group was 57% compared to 86% patient's survival in the good prognosis sub-group (P<0.0001). The estimated hazard ration for survival after therapy in the poor prognosis sub-group as compared with the good prognosis sub-group of patients defined by the 14-gene survival predictor signature was 5.314 (95% confidence interval of ratio, 2.775 to 17.79; P<0.0001).

Next we determined that application of small gene clusters comprising 5 and 3 genes (Table 75) that appear highly informative in classification of breast cancer patients with ER-negative tumors into good and poor prognosis sub-groups with statistically distinct probability of survival after therapy (FIG. 64B), also are informative in classification of sub-group of ER-negative patients with lymph node-negative disease. In the group of 42 breast cancer patients with ER-negative tumors and lymph node-negative disease (FIG. 63C), the median survival after therapy of patients in the poor prognosis sub-group defined by the survival predictor algorithm was 5.2 years. Only 34% of patients in the poor prognosis sub-group survived 10 years after therapy compared to 74% patients in the good prognosis sub-group. The estimated hazard ration for survival after therapy in the poor prognosis sub-group as compared with the good prognosis sub-group of patients defined by the survival predictor algorithm was 3.237 (95% confidence interval of ratio, 1.139 to 6.476; P=0.0243). Thus, the survival predictor signatures identified in accordance with the methods of the invention are highly informative in classifying breast cancer patients with lymph node-negative disease and either ER-positive or ER-negative tumors into good and poor prognosis sub-groups with statistically significant difference in the probability of survival after therapy (FIGS. 63B&C).

Therapy Outcome Prediction in Breast Cancer Patients with Lymph Node-Positive Disease Using Survival Predictor Signatures.

Breast cancer patients with invasion into axillary lymph node are considered as having a poor prognosis and usually treated with adjuvant systemic therapy. The patients with poor prognosis are thought to benefit most from adjuvant systemic therapy (see Introduction). In the cohort of 295 breast cancer patients, ten of 151 (6.6%) patients who had lymph node-negative disease and 120 of the 144 (83.3%) patients who had lymph node-positive disease had received adjuvant systemic therapy (van de Vijver, et al. 2002). This treatment strategy was clearly beneficial for patients with lymph node-positive disease, because sub-groups of patients with distinct lymph node status in the cohort of 295 patients had statistically indistinguishable survival after therapy (data not shown). Next we determined therapy outcome prediction using survival predictor signatures identified in accordance with the present invention to be informative in breast cancer patients with lymph node-positive disease.

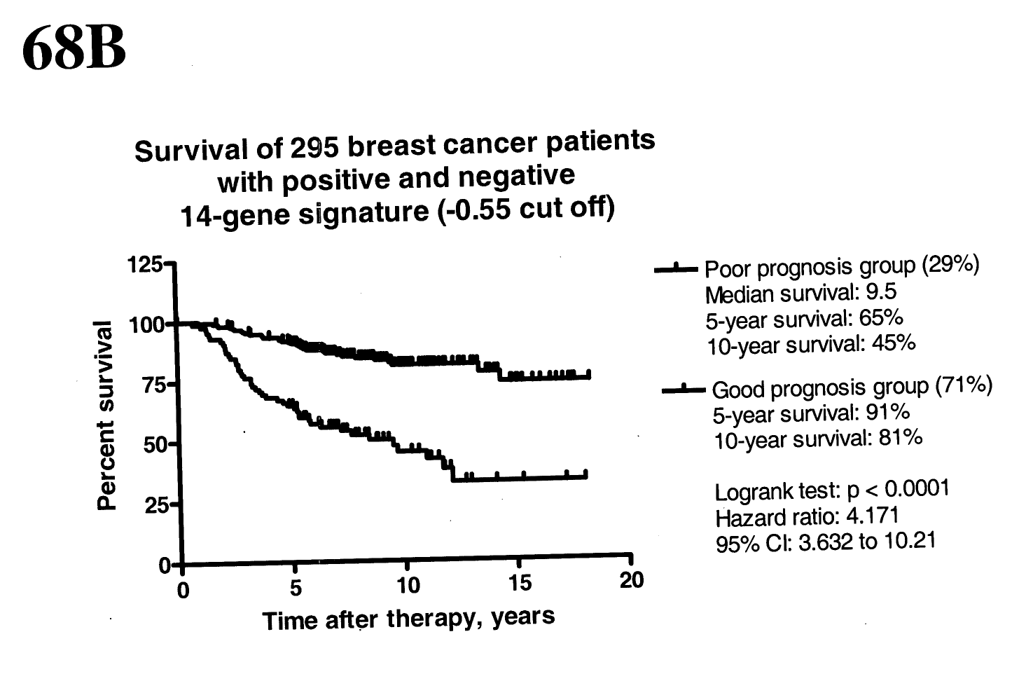

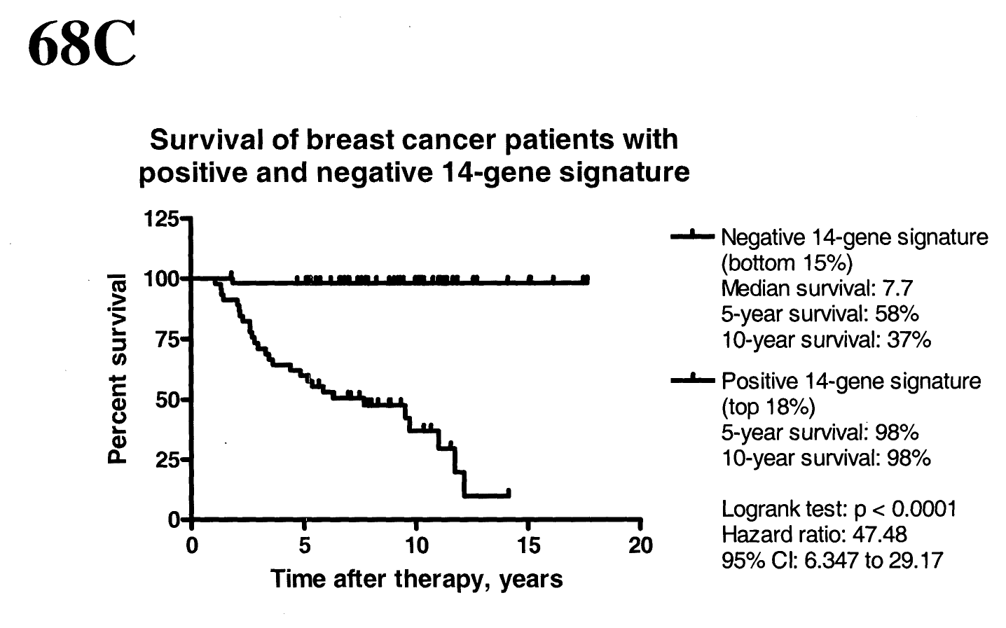

Kaplan-Meier analysis show that application of the 14-gene survival predictor signature identify three sub-groups of patients with statistically distinct probability of survival after therapy in the cohort of 144 breast cancer patients with lymph node positive disease (FIG. 66A). The median survival after therapy of patients in the poor prognosis sub-group defined by the 14-gene survival predictor signature was 9.5 years (FIG. 66A). Only 43% of patients in the poor prognosis sub-group survived 10 years after therapy compared to 98% patients in the good prognosis sub-group (P<0.0001). Large statistically distinct sub-group of patients with an intermediate expression pattern of the 14-gene signature and an intermediate prognosis was identified by Kaplan-Meier survival analysis (FIG. 66A). The patients in the sub-group with an intermediate prognosis had 86% 5-year survival and 73% 10-year survival after therapy (FIG. 66A). Thus, 14-gene survival predictor signature appears highly informative in classification of breast cancer patients with lymph node-positive disease into good, intermediate, and poor prognosis sub-groups with statistically significant difference in the probability of survival after therapy (FIGS. 66A).

Using the 14-gene survival predictor signature we identified two sub-groups of patients with statistically distinct probabilities of survival after therapy in the cohort of 117 breast cancer patients with ER-positive tumors and lymph node positive disease (FIG. 66B). The median survival after therapy of patients in the poor prognosis sub-group defined by the 14-gene survival predictor signature was 11.0 years (FIG. 66B). 10-year survival after therapy in the poor prognosis sub-group was 68% compared to 98% patient's survival in the good prognosis sub-group (P=0.0026). The estimated hazard ration for survival after therapy in the poor prognosis sub-group as compared with the good prognosis sub-group of patients defined by the 14-gene survival predictor signature was 6.810 (95% confidence interval of ratio, 1.566 to 8.358; P=0.0026).

Next we determined that the small gene clusters comprising 5 and 3 genes (Table 75) also are informative in classifying sub-groups of ER-negative patients with lymph node-positive disease. In the group of 27 breast cancer patients with ER-negative tumors and lymph node-positive disease (FIG. 66C), the median survival after therapy of patients in the poor prognosis sub-group defined by the survival predictor algorithm was 4.4 years. Only 24% of patients in the poor prognosis sub-group survived 10 years after therapy compared to 82% patients in the good prognosis sub-group. The estimated hazard ration for survival after therapy in the poor prognosis sub-group as compared with the good prognosis sub-group of patients defined by the survival predictor algorithm was 3.815 (95% confidence interval of ratio, 0.9857 to 9.660; P=0.0530). Thus, survival predictor signatures identified in accordance with the present invention also is informative in classifying breast cancer patients with lymph node-positive disease into good and poor prognosis sub-groups with statistically significant differences in the probability of survival after therapy (FIGS. 66A & 66B).

Estimated Long-Term Survival Benefits of Using Gene Expression Profiling as a Component of Multiparameter Therapy Outcome Classification of Breast Cancer Patients.

Next we estimated the potential clinical benefits of applying gene expression survival predictor signatures identified in accordance with the methods of the present invention for classifying breast cancer patients at the time of diagnosis into sub-groups with distinct probabilities of survival after therapy. In our estimate we used the assignment of the patient into a poor outcome classification sub-group as a criterion of treatment failure and reason for prescription of additional cycle(s) of adjuvant systemic therapy. We have made the estimate of potential therapeutic benefits in the cohort of 295 breast cancer patients (van de Vijver, et al. 2002) and based our estimate on the assumption that the use of additional cycle(s) of adjuvant systemic therapy would be prescribed to patients classified within a poor prognosis sub-group. In the cohort of 295 breast cancer patients, ten of 151 (6.6%) patients who had lymph node-negative disease and 120 of the 144 (83.3%) patients who had lymph node-positive disease had received adjuvant systemic therapy (id.), indicating that a major difference in treatment protocols between LN+ and LN− sub-groups was the application of adjuvant systemic therapy in patients with lymph node positive disease. We accepted the actual 5- and 10-year survival in the corresponding classification categories as the expected therapy outcome for a given sub-group. We assumed that each additional cycle of adjuvant systemic therapy would result in the same therapy outcome as was actually documented in the most relevant sub-groups of the 295 patients. Therapy outcome for patients classified into poor prognosis sub-groups and treated with additional cycle(s) of adjuvant systemic therapy is expected to be in 37% of patients in good therapy outcome category for ER+ LN+ and ER+ LN− poor signature sub-groups and in 41% of patients in good therapy outcome category for ER− LN+ and ER− LN− poor signature sub-groups (Table 76). Finally, we assumed that patients classified into good prognosis sub-groups would receive the same treatment and would have the same outcome as in the original cohort of 295 patients (van de Vijver, et al., 2002). Based on these assumptions we calculated the number of patients that would be expected to have good and poor survival outcome after therapy and estimated the expected 10-year survival in each classification sub-groups (Table 76).

The estimate of potential therapeutic benefits provided in Table 76 is based on the cohort of 295 breast cancer patients (van de Vijver, et al. 2002) and premised on the assumption that additional cycle(s) of adjuvant systemic therapy would be prescribed to patients classified into poor prognosis sub-groups. In the cohort of 295 breast cancer patients, ten of 151 (6.6%) patients who had lymph node-negative disease and 120 of the 144 (83.3%) patients who had lymph node-positive disease had received adjuvant systemic therapy (id.). We accepted the actual 5- and 10-year survival in the corresponding classification categories as the expected therapy outcome for a given sub-group. We assumed that each additional cycle of adjuvant systemic therapy would result in the same therapy outcome as was actually documented in the most relevant sub-groups of the 295 patients. Therapy outcome for patients classified into poor prognosis sub-groups and treated with additional cycle(s) of adjuvant systemic therapy is expected to be in 37% of patients in good therapy outcome category for ER+ LN+ and ER+ LN− poor signature sub-groups and in 41% of patients in good therapy outcome category for ER− LN+and ER− LN− poor signature sub-groups.

TABLE 76
Estimated therapeutic benefits of using gene expression survival predictor
signatures for classification of breast cancer patients

| | | | | | | Estimated |
|---|---|---|---|---|---|---|
| | | | Number | Good | Good | increase in |
| Classification | 5-year | 10-year | (%) of | outcome | outcome | 10-year |
| category | survival | survival | patients | (current) | (projected) | survival, % |
| LN− negative | 82% | 69% | 151/295 | | | |
| | | | (51%) | | | |
| LN+ positive | 85% | 72% | 144/295 | | | |
| | | | (49%) | | | |
| LN− Good | 92% | 82% | 95/151 | 95 | 95 | 0.00 |
| signature | | | (63%) | | | |
| LN− Poor | 64% | 46% | 56/151 | 0 | 17 (56 × 0.3) | 23% |
| signature | | | (37%) | | | |
| LN+ Good | 98% | 98% | 43/144 | 43 | 43 | 0.00 |
| signature | | | (30%) | | | |
| LN+ | 86% | 73% | 67/144 | 0 | 20 (67 × 0.3) | 10% |
| Intermediate | | | (47%) | | | |
| LN+ Poor | 68% | 43% | 34/144 | 0 | 10 (34 × 0.3) | 13% |
| signature | | | (24%) | | | |
| Overall | | | | 138/295 | 185/295 | 5% |
| | | | | (47%) | (63%) | |
| ER+ tumors | 90% | 77% | 226/295 | | | |
| | | | (77%) | | | |
| ER− tumors | 62% | 47% | 69/295 | | | |
| | | | (23%) | | | |
| ER+ LN− | | | | | | |
| Good | 97% | 86% | 69/109 | 69 | 69 | 0.00 |
| signature | | | (63%) | | | |
| Poor | 76% | 57% | 40/109 | 0 | 15 (40 × 0.37) | 17% |
| signature | | | (37%) | | | |
| ER− LN− | | | | | | |
| Good | 74% | 74% | 16/42 | 16 | 16 | 0.00 |
| signature | | | (38%) | | | |
| Poor | 50% | 34% | 26/42 | 0 | 11 (25 × 0.41) | 44% |
| signature | | | (62%) | | | |
| ER+ LN+ | | | | | | |
| Good | 98% | 98% | 43/117 | 43 | 43 | 0.00 |
| signature | | | (37%) | | | |
| Poor | 86% | 68% | 74/117 | 0 | 27 (74 × 0.37) | 16% |
| signature | | | (63%) | | | |
| ER− LN+ | | | | | | |
| Good | 82% | 82% | 11/27 | 11 | 11 | 0.00 |
| signature | | | (41%) | | | |
| Poor | 47% | 24% | 16/27 | 0 | 7 (16 × 0.41) | 100% |
| signature | | | (59%) | | | |
| Overall | | | | 139/295 | 199/295 | 6% |
| | | | | (47%) | (67%) | |

One of the most interesting end-points of this analysis is the prediction that patients with ER− LN− and ER− LN+ breast cancer classified into poor prognosis sub-groups would be expected to show a most dramatic increase in 10-year survival after therapy (Table 76). This prediction is consistent with the generally accepted notion that breast cancer patients with poor prognosis would benefit most from adjuvant systemic therapy (see Introduction). The estimated modest increase in the overall 10-year survival (Table 76) may translate every year into ˜7,000-9,000 more breast cancer survivors after 10-year follow-up. Our ability to accurately segregate at the time of diagnosis breast cancer patients with low probability of survival after therapy should lead to more rapid development of novel efficient therapeutic modalities specifically targeting most aggressive therapy-resistant breast cancers.

While the invention has been described with reference to specific methods and embodiments, it will be appreciated that various modifications may be made without departing from the invention, the scope of which is limited only by the appended claims. All references cited, including scientific publications, patent applications, and issued patents, are herein incorporated by reference in their entirety for all purposes.

1. A method for identifying a subset of genes, comprising:
identifying a first reference set of expressed genes, said first reference set consisting of genes differentially expressed between a first sample and a second sample; wherein said first and second samples differ with respect to a phenotype; identifying a second reference set of expressed genes, said second reference set consisting of genes that are differentially expressed between a third samples and a fourth sample; wherein said third and fourth differ with respect to said phenotype; identifying a concordance set of expressed genes, said concordance set consisting of genes common to said first and second reference sets wherein the direction of said differential expression is the same in said first and second reference sets; and identifying a subset of genes within said concordance set, wherein said subset is selected so that a first correlation coefficient, correlating for said genes within said subset a first expression differential between said first and second samples to a second expression differential between third and fourth samples, exceeds a predetermined value.

2. The method of claim 1, wherein said first correlation coefficient is selected from the group consisting of a correlation coefficient ρ_{x,y}, a Pearson product moment correlation, and a square of a Pearson product moment correlation coefficient.

3. The method of claim 1, wherein said differentials are logarithmically transformed prior to calculating said first correlation coefficient.

4. The method of claim 3, wherein said first correlation coefficient has an absolute value ≧0.8.

5. The method of claim 4, wherein said first correlation coefficient has an absolute value ≧0.9.

6. The method of claim 5, wherein said first correlation coefficient has an absolute value ≧0.95.

7. The method of claim 6, wherein said first correlation coefficient has an absolute value ≧0.995.

8. The method of claim 1, wherein said gene expression data from either or both of said first reference set and said second reference set is independently selected from the group consisting of mRNA quantification data, cRNA quantification data, cDNA quantification data, and protein quantification data.

9. The method of claim 1, wherein at least one of said first sample and said second sample comprises a cell line.

10. The method of claim 9, wherein said cell line is selected from the group consisting of a tumor cell line, a pluripotent precursor cell line, an omnipotent stem cell line, and a differentiated cell line.

11. The method of claim 10, wherein said cell line is a tumor cell line.

12. The method of claim 10, wherein said cell line is a pluripotent precursor cell line.

13. The method of claim 10, wherein said cell line is an omnipotent stem cell line.

14. The method of claim 9, wherein said first sample comprises a cell recovered from an orthotopic implant.

15. The method of claim 14, wherein said second sample comprises a cell recovered from an ectopic implant.

16. The method of claim 9, wherein at least one of said third sample and said fourth sample comprises a cell recovered from a patient.

17. The method of claim 9, wherein at least one of said third sample and said fourth sample comprises a cell recovered from a healthy donor.

18. The method of claim 16, wherein said cell is a tumor cell.

19. The method of claim 18, wherein said tumor cell is recovered from an organ selected from the group consisting of a prostate, a breast, a colon, a lung and an ovary.

20. The method of claim 1, wherein said phenotype is selected from the group consisting of recurrence, non-recurrence, invasiveness, non-invasiveness, metastatic, localized, tumor grade, Gleason score, survival prognosis, lymph node status, tumor stage, degree of differentiation, age, hormone receptor status, PSA level, histologic type, and disease free survival.

21. The method of claim 1, wherein any of the group consisting of said first sample, said second sample, said third sample, and said fourth sample comprises a plurality of independent samples, and at least one of said first and said second differential is an average over said plurality of independent samples.

22. A method of correlating gene expression with a sample phenotype, comprising:
identifying a subset of genes according to the method of claim 1;
and determining the sign of a second correlation coefficient, said second correlation coefficient correlating for said genes within said subset said first or said second expression differential to an expression differential obtained from an unclassified sample, whereby the sign of said second correlation coefficient establishes a positive or a negative correlation with said phenotype of claim 1.

23. The method of claim 22, further comprising determining the magnitude of said second correlation coefficient and using said magnitude to assess the reliability of said established correlation.

24. The method of claim 22, wherein said subset consists essentially of the genes identified in Table 5, Table 7, Table 8, Table 9, Table 10, Table 13, Table 14, Table 15, Table 16, Table 18, Table 19, Table 20, Table 21, Table 22, Table 24, Table 25, Table 26, Table 27, Table 28, Table 29, Table 30, Table 31, Table 32, Table 33, Table 34, Table 35, Table 36, Table 37, Table 38, Table 41, Table 43, Table 44, Table 45, Table 46, Table 49, Table 50, Table 51, Table 52, Table 53, Table 55, Table 56, Table 57, Table 58, Table 61, Table 62, Table 65, Table 66, Table 67, Table 68, Table 69, Table 73, or Table 75.

25. The method of claim 24, wherein said subset consists essentially of 90% of the genes identified in Table 5, Table 7, Table 8, Table 9, Table 10, Table 13, Table 14, Table 15, Table 16, Table 18, Table 19, Table 20, Table 21, Table 22, Table 24, Table 25, Table 26, Table 27, Table 28, Table 29, Table 30, Table 31, Table 32, Table 33, Table 34, Table 35, Table 36, Table 37, Table 38, Table 41, Table 43, Table 44, Table 45, Table 46, Table 49, Table 50, Table 51, Table 52, Table 53, Table 55, Table 56, Table 57, Table 58, Table 61, Table 62, Table 65, Table 66, Table 67, Table 68, Table 69, Table 73, or Table 75.

26. The method of claim 25, wherein said subset consists essentially of 80% of the genes identified in Table 5, Table 7, Table 8, Table 9, Table 10, Table 13, Table 14, Table 15, Table 16, Table 18, Table 19, Table 20, Table 21, Table 22, Table 24, Table 25, Table 26, Table 27, Table 28, Table 29, Table 30, Table 31, Table 32, Table 33, Table 34, Table 35, Table 36, Table 37, Table 38, Table 41, Table 43, Table 44, Table 45, Table 46, Table 49, Table 50, Table 51, Table 52, Table 53, Table 55, Table 56, Table 57, Table 58, Table 61, Table 62, Table 65, Table 66, Table 67, Table 68, Table 69, Table 73, or Table 75.

27. The method of claim 26, wherein the subset consists essentially of 70% of the genes identified in Table 5, Table 7, Table 8, Table 9, Table 10, Table 13, Table 14, Table 15, Table 16, Table 18, Table 19, Table 20, Table 21, Table 22, Table 24, Table 25, Table 26, Table 27, Table 28, Table 29, Table 30, Table 31, Table 32, Table 33, Table 34, Table 35, Table 36, Table 37, Table 38, Table 41, Table 43, Table 44, Table 45, Table 46, Table 49, Table 50, Table 51, Table 52, Table 53, Table 55, Table 56, Table 57, Table 58, Table 61, Table 62, Table 65, Table 66, Table 67, Table 68, Table 69, Table 73, or Table 75.

28. The method of claim 27, wherein the subset consists essentially of 60% of the genes identified in Table 5, Table 7, Table 8, Table 9, Table 10, Table 13, Table 14, Table 15, Table 16, Table 18, Table 19, Table 20, Table 21, Table 22, Table 24, Table 25, Table 26, Table 27, Table 28, Table 29, Table 30, Table 31, Table 32, Table 33, Table 34, Table 35, Table 36, Table 37, Table 38, Table 41, Table 43, Table 44, Table 45, Table 46, Table 49, Table 50, Table 51, Table 52, Table 53, Table 55, Table 56, Table 57, Table 58, Table 61, Table 62, Table 65, Table 66, Table 67, Table 68, Table 69, Table 73, or Table 75.

29. A kit comprising a set of reagents useful for determining the expression of a subset of genes, said subset consisting essentially of the genes identified in Table 5, Table 7, Table 8, Table 9, Table 10, Table 13, Table 14, Table 15, Table 16, Table 18, Table 19, Table 20, Table 21, Table 22, Table 24, Table 25, Table 26, Table 27, Table 28, Table 29, Table 30, Table 31, Table 32, Table 33, Table 34, Table 35, Table 36, Table 37, Table 38, Table 41, Table 43, Table 44, Table 45, Table 46, Table 49, Table 50, Table 5 1, Table 52, Table 53, Table 55, Table 56, Table 57, Table 58, Table 61, Table 62, Table 65, Table 66, Table 67, Table 68, Table 69, Table 73, or Table 75, and instructions for use.

30. The kit of claim 29, wherein the subset consists essentially of 90% of the genes identified in Table 5, Table 7, Table 8, Table 9, Table 10, Table 13, Table 14, Table 15, Table 16, Table 18, Table 19, Table 20, Table 21, Table 22, Table 24, Table 25, Table 26, Table 27, Table 28, Table 29, Table 30, Table 31, Table 32, Table 33, Table 34, Table 35, Table 36, Table 37, Table 38, Table 41, Table 43, Table 44, Table 45, Table 46, Table 49, Table 50, Table 51, Table 52, Table 53, Table 55, Table 56, Table 57, Table 58, Table 61, Table 62, Table 65, Table 66, Table 67, Table 68, Table 69, Table 73, or Table 75.

31. The kit of claim 30, wherein the subset consists essentially of 80% of the genes identified in Table 5, Table 7, Table 8, Table 9, Table 10, Table 13, Table 14, Table 15, Table 16, Table 18, Table 19, Table 20, Table 21, Table 22, Table 24, Table 25, Table 26, Table 27, Table 28, Table 29, Table 30, Table 31, Table 32, Table 33, Table 34, Table 35, Table 36, Table 37, Table 38, Table 41, Table 43, Table 44, Table 45, Table 46, Table 49, Table 50, Table 51, Table 52, Table 53, Table 55, Table 56, Table 57, Table 58, Table 61, Table 62, Table 65, Table 66, Table 67, Table 68, Table 69, Table 73, or Table 75.

32. The kit of claim 31, wherein the subset consists essentially of 70% of the genes identified in Table 5, Table 7, Table 8, Table 9, Table 10, Table 13, Table 14, Table 15, Table 16, Table 18, Table 19, Table 20, Table 21, Table 22, Table 24, Table 25, Table 26, Table 27, Table 28, Table 29, Table 30, Table 31, Table 32, Table 33, Table 34, Table 35, Table 36, Table 37, Table 38, Table 41, Table 43, Table 44, Table 45, Table 46, Table 49, Table 50, Table 51, Table 52, Table 53, Table 55, Table 56, Table 57, Table 58, Table 61, Table 62, Table 65, Table 66, Table 67, Table 68, Table 69, Table 73, or Table 75.

33. The kit of claim 32, wherein the subset consists essentially of 60% of the genes identified in Table 5, Table 7, Table 8, Table 9, Table 10, Table 13, Table 14, Table 15, Table 16, Table 18, Table 19, Table 20, Table 21, Table 22, Table 24, Table 25, Table 26, Table 27, Table 28, Table 29, Table 30, Table 31, Table 32, Table 33, Table 34, Table 35, Table 36, Table 37, Table 38, Table 41, Table 43, Table 44, Table 45, Table 46, Table 49, Table 50, Table 51, Table 52, Table 53, Table 55, Table 56, Table 57, Table 58, Table 61, Table 62, Table 65, Table 66, Table 67, Table 68, Table 69, Table 73, or Table 75.

34. The kit of any one of claims 29-33, wherein said reagents are affixed to a solid support.

35. The kit of any one of claims 29-33, wherein said reagents comprise primers for a nucleic acid amplification reaction.
